(19) United States
(12) Patent Application Publication
(10) Pub. No.: US 2008/0138328 A1
(43) Pub. Date: Jun. 12, 2008

(54) Novel Use of the Kinases Rsk4 and Pak5

(76) Inventors: Borlak Jurgen, Immensen (DE); Monica Niehof, Hannover (DE)

Correspondence Address: BUCHANAN INGERSOLL & ROONEY PC, P.O. BOX 1404, ALEXANDRIA, VA, 22313-1404, US

(21) Appl. No.: 11/632,894

(22) PCT Filed: Jul. 14, 2005
(86) PCT No.: PCT/EP05/07820

§ 371 Date: Aug. 14, 2007

(30) Foreign Application Priority Data
Jul. 19, 2004 (EP) 04016948.4

(51) Int. Cl. A61K 38/45; C12Q 1/48; A61K 31/70; C12N 9/00; C12Q 1/68

(52) U.S. Cl. 424/94.5; 435/15; 435/6; 514/44; 435/183

(57) ABSTRACT

Type 2 diabetes is one of the most common diseases. In the year 2030 366 million patients are expected, which is a doubling in comparison to the year 2000. Besides a defective regulation of the glucose metabolism patients suffer from diseases of the kidney and the brain, which are named as diabetic nephropathies and diabetic neuropathies, respectively. Worldwide large efforts are undertaken to understand the causes of this disease. There is evidence, that a dysfunctional HNF4a plays an important role in the onset of diabetes. With the help of molecular biological and molecular genetic methods new HNF4a target genes (RSK4 and PAK5) were identified. These two genes encode for kinases. In a diabetic animal model RSK4 and PAK5 were selectively regulated. The identification of these genes and their fundamental importance for a therapy of type 1 and/or type 2 diabetes and/or nephropathies and/or neuropathies, including diabetic nephropathies and diabetic neuropathies is described. This invention has a high economic potential, since agents can now be identified for the control of genes, which regulation is disturbed by these diseases. Therefore new therapeutic concepts are possible for the treatment of type 1 and/or type 2 diabetes and/or nephropathies and/or neuropathies, including diabetic nephropathies and diabetic neuropathies.

The invention concerns a novel use of the kinases RSK4 and PAK5 to screen for and to identify anti-type 1 and anti-type 2 diabetes mellitus and/or anti-nephropathic and/or anti-neuropathic drugs, including anti-diabetic nephropathic and anti-diabetic neuropathic drugs.

### BACKGROUND OF THE INVENTION

Diabetes mellitus is among the most common of all metabolic disorders, affecting up to 11% of flue population by age 70.

Type 1 diabetes (insulin dependent diabetes mellitus or IDDM) represents about 5 to 10% of this group and is the result of a progressive autoimmune destruction of the pancreatic cells with subsequent insulin deficiency.

Type 2 (non-insulin dependent diabetes mellitus or NIDDM) diabetes is one of the most common diseases. In the year 2030 366 million patients are expected, which is a doubling in comparison to the year 2000. This type represents 90-95% of the affected population more than 100 million people worldwide (King, H. and Zimmer, P. Wld Hlth. Statist. Quart. 41, 190-196 (1998)), and is associated with peripheral insulin resistance, elevated hepatic glucose production, and inappropriate insulin secretion (DeFronzo, R. A., Diabetes 37, 667-687 (1988)). Besides a defective regulation of the glucose metabolism patients suffer from after effect diseases of the kidney and the brain, which are named as diabetic nephropathies and diabetic neuropathies, respectively. Family studies point to a major genetic component (Newman, B., et al. Diabetologia 30, 763-768 (1987)). However, few susceptibility genes have been identified. Worldwide large efforts are undertaken to understand the causes of this disease. There is evidence, that a dysfunctional HNF4α plays an important role in the onset of diabetes.

The orphan nuclear receptor HNF4α is an essential transcription factor for liver development and hepatocellular differentiation (Chen, W. S. et al. Disruption of the HNF-4 gene, expressed in visceral endoderm, leads to cell death in embryonic ectoderm and impaired gastrulation of mouse embryos. Genes Dev 8, 2466-2477 (1994); Li, J., Ning, G., & Duncan, S. A. Mammalian hepatocyte differentiation requires the transcription factor HNF4alpha. Genes Dev 14, 464-474 (2000); Hayhurst, G. P., Lee, Y. H., Lambert, G., Ward, J. M., & Gonzalez, F. J. Hepatocyte nuclear factor 4alpha (nuclear receptor 2A1) is essential for maintenance of hepatic gene expression and lipid homeostasis. Mol Cell Biol 21, 1393-1403 (2001); Parviz, F. et al. Hepatocyte nuclear factor 4 alpha, controls the development of a hepatic epithelium and liver morphogenesis. Nat Genet 34, 292-296 (2003)) and plays an essential role in a transcription factor regulatory circuit (Schrem, H., Klempnauer, J., & Borlak, J. Liver-enriched transcription factors in liver function and development. Part I: the hepatocyte nuclear factor network and liver-specific gene expression. Pharmacol. Rev 54, 129-158 (2002)) to control a wide range of metabolic processes. This factor also targets genes in other organs including kidney, intestine and colon (Sladek, F. M. & Seidel, S. D. Hepatocyte nuclear factor 4 alpha in Nuclear Receptors and Disease. Academic Press, London. 309-361. (2001)), promotes expression of an epithelial phenotype, triggers de novo formation of functional tight junctions and contributes to epithelial cell polarity (Chiba, H. et al. Hepatocyte nuclear factor (HNF)-4alpha triggers formation of functional tight junctions and establishment of polarized epithelial morphology in F9 embryonal carcinoma cells. Exp Cell Res 286, 288-297 (2003)). HNF4α dysfunction is associated with metabolic disorders including diabetes, whereas patients with diabetes harbor a high risk for nephropathies and neuropathies.

### SUMMARY OF THE INVENTION

With the help of molecular biological and molecular genetic methods new HNF4α target genes (RSK4 and PAK5) were identified. These two genes encode for kinases. In a diabetic animal model RSK4 and PAK5 were selectively regulated. The identification of these genes and their fundamental importance for a therapy of type 1 and/or type 2 diabetes and/or nephropathies and/or neuropathies, including diabetic nephropathies and diabetic neuropathies is described. This invention has a high economic potential, since agents can now be identified for the control of genes, which regulation is disturbed by these diseases. Therefore new therapeutic concepts are possible for the treatment of type 1 and/or type 2 diabetes and/or nephropathies and/or neuropathies, including diabetic nephropathies and diabetic neuropathies.

In one aspect, the invention features compounds that are agonists of a normal (functional) bioactivity of the kinases RSK4 and PAK5 and their use in treating type 1 and/or type 2 diabetes and/or nephropathies and/or neuropathies, including diabetic nephropathies and diabetic neuropathies. For example, to ameliorate disease symptoms involving insufficient expression of a RSK4 gene and/or a PAK5 gene and/or inadequate amount of functional RSK4 and/or PAK5 bioactivity in a subject, a gene therapeutic (comprising a gene encoding a functional RSK4 or PAK5 protein) or a protein therapeutic (comprising a functional RSK4 and/or PAK5 protein) can be administered to the subject.

In another aspect, the invention features compounds that are antagonists of a disease causing RSK4 or PAK5 bioactivity and their use in treating type 1 and/or type 2 diabetes and/or nephropathies and/or neuropathies, including diabetic nephropathies and diabetic neuropathies. For example to ameliorate disease symptoms involving expression of a mutant RSK4 or PAK5 gene or overexpression of a normal RSK4 or PAK5 gene in a subject, a therapeutically effective amount of an antisense, ribozyme or triple helix molecule to reduce or prevent gene expression, as described herein, may be administered to the subject. Alternatively, to ameliorate disease symptoms involving the regulation via an RSK4 or PAK5 protein of an upstream or downstream element in a RSK4 and/or PAK5 mediated biochemical pathway (e.g. signal transduction), a therapeutically effective amount of an antagonist compound (e.g. small molecule, peptide, or peptidomimetic) that can prevent binding of the wildtype RSK4 and/or PAK5 protein, can induce a therapeutic effect. Further, to ameliorate disease symptoms involving a mutant (nonfunctional) RSK4 and/or PAK5 protein, a therapeutically effective amount of an anti-RSK4 antibody and/or anti-PAK5 antibody, as described herein, may be administered to the subject.

In yet another aspect, the invention provides assays, e.g., for screening test compounds to identify antagonists (e.g. inhibitors), or alternatively, agonists (e.g. potentiators), of an interaction between a RSK4 and/or PAK5 protein and, for example, a protein or nucleic acid that binds to the RSK4 and/or PAK5 protein. An exemplary method includes the steps of (i) combining a RSK4 or PAK5 polypeptide or bioactive fragments thereof, a RSK4 and/or PAK5 target molecule (such as a RSK4 and/or PAK5 ligand or nucleic acid), and a test compound, e.g., under conditions wherein, the test compound, the RSK4 and/or PAK5 protein and RSK4 and/or PAK5 target molecule are able to interact; and (ii) detecting the formation of a complex which includes the RSK4 and/or PAK5 protein and the target molecule either by directly quantitating the complex or by measuring inductive effects of the RSK4 and/or PAK5 protein. A statistically significant change, such as a decrease, in the interaction of the RSK4 and/or PAK5 and RSK4 and/or PAK5 target molecule in the presence of a test compound (relative to what is detected in the absence of the test compound) is indicative of a modulation (e.g., inhibition or potentiation of the interaction between the RSK4 and/or PAK5 protein and the target molecule).

Positive tested compounds can be used as anti-type 1 and anti-type 2 diabetes mellitus and/or anti-nephropathic and/or anti-neuropathic drugs, including anti-diabetic nephropathic and anti-diabetic neuropathic drugs. Those drugs can regulate the kinases RSK4 and/or PAK5. Instead of the kinases RSK4 and/or PAK5 also RSK4 and/or PAK5 encoding DNA or polypeptides having RSK4 and/or PAK5 activity can be used.

Yet another aspect of the present invention concerns methods for modulating the transcription of certain genes in a cell by modulating RSK4 and/or PAK5 bioactivity, (e.g., by potentiating or disrupting a RSK4 and/or PAK5 bioactivity). In general, whether carried out in vivo, in vitro, or in situ, the method comprises treating the cell with an effective amount of a RSK4 and/or PAK5 therapeutic (agonist or antagonist of a RSK4 or PAK5 bioactivity) so as to alter, relative to the cell in the absence of treatment, the level of transcription of certain genes. Accordingly, the method can be carried out with RSK4 and/or PAK5 therapeutics such as peptide and peptidomimetics or other molecules identified in the above-referenced drug screens which agonize or antagonize the effects of a RSK4 or PAK5 bioactivity (e.g. transcription) of a gene which is regulated by a RSK4 and/or PAK5 protein. Other RSK4 or PAK5 therapeutics include antisense constructs for inhibiting expression of RSK4 and/or PAK5 proteins, and dominant negative mutants of RSK4 or PAK5 proteins which competitively inhibit interactions between ligands (e.g. proteins) and nucleic acids upstream and downstream of the wild-type RSK4 or PAK5 protein.

A further aspect of the present invention provides methods for determining whether a subject is at risk for developing type 1 or type 2 diabetes or nephropathies or neuropathies, including diabetic nephropathy or diabetic neuropathy. The method can include detecting, in a tissue of the subject, the presence or absence of a genetic lesion characterized by at least one of (i) a mutation of a gene encoding a RSK4 or PAK5 protein; (ii) the mis-expression of a RSK4 or PAK5 gene; or (iii) an error or mutation in the promoter regulating a RSK4 or PAK5 gene that may lead to aberrant expression. In preferred embodiments, detecting the genetic lesion includes ascertaining the existence of at least one of: a deletion of one or more nucleotides from a RSK4 or PAK5 gene; an addition of one or more nucleotides to a RSK4 or PAK5 gene, a substitution of one or more nucleotides of a RSK4 or PAK5 gene, a gross chromosomal rearrangement of a RSK4 or PAK5 gene; an alteration in the level of a messenger RNA transcript of a RSK4 or PAK5 gene; the presence of a non-wild type splicing pattern of a messenger RNA transcript of a RSK4 or PAK5 gene; a non-wild type level of a RSK4 or PAK5 protein; and/or an aberrant level of a RSK4 or PAK5 protein.

For example, detecting the genetic lesion can include (i) providing a probe/primer comprised of an oligonucleotide which hybridizes to a sense or anti sense sequence of a RSK4 and/or PAK5 gene or naturally occurring mutants thereof, or 5′ or 3′ flanking sequences naturally associated with the RSK4 or the PAK5 gene; (ii) contacting the probe/primer with an appropriate nucleic acid containing sample; and (iii) detecting, by hybridization of the probe/primer to the nucleic acid, the presence or absence of the genetic lesion; e.g. wherein detecting the lesion comprises utilizing the probe/primer to determine the nucleotide sequence of the RSK4 and/or the PAK5 gene and, optionally, of the flanking nucleic acid sequences. For instance, the primer can be employed in a polymerase chain reaction (PCR) or in a ligase chain reaction (LCR). In alternate embodiments, the level of a RSK4 and/or PAK5 protein is detected in an immunoassay using an antibody which is specifically immunoreactive with the RSK4 and/or the PAK5 protein. Other features and advantages of the invention will be apparent from the following detailed description and claims.

### DETAILED DISCLOSURE OF THE INVENTION

In an chromatin immunoprecipitation assay the two HNF4α target genes RSK4 and PAK5 were identified. Both proteins are kinases, which are involved in cell cycle regulation and signal transduction. Notably, HNF4α, RSK4 and PAK5 were specifically regulated in streptozotocin-induced diabetic rat kidney and brain. This provide a molecular rational for diabetic nephro- and neuropathy, frequently seen in patients with HNF4α dysfunction.

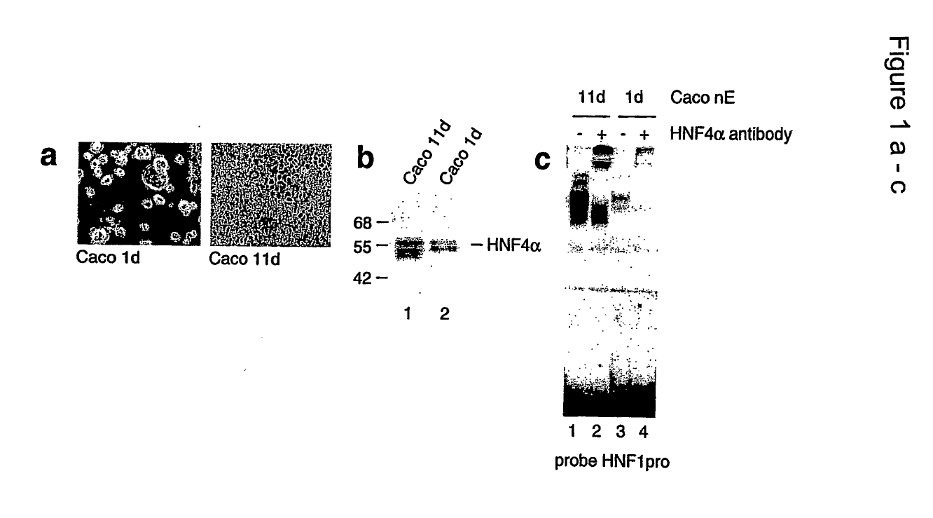

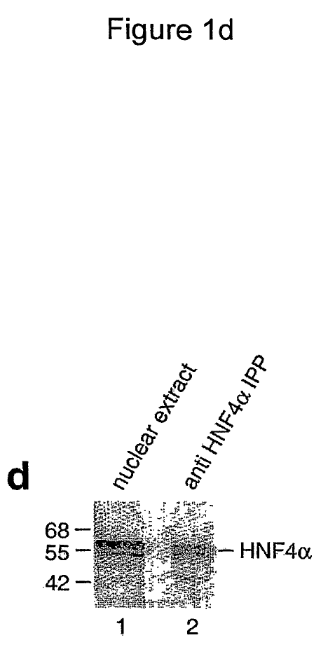

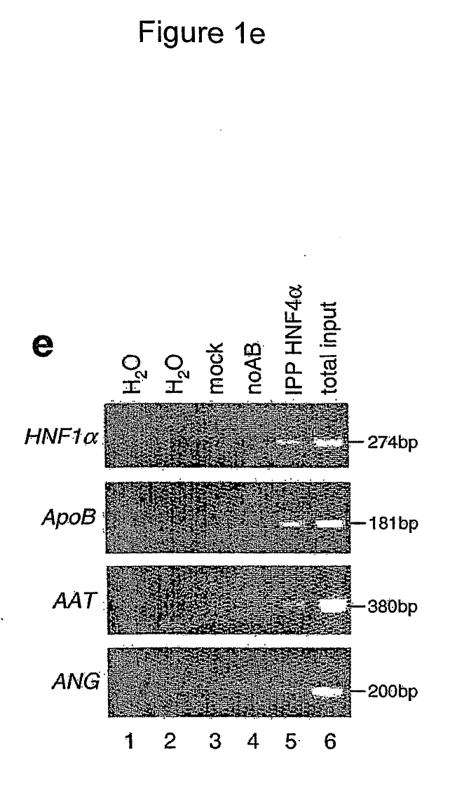

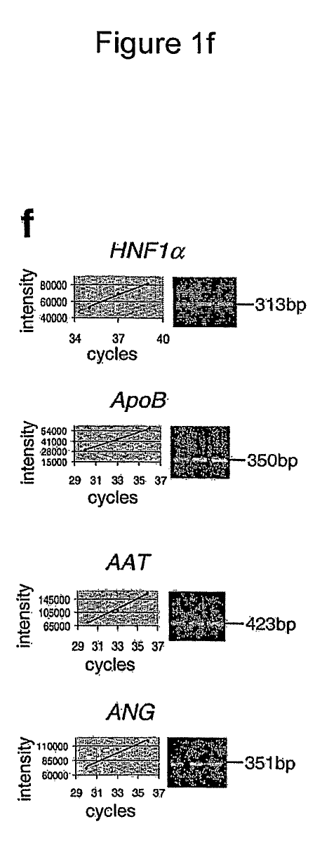

The ChIP-cloning procedure was used to identify novel HNF4α target-genes after formaldehyde crosslinking of nucleo-protein complexes in highly differentiated Caco-2 cell cultures (Hu, C. & Perlmutter, D. H. Regulation of alpha1-antitrypsin gene expression in human intestinal epithelial cell line caco-2 by HNF-1alpha and HNF-4. Am J Physiol 276, G1181-G1194 (1999)) (FIG. 1a). At day 11 in culture, the HNF4α protein expression was abundant (FIG. 1b), as determined by western-blotting experiments. Further, EMSA-experiments evidenced a marked increase in HNF4α DNA-binding to the A-site of the HNF1α-promoter (HNF1pro) (FIG. 1c). The A-site is an established recognition site for HNF4α (Schrem, H., Klempnauer, J., & Borlak, J. Liver-enriched transcription factors in liver function and development. Part I: the hepatocyte nuclear factor network and liver-specific gene expression. Pharmacol. Rev 54, 129-158 (2002); Sladek, F. M. & Seidel, S. D. Hepatocyte nuclear factor 4alpha in Nuclear Receptors and Disease. Academic Press, London. 309-361. (2001)). The ability of the HNF4α-antibody to immunoprecipitate HNF4α was confirmed by western blotting (FIG. 1d) and specificity of the ChIP-assay was tested by screening of immunoprecipitated DNA for enrichment of promoter sequences of well-known HNF4α target-genes (Schrem, H., Klempnauer, J., & Borlak, J. Liver-enriched transcription factors in liver function and development. Part I: the hepatocyte nuclear factor network and liver-specific gene expression. Pharmacol. Rev 54, 129-158 (2002); Sladek, F. M. & Seidel, S. D. Hepatocyte nuclear factor 4alpha in Nuclear Receptors and Disease. Academic Press, London. 309-361. (2001)). Specifically, by PCR-amplification it was confirmed that immunoprecipitated DNA contained HNF1α; apolipoprotein-B (ApoB), α1-antitrypsin (AAT) and angiotensinogen (ANG) (FIG. 1e). Therefore evidence for enrichment of known HNF4α target genes in immunoprecipitated DNA and document specificity for the experimental procedure is provided. Further expression of HNF4α target genes by RT-PCR is evidenced (FIG. 1f) as well as for each ChIP-assay detection of the HNF1pro-site served as a positive control. Therefore the experimental approach prior to the cloning of immunoprecipitated DNA was thoroughly validated. ChIP-assays yielded clones with inserts up to 1800 bp. The inserts were sequenced with vector-specific primers, and the genomic sequences were identified by database searches (GenBank, maintained by NCBI). Novel targets were distributed amongst different chromosomes, which demonstrates the utility of ChIP-cloning procedure and identified multiple chromosomal targets for this factor. Some of the cloned fragments were within intronic regions and this agrees well with finding by others (Greenbaum, S. & Zhuang, Y. Identification of E2A target genes in B lymphocyte development by using a gene tagging-based chromatin immunoprecipitation system. Proc Natl Acad Sci U.S.A 99, 15030-15035 (2002); Solano, P. J. et al. Genome-wide identification of in vivo Drosophila Engrailed-binding DNA fragments and related target genes. Development 130, 1243-1254 (2003), Martone, R. et al. Distribution of NF-{kappa}B-binding sites across human chromosome 22. PNAS 100, 12247-12252 (2003)). Proximal promoter sequences were also analyzed. Cloned fragments as well as promoter sequences were checked for putative HNF4α binding-sites and primer pairs were designed to confirm experimentally predicted sites. Independent ChIP-experiments followed by PCR-analyses with clone-specific or promoter-specific primers enabled robust identification of HNF4α target-genes.

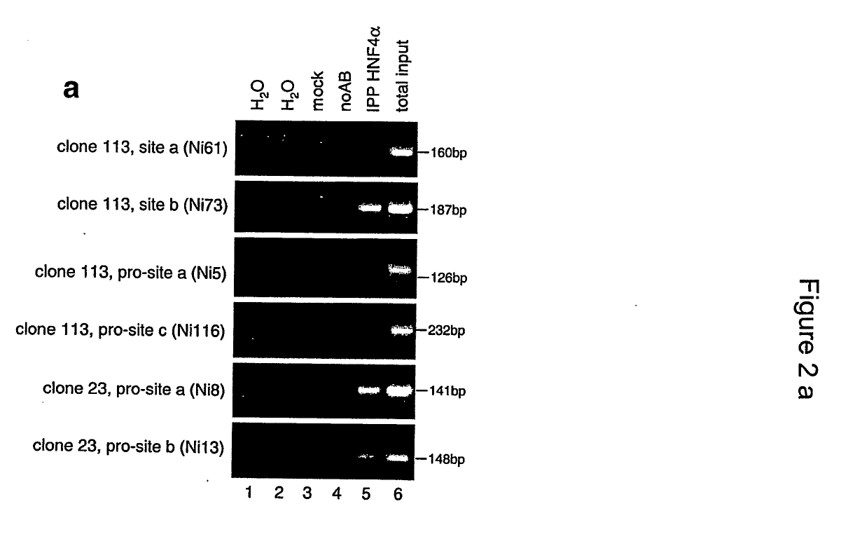

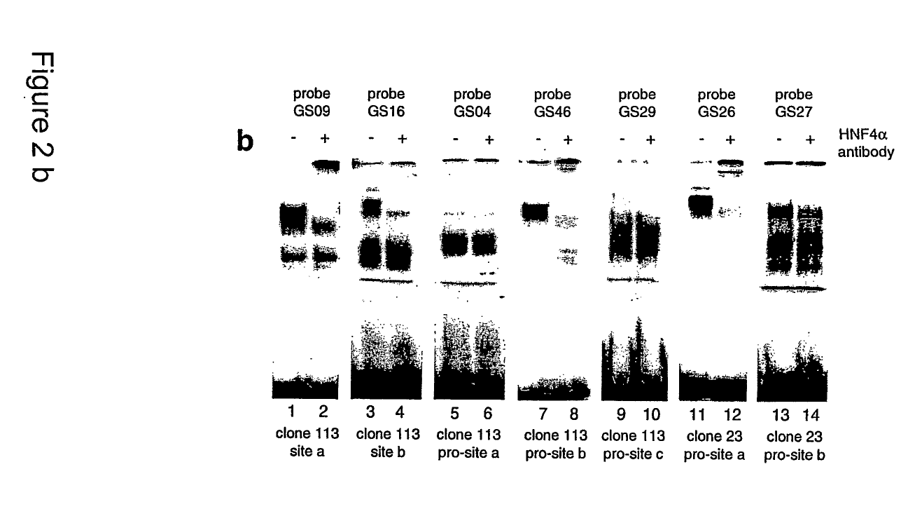

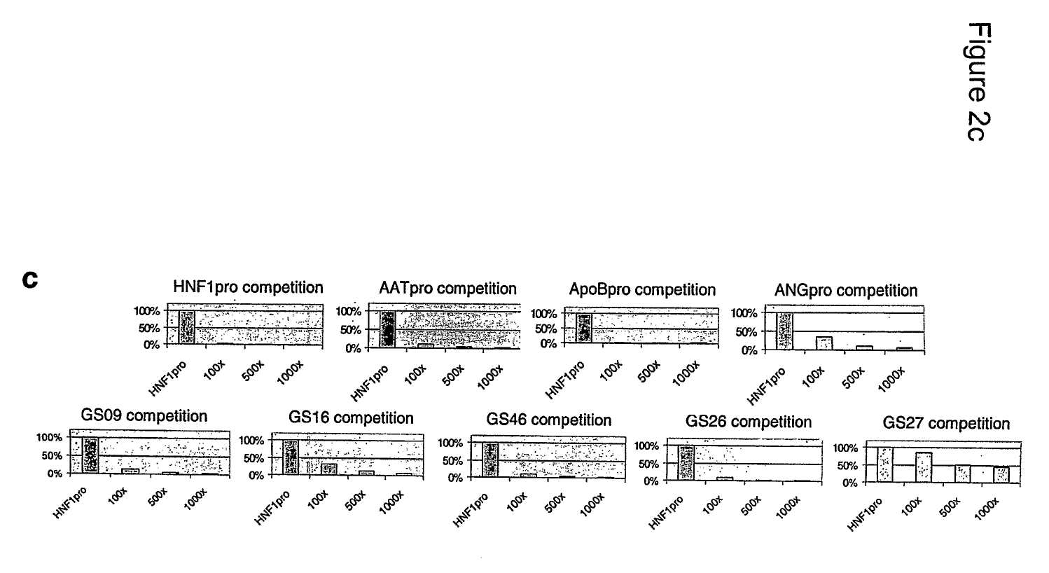

Here a detailed report of the identification of two novel kinases targeted by HNF4α is given. Binding in vivo of HNF4α was confirmed for two recognition sites of clone 113 (FIG. 2a). Additionally by employing a bioinformatic approach two promoter binding-sites within clone 113 and two promoter binding-sites within clone 23, which were specifically bound by HNF4α in vivo (FIG. 2a) were predicted. Further the ability of HNF4α to bind to cognate recognition sites by EMSA with ³²P-labeled probes encompassing the predicted HNF4α-sites located in clone 113 (GS09, GS16), in the promoter of clone 113 (GS04, GS29) and in the promoter of clone 23 (GS26, GS27) (FIG. 2b) was studied. Supershift experiments with a specific HNF4α antibody resulted in strong binding of HNF4α with probe GS09, GS16 and GS26 and weaker binding with probe GS27. No binding of HNF4α was detected with probe GS04 and GS29. Competition experiments with probes specific for several HNF4α target-genes were carried out to estimate binding affinity of HNF4α to the newly identified targets (FIG. 2c). The HNF1pro-site was used as labeled probe and competition was first analyzed with different known cognate recognition-sites, namely HNF1pro itself and HNF4α binding-sites within AATpro, ApoBpro and ANGpro. These sites were distinguishable in their binding affinity (FIG. 2c). With the AATpro-site (100×, inhibition to 10%) and the ApoB-site (100×, inhibition to 2.5%) binding was comparable to the HNF1pro-site itself (100×, inhibition to 2.2%), whereas competition with the ANGpro-site (100×, inhibition to 34.6%) was less efficient. By comparison, competition with the GS09 (100×, inhibition to 13.7%) and the GS26 (100×, inhibition to 9.5%) resulted in strong binding, whereas competition with the GS16 (100×, inhibition to 32.5%) was less efficient and binding affinity with the GS27 (100×, inhibition to 85.8%) was minimal. Competition experiments therefore complement findings obtained by supershift experiments. Clone 113 itself and two sites in the promoter of clone 23 were thus confirmed for in vivo and in vitro binding of HNF4α. Notably, HNF4α may contact DNA through various protein-protein interactions, particularly through cooperative binding with synergized factors as formaldehyde crosslinks leads to both, protein-DNA and protein-protein complexes. Therefore in ChIP-experiments a three-dimensional, higher order structure can be crosslinked. HNF4α in vitro binding to the putative sites (pro-site a and pro-site c) in the promoter of clone 113 was not confirmed, but the surrounding regions of 126 bp and 232 bp were confirmed as in vivo binding sites (see FIG. 2a). The promoter harbors an adjacent HNF4α binding-site (pro-site b, GS46, localized about 800 bp upstream to site a and about 400 bp downstream to site c), exhibiting strong in vitro binding of HNF4α (FIG. 2b+c), which would allow for immunoprecipitation of pro-site a and pro-site c through protein-protein crosslinks. Additionally, HNF4α may also contact another site within the in vivo confirmed fragments, which was not predicted by our computational approach.

Further, gene regulation of a broad range of CYP isozymes depends on promoter activation by HNF4α (Jover, R., Bort, R., Gomez-Lechon, M. J., & Castell, J. V. Cytochrome P450 regulation by hepatocyte nuclear factor 4 in human hepatocytes: a study using adenovirus-mediated antisense targeting. Hepatology 33, 668-675 (2001)). Notably, treatment of hepatocytes with Aroclor 1254 resulted in induction of HNF4α and several CYP monooxygenases (Borlak, J. & Thum, T. Induction of nuclear transcription factors, cytochrome P450 monooxygenases, and glutathione S-transferase alpha gene expression in Aroclor 1254-treated rat hepatocyte cultures. Biochemical Pharmacology 61, 145-153 (2001)). Therefore HNF4α-binding in liver nuclear extracts of control and Aroclor treated rats were analyzed by EMSA. Binding of HNF4α to HNF1pro as well as to the newly identified binding-sites (GS09, GS16, GS46, GS26, GS27) was significantly increased after Aroclor treatment (FIG. 2d), thus providing further evidence for our novel targets to be strictly regulated by HNF4α.

A summary of the cloned HNF4α targets is given in Table 1. Clone 23 contained two ChIP-verified HNF4α binding-sites in the promoter region (around −1430 and −2053) and was identified as RSK4, a novel member of ribosomal S6-kinase-subfamily (Yntema, H. G. et al. A Novel Ribosomal S6-Kinase (RSK4; RPS6KA6) Is Commonly Deleted in Patients with Complex X-Linked Mental Retardation. Genomics 62, 332-343 (1999); Kohn, M., Hameister, H., Vogel, M., & Kehrer-Sawatzki, H. Expression pattern of the Rsk2, Rsk4 and Pdk1 genes during murine embryogenesis. Gene Expr. Patterns. 3, 173-177 (2003)). Because RSK4 gene deletion was found in some patients with X-linked mental retardation, a role for this kinase in neuronal development was suggested (Yntema, H. G. et al. A Novel Ribosomal S6-Kinase (RSK4; RPS6KA6) Is Commonly Deleted in Patients with Complex X-Linked Mental Retardation. Genomics 62, 332-343 (1999)). RSK4 expression during mouse development is, however, ubiquitous (Kohn, M., Hameister, H., Vogel, M., & Kehrer-Sawatzki, H. Expression pattern of the Rsk2, Rsk4 and Pdk1 genes during murine embryogenesis. Gene Expr. Patterns. 3, 173-177 (2003)), which suggests additional roles for this gene in development. Clone 113 with two ChIP-verified HNF4α promoter binding-sites around −951 and around −2181 was annotated as PAK5. This kinase was recently cloned and characterized as a novel member of mammalian p21(cdc42/rac1)-activated-kinase-subfamily (Dan, C., Nath, N., Liberto, M., & Minden, A. PAK5, a New Brain-Specific Kinase, Promotes Neurite Outgrowth in N1E-115 Cells. Mol. Cell. Biol. 22, 567-577 (2002); Pandey, A. et al. Cloning and characterization of PAK5, a novel member of mammalian p21-activated kinase-II subfamily that is predominantly expressed in brain. Oncogene 21, 3939-3948 (2002)). Until now, its role was confined to induction of neurite outgrowth (Dan, C., Nath, N., Liberto, M., & Minden, A. PAK5, a New Brain-Specific Kinase, Promotes Neurite Outgrowth in N1E-115 Cells. Mol. Cell. Biol. 22, 567-577 (2002)), whereas PAK kinases in general play a role in neurodegenerative diseases (Kumar, R. & Vadlamudi, R. K. Emerging functions of p21-activated kinases in human cancer cells. J Cell Physiol 193, 133-144 (2002)).

It is of considerable importance that HNF4α targets kinases important in neuronal development and function. Thus, next to its role in liver metabolism, HNF4α may also play a role in brain function. Specifically, HNF4α regulates several genes involved in glucose metabolism (Schrem, H., Klempnauer, J., & Borlak, J. Liver-enriched transcription factors in liver function and development. Part I: the hepatocyte nuclear factor network and liver-specific gene expression. Pharmacol. Rev 54, 129-158 (2002); Sladek, F. M. & Seidel, S. D. Hepatocyte nuclear factor 4alpha in Nuclear Receptors and Disease. Academic Press, London. 309-361. (2001)) and participates in the glucose-dependent insulin secretory pathways. HNF4α dysfunction does, however, lead to multifactorial Type 2 diabetes (Silander, K. et al. Genetic Variation Near the Hepatocyte Nuclear Factor-4alpha Gene Predicts Susceptibility to Type 2 Diabetes. Diabetes 53, 1141-1149 (2004); Love-Gregory, L. D. et al. A Common Polymorphism in the Upstream Promoter Region of the Hepatocyte Nuclear Factor-4alpha Gene on Chromosome 20q Is Associated With Type 2 Diabetes and Appears to Contribute to the Evidence for Linkage in an Ashkenazi Jewish Population. Diabetes 53, 1134-1140 (2004)) with patients developing diabetic neuropathies. Moreover, one form of a rare monogenetic disorder termed maturity onset diabetes of the young (MODY) was mapped to mutations within the HNF4α gene (MODY-1), thus confirming its role in pancreatic β-cell function (Schrem, H., Klempnauer, J., & Borlak, J. Liver-enriched transcription factors in liver function and development. Part I: the hepatocyte nuclear factor network and liver-specific gene expression. Pharmacol. Rev 54, 129-158 (2002); Sladek, F. M. & Seidel, S. D. Hepatocyte nuclear factor 4alpha in Nuclear Receptors and Disease. Academic Press, London. 309-361. (2001)). Patients with diabetes harbor a high risk for progressive neuropathies for uncertain reasons (Vinik, A. I., Park, T. S., Stansberry, K. B., & Pittenger, G. L. Diabetic neuropathies. Diabetologia 43, 957-973 (2000)). Though the precise role of HNF4α in brain function is unknown, gene expression of this transcription factor in human and rat brain is demonstrated (FIG. 2e). Interestingly, expression of the splice variant HNF4α7 was also reported for mouse brain (Nakhei, H., Lingott, A., Lemm, I., & Ryffel, G. U). An alternative splice variant of the tissue specific transcription factor HNF4alpha predominates in undifferentiated murine cell types. Nucleic Acids Res 26, 497-504 (1998)). Next to its expression in brain tissue RSK4 is expressed at similar level in kidney, but lesser in pancreas and placenta (Yntema, H. G. et al. A Novel Ribosomal S6-Kinase (RSK4; RPS6KA6) Is Commonly Deleted in Patients with Complex X-Linked Mental Retardation. Genomics 62, 332-343 (1999)). Gene expression of RSK4 is demonstrated in Caco-2 cells, in human and rat liver and in rat kidney (FIG. 2e). Unlike RSK4, PAK5 is abundantly expressed in pancreas and minimal expressed in liver and kidney (Dan, C., Nath, N., Liberto, M., & Minden, A. PAK5, a New Brain-Specific Kinase, Promotes Neurite Outgrowth in N1E-115 Cells. Mol. Cell. Biol. 22, 567-577 (2002)). Expression of PAK5 in RNA extracts of human and rat liver and rat kidney is demonstrated (FIG. 2e), but not in cultures of Caco-2 cells. Failure to detect PAK5 gene expression in Caco-2 cells suggests lack of synergistic transcription factors acting in concert, even though in vivo binding of HNF4α to PAK5 recognition sites was confirmed (see FIG. 2a).

HNF4α is a dominant regulator of the epithelial phenotype and is highly expressed in kidney (Sladek, F. M. & Seidel, S. D. Hepatocyte nuclear factor 4alpha in Nuclear Receptors and Disease. Academic Press, London. 309-361. (2001)). Further, in diabetic patients nephropathy is a frequently observed complication and treatment of rats with streptozotocin (STZ) results in diabetic nephro- and neuropathy (Bianchi, R. et al. Erythropoietin both protects from and reverses experimental diabetic neuropathy. PNAS 101, 823-828 (2004), Gross, M. L. et al. ACE-inhibitors but not endothelin receptor blockers prevent podocyte loss in early diabetic nephropathy. Diabetologia 46, 856-868 (2003)). A significant reduction of HNF4α gene expression in liver and PAK5 gene expression in brain extracts of STZ-induced diabetic rats is evidenced (Table 2). Moreover, HNF4α itself, RSK4 and PAK5 gene expression was also significantly repressed in rat kidneys (Table 2). Therefore diabetic neuropathy and nephropathy is demonstrated to be linked to RSK4 and PAK5 deregulation, as a result of HNF4α dysfunction. Moreover, treatment of rats with Aroclor 1254 led to significant induction of RSK4 mRNA in rat kidneys (Table 2) thus providing further evidence for a coordinate regulation of RSK4 and HNF4α gene expression.

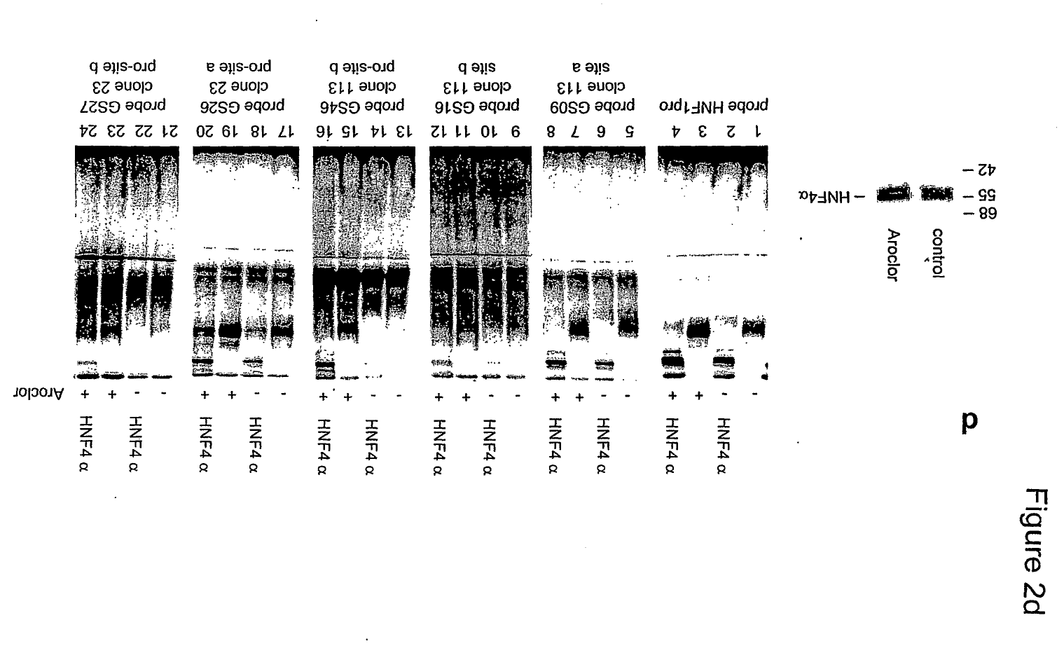

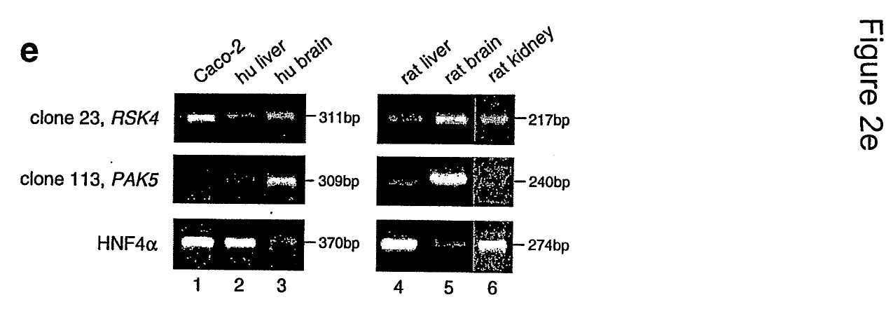

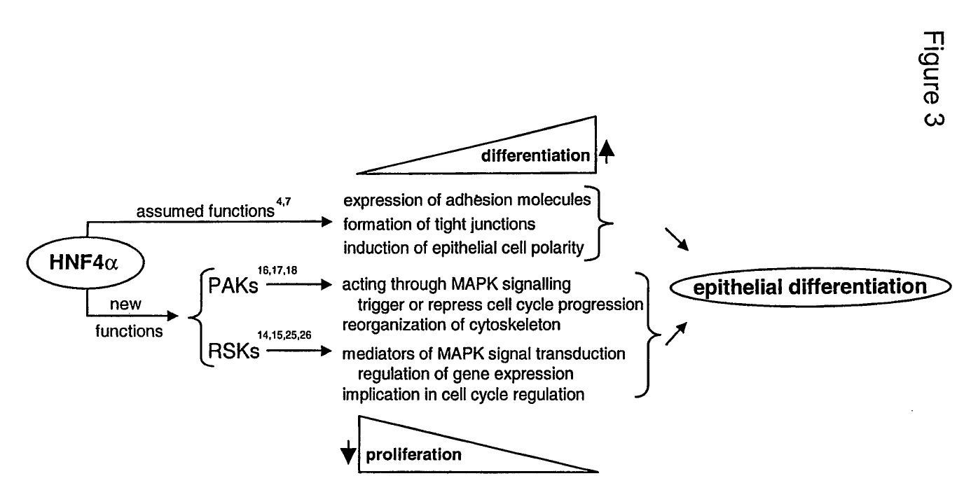

Noteworthy, RSK family members function as downstream mediators of MAP/ERK signal transducers of cell survival and cell cycle regulation (Nebreda, A. R. & Gavin, A. C. Perspectives: signal transduction. Cell survival demands some Rsk. Science 286, 1309-1310 (1999); Roux, P. P., Richards, S. A., & Blenis, J. Phosphorylation of p90 Ribosomal S6 Kinase (RSK) Regulates Extracellular Signal-Regulated Kinase Docking and RSK Activity. Mol. Cell. Biol. 23, 4796-4804 (2003)), whereas PAK kinases play key roles in the stimulation of MAPK signaling pathways to support or to repress cell-cycle progression (Kumar, R. & Vadlamudi, R. K. Emerging functions of p21-activated kinases in human cancer cells. J Cell Physiol 193, 133-144 (2002)). PAK kinases also regulate cytoskeletal dynamics by disassembly of stress fibers and focal adhesions (Kumar, R. & Vadlamudi, R. K. Emerging functions of p21-activated kinases in human cancer cells. J Cell Physiol 193, 133-144 (2002)). Thus, RSKs and PAKs are positive and negative regulators of cell cycle. Conceivably, cellular differentiation, as fostered by HNF4α, is opposite to cell proliferation. In conclusion, HNF4α has a unique role in targeting RSK and PAK family-members. This suggests a novel role for this liver enriched transcription factor in repressing cell-cycle progression to enable cellular differentiation (FIG. 3). This provides the missing link between HNF4α dysfunction and late stage complications in diabetes frequently seen in patients with progressive stages of disease.

### Methods

Experiments were performed after the following methods.

### Caco-2 Cell Culture:

Caco-2 cells were obtained from and cultivated as recommended by DMSZ. Essentially, cells were cultured in DMEM supplemented with 10% FCS and 200 μg/ml penicillin, 200 μg/ml streptomycin and 615 μg/ml L-glutamic acid. Cells were used between the 5. and the 20. passage and were checked for purity and morphological abnormalities by phase contrast microscopy. Caco-2 cells were seeded with a density of 4×10⁶ cells per 75 cm² flask and harvested after 11 days.

### Isolation of Nuclear Extracts:

The use of animals was approved by the local government of Hannover with project license 02-548. Sprague Dawley rats (n=3) were treated with a single i.p. dose of 100 mg Aroclor 1254 per kg bodyweight and killed 72 h later. Nuclear extracts from rat liver were prepared as described by Gorski, K., Carneiro, M., & Schibler, U. Tissue-specific in vitro transcription from the mouse albumin promoter. Cell 47, 767-776 (1986), whereas nuclear extracts from Caco-2 cells were isolated by a modified method of Dignam, J. D., Lebovitz, R. M., & Roeder, R. G. Accurate transcription initiation by RNA polymerase II in a soluble extract from isolated mammalian nuclei. Nucleic Acids Res 11, 1475-1489 (1983). Eleven days after seeding cells were washed twice with ice-cold PBS, scraped into microcentrifuge tubes and centrifuged for 5 min at 2000×g, 4° C. Cell pellets were resuspended in hypotonic buffer (10 mM Tris pH 7.4, 2 mM MgCl₂, 140 mM NaCl, 1 mM DTT, 4 mM Pefabloc, 1% Aprotinin, 40 mM β-glycerophosphate, 1 mM sodiumorthovanadate and 0.5% TX100) at 4° C. for 10 min (300 μl for 1×10⁷ cells), transferred onto one volume of 50% sucrose in hypotonic buffer and centrifuged at 14000×g and 4° C. for 10 miin. Nuclei were resuspended in Dignam C buffer (20 mM Hepes pH 7.9, 25% glycerol, 420 mM NaCl, 1.5 mM MgCl₂, 0.2 mM EDTA, 1 mM DTT, 4 mM Pefabloc, 1% Aprotinin, 40 mM β-glycerophosphate, 1 mM sodiumorthovanadate, 30 μl for 1×10⁷ cells) and gently shaked at 4° C. for 30 min. Nuclear debris was removed by centrifugation at 14000×g at 4° C. for 10 min. The extracts were aliquoted and stored at −70° C. Protein concentrations were determined according to the method of Smith, P. K. et al. Measurement of protein using bicinchoninic acid. Anal. Biochem. 150, 76-85 (1985).

### Western Blot Analysis:

Nuclear extracts or HNF4α immunoprecipitated complexes (collected with Protein G-Sepharose, Pharmacia) were separated on a 12% SDS-polyacrylamide gel and blotted onto a PVDF membrane in 25 mM Tris and 190 mM Glycin at 4° C. for 3 h at 350 mA. The antibody directed against HNF4α was purchased from Santa Cruz Biotechnology (sc-8987x rabbit polyclonal IgG). The antigen-antibody complexes were visualized using the ECL detection system as recommended by the manufacturer NEN Life Science Products and recorded with Kodak IS 440 CF.

### Electrophoretic Mobility Shift Assays:

Nuclear extracts were used as described in the figure legends. Binding buffer consisted of 25 mM HEPES, pH 7.6, 5 mM MgCl₂, 34 mM KCl, 2 mM DTT, 2 mM Pefabloc, 2% Aprotinin, 40 ng of poly (dI-dC)/μl and 100 ng of bovine serum albumin/μl. Oligonucleotides and nuclear proteins were incubated for 30 minutes on ice. Free DNA and DNA-protein complexes were resolved on a 6% polyacrylamide gel. The oligonucleotides were purchased from MWG Biotech and used as ³²P-labeled probes, for sequence information see Supplementary Table 1. Super shift experiments were done with an HNF4α specific antibody (sc-6556x, Santa Cruz Biotechnology).

### Crosslinking and Chromatin Immunoprecipitation (ChIP):

We employed two rounds of sequential chromatin immunoprecipitations (Weinmann, A. S., Bartley, S. M., Zhang, T., Zhang, M. Q., & Farnham, P. J. Use of chromatin immunoprecipitation to clone novel E2F target promoters. Mol Cell Biol 21, 6820-6832 (2001)) to increase purity and specificity of target DNA for ChIP-cloning and for validation of ChIP-derived clones. Other investigators employed a single immunoprecipitation step with obvious limitations for validation of targets (Tomaru, Y., Kondo, S., Suzuki, M., & Hayashizaki, Y. A comprehensive search for HNF-3[alpha]-regulated genes in mouse hepatoma cells by 60K cDNA microarray and chromatin immunoprecipitation/PCR analysis. Biochemical and Biophysical Research Communications 310, 667-674 (2003); Horak, C. E. et al. GATA-1 binding sites mapped in the beta-globin locus by using mammalian chip-chip analysis. PNAS 99, 2924-2929 (2002)). Further, our highly sensitive PCR-assays did not always confirm exclusive HNF4α bound DNA after a single immunoprecipitation step, but rather resulted in signal enrichment compared to the no antibody control. We, therefore, performed two consecutive rounds of immunoprecipitation to obtain clean signals in relation to the no antibody controls (see FIG. 1). With some modifications all CHIP procedures were carried out as described by Weinmann, A. S., Bartley, S. M., Zhang, T., Zhang, M. Q., & Farnham, P. J. Use of chromatin immunoprecipitation to clone novel E2F target promoters. Mol Cell Biol 21, 6820-6832 (2001). Caco-2 cells were treated with 1% formaldehyde at room temperature for 10 min under constant agitation. The reaction was stopped by the addition of glycine to obtain a final concentration of 125 mM. Cells were washed twice with ice-cold PBS, detached with trypsin and collected by centrifugation. Cells were resuspended in lysis buffer (5 mM PIPES, 85 mM KCl, 0.5% NP40 and 1× complete protease inhibitor, Roche) and incubated on ice for 20 min. The nuclei were collected by microcentrifugation and then resuspended in nuclei lysis buffer (1% SDS, 10 mM EDTA pH 8.0, 50 mM Tris-HCl pH 8.1 and 1× complete) and incubated on ice for 10 min. The samples were sonicated on ice until crosslinked chromatin was fragmented to approximately 0.2-1.6 kbp. Protein A-Sepharose CLB4 (Pharmacia) was blocked with 1 mg ml⁻¹ BSA and 1 mg ml⁻¹ herring sperm DNA (Promega) and washed extensively before use. Chromatin preparations were precleared by incubation with ‘blocked’ Protein A-Sepharose for 1 h at 4° C. The Protein A-Sepharose was removed by centrifugation and the precleared chromatin was diluted 1:3 with immunoprecipitation (IP) dilution buffer (0.01% SDS, 1.2 mM EDTA pH 8.0, 1.1% Triton X100 and 1× complete). Precleared chromatin from 2.5×10⁷ cells was incubated with 1 μg goat polyclonal HNF4α antibody (sc 6556, Santa Cruz) or no antibody and rotated at 4° C. overnight. Immunoprecipitates were recovered by incubation with a secondary antibody (rabbit anti-goat) and ‘blocked’ Protein A-Sepharose, washed twice with dialysis buffer (50 mM Tris-HCl pH 8.0, 2 mM EDTA, 0.2% sarkosyl) and four times with IP wash buffer (100 mM Tris-HCl pH 9.0, 500 mM LiCl, 1% NP40, 1% deoxycholic acid). Prior to the first wash, the supernatant from the reaction with no antibody was saved as total input chromatin and was processed with the eluted immunoprecipitates at the beginning of the cross-link reversal step. Elution was done with 30 μl elution buffer (1% SDS, 50 mM NaHCO₃) and samples were diluted 1:10 with IP dilution buffer. Two samples were pooled for a second immunoprecipitation step with the HNF4α antibody. After further recovering and washing steps, elution was done two times with 150 μl elution buffer each. Cross-links were then reversed by the addition of NaCl to a final concentration of 300 mM, and RNA was removed by the addition of 10 μg of RNase A per sample followed by incubation at 65° C. for 4-5 h. The samples were then precipitated at −20° C. overnight by addition of 2.5 volumes of ethanol and then pelleted by microcentrifugation. The samples were resuspended in 100 μl Tris-EDTA (pH 7.6), 25 μl 5× proteinase K buffer (1.25% SDS, 50 mM Tris-HCl pH 7.5, 25 mM EDTA pH 8.0) and 125 μg proteinase K (Roth) and incubated at 55° C. for 2 h. The cleanup of DNA was done by extraction with phenol-chloroform-isoamyl alcohol (25:24:1) and subsequent ethanol precipitation. The pellets were resuspended in 30 μl H₂O and analyzed by PCR. A mock probe, containing buffer without chromatin, was treated categorically through the whole immunoprecipitation procedure and through DNA isolation to control for DNA contamination.

PCR was conducted in a mixture containing 2 μl of purified DNA or 2 μl of a 1:200 dilution of the total input sample, 0.5 mM of each primer, 0.25 mM dNTP mixture, 0.625 U Thermostart-Taq (Abgene) and 1×PCR-buffer (Abgene, with 1.5 mM MgCl₂) in a total volume of 20 μl. For the PCR primers used to analyze immunoprecipitated target genes see Supplementary Table 2. The PCR-amplification products were run on a 2.0% agarose gel and analyzed by ethidium bromide staining.

We performed numerous independent ChIP-experiments but did not always identify the full complement of selected HNF4α target genes as our positive controls. Indeed, the second immunoprecipitation step resulted in very low yield of immunoprecipitated material. Further, formaldehyde crosslinking of nucleo-protein complexes did impact recognition of HNF4α bound DNA. Thus, the spectrum of HNF4α positive targets varied after immunoprecipitation. Consequently, for a given clone we demanded at least three independent confirmations from a series of independent ChIP-experiments until a target was considered to be confirmed.

### Cloning:

The immunoprecipitated DNA was treated with T4 DNA polymerase (New England Biolabs) to create blunt ends, purified, and cloned into the zero-blunt vector (Invitrogen) using the zero-blunt PCR cloning kit (Invitrogen) according to the manufacturers recommendations. Colonies having inserts were identified by restriction enzyme digestion using enzymes in the polylinker.

### Sequence Analysis:

Plasmid-DNA was purified with QIAquick PCR Purification Kit (Qiagen), subjected to cycle sequencing with vector-specific primers using BigDyeTerminator v3.1 Kit and injected into ABI 3100 Genetic Analyzer (Applied Biosystems). Sequences were identified by database searches (GenBank version Build 34, maintained by NCBI) for human genomic matches. Detailed sequence information is given in Supplementary Table 3.

### Bioinformatic Searching for HNF4α Binding-Sites:

Cloned fragments and respective proximal promoters (−1 to −3000 bp) were checked for putative HNF4α binding-sites with two different bioinformatic weight matrix-based tools, Transfac matrix (Biobase, www.biobase.de) V$HNF4_{—}01 with cut-off core similarity 0.75 and matrix similarity 0.78, and Genomatix matrix (Genomatix, www.genomatox.de) V$HNF4_{—}01 with cut-off core similarity 0.75 and matrix similarity 0.82 or V$HNF4_{—}02 with cut-off core similarity 0.75 and matrix similarity 0.76.

### RT-PCR:

Total RNA was isolated using the nucleospin RNA Isolation Kit (Macherey-Nagel) according to the manufacturers recommendations. 4 μg total RNA from each sample was used for reverse transcription (Omniscript Reverse Transcriptase, Qiagen). PCR was done in a mixture containing a cDNA equivalent to 25 ng of total RNA, 0.5 mM of each primer, 0.25 mM dNTP mixture, 0.625 U Thermostart-Taq (Abgene) and 1×PCR-buffer (Abgene, with 1.5 mM MgCl₂) in a total volume of 20 μl. PCR-reactions were carried out with a thermocycler (T3, Biometra). Reactions were done within the linear range of amplification and amplification products were separated using a 1.5% agarose gel and stained with ethidium bromide. For detailed oligonucleotide sequence information and PCR-program see Supplementary Table 4.

Real-time semi-quantitative PCR: Real-time RT-PCR measurement was done with the Lightcycler (Roche Diagnostics). For detailed oligonucleotide sequence information and PCR-program see Supplementary Table 5. The PCR reaction was stopped after a total of 30 to 50 cycles and at the end of each extension phase, fluorescence was observed and used for quantitative measurements within the linear range of amplification. Exact quantification was achieved by serial dilution with cDNA produced from total RNA extracts using 1:5 dilution steps. Gene expression levels were normalized to mitATPase6 as stable expressed housekeeping gene.

### Diabetic Disease Model:

Kidneys of streptozotocin (STZ) treated Sprague Dawley rats (6 months) were kindly provided by R. Amann. Conclusive evidence for this treatment to result in diabetic nephropathy was recently published (Gross, M. L. et al. ACE-inhibitors but not endothelin receptor blockers prevent podocyte loss in early diabetic nephropathy. Diabetologia 46, 856-868 (2003)). Further, liver, kidney and brain of STZ-treated rats (3 months) were kindly provided by P. Rösen, German Diabetes Research Institute, Düsseldorf.

### Statistics:

Data are given as means ±SD. Student's t-test or analysis of covariance (ANCOVA) with interaction between factor and covariable was applied for data statistical analysis. The results were considered significant when the p value was less than 0.05.

### EXAMPLES

In the following the invention is elucidated by embodiments.

### FIGURE LEGENDS

### FIG. 1

HNF4α chromatin immunoprecipitation assay with HNF4α target genes.

(a) Phase contrast photomicrographs of cultured Caco-2 cells at day 1 and at day 11 (200×). (b) HNF4α western blotting analysis of 30 μg Caco-2 cell nuclear extracts prepared at day 1 (lane 2) or day 11 (lane 1). (c) Electrophoretic mobility shift experiment with 2.5 μg Caco-2 cell nuclear extracts prepared at day 1 (lane 3 and 4) or day 11 (lane 1 and 2) and an oligonucleotide corresponding to the A-site of the HNF1α promoter (HNF1pro) as ³²P labeled probe. For supershift analysis an antibody directed against HNF4α was added (lane 2 and 4). (d) HNF4α western blot analysis of 30 μg total nuclear extract (nuclear extract, lane 1) and of HNF4α immunoprecipitated complexes (anti HNF4α IPP, lane 2) of Caco-2 cells. (e) ChIP experiments were performed with cultured Caco-2 cells and an antibody against HNF4α (IPP HNF4α, lane 5) or no antibody (noAB, lane 4). The no antibody control was used to monitor unspecific binding of DNA. Following DNA purification samples were subjected to PCR with primers designed to amplify promoters of different HNF4α positive targets. The primers annealed proximal to the HNF4α binding-sites of the HNF1α promoter, the apolipoprotein B (ApoB) promoter, the α1-antitrypsin (AAI) promoter, and the angiotensinogen (ANG) promoter, all of which are well-known HNF4α-targets. A mock probe (mock, lane 3) and a portion of the total input sample (total input, lane 6) were also examined by PCR. A mock probe, containing buffer without chromatin, was treated categorically through the whole immunoprecipitation procedure and through DNA isolation to control for DNA contamination. Routinely, two reactions containing H₂O instead of template were included in each PCR as negative control (lane 1 and 2). (f) Gene expression of HNF1α; ApoB, AAT and ANG in cultures of Caco-2 cells was analyzed by RT-PCR as detailed in the supplementary material. A linear range of amplification cycles was shown.

### FIG. 2

Confirmation of ChIP clones by examination of HNF4α binding in vivo and in vitro.

(a) Independent ChIP experiments were performed with cultures of Caco-2 cells and an antibody against HNF4α (IPP HNF4α, lane 5) or no antibody (noAB, lane 4). Following DNA purification samples were subjected to PCR with primers designed for putative HNF4α binding-sites of clones and their promoters (clone 113, clone 113 pro-site a and pro-site c, clone 23 pro-site a and pro-site b). A mock probe (lane 3) and an aliquot of the total input sample (lane 6) were also examined by PCR. Routinely two reactions containing H₂O instead of template were included in each PCR as negative control (lane 1 and 2). (b) Electrophoretic mobility shift assays with 10 μg Caco-2 cell nuclear extract and oligonucleotides (GS09, lane 1 and 2; GS16, lane 3 and 4; GS04, lane 5 and 6; GS46, lane 7 and 8; GS29, lane 9 and 10; GS26, lane 11 and 12; GS27, lane 13 and 14) corresponding to putative HNF4α binding-sites within the identified clones and promoters as ³²P labeled probe. In supershift assays an antibody directed against HNF4α (+) was added (lane 2, 4, 6, 8, 10, 12, 14). (c) Competition experiments. Electrophoretic mobility shift experiments were carried out with 2.5 μg Caco-2 cell nuclear extracts and an oligonucleotide corresponding to the A-site of the HNF1α promoter as ³²P labeled probe. The A-site of the HNF1α promoter (HNF1pro), the HNF4α binding-site of the α1-antitrypsin promoter (AATpro), the HNF4α binding-site of the apolipoprotein B promoter (ApoBpro), the C-region of the angiotensinogen promoter (ANGpro) and the putative HNF4α binding-sites (GS09, GS16, GS46, GS26, GS27) were added as 100 fold, 500 fold and 1000 fold molar excess. Dried gels were analyzed with a Molecular Imager (BioRad) using the Quantity One software (BioRad). HNF4α binding to the A-site of the HNF1α promoter as ³²P labeled probe was set to 100% and competition was quantified for each oligonucleotide. (d) HNF4α western blot analysis (left) of 30 μg liver nuclear extract of control and Aroclor treated rats. Electrophoretic mobility shift assay with 2.5 μg rat liver nuclear extract of control (lane 1, 2, 5, 6, 9, 10, 13, 14, 17, 18, 21, 22) or Aroclor treated (lane 3, 4, 7, 8, 11, 12, 15, 16, 19, 20, 23, 24) animals and oligonucleotides corresponding to different potential HNF4α binding-sites (HNF1pro, lane 1-4; GS09, lane 5-8; GS16, lane 9-12; GS46, lane 13-16; GS26, lane 17-20; GS27, lane 21-24) as ³²P labeled probe. In supershift assays an antibody directed against HNF4α was added (lane 2, 4, 6, 8, 10, 12, 14, 16, 18, 20, 22, 24). (e) Gene expression of clone 23 (RSK4), clone 113 (PAK5) and HNF4α was analyzed by RT-PCR in extracts of Caco-2 cell cultures (1), human liver (2), human brain (3), rat liver (4), rat brain (5) and rat kidney (6). In the case of PAK5 half of the PCR reaction was loaded on the gel for human and rat brain, in the case of HNF4α half of the PCR reaction was loaded on the gel for Caco-2 cells, human liver, rat liver and kidney.

### FIG. 3

Function of HNF4α in cellular differentiation and cell cycle control.

For details see text.

TABLE 1
Summary of clone information

| | | | Swiss-Prot/ | | mRNA | HNF4α promoter | molecular | |
|---|---|---|---|---|---|---|---|---|
| clone | gene name | localization | mRNA NCBI | reference | expression | binding-sites | function | biological process |
| 23 | RSK4 | chromosome X | Q9UK32/ | 1, 2 | Caco-2 cells, | -1430 (pro-site a) | protein kinase | cell communication/ |
| | also named | 1. intron | NM_014496 | | hu liver, hu brain, | ChIP confirmed | activity | signal transduction |
| | RPS6KA6 | | | | rat liver, rat brain, | EMSA binding | | |
| | | | | | rat kidney | -2053 (pro-site b) | | |
| | | | | | | ChIP confirmed | | |
| | | | | | | EMSA binding | | |
| 113 | PAK5 | chromosome 20 | Q9P286/ | 3, 4 | hu liver, hu brain, | -951 (pro-site a) ChIP | protein kinase | cell communication/ |
| | also named | 3. intron* | NM_045653 | | rat liver, rat brain, | confirmed | activity | signal transduction |
| | PAK7 | site a | | | rat kidney | EMSA no binding | | |
| | | ChIP confirmed | | | | -1766 (pro-site b) | | |
| | | EMSA binding | | | | EMSA binding | | |
| | | site b | | | | -2181 (pro-site c) | | |
| | | ChIP confirmed | | | | ChIP confirmed | | |
| | | EMSA binding | | | | EMSA no binding | | |

*coincides with the 1. intron behind the 1. translated exon; 1) Yntema, H. G. et al. A Novel Ribosomal S6-Kinase (RSK4; RPS6KA6) Is Commonly Deleted in Patients with Complex X-Linked Mental Retardation. Genomics 62, 332-343 (1999); 2) Kohn, M., Hameister, H., Vogel, M., & Kehrer-Sawatzki, H. Expression pattern of the Rsk2, Rsk4 and Pdk1 genes during murine embryogenesis. Gene Expr.Patterns. 3, 173-177 (2003); 3) Dan, C., Nath, N., Liberto, M., & Minden, A. PAK5, a New Brain-Specific Kinase, Promotes Neurite Outgrowth in NIE-115 Cells. Mol.Cell.Biol. 22, 567-577 (2002); 4) Pandey, A. et al. Cloning and characterization of PAK5, a novel member of mammalian p21-activated kinase-II subfamily that is predominantly expressed in brain. Oncogene 21, 3939-3948 (2002).

TABLE 2
HNF4α target regulation in STZ-induced diabetic rats and after Aroclor treatment

| | | | number | | p-value versus control | |
|---|---|---|---|---|---|---|
| gene | organ | treatment | of animals | mean ± SD | (two tailed/one tailed) | statistic method |
| HNF4α | liver | control | n = 8 | 0.8472 ± 0.2893 | | t-test |
| | | STZ, 2 months | n = 9 | 0.5364 ± 0.2409 | 0.0288/0.0144 | |
| HNF4α | kidney | control | n = 8 | 0.8443 ± 0.3276 | | t-test |
| | | STZ, 2 months | n = 7 | 0.5464 ± 0.1027 | 0.0389/0.0195 | |
| RSK4 | kidney | control | n = 3 | 0.4777 ± 0.2053 | | analysis of covariance (ANCOVA) with |
| | | STZ, 6 months | n = 3 | 0.3621 ± 0.0764 | 0.0710/0.0355 | interaction between factor and covaribale |
| RSK4 | kidney | control | n = 6 | 0.1535 ± 0.0615 | | t-test |
| | | aroclor 1257, 48 h | n = 6 | 0.3244 ± 0.0749 | 0.0015/0.0008 | |
| PAK5 | kidney | control | n = 8 | 0.1170 ± 0.0552 | | t-test |
| | | STZ, 2 months | n = 7 | 0.0224 ± 0.0191 | 0.0015/0.0008 | |
| PAK5 | brain | control | n = 6 | 0.2723 ± 0.1292 | | t-test |
| | | STZ, 2 months | n = 7 | 0.1330 ± 0.0699 | 0.0309/0.0155 | |

Supplementary Table 1 (Shift-probes)

| | | |
|---|---|---|
| gene/clone | oligo-name | sequence |

| | | |
|---|---|---|
| HNF1α | HNF1pro | AAGGCTGAAGTCCAAAGT |
| | | TCAGTCCCTTC |
| α1-antitrypsin | AATpro | GCCAGTGGACTTAGCCCC |
| | | TGTTG |
| apolipoprotein B | ApoBpro | GGAAAGGTCCAAAGGGCG |
| | | CCTTG |
| angiotensinogen | ANGpro | TGCAGAGGGCAGAGGGCA |
| | | GGGGA |
| clone 113, site a | GS09 | TGTTGGGTACAATGTTCA |
| | | ATATT |
| clone 113, site b | GS16 | GCACATGTACCTTAGTCA |
| | | GCTTT |
| clone 113/pro-site a | GS04 | GAGCTGGGACAAAAACTC |
| | | AGACT |
| clone 113/pro-site b | GS46 | AATATTGAACTTTAATCA |
| | | CTTAA |
| clone 113/pro-site c | GS29 | AAAACATTCCTTTGTCCA |
| | | CCACA |
| clone 23/pro-site a | GS26 | GCTGTTGACCTATGCCCT |
| | | CCATT |
| clone 23/pro-site b | GS27 | ACTGCCAACCTTTTGAGC |
| | | GCTGG |

Supplementary Table 2 (ChIP-PCR primer and amplification settings)

| | | | |
|---|---|---|---|
| gene/clone | primer | fragment length | annealing |

| | | | |
|---|---|---|---|
| HNF1α (Ni16) | fwd: CATGATGCCCCTACAAGGT | 274 bp | 60° C. |
| | rev: ATTGGAGCTGGGGAAATTCT | | |
| ApoB (Ni56) | fwd: GAGGCTCTTCAAGGCTCAAA | 181 bp | 60° C. |
| | rev: AACCGAGAAGGGCACTCAG | | |
| AAT (Ni75) | fwd: GGAACAGCCACTAAGGATTCTGCAGTGAG | 380 bp | 60° C. |
| | rev: TCACTGTCCCAGGTCAGTGGTGGTGCCTG | | |
| ANG (Ni1 17) | fwd: GGGAGATGTACCCCCAAGAG | 200 bp | 60° C. |
| | rev: GCTTGTGTGTTTTCCCCAGT | | |
| clone 23 | fwd: CCAACTCAGGACCTTGGAGA | 141 bp | 60° C. |
| pro-site a (Ni8) | rev: GGCCAGCTTTGCTTCATTAG | | |
| clone 23 | fwd: GAGCTGCTGTGCCTGGTACT | 148 bp | 60° C. |
| pro-site b (Ni13) | rev: TTTTTGCTGACGGGAGAGAT | | |
| clone 113 | fwd: TCATCACGGACATAAAGATGGA | 160 bp | 60° C. |
| site a (Ni61) | rev: GCATAGTGGTGGGGGTTCT | | |
| clone 113 | fwd: AGCAGAACCCCCACCACTAT | 187 bp | 60° C. |
| site b (Ni73) | rev: TCACCCAGAAAGTTCCCTTG | | |
| clone 113 | fwd: CCGGTCAAGTCTGAACCAGT | 126 bp | 60° C. |
| pro-site a (Ni5) | rev: TTAACTAGGCAAGCCCAAGC | | |
| clone 113 | fwd: TAGTCCCTGTGGCTGCAGTA | 232 bp | 60° C. |
| pro-site c (Ni116) | rev: TCTCAATGGCTGATTACAGGTT | | |

amplification settings (T3 Thermocycler, Biometra):
initial denaturation at 95° C. for 15 min (Thermostart activation), denaturation at 94° C. for 30 sec, annealing at different temperatures for 45 sec, extension at 72° C. for 45 sec, final extension at 74° C. for 10 min, 45 cycles

SUPPLEMENTARY TABLE 3
(sequence information clones)

| | | | | bp relative to transcription |
|---|---|---|---|---|
| | gene | | | start site NCBI GenBank |
| clone | name | Acc Number | chromosome | Version Build 34 |
| 23 | RSK4 | NM_014496 | X | +13768 to +14368 |
| 113 | PAK5 | NM_045653 | 20 | +246494 to +246839 |

Supplementary Table 4 (RT-PCR primer and amplification settings)

| | | | | |
|---|---|---|---|---|
| | | | fragment | |
| gene/clone | accession number | primer | length | annealing |

| | | | | |
|---|---|---|---|---|
| HNF1α | NM_000545 | fwd: TCTACAACTGGTTTGCCAACC | 313 bp | 60° C. |
| human (Th#55) | | rev: GGCTTCTGTACTCAGCAGGC | | |
| ApoB | NM_000384 | fwd: TTCAGTGTGGACAGCCTCAG | 350 bp | 60° C. |
| human (Ni94) | | rev: CATGGTTTGGCCCATATTTC | | |
| AAT | NM_000295 | fwd: AGGGCCTGAAGCTAGTGGAT | 423 bp | 58° C. |
| human (Ni24) | | rev: GGTGCTGTAGTTTCCCCTCA | | |
| ANG | NM_000029 | fwd: GGATGAGAGAGAGCCCACAG | 351 bp | 60° C. |
| human (Th#83) | | rev: CTCACTCCATGCAGCACACT | | |
| clone 23 | NM_014496 | fwd: GGATTTTCTCAGGGGAGGAG | 311 bp | 60° C. |
| human (Ni69) | | rev: AATCAGCACTCTGGGAATGG | | |
| clone 113 | NM_020341 | fwd: GAATCAGACAAGCCCTCAGC | 309 bp | 55° C. |
| human (Ni72) | | rev: CCAGACGGGTACTGGTGACT | | |
| HNF4α | NM_178849 | fwd: CTGCTCGGAGCCACAAAGAGATCCATG | 370 bp | 50° C. |
| human (Th#588) | | rev: ATCATCTGCCACGTGATGCTCTGCA | | |
| clone 23 | XM_228473 | fwd: AATTGGCCCTAGCTTTGGAT | 217 bp | 60° C. |
| rat (Ni126) | | rev: CTCTGAGAATGGCCTCTTCG | | |
| clone 113 | XM_230625 | fwd: AGACCCCAGGGAATATTTTGG | 240 bp | 56° C. |
| rat (Ni127) | | rev: ATGACCACCCAGAGCTCATC | | |
| HNF4α | NM_022180 | fwd: GCCTGCCTCAAAGCCATCAT | 274 bp | 55° C. |
| rat (Th#84) | | rev: GACCCTCCAAGCAGCATCTC | | |

amplification settings (T3 Thermocycler, Biometra):
initial denaturation at 95° C. for 15 min (Thermostart activation), denaturation at 94° C. for 30 sec, annealing at different temperatures for 45 sec, extension at 72° C. for 45 sec, final extension at 74° C. for 10 min, use of various cycle numbers to demonstrate linearity (40 cycles for tissue comparison)

Supplementary Table 5 (Real Time PCR primer and amplification settings)

| | | | | | | |
|---|---|---|---|---|---|---|
| | | | fragment | | | |
| gene/clone | number | primer | length | annealing | extension | fluorescence |

| | | | | | | |
|---|---|---|---|---|---|---|
| clone 23 | XM_228473 | fwd: AATTGGCCCTAGCTTTGGAT | 217 bp | 68° C. | 9 sec | 82° C. |
| rat (Ni126) | | rev: CTCTGAGAATGGCCTCTTCG | | | | |
| clone 113 | XM_230625 | fwd: AGACCCCAGGGAATATTTGG | 240 bp | 68° C. | 10 sec | 84° C. |
| rat (Ni127) | | rev: ATGACCACCCAGAGCTCATC | | | | |
| HNF4α | NM_022180 | fwd: GCCTGCCTCAAAGCCATCAT | 274 bp | 55° C. | 11 sec | 88° C. |
| rat (Th#84) | | rev: GACCCTCCAAGCAGCATCTC | | | | |
| MitATPase6 | AF115770 | fwd: CTAAAGGACGAACCTGA | 315 bp | 55° C. | 13 sec | 83° C. |
| (Th#643) | | rev: TGGCCTGCAGTAATGTT | | | | |

amplification settings Lightcycler (Roche Diagnostics):
denaturation at 92° C. for 120 sec, annealing at different temperatures for 8 sec, extension at 72° C. for different times, fluorescence at different temperatures

1. A method to screen for and to identify anti-type 1 and anti-type 2 diabetes mellitus and/or anti-nephropathic and/or anti-neuropathic drugs, including anti-diabetic nephropathic and anti-diabetic neuropathic drugs, comprising the use of kinases RSK4 and/or RSK5.

2. Method as in claim 1, wherein drugs regulate the kinases RSK4 and/or PAK5 and are used for the treatment of type 1 and/or type 2 diabetes mellitus and/or nephropathies and/or neuropathies including diabetic nephropathy and diabetic neuropathy.

3. Method as claimed in claim 1 and 2, comprising the use of RSK4 and/or PAK5 encoding DNA.

4. Method as claimed in claim 1 and 2, comprising the use of a polypeptide having RSK4 and/or PAK5 activity.

5. Method for preparing a medicament for the treatment of type 1 or type 2 diabetes mellitus or for the treatment of nephropathies and/or neuropathies including diabetic nephropathy and/or diabetic neuropathy, comprising the use of the kinases RSK4 and/or PAK5.

6. A method of identifying anti-diabetic compounds and/or anti-nephropathic compounds and/or anti-neuropathic compounds, wherein RSK4 and/or PAK5 is incubated with a compound to be tested and change in RSK4 and/or PAK5 activity is determined.

7. A method for treating type 1 or type 2 diabetes or for the treatment of nephropathies and neuropathies including diabetic nephropathy and diabetic neuropathy in a subject, comprising administering to the subject, a therapeutically effective amount of a compound that agonizes the kinases RSK4 and/or PAK5 bioactivity.

8. A method of claim 7, wherein the compound comprises a nucleic acid encoding a functional RSK4 and/or PAK5 protein.

9. A method of claim 8, wherein the compound additionally comprises an expression vector.

10. A method of claim 7, wherein the compound comprises a functional RSK4 and/or PAK5 protein.

11. A method for treating type 1 or type 2 diabetes or for the treatment of nephropathies and neuropathies including diabetic nephropathy and diabetic neuropathy in a subject, comprising administering to the subject a therapeutically effective amount of a compound that decreases or prevents expression of a mutant RSK4 and/or PAK5 bioactivity.

12. A method of claim 11, wherein the compound is selected from the group consisting of: an anti sense molecule, ribozyme or triple helix molecule.

13. A method for treating type 1 or type 2 diabetes or for the treatment of nephropathies and neuropathies including diabetic nephropathy and diabetic neuropathy in a subject, comprising administering to the subject, a therapeutically effective amount of a compound that reduces the overexpression of a normal RSK4 and/or PAK5 gene.

14. A method of claim 13, wherein the compound is selected from the group consisting of: an antisense molecule, ribozyme or triple helix molecule.

15. Substances which regulate the kinases RSK4 and/or PAK5.

16. Substances according to claim 15 which regulate the kinases RSK4 and/or PAK5 to a normal (functional) level.

17. Substance according to the claims 15-16, wherein HNF4α is selected for regulating the kinases RSK4 and/or PAK5.


SEQUENCE LISTING (4 sequences)

SEQ ID NO: 1 - DNA, 126932 bp, Homo sapiens
  promoter (1)..(3000): SITE
ggcattttggtcaaagccatttaacaagtctctagaaagtttcaaacattcccacatcttcctgtcttcttttgagccctccaagctgttccaacctctgcctgttacatagtttgaaagtcgcttccatattttccagtatgtttatagcagcaccccactaccttggtaccaatttattgtattagtctgttttcatactgctataaagaactgctcaagactgggtaatttatttaaaaaatagaggtttaattgactcacagttcatcatggctggggagccccaggaaacttacaatcaaggcaaaggggaagcaaggtaccttcttcacatggcgtctggaagaagtgcccaacaaaggaaggaagagcgccttataaaaccatcagatcttgttagaactcaggcactatcatgagaacagcatggtggaaaccacccccatgattcacttacctccacctggtccctcccttgacacatggggattatggggattatggggattcaattcaagatgagatttgggtggggacacaaagcctaaccatatcaggtatggatgggatatgggaatacctagaacgctgaaaaagaaagagggtgggactgaaccttgaagttccaataggcagaattgggaagaggagggatgatccagtaaaggagacagaaagatgtataagagaaacaatgatgtaggagaaaaacaaacattatttcgttccagaggccaagtaaagaaaatgtttaaagaaagagtaagtcatgaacttacatcaaatgccagcaagtgttcaagcaagctgtcaataacatgtttttgctgacgggagagatccagtagagagaaaaaaatagatgatgttagtgggggggcgggttgcagaacaaagcctttgagtaggcaagaagggatggatgagacccagcgctcaaaaggttggcagtaccaggcacagcagctcaggcctgtaatcccagcactttgagaggctgaggtgggcggatcacaaggtcaggagttcgagaccggcctgaccaacatggtgaaaccctgtctctactaaaaatacaaaaattagccgggcgtggtggcgcacgcttgtaatcccagctactcgggaggctgagtcagaagaatcgcttgaacccgggaggcagaggttgcagtgagccaagatcgcgccactgcactccagcctgggcaacagaatgagactctgtctcaaaaaaaaaaaaaaaaaaaagaaaaagaaaaaaagttggccctagttaatactgtcagttcagccaaatatgagtttatgggtgtgtgcgtgcacacacagacacacacactctcacacattttcacgatttatagccccatgcacccactactttcgaagctttgacttttagagaaccctgttcgttaagctcactttctcctcgagattttcagcagagtaagtgagaggacattatcagttatccttgtttgtaattcagtgcaatctctgtggccagctttgcttcattagctcttgagaccagatgatgaaataaatggagggcataggtcaacagccctaggtgtagaagggtaaagggggtggtgtgccttttatgaacgttagaaacctctccaaggtcctgagttggtttagccgctgttacagttgctcttttaagtctttgaagtgtcaaatgtggaataacttttaaactttcagctctgaagtgttttgatgagaagcatcttgattatcccccgtataagattatactttatctctgttatatcgccagctcccagtacaatgtgtggcttagagtagcgaagtagtaaagctcgctttgaagaaggtactaaaaactatgattattgtcactacagacccaacattctcagagggggctggaggatgggctagcttcggaatcatgcctttgttgccacaagtggagttcattaagtggcatatgtttgcctgtgtgtgtgcatgtgtgaagagaaacattgtgtgttatcaatagtgtgaaggcaaccagcaggagcatcctgagagggacaagtaaggtgggggctagagaggacaggtatatttatttttttaaatgggggttatcctgcctttaccttctaggaggataatgtatgatgtactaaaatccctgccctagtatttgtcactaagtatcgaaatgggcgtggtttggcatataagacacctgctcttcgtgagccctgcaaagagatactcttggagtcctacccttaccctgattcctcaagtgctctggagggtcccccagttccagcatcctttttgagaatcctgccgcccgccagcaatgctgctccgtggacttgagagggcctgctgagcatgtgtgaagagcgctgcgcatgctccgtggagtgggcaggtttaccgaggcgctagcaggggcttttactactgttcagtcgcctccccagcccagcacgcccccctcccctctatcccaccacccaccacgcggtcccccgcatccaccccgcccctctatcccgcccccttcaccccaagctggggaatctccattgacgtttgggtgacgccgctgtgccgccgccgtgggggggtccaggcgccgcacaggtaacgccgccgctgccgccatattgacagagcagggagcggggaggggacccgaggaagtggggtgttgggggagtcaccaccgagcggctgtcgttgtggtggcagtggtgagcgcgagccgagcggcagctcttggagaggcagcacagagttgtcttctctatgggaatcaccgggcggcggcggcggcgacccccagctcccagcctctgagagacccggcggcgagggcagcgctcgctgaaggcggggctgggggtcgcggcggcggcggcggcggcggcggcggcggcgggccagttcaatagacaggatgcgcggccgcgccagcggtaggcggcagctcctgtttgagtgctcctgaaggggagatgctaccattcgctcctcaggacgagccctgggaccgagaaatggaagtgttcagcggcggcggcgcgagcagcggcgaggttagtgtggtggccaagacgggctggagccaacccccgcctccttcccctcaagaccttggctggggacggagagaggagaggcgtagggccgggaagcgggctcccgggcaagcccccttgatgctgcctccactgccctttcgctggggagcgggcggggatgagacggcgggctcgggcttgcggagtgagtgcagcctgggcccgggctcggccttgggcggagcgcctgcctggcgggccacgccagagttgttgtgactaagtttcttcactttccgacccgaaggggaactgggggactggcggggtgctgtgcctgactccgcgcgtccctttttctccgccgcgcctagtttgctggctgggaacattttgggggacagctgcggactccagctcgctgttgaaccccggacgctttacgaacccagtgcaacaaacacaccaaggtgactaagtaactctggggacatttgcacataggcacgagtgttccctggtaacagctttctcgtttctctgtggcttgttatggttccttgtaatgacccatgtcgagatacactggcgacctatctccagtcgccaggtttcaggtgatgttctactgcttcctctcaggtccccttgattctactgcactcttgctccaccccttctcctttccacaaccgctgtccctgccccgttggtctgtaaaagaagacgcaaagtaaaccccagcccttataaataagtcgaatctttggtaagacttagcgaggttcaaccaaaatactttggttcacctctcttcccactcttcttactctgaaactgttctggttgggctttggtcagctgtgcagtgagaacttagatgtacacaggtgctttgtttttattgttgttataaaagcgggcagcatttaaacacaaaaacgtgtcttttgtgatagaaagttggatccttttctgataacttaatcgactgcgtactactccagtagaggccaccatacacctcctaaaaatagttggtgaaattgattcacagtaggatattttgaggtaagactctcaatcctcgacatctaaagggtgcaactttgtcttcaagtacttgctccatttaaaaaaaatacacatttggctgttgtatgattaccttgtttttttttctttaaatttttttgttatttatcaactaaactggggttaagattacatgctagaataacatgtggtaaggaatatagtggaaataaattcagaagtaccctaaatctgaaccctctatacttacaggaaatattttactagtaaaccaagcaattccaatatacgttttaatattttacatctcgtaagaacaagataaatgaataggcgttcctttcttgttgaaatgtgggaatgctaactgtttgcacaattgtctagcaagtctattgaaggggagaggaactttgttatattcatgtatgtaacttactgaacttataggcatttagtttataaaaacagttaaatgctttgatataaggatgggatgggaggtgggaatccttttgcatgcctactattttattatttctgttaactttgaatgtagtatgttttaaattgtaattaccacatatggtttttttattgtttgcagtatgttttaggtattgtagagatttaacaatgggtctctctacctcagcaatttggaaaaatactcgagtggaaattgttaatccctatgaagtaaaacgaaaggtgaaggtaatgtggtgaagatagtatttctgccttctttctctaaaacagttatgattggttagatttaaccctgcaagttacttaataaaaaatattataggtgaaaactaaatgctacatttgaacagacccacttagtacttagaagagtttgtttgtttttaattatattcttttgataagaagtaaacaagtttacttttgataatttatattttaattacaaatttgttgatgttggctttaaaaatgctaattttgagtacaagctctacaaccctcaattgtgataattttaggactaagagtgtttttgttgtagaatatatatttttaaaatgtgctagaatccttaaagaaaatgtaggtaagaatctgttaactttaatgtcaaaagaaataatttccgtccatttctgtgtgtgttgaatctggtaagattaaagattatagacactaccatatattttggagtctttttgtatcttttacctgtcttgcttgcatggacagttgttaacctttgaattatcattcagaagtgtgtaaattggttgtttgagactcttaacatgttttcccacaggaaaaacttattaagggtacttatatttttagatcagtacataatagcgaggtataatgatgccgagcctaaccactgtttgtaaaattgttctgtggtgaaatgtgtcaggtttcaaccaataactagcttggttccagtgagaggtagagaatgggcctgaaaaggggaaacctggaatcttttggtttctgggaagagagttcttagaagaggggaaatagggtaagcaagaaactgattgtgagagctcttgcagggactgtctcaatgaggtctactgggaataggaacttcaaagggcatcacccctcacgcagtcacaatttaagcttttgactggattctgtcaggatcaagaaagaggttttacttgagatgggatgggtattggtcattcctgggcaaggatcataaagaacaacctgtttgactaatgtgaacttttcatgatttaatgccatgcatgttacagtttgttcgcttagttgtttgttttttggtgaggaaccaagtgcttctttatcaagttgatgatgtaattttcacatttcatatcatagtaaacgtgtagtacttttatattatttctttagcaatgctgtttttatttaagcaagttagttttttttttttttttttttttttgagtcagggtctttctatgtcattgtacattgtccaggctggcctcgaactcctggactcaaacaatcctcatgcctcaggttcccgagtagctgggactacaggtgctcacaaccatgcctgtctaaacaaattagttttgagagtatgtagtttactgataaaaccaatttgggaaaaacttctagttttaattaattttcatgagaccctatagtaatagaggaaagaacagctttgaaggcaggtaaatctgggttcttgtccgagctccatgccatactcatgatgtgacacaatccacgttacctaacctctctgaacctcactttctttgtctataagttggagaaaaaacatcttcaagagttgttatgtaaagaaaattcagtgctgggtttaataaacttagcaagatgttttgcacttagtctctagtacacaatagttccttctcttttcccacagtcatcatgcctcttcccatgcccctaaagtctgaactattgtcatctatgttaaaatcctgggattcagataaccatggccttagccgcagccttccatttcgtaagcagtttttcttaagaccccttcctaaatttgatcatttcatctaagtatagtggtgctctcactgtgaatgtttgtaatcacagatagggtgatccagagataaaatacaaagttatatttagaaacagtggaaggataagaagagacagataatttcatttattatatctggctgatgaggtgtttcattgaaatttttaattttttaaaaaaattctgaactcttgtcatggtaggaattggttcaattcatgaagcatttattgagtacaaggcagtgctgtacatagtgctgtaatgtgaaatgcctaaaaatataccacagagtcccaccctacccccacaggtttacattcaggtagtaagacaggcatgtatggaaattgttattgtaaaaggcacaagattatcaacaatggggtggtggtggtggtggtgggggtggtggtggtttaggaatggatctggtaaggctttgtgaaggaggtggaatttgagcagggcattagaaagtgataatattttcacagacagatgaaaggagcatatttcacgtggatgggcacaatataagaaaaggcatggaaatgttctgaaagaaggaagaatctgatactgttgtaagtctgtgatgtgggagaggggatatagtgagagataaggaatgtattaaaggtaagctaggcccaggttgaatgggactttttaagccagtttaagggttcaattttatctcataaataggagccactgaaggtttttgaggacatgagtgacattattagatttgtgctttgaaaaataattgacggtagtgaggaatgggggagatatggaaggtggggaattccagctaggaggctattgtgttagtggaagtagtagtgagatttttgaactagtacagaaggggtagaaaagagatgactgatgggagagacctggcagaggtagaatagttacaatttggtaactgattagatgttggagagtaaaaagaaataacaaacaattgagattagaagcgtcctggtaggaattaattcaatgtaacaggtatttaatgcatacctgccaatataagtctctgtattagccaccagactaatataaaaataaatagatctttgcagtatagggagatagaaagctaaaatgaaatctcccctttctaatattgtattgaataatagcattccactctgaaggtttctgatcgtgcttagaatgcttaaattctttactaccaaaggtgaatacacttattactagctaaaacatttttaattctttttaaatgggccttaggcctgtaattcttgtttcaccaatgttcatcttcagtagtcagcagccgtgagtacagagtcctattctgaaaataatgaccagtacttatttctggaatgtggaaggagagcagggacatctgtattttttctatgctttcaccaggtttatttggaatagacaagcggggaggagtcagagggcagtggcccctccctgccacaggtgccttctcagttcctccaagtcaatctttctttaagccagtctgatctggaaatattcaggtcagttccttcagggcaggtcctctatatttccctaccttgattagttcatcaagagagctaatcattacctctgtgttccatgataaggcctactgatagtgtagttgcttttctgggccatattgttttagtacctaccctctcatagtctctctcatctagaattttgatgaggaaatgcaagatttttttcccttcctgtaatttctttcttggtgctatcattgtatgtagcagcggttcttaagatttgataagcacttggaatcttgaggtataagcctttcagtactttgtcagaatttgctatgtaatgtttattcatttatgttatttccaaggattatttactgttgctgccaatatttttttttgttgtgtattttctttatttttgtttgtttttaggtaggaaaggagttagatgggagagagatttagagagctgagctaaatctggcttttgcagataagagacgttttctaaattgccctggagacattctgtaactgatttcactgctagattttaatctgtatctctacattagaaacaggtcctgctgtagttttgtgtgcacgtgtgtgtgtgtgtgtgtgtgtgtgtgtgagagagagagagagagagagagagagacagagaaagagggaaggtggagtagagtgcatgtgtgttacttgtgatttgcttcattttaccaagcatttgagatggtatatgggaaatataatctgataactattttgatattgaggataagggaaaacaaagtaggagatatatgaaaagggtggagggtaggggatgaagggtaaaatagagcacaaataagcaggctgtaagggtctatgacatgtttaaaatatagttgaattataaatttggccttgagttttcttagctggtttttctcaactctggctgtgtgttatatttacctgggagtgtaaaaaaattatccatgccagtgttccactcagagattcacatttaactaatctagagggggcctgggcatctattccccagatgcttatgatgcacagccagggttaaaaaccacagctctaggccaaagtaaaaaaagaaatagtcacgtagttgtcattattcaataggaaaaaaagaatattaatttgtaagctagcaattagctctgcagtaaattatctgtgaattgtgatatttttctgtgtgttttattttgttgttgttttaagatatgactggagccaaattgggttgtttgaactgccctgtatgttcttgctctttagttttgaggaatttgcttttccagtagcattaaaaacaaaatttagtaccgtagctagttttgatatagttcagccatataactaagaggttaaataacttgttcacccattttactagctgagtaaggagatagtgactctgttttgaatgacaaatatcccattacatctctaacaaaaaatttcaaaacccatgggtaattattactaagaaatccctctgcctctcctgtaaagtgattgaagacaaactttaagaggaaatatagcaatttaaacacccaatacaaattttttaacctgaaaaaacgttggaatccattccatttaaaggtatcgaatcttaagggagaaagagataatagcgtttattgagcatgaactatgtaccagctagtactagatgcatcatagattatctcagttttattcattcttcaaaacatcttggtaagacagttatgtctttttaacagaatagaaagatacatgcaagttagtaggaaatacgtctggaatttgaacctagattattttaaattcatgctttttcctctatacattgctatctcccccctaaggacaatattgaaaataaggagagaatatagatacgttgttagcaaccatattagatcagcagtcagatgtgctgattcaatagccatgcctgtctatttaagttaaataaatgtcaacaaaatcccagcaagtctgtttttttgttttgttttgttttgtaaacagacaagcatgttctaaaatgtatatgtaaaagtacaagacctagaagctctaaaacaattttgaaaaagaaaaataaaataaggaattactctattggatattacgttttactatgtaactataataaagacaatgtggtattggtggaagaatagacacgtagatcagtagaccagaatagagtacacagaaatagagccacacaaatacactcaactgaattttgagaaaaaatgcaaaaaaataaaaaaataaagaacaagatcagcttttaaaatctgaggaaattttagggggaaagttgctacctttttcaaaaaggcagaacttgaaatttgcactttctttctgaagattctggggaaattattacataaaacatttttgctattttcagttactacagttgaaattagtcattaagacataagtgaaaatggaacatgaattatcctaaaaatcataagcttgataagagttgaggtatagggatttattactctacttaaaggatcagagtggacagaacttcagaaaacaaaggagaaaaacgtcatatgaggggaaatgtggactgaatgtcaggtttcaaatgacttttagtttttcttaaaatgagagaggtttggtgtatgtgttaaagaaattgtactaattgagaatagtcaaaattcaaaattaagggaaatagataagccattaggtctgatttgataaaaatattttgcattcatgattgttcacctggagtagatgcagtgatgaatgaatgctgttggaaacaattacatttcaaacttaatgggcatatgcagaaaaccatatgttgattatagagacatatacaagaatgatagaacatatataagataatttgtgtatgattttgtactttattttacagatggagaagagtaactctagaccttctggcacaatcataaaacatttattgtgtttatcatttgtagagcaattacattggaataagtgaatacttatagaatctcttaagggaggtcagtcaatacggattttcaaaaaagacatggtcattcagcaacttcatatctatgaacaatattaatttgattttatttggtctttattcatgatattatgtgtatcagtagttctttcttttttattgctgtgcactagtctgttatgtgaatgtaccacagttgatttgttcattaataagttgaaggacatttggatttgggttgttaccagtgtttggtgattatgaataaagccattatacacatttgtatgaaaatttcggtttggacataacttttcacttatattgggtaaacacttaggagtgaggttgctgcgtgagaattccagctgctctgagtacttgccagcacttagtcttgtcagattaagtgaaaaaaaatagctattctaattggagtgtaggggtatctaattgtgattttaacttgcatttccctaatgacacatgttgctaagcatattttcatatgcttatttgccatccgtatatattctctgatgagttgtcttttcaaattttttgaaattgttcaagttgggttgttttcttattattgaattttgagagttctttatattccaaatgcaagtcctttattagataccgatttccaaatatttttcccacatggtctgtcttttcattctcttaataaagagtcgaggtttttcatcttagcgaagtccagtttatcagttttttttttcttttatgaattgtgtttttggtgttgtatctaagaaatcctagcctaatccaaagttacagaatttttcccttaagttttcttctagaagttttttagttatatttcttaatttaggtctttgatccatttctagttaatttttgaatatggtgggtagtgtgatagaagtctacttttttacacaggtatgtccagttgctctgccaccatttgttgaaaagactacctgttctctattgaattgcctttgtactatatcaaaaatcattttgtcacatatgtgtagttttcataataattttaatgaattagtcctgtggaacttcagaattgcacttactgagcatagatatatggtgttaataaaattaattaatacaattaataaaattcttgaggttgtttctgtattgggtttagtgaaatattctttgttaaaaaacaaaataatttctgtgatatattttctttgcagtcattaattttcaaaacattaagtgactgttcttagacccttttatagatgaggaaacctaatggtgctagaagttttatgtgaactttctcattctgcagtgatacttctctgcttatctcctcccccatatttgtacattacccctaacattttttatatagcagtttgtgacctgttagttgcttagtattagacatgaaagctacaagactgaataagcttcagttcatattctagaaactatctctttgccaagaaaatgggagcctggattgttggagctttggtaaccatcattaacatagatttaattttgctctttttaaataaaattattttggcttggcacggtggcttatgcctgtaatcccagcactttgggaggcagcgtgcgaggaatacttgagcccaggagtttgagaccagcctgggcaacatagtgagaccctgtctctaaaaagaataaaaaattagctgtgtgtggtggtgcatgcctgtggtcccagctacttgtggagctgaggtgggaggatcacttgagcctgggaagtcaaggctgctgtgagccatgattgtgccactgcactccagcctgggtgacagagcgagaccttgtctcaaaaaatttatattttagtttttaaaattgacaaataattgttgtatattccttttggttttaagaagagaatgatattcgttctttgagaaacattttaaaactaccttctttaaccttggttaatgatactcagtgcctcatgtcttcctatgtgaaagaggaaatatgagaataatgtaggttgggtttgtattgagaatattgtgctaattaagttgattttaaacttaaggaaatataaacagttgggttagccatctcagattctttacaaattcagaataccatagaggataatagctggttccctagagctagaatgcccgagtttaaattctggctctttatttatttactgtgtgaccttggacaagttacttctctgtagtttacttggtcgctctatctgtaaaatggggataacagtagcatataatatatgtaaagcacttagaagagtgtcttttccattgtaagcactcagtaaatgttattatttttaaatatatcacatctgcaatctgagttattgaagattaacaggcttgcaagttaaaaaaatcaactattgtataatgatatttaagtatcatatataattatgcagttattagtatttcttctttaaaggtatttatatacaaatacacatttctcctttttgaccatcttcttctacctgtgacttgaattttgaaacattttattcttccttgattagctgtagtaaggctgtctttttttcgtattcatttctcaagaggatggtactgcttttgctgaacttatattttctttattcactgagcttctgttttctgtagtctattgacttgtgattttctctgagtagttttttgacagcaaacatttattaatggaaaaaaacccacacatatcaggtagaatttgacctcaagctaaggttgtgtaatagctgtctatttttgacttttttccacatataaacccattttagtttttaaattttttcctttattctccattgtttatgaagtgatgaaagaatagagtaatatggtgtaggacatttaggaactgtgactcactgggataaagttgttttatcctgtcactagcttgctgtgtgtttttaggtatgtgttatttaattaatcagtttcttcacatataaaatgggaattaacatgtttatgattattttgagaagtaacacaattgtactttgtatataatgtttgtcaagaataataaagttctttttcctttcaaaaaatgactaaagatttaggtgttctagacaaagtacttctttaaccctgatatataactcagtggacaatttaaacttaattcataccccaaaggtagtattttaatgtgattatatagacttcatatcttgaagaggttacaacagtgagaattactacctcagaatgtcatgtgtgtattcaggttgcatatgtacatgacttcatttataccacatgaatacttatatttttggaaagtaaacttcccaaatgcatgtgttgtagcccatatgagactggccagacagccagtgcctttgcattgggaacactttggagctattcaaaaccaataaagtcattagctctgatttcagctaagcagagtaaacaaattcctgcctgaatagaaaccaggaagctgcatttctgtcattgaaacaaggttgtaaaaactcaaatgttctggaaataccttctaatgtttatgcaatattgacatacttagtttaactcagtagtaacagtagttactcagtgactcagtagtaactcagtagtaatagtagtacaactactagtcctttttaaaagcatgatgataattggattttttttaattggttctaaatattattttgcatgcactcaccaaattatgattaaatatgtatagtgttgtgttactagatagggttcatttggttattgctctcaatttctgtcttgataactgaaaactaattaactgtgtatttacgcatttttccataatgtacaaagaaatatctgaactgaaggacaatatattatgtcacaaattccaacaaatttagccttatagaaagatttttaaaaatattttggttgttaatgttttaattccatactcctgtggcattattcacaggttcactaatagatctttcccacccttttgcttttctaatatattaatacccgatttcctcctactcaaataagccatcaagaaagtagagcctcggtatgtatggcagttcagtttgtgccgtattcttgctttgcatagctccccttactcttggctatgtaatacctagcatataaatgctatgtaaatagctgttaatagagataacattattgctatgtaatacctagcagtataaatgccatgtaaatagttgttagtggagataatttaaaatatagaggaagagatttaacagataaataatagagatcatttaaagtacagaggaggatgtgcatagttatatgcaaatgccacatcattttatatacaaaacttgagcatctgtggattttggcatccacgagaggtcctggaaccaatctcccacgtatactgaggttcataatgtgatttccatgcaagatatgaaatacaaagaatttctaacattttttttcttgaaaacaattttatgactaacccaaataatatatttttaatgtgattttgaaaaagctgaattcctctttgctgttagcatttaagagtcagaaatgggaagtaaaatgacttactgtaacttcgtcatgaaattatgtttgaaagttggttttaaacatttttagtaagaaaactaataaaaatcgaaggaaaaataatggccattgcttattattgaagttattttataaattttattcatttagatcccagattttagtcattccattttgtaaatatttttccagtgtctattatgttccagatattatgctaggtactggggtaaccaagagtaatatgatgagatccctttcctccaggagattacagtatgatagagagatggaagagacacttaacaagtaacacagtgtttactgatagaagtcttcatagggtaaggtggaaggaaaaagggaaagtgttaacgttgagtcttgataaagatgagttgacattcaaaagcacaggtaattcaaaatggcaaatactccaaagatcattcatatttaactttggacttggttttatgccttaatttgaacatggatatgttttaccagctaactgcttttacaacagagcctcatgattgcatttaaatacctgactaaagtaatcgcttggaggctgtatactcttgtcccattctgagactcttttaaaaaaaagacatctcttttaaattgatttgaaacataattgccctttcttaagtcctccctgaagcagtgacaataattaggtgggtgtatttcaaacttcttgaggtacaaactacaagaatattgtataattagaaagccagaaaggatcaaagtaaatcatttaaaatgagtattgtagttgttatcagatttctcataagcagtatgttaactatataatttaatctaaaagttcaaaaagattaaagtgttttggggaaatgaccatagatattaataaacctttgaaaatatttggtagtataaaagacacagtacaaattaaaacttgcctggtgaatccaaaaaaacaaaccaactaaatcttatttttggaaatgtcttgcttaaaagactgttagataaattgataaagtgtgttatagtaaaaatatacaaagatacttttttatttctctccaatttgtgtttcttaaagcaaagagtatgttcatgagtatgtatatacctttatgggttggtttgggagattcatttcacgttgaccaaaagtgatcctatcataattttaaaagtgatctttccacaacagtatgacatatcaaaactacactagggctgggcactgtggctcatgactgtaatccctgtgctttgggaagctggggcaggaggattatttgagcctaggagtttgaggttacaatgagctgtgatcgcactgccactgcactgcagcctggggcgacacagcgagatcctatctcaaaaaataaataaatgatacaaaaaaactaaattaagactattgagtcaatactattatgatagtgatatggcagtaagttttgattcacatattttttattctctatacaggcagcattattcacctctgatgaattattagctaaaatgtttttatttttttctgtattgtttttatcagaaaaacattcacacatcatgcatttgttgagcacatgtcagacactgtgccagctgctttaggaacattgtatgaatcctgacaatcctaggttgctcattttacagatgaagaaattgcaactaagaaagcttaataaacttttcaaaggcagtaaaagtagagctttagagtttgaatcttggtctgcttgacactgaagaattgctgttttcatgacatactatgcctgcctaatgaatgaaacacagcatcattcctcaaggagctttaagtctagatgcacatgacaagaaatttacctgatgtgatgtattactaatcttatttttagcattaaaattttaaattgtcttatatatataaatatatatatgaaacataaaatttgccatcttaaccattttaaagaatacagttcaaaagtgttgagtacattcacattattgtgcagccaatctacaggactgtttttaccttgcaaaactagaactccttacacgttacacaaaagctttccattctcccctttttgccagccccagcaaccacctttctactttccatctctatgaatttgactattctagataccttatataaatggaaccatatagtatttgtcattttatgactggcttatttcaattcatataatatcatcatggttcatgttgcagcgtgtgtcagaatttcctttctttttaaggttgaatgatattccattgtatgtatataccacattttgtttaacttaccatctgtcgataaatacttgggttgcttctaccttttggctatttgtaaataatgctgctatgaacataggtgtacaaatatctttttgagaccctgctttcgatttttttggttctataccctgaagtgggattgctagatcatatggtaattctattttaaatttttaaggaattgctatactgctttttatagcagctataccattttacgttgtcaccaacagtgcacagaggttccaatttctccacatccttatgtatggtggaattattttgaaaaagtaaattccaggatatattctggatttactttccatttcaacaaagccatgctgttgataattcatgttgagagggttagttcttaagatactgagatatccctgattgttaaaggaatatgaaattatgttcttgcggtgattcgtcaaggatctaggaccagaaataccatttgatctagcaatcccattaatgggtatatacccaaaggattataaatcattctaatataaagacacatgcacacgtatgtttattgcagccctatttattgcagcactatatataaacactatatataaacacgcatgtttattgcactactatttattgcagcaaacacttggaactaacctaaatgcccatcagtgatagactggataaagaaaatgtggtacatacacatcatggaatactatgcagccataaaaagaatgagttcatgtcctttgcagggacatggatgaaactagaaaccatcatccttagcaaactaacacagggacagaaaaccgaacaccacatgttctcactcataagtgggatttgaacaatgagaacacatggacacagggaggggaatatcacacactggggtatgtcgggggttggcgggaaaagggcggggagagtattcggacaaatacctaatgcatgtggagcttaaaacctagatggcgagttgataggtggagcaagccaccatggcacatgtatacctgtgtaacaaacctgcacgttcatcacatgtatcccagaacttaaaataaaattttaaaaaaaggaattacgttctctatactgtgcttaagttagtgattgatttttcggaaagcaggattaaaggacaaaaatagaactgctcaattttaagtttattgaaactaaaattaaaaatttggccccacttaaacatttgtcctgggtcacttactagtatatagtttacagttttatttctaccaaatgagaacaaatatgtgagtttaataagtaaagatctattttaaaatgattaaactgtaaaatttgtctctggaaagtctgtttcaggggagaatttttgatcatagacacttttgattgtattacctagtgctgcaaaaaattaccataaatttagcagctttaaccaagacacatttattatctcacagtttctatggatcaggaatctgggcttggctcaactagatcctctgcttagggccctaccaagctgtaatcaacacatgggtcagcttggtcctctgcttcgattcccacaaaaatgtaatcaaggtttcagctgttctgcattgttatatagaggcttgactgggtgaataatctacttttacactccctcagattcttggtagagttcctttccttgtggttgcaggactgagggccctggcttcttgctggatgttgagtggaggctgctttcaactcctagaggctgcctgtagttcctagaggatggccacagtccctcgttatggggactttctcaacatggtcacttaattacttcaatcaagtcagcaaggaaagcctctagagtcttatatgacacagtgtaatcatatatgtaaaatctcatcgcctttgccatattctattggttggaattcagtgataggccatgttcacattcaaggggatggggattatagaagggcataaacagcaagaggtgaggatcatgggggcctatcttagagtgtccaacacattgcttaattggtaggtttctttctctgttttattttgtcctgggcttaatatataggcatagattttttttgttttgttttttgtttttttttgcctttgagccagagtctcactctgtcgcccaggctggaatgcagtggcgtgatcttggctcactgcaacctccacctcctgggttcaagtagttctcctgcctcagcctccccagtagctgggattacaagtgcatgctgccacacccagctaatttttgtatttttagtagagatggggtttcactatgttggccaggctggtgtcgaactcctgacctcaggtgatccacccgcctcagcctcccaaagtgttgggattacagacgtgagccactatgcctggccggatctttattttaggtagatgtggacccacctttatctttagctctaatttacttttgttatttaaataaagtgaaaaaattgaatagaagaagtcttctttacaaaagctatcagtgactaatatgtaaatgtaaagcttatttgagagacatattttctttttttttttttttttttttagaaggagtcttactctgccatccaagctggagtgttgtggcataatctcggctcactgaaacctccacctcctgggttcaagcagttgtgcctcagcctcccatgtagctgggattacaggcgtgtgtcactatgtacagctaatttttatatttttagtagagacgggtttcaccatgttggccagactgatctcgagctcctgatctcaggtgatgcacctgccttagtttcccaaagtgctgggattacaggcatgagccacagcacctggccaatatttttttttactaggttgattttggggatcctggagaatacttgaaaatatataatattgtcaaatttattgtggaaactgctgtcctgtgtatgttttgttaatcatttgcatcacccatatttattctcactttttatctagttcttatttgtgtatctctctactgtcttcttcggaaatgtgtatctctctactttcttcttcagaggtgcacacaaataaggaaaaagtagattttattctagttagatgtgggcctacttctaagtttcaatatatctttcagcaccaagctacaagctctgttaacttcctccttttgtgtaacactaaggaacaggtttatgtatcatagataacagtaaactcttttactaaactatttgtattcatatgttcactatcccataccacctccaattccaaggatctgaaagggatttttttttttttaatttctcagttcagttttccttgatgagttgctttggtcaataatagtaagaaagcgataaatttgaagtgacctatgtgattttctgggatcagatgagtacagcaactcagtttaggatttctgtttgtcccgcatatttgtaagtgctttaagtcttttctgataaaatgtgaaaagaatttttaaagtagtcttctctaagccctggtacgagagtctgttcacttggattttgaaatatctgagtaaattattactacctttattgttgatgataaacattccagaatcatatgtagaatttttttcctctcatttggcacttaattggggaaagcagcatgtctgcttatcttttcaaattacaaattttatttttgaaataaagatttattatcccagagaattcagaaattatttttgtttttatgtggatgaaagtgatttaaattatagtaagtatcaatgagtatacactgtggttctccgtaggatgaatattcttatattgaaaacctgataccttgagggtagatgagatgggagcaatggagtgttctgtttctaaaggccatttctgactgagataatttccaggatagagtgatcttcttgactcattcacattcattttatttctagttccttcagacccacagcttcatgataaattagtcataagatgatatgaataggaagtaaccagtaactaaggtaacagaggaaagcaatatttttctttttttatggataatcctcttcctatgcataatccttttttctatggataattctgagagtcattttatggcaaaaatttaattatcacgattctgtcttaatattgatttctggactgttttccgaatgctcttctttctatatgcttacaatgaatctgtcacagttctttttttaatcccacatagtctgttttgttgagctaaggtggtagatggtgttatcatttaatgaaatgggaaaccctgaagaaagagcagatatgaagcaggggtcaagaattcctttttgtattggttatttttgagagacatctgagataaacatgtggaaatgtcaagtatgcagttggatatatggatcaggaactcagagttagtcactgagtaaacgtgaatcatgttagtaaattagctgcgtggggaaagaatgtggagtgggatgagaaggtgatcctagtccatttcctgaggaattacagtgtttcaagggtattgaaaaaaagcagcaaaggaaattggagtaaaataaaaggagtgtatggtattacagaagctaaagactcaaataggagagtggtcacctgtgacatgctgcagagagatcaaataagataatgactgatggatccgttagcaaagtagacatcattactgactttattgaccattgctggttttcaatgggatttttttttagaatagagatgctagttgagtaaataatgctaatttactttttaataaattggtttccaatttttcatatagtaaaattcactctttttcatgtagggttctgagattaaaaaatgtatagagttgtgtaaccatcatcacaatccagataaagagttgttcgatgacccaccctccccagcacacacacacacatacacacactcttcccatgtgatgcctctttgtagtcagcctcttccctatccctaactccttgcaaccactaatttcttctccattccttttagttttaccttttacagaataacatataaatggaatcctacagaatgtagccttttgaatcttgcaccttttatttacaatgtgtttgggattcatccatgttgttgaatgtatcagtatattattcttttttattgctgagtgatactcgtcttcctctacccccaacccgttgaaccaattatctttattctttccctacagttttgctttattcaaagtatcttatacatgaagtcatacagtatttagccttttggatttggcttctttcagttagcagaaacgcttttaagattcatcgatgttgttacaagtatcaacagttctcttctttttattgatgagtaaaaagtagtattctttttatccactcatcagttgaagaccatcttgattgtttccaatttatgacaatgataaataaagctgctgtaaaaatgtatgtacagatttgtgtcttaacatgtttccactgatttcgggttaatgttctctgttttctggtctccatgatttcttacgagaaatctgcagtcattcagtttgatgttcctctttatgtaatgtgtggtttttctctcactcctttcaatattttccccatttttcctttggtttaaagtagtttacttaagatgtgtcttggggtggatttctttaattttattctgtttgggatttactgaacttcttgaaactgtaattttttttttcaaatttgggaaatgttcagccattatttatcttgaaaatacagctttagtgagatagaattcacataacatataattcacccatttaaaatatacaattcaatactttttattatattcacaaatatgtacaactatcacaacagtaaattttaaaagttcttcatcacatcaaaaaggaacattgtaccctttagttatgacctccatctccacatacctcaccccaaccaaccattagtctgctttctgtctctatacatttccctgttttcattatttcatatgaatgaaattgcctttatttcgttaagtgattttttcagtattactctgttattttatgggatgttaatgacatggattttagaatgtctcatagtgtttcatatgtctctgaggctctgtttatttttcatcattttcactcttttcttcatattggatgacatctattgatctgtcttaaagtttatttcctcagccacctgcattcttttattgagtctatccagtatttttaaaatttttcatatgatttttagttctagaattacttacatttgttttttttcatagtttctatttctctgctgagaaccttttatcatttcattcaggtgtgtttatttttatccagaaaagcatagttatagtagctgctttaagattcttgtcagatagttcaactttggagccatcttggagttgatacttgttgataatttgtttgtttcagcaggcaatctacctagttaggttgactccaaagttatgtcttgtcctctctggataatggttccaatgttaattcagttttcagagagtttgggtcagcactctgcatgtgccactcattggatgtgctaattcagtggttagtctgagacgagcaattattggtattgtagttcagtactcaaagactttgttatgtttctttagtcctttctgcacacatgcagttttttgttgtgaccaggacttgtgttggttcatacacagaattaggggatctgctctcttcattctatcctctctgatttcttccacattcttcaattcccatgggccctttattctgttctgctggccacaaatatgagtttctctcagagtcttagctttttatgctggaatataactttacactattggagctgccttttggatgaagtaaagaagagagtgcaaaaaaaataacagggattaccttcacatttttcttataaaagggacctcttttcctgcttcctctggccagagagagagattttctcttgatcagggcttgccttggagttggactcagcatcaaaaaagagaaagtgtgtacatggggaatcttggattctccttatgtttacaggctcaaaagtgcgtcttttcctggtcctttgtctagaaaatgggatttttcttggaatttttgttgatgtctactgtgtaattttgtgactcattctctcctctgctcaaattcaggagataaaggtagaaaataaacctagggaattcatcagagttaattttcaagttttgatcttccctaattaatttgtttttgtttcattttcagagttcttatgtagtttctttttgtattctacataaggattttagtttataatagggggagataggcttatgagcttatttcattttgactgtcacaagaaactctaatttactttttaatttttttttctgttgacttacaaatgggcagtggtaaaaatcctttttatctagttcgtcttcctgttgcctaattttcaaacatcttagttcagtgtttggcagattgtaagggtgcagataggaaatattttaagctttgcagacatagggtctctgtcacaactggtcaactctgctgttgtagtgcaaaagcagccatagataagatggaaacaaatgggtctggctgtgttctagtaaaatttttacagaaacggacaagaggtcagatttagctcatgggttatagtttgtagaaccttgatctaagtcttctactttactgtttcttgaattagttgttaaagaatgagtagtattacctactgagctctgcatgttgacttgttttgtttccatgattacttacatctttctcctatagtttattttttcttctgtatagtatgtcatgatgtttaattaactgaatgaaagaggaattcacaacagtgcaattatataaaactgttcttattataatttgctttgaatatacccccatggtagatttctgtggcattttttttcctgtcataaatacttggttggggacaatttaatgaaagtacataggttatatacttagtgtatagttatctgttattgcaaaactctaaacaatatgaaattaagcataaagctctgaaaatcaaagtttctcaaggataatctgggaaccatgttaatagtgttccttttctaagtacttctatgtgtcctttgtccttttgtttttcagtaagtgtggatcttgttagattgtggaaggaaatgcctagggttatatttttcttagttatcactcttgttttaatacttttgaacctcaatttttgaacaatgtttttcaggtaaatggtcttaaaatggttgatgagccaatggaagagggagaagcagattcttgtcatgtaagtatttatgttaatttattaagccacatttttagcatataaggaaaaagaatacagggaaaggagaaaagctcctatcaaacaaatatattcaggtaagttggtaataagcaaatggaattattaataactcaactgcagtttaaaatataggacaggttttcctgacagaaatacttatgcatgcatttttaaaaataaacagctttattgagacataatttacatgctattgaattcacccattttaagtgtaatggataaaattttcagttgccagtgtatgagtaactgaattattatacttaaatatagaatcctttgagaagattatcttcaacctcagggtagaaaggatatctgacttggaaatattgaactgtataagcataacttgaaatcatttaaaatcaatttcctggcttttagaaaggattatgtgtaaattatttcagaagtttctaactagttttaaagactgcttagccattagaccattctatgtaatgcctagatactagagtcttattttacaagatctagttttacagagagcattcttaagatttgtagggagaatctgactaaatcagcatctacatttatgttgttattttatggttgttatttctatagatattttcatagagttatagattttttttttttttttttttttgagacggagtctcgctctgtcgcccaggccggactgcggactgcagtggcgcaatctcggctcactgcaagctccgcctcccgggttcacgccattctcctgcctcagcctcccgagtagctgggactacaggcgcccgccaccgcgcccggctaattttttgtatttttagtagagacggggtttcaccttgttagccaggatggtctcgatctcctgacctcatgatccacccgcctcggcctcccaaagtgctgggattacaggcgtgagccaccgcgcccggccgagttatagattttttaagctggaaagaacctcagagatattatgtaatacttcattgtatggattagtctactgaaacttggagagtctgtgatttatactaggtgataaaagtagctagtagcagaatcaagacttagggcagtatgcttccttttaggtcagatatattatgtagacctgaagaagtagaagatgaggagaacattatcctttttctatttggttgtggatgaaatcaactttgatgacccctccaccaaaacttgttcataattggaattagctcagatagaaaccagcagagggatttaaattgggtttgtccaaaactctcctttgaaatgttctatgtaactatttctgcagttagtaaccatgtaacaacacttttaattttgattgacaatatgagagatacaatgttgtctttgtagcttttgcagtttactgaatattaataatatattgtaattttgagtataattaaatgtaaactatcaaaaccattatctagagatttgtctacagttcatatggactaataggcatagtaatattaatgttgccagtttataaaagataaaggagtataaggaactattaaaggcagtggagaaggagaaggctgcttattatattttagcattaataaattcacaaggatatcatggcttacaccacaggggaacagtgtgtgtggtgcataaactcaaggacacatacacagggtgattggcaaatatttacctttataattttagtagaagataagtcaggaagttcagtttttagtggaaaggcagtaaaaatatacagtgctagttacttacatgatctaccttatgcaatcaacttcagtggctaaaaggtagagtgtgaaattttggagagtaaaggacctagaatcacttccaaggatttgatttagtttgtggcctgagttatctggacttgaaaccatgtactttgggtgtgtgatggttaaagtactcaatttgtgtcaataaaactacatgagtgattaaaaacaccatatgtatttatcccttaaattttttaaatatacagctacgatttttttctttgtgattgtcatgtgtttttttgtggttggcttttttttttactgttttaaaatagtgtaggattgtagagactgctctgtgatggtaagtattaggtaacaggttgctgataaactatatctaatatcacatttcaccacctagtatcctacttctttcagcaatgtcaaggttagaaaaagtttgttatgatttgtctcctttagtagttggctgacatgagttaactttttatttgttgtcaaaataagcataactgccttatatttggtagtttatgatccattggtatttatttttaaaactctacccagtatctgttttttctgaaataaaatacatatttttaatgctatcaatatcttaaaaatttcaaagaatattcagttagcaattagattctttgttgattacattagtgcaaggtcagcaacatgtttagtctctaaagtgatttatatttttattatgcagagatgtaaatgtcatagtgaactgaaaatgtatattttgaaaaacaatgctggatatcctgatttttttaagccctgaatttcttattttaaaaaccattcttgacatgtgtctgttaagaattctttcaaaaatacttttttacaagtgtgttttacttagactcagtggaaccacaggtaggagctatttcaagctgagaatgcctgctttctgtatggtggatttttttttcctctatttgtctttctattctgatgcacatttagccttagtatgcttcagtattttttctcttgcaatgctgatgtttacttctaatttctattcagttacttaaaaaactggaaaccttgtcctacagatatgcaaaactctcagatgaataattattgttcaactaaaaaatgttggtcagccactataaagtcgagttatgctgcaccatcaggtatgcaaagataagtactacagagagtatctgactcaggatggttcaacttatgagtttcaactttatagtggtgcaaaagtgatattcagtatgttcctcaacttacaataggattaagtttggataaactcactgtaagttgaaagtgaattttcaacttacaataggtttatggggacaacactgtcataagtccaggaacatctgtacaaataagttcagtaacttcaatttttatggatgttttgtggttttcaaagttctcagttgatccttataactgagatctgaaatagagtaagtattactgccaatttgtagatgagggaactaaggagctgaccctcgtaagtagaataaaccaaaccaagtgggaactagtttgccggcattctttggttttcagtcataatgcagacttttacaaaactaagtgttctgcagataccttccattgtgtacataattttgatgtgcttagttcaccctcatggcattttggcatttatattggtattacgcttagctttctttcaaagtacactaatatttgacatggttttcaatgaccccatattattcactgaatgagaagggtatatcgagctgctcttctgggtctttgcatatatgtttcagcaaatccacaactaaatgattcttggatttttagggtgatcttttcaccaagctggcctgtgtacctacaaataaagctgatgtttgaatagatgaatgttttattattaaataaaaatgaacaagttcaggctttaaaaatacttcttttgttttgatttgttttctggaggttagttccacagtcatatgaaaactgctgtaattgagttataatagtatattgctattaggttgtcatcagctttttctcttttcaattaacaggtgattccatcttcctaaacaaatcagttatcaaatgaatggagcaatagtagatttgacagttcattttgtactttaacagtgtgttttcccagtgttacagcactgttggccctctggattaaaaagatgatcacagattatagaggtcagaacatactattttggttgtggttgagccttcatactttttctgtatatcactgattactttatggtcgcaaaaaaataataaaacctaattaatagtcatcctggcctcagattcagattttttatttgatgtagaaagactaggggtccttatttagaggttattaaatcttcatttcacagtagcaacattattttcaaagatcagctaagattatgtaccatgtgtagtttgctagggctaccctaaccaaataaccacagatgggtggcttaaacagcagaaatttattttcccaagatgctgcattctataagattgagatcaaggtgttggcagggttggtttttctgaatcctctttccttagcttatagatgagtcttctccttgtgcctttacgtggtcattcctctgtaggtgtaggtattctaatttcctcctcaaataaggatgtcagtcatattgaattagaacccacccatatggcctcattttaccttaattacttctttaagggccctatccctaaatacagtcatgttctgaggtactgggagttaggactttaacatattaactgttaggggacataatttagcccatagcatacttgtagctttttgtttctttgacggatgtgatgtttctgacaaagttctctatcctcaaattcacagtttatttctgtatccccagtgtgcagcacagtgtctgataataggatatgtgcttaatgaatgttgatttgctgggtgaataataagagtgcatatatgggaagataaggtgatatagcacaactttatcataccaggatagggaatttttttaaggatgatttactctcaggaaaaaagaaatatatcacttataagtacattaccacctatttcaggctagattgctgctactatcaagcttttgaaatggtgcagaacctgaagacagggaggtggagggaagaaagagggtttcagttgacaaaaagggacattataatagtaaaaatcaataaacatagccttgggatggctgagaaattttaggcaatagacaagaatggggcagggagcagatagaataagacagaataagtattggaaatggtgacacaggagggtatcacatactttgagagtaaaaaagagctagtggcgtagaaatgataaaagtgttaagtagctatatataaacacatacacacatgtgtatatgtgtgtgcacgcatgtatgcacatatgtgtatgtatttgtgtgtatatctattatgtatctgtataactataaatcttagaagcacccaaatattgtttttcaatcctggccatgtgtccgaatcaactggggaggcttaaaaaatacacatgcttacgttttcctgtagttctactgaaccacaatctctgagggtgggacccaaacaagtgcagtaaataaatatttcttctacaggtgattcttatgggcaaccagggcattacaaattgtctgtgtaaaggcacagtagtcataatttggtgatataatatgtggtagttgttggcctaggaacatatgtctttatttaaaaaggcaaatggctaagcattaggttaatattcaggggttattgtgtcctttatttgtgttgagtacaacacatactcagcacagtgtaataaaaatactgacaatggataatatggaagaagtttatattcttcacagtgtatgggaagatatgcttattttatttagaaatattgttatcacaaagttggttatattcaaagctgacacaatttcattagccagaatgaatctcctgtttccttatttcctttatatatttaaaaaaagaaatcgtgggtatatatattttttgttttgtagactatatttaagaataattttgagatttcttattgtttaattttacctttaaaatctttattttctattgtatatatttaaggtgtacaatgggatgttttcatatacattgtaaaattattatgattgttaagcaaattaacatccatcatctcacagttgccctattttttggtggtaagcactgtcttcattttaaaaagtatatattttttcctgtttcttttataatattgagcagatttctttgtctttctgctctcatgagatcaaaaaagtattgtgctaagcattaggaataaagagatcaatgtggtgtaatacctgcatttaagaagcccacagtccagtgggaaagaaagacatggtaattagttgttcggacacagtgtaacattttacattggctgtctcctgggaaaagtgatttttctcatctagttgcattttcatattgggagctttttagagtactgctgctgaaaattgaaagacattgcattttagaggatgggcatgtgagcattattaataaaagttaaaagggcattggacaaacattttcatttggaaaacctgcttaggtttggtgaaactaactatagtacaatttgtttcctaactctgaacacttctaaaaaaactgatgccaatacttatagtgataatctaaacataagcagggacctgaagtaccttcttgaacataaaaatattatattgtgttcagtatagaatcacagacattgaggggaccttagagacccatctaacaaaacccctttgtcatgtgagaaaactgaaggagagagtgatttgtgatttagccaaggtttcttagcttataggtgcatacagatatattatactttaatatagtccccaaatgtcagctacaaagcatctatatttgcattaaaaaaggttgtttacttttttaaaaaaggacctctttcaaatagggaaattcacttccatagccgattgattttaatttaatttttattttcagtaaacatttataaattaccatcactatttaaaagggtctggtgaaatactattttgatgtataagaaacatctgatatactttcaaaatcagttattcttcatttcttgttgatttgctggtagttcctaacacctacaattaaaatttctttgcctaggtaccccccttgagatagtgaatgcctgactacctatattccatggaagttattttagctgctatgaagtctagctcatttgggaccatactcttgttcactttggcttcaatcaccattgactaatcagttaattgaagagtgaatagattgagaacattcttggcatccaggctaaaattagtatgctataatggaatgctaccaagatttatagacattgtgattaagtagtcatgtaaagttaaattctgtgggtcttagttttcttctttatacagtcagagagttggataagatatcccttccaactctgttattttgtgatatacgtctgtcatcctagctattgtgcttctgtagggactagtattaattacagctcacttgaatgtagtcatcacagctgagtggagtcctgcatcaaggggaagaaccactcactgggtggctttctttctgcttcacccaggtggtacgaaatctggcatataatttatgtataaaatttcagtaaacgggccatagcctggaaacatttgttggggctctgcagattttgttggtctttgccagtttctttttcattgatgtggttagttggccatttaaacatttaaaatagtatagtgaaatactatttaaaaaagctcttgaggtcacgttttttggtcatgccccacaatttacaaggttttattttgactttttctgcctctacctcattagtcacagtccattgaagggtcttgagactgttgttaacaattttattatattgctaccaagctggaagggaaggctgaagcatagatttctttttacattttatttacatgaaattatgtagtagggaaggtggtggtggtgcatctgtgtgtgctgtatgtgttagaaaacagggaaataaggatactggtagcccataaaactctctgctacattgagggagttgggtatttctattatgacttctgattttaaatggaactaatcagaagatgttgaaaatggattcaagtattgtttggatttctttttacaggatgaaggagttgttaaagaaatccctattactcatcatgttaaggaaggctatgagaaagcagatcctgcacagtttgagttgctcaaggttcttggtcaggggtcatttggaaaggtaatgaataatttttttccccatttgttcctcttctaaacatttagcaaaaaaagtgaacacatacgtagaaatctgattaatgagtcgtttctaaacttctgaacggatgctcacaaaggtaattctttaattttattcctttcatttctgttgtacttttattaccatttctttgtacttctctgtaacaatcaaggttttccaattccctaggcttactgatttcttgagggaaaaatatagcatactctttattagctgcctaaatcaaagtagcttgatttttgtaaaaaatggtattcctatctccttgttcccatgctggcatgaatacagcttgttttctcctggtgatttaatctacatttgttagctatgacacagaaatcagaatttatgtcttaattcatgtatgaattcgtctgtcatctgttaaagtagaacaagggtgtgtaaagtagaacaagggtagttactgggtgatatgtggtttatttaagtcttcagagagcttacatttcaaggcagaaagcaaaattgagtaagaataactattatagtcgttactaaagttgtttattaagcacttaataacatggttttagaaaagaagtgtataggtctggttcctgacctttatatacctaacatctaatacatgaaaattattgcatacacatagtaacagcctttagaaaaacagcaatttagaaaaaccgcaatttaaggggagaaagataaagagaaactactaatttcaagtggctgagatttagttttaaaggggaaaaataaggcaattgagcataggttatttatagtgctctggaacattttaaaataaagacatttttattttattgggttttagaaatggggtcttgctctgtcacccaggctgtagtgcagtggcatgattgtagttcactgtagccttgatctcctgggctcaagtgatccttcatccccagcttctcaaggagctgatactacaggtgtgcaccaccacacccagctaattaaaaaaaaaaatttctggacacaaggttttgctgtgttgctcaggctggtctcgaactcctagccttaagtgatcttcccacgttagcctcccaaggaaagacatttattgaacgctcagtgtgttcacaacaatgtataagcagtattagtgactataaaatagatgtcatcttaaagtaacctgatatttatgcattatagaatataacgtgaaatataaagaaacattttcaggacacaaaaaagacaaactgagatgaattatggcttggcagtgaattggtacaggagatatataagacagggagataggacttgaacagtgttctgaggagagaaaaactagaaggaactgttaatagattggtggtggtggtggtggtggtggtgatgaagttggtggggaatgtttatagaactgatctgtagagtaggcatgtatattatatacaactttattttaaagtttctaaaagtgtaatattcttttagctattttgcactttatagctatttttatggtgttaattttagaagaacagagatgaagttattttatattccagatgtataaatgcttaaatatttgacttaaataaaatctcaccaggcaaacatttcaagctattaaaattacctgctttgtcttttgatgtatgtatttactcatatatgcttttgtttatcttcagctttgttccagaaaggatttaaggctttcatagatgaatattaaataaaattaaaaataagtagtgaagatgatggaaacagaaagctcagagcaggaaagattagatgaagccaggagtacactctgttcacaaaagcatgacataagggtctgtaagtccttttctagtaggtcaaaaattagacttaagttttctagcagccattataaagaagctaatatatttgagtgtattataattgacagtattttaaaggtggaaacggattaaacagaccagagaaatttctctcatgggttttcttaaagaaaatactgtataacataatgaaatatattcaataacatcatgttaaatatagcaacatgatgaaaagttgtcaatgtagtgcttaaattcagggaaatcttagctttgtctgtgctgggatttagttttaagaagaatatcattgaaaccttttaattcctaatattaaagtacaaaatagtgatctttatgtagttcatcaaccaaagaaatattaaggaggtggcttttttttccccctaggatatgaacatacttgcttgtcagtgttacatcatttgcggaggcattgtgtaggtgtgacttgttatatgctgaacctattcaggaatttggccaaggctgtgaatcagataagtgtaggagcaattccaagagactttgagctgcagagtcttaagtttttcttcaaaagttccattgttaaaatgcagtaaatgggattttagcccatataaagccttataacattaaatgtttttattgtctatttaacagtgggggaaccctgctaaagtcaacttaaaattgtagtttaaggaagcatgcattattatcatgaaaggtgcatatacaattctgtcctaagcacaccaagtataatgctattaatgatcattaatgtatgggttgaaaggtatactaaacaacttacattaattattttaaaacacaaacacatgaaaatacctttgaaattgtctgattagtcgtccttatatatcatgtgccaaataatatttatataaaacatacattttaatatttgtggattataaaaaaatacttttgcttttgtgatagtaaagactttaagtacaggacttaaaaatgaatttttgaaaatatcgaatacttgtactcttaaataagtgatcaactctcaagtgatagtttttaaaaacatatgcttacatcttaaaatttcctgaagttggttctttctcttcaaacagcatttaaatatgtgtgatcttctagttcactgcagttactgaatattcttataggaaaatataaacgtccatgtgattgagagaaaattatattggagtaactcacccattcttttggacaatttcccatgattggattagggtgactgaaaagaagcctcttatcaatatagatacgtttttaggtccactagtttgaagccatgcaacaacttttttactttaattatttctttaagcaccctatcctcaaatacagtcatgttccaaggtactgggagttaggactttaacatattaactttgaggggacataatttagtccatggcgtacttgtatctatttctttaaagggcatgatgtttctgacaaatttctctgtcctcaagttcacagtttatttctgtatcccccagtgtccagcacagtgtctgatacagaatatgtgcttaatgactgttaatttgttgattgaataactaatgagtacatatatgggaagataaggtgatatagcacaactttattataccaggatagggaaatttttcaaggatgatttaggaaaaaagaaatagatcacagataagtacattgtcaccttatttcaagttaggttgttactactgtcaagcttttggaatggtgcagaacctgaagaaagtgaggtggaaggaggagggagagtttcagatgacaaagaaggacattataatagtaaaaatcaataaacatagccttgggaaggttgagaaatgttaggcaatagacaagaatgggatggaaaaagaactacttttgtagttgattttgggaagggggcagtgagatacgatattttctcatgctaggaaagtcactcataagacctgaaaaaaaagaaaaaacagatcaggtgttaaacaggttacttctctagactgcgaatgatggatcctattaaatgccatattgggatactactgaatattagaagaactaggggaaaaaagtggctcctttatgcaacagtgtcttcagaaagcctagactctttctttcttccttgcttgcctcttctcactcttcatattccgttctttacaatcccactaagggctatgttttgtaagagtggttctgaaattgtggtctccgtacccctggacaatcccaagacttttcagggagtcctcaagtcaaaactacttcacagtcatcctaagatattgtttgtacaactgctggcatgttggccaatcaaggcagtgacttcaaatggtaccagtagtcattgtagtcttcattttattacatatgcagtttaagaatgtccttgatgagacattaacaattattacatttattaaatctcaatccttgaatacccttcttttgaataatatgcgatgaaataggaagtacatgtgaagccccttagccacgtaacaaaatagaatggttgtctcaaggcagaggacttgtgcaaattttttgagttgtgagctgaaccaaccactgtttttatggatcaccacttttccttgaaaggacaataaactatggttacctaaatatgcttatttcgtaggtgttttctcaaaaatgaatggagtgagcctgttcatagaaaaacaactgatactctttttgtcaatggtagaatctgagctttccagcaaataattgaatttaggcaaatgtatatttgccacattgacgtcattttcttgatgcagctatacttatttttattatgcttcccttcattgcacttcacagatatccttcacttcattgcgcttcacagatatgtatgtgtgtatatatatgtatatgtgtgtgtgtatatatatatatctatatatatcctatatatctatatctatatatatcctatatatctatatctctatatatcctatatagctatatatatatctatatatcctatatatatctatatatcctatatatctatatatatcctatatatatctatatatatctatcctctatatatctatcctctatatatctatcctctatatatctatcctctatatatatatcctctctatatatcctctctctatatatcctctctatatatatcctctatatatatatcctatatatatcctctatatatatatcctatatatatcctctatatatatatcctatatatatcctctctatatatcctatatatatcctctatatatatatatcctatatatatcctctatatatatatatcctatatatatcctctctatatatatatcctatatatatcctctctatatattctctatatagtccgtgtctcatattttggtaattcttgcagtgtttcaaatgttttccttactgttatatctgttatggttctttgtggtctgtgatctttgatgttagtgttgtcattgtttcggggtgccatgaaccacgcacacataagacagtgagcttcattgataactggtgtgtgtgttctggctattttactgaccagctgtttccccatctctctctgtctccttgggcccctctattccctgacctgcaagaatattgaaattggtctaagtaataactctatagtggccttggagtgttcaagggaaaggaagtgttctgcatttgtcactttaaatcaaaagctacaaattattaagcttggtgaggaaggcatgtcaaaatctgaggtaggctaaaagctaaggttcttgtgccaaacagccaagttgtgaatgcaaaggaatgttcttgaaagaaattaaaattgctactccagtgaacatataaataagaagaaagtgaaacagccttattactgatatggagaaagttttagtggtctaaatagaagatcaaagcagccacaacactccctctagccaaagcctaatctagagcaattccctagctctctttaattctgtgaaggctgagagaggtgaggaagctgcacaagaaaagtttgaagccagcaggggttggctcatgaagagaaaaaggaaagaagccattttcgtaacataaaagtgtaaggtaatgctacagatgctgatgtaaaagctgtagcaggttagtcagaagatctagctaaagtaactcttgaaggtggttacactaagcaacaggttttaattgtagaagaaacagccttctgttggaagaagatgccatctagcctttcatagctagagcgaagtcagcgcctagcttcatagcttggaaggacaggctgactctcttgatagaagctagtgatcctggaacttttaagttgaagccaatgctcatttgccattccaaaaattctagggcctttaaaaattatgttaaatctactatgcctatgctctataaatagaacaaagcctgaatgacagcacatctgtttatagcatggtgtgctaagtattttaagcccactgttgagacctactgttcagggaaaaaaaaagacatttctcaaaatattactgctcattggcaatactcctagttacccaagagccctgatagagatgtacaaggagattaatgttgttttcatgcttgctattacagtatccattctgcagcccatgaaacaaagaataattttgactttcaagccctattatttaagaaatacattttgtaaggctatagttgctgtagatggtgattgctctgatgaatctgggcaaagcaaattgaaaacctctggaaaggacttgctattctagatgtcattaagaacatttgtgattcatgggaggaagtcaaaatatcaacattaacaggagtttggaagaagttgattccaacccttatggatgacttggaggggctcaagacatcattggaggaaatcactgaaaatgtggtggaaatagcaagacaattagaagtggaactaaagatgtgacggaagtgctgcagtctcatgataaaacttgaatggatgaggagttgctccttgtggatgagcaaagaaaatagtttttttgaggtgaaatctacttctggtgaagatgctatgaacattgttgaaatgacagtaaaggctttagaatattacgtagacttctttaataaagcggtaggatttgagaggactgagttcacttttgaaagagattctactgtgtataaaatgctatcaaacaccatcaaattctatagaaaattatttcatgaaaggaagtcaatctgtgcaacaaacttcatcattgtcttattttaggaaattgccacagccaccctaaccttcagcagccaccaccctgatcactcagcagccatcaacattgaggcaagaccctccaccagcaataagattatatctcactgaagactcagatgatcattagcattttttagcaataaagcatatttaattaaggtatatagggtcataatatttgtttgctctttattaaggtaacaaaaagtgggcctgggtcttaaaaggaggtatgtagctcaagtttgtctttcattatgttatttatctcttacatcacaaaattctcaaagttcagtaccaggtcatctattttaaaaattttcctttctatagtagtatttatatgcattatgatttattaaatcataacagtttttttgattaaaaactaaattaaatctaagaaaagtgagaaaataataatgtaaattatagaataatttataggagaaggtataacttttttcatttatttttttaaattgacaaatagttgtacatattcgtggggtacatagtgaagtttcaatacgtataatgtatggggatcagatcagggtaattttgcatccttcaacaaatctctcactatctctcccttccctctatgggaaggcatatgtttataaaattagagaaaatgcaaggatataagtgtatgctggtgtgtttgtgtagagcaagaatgactagaaatgtgaattttcctttttttttttgttcaattttctattaggtttttcttgttagaaagaagaccggtcctgatgctgggcagctctatgcaatgaaggtgttaaaaaaagcctctttaaaaggtagaaaattagaaagtttattaaaatagaatttgacaaagtgtagtttaagaaatatgctgtgtattacttgcataaggtgaatagatgtagctatttttaacattggtatttgtggcagtttttctaacctgtaggtaatattactagatttgatattggcatatgccttaacttagtgagagtgcacccccgtaaaaatttgatgctagaaaaagtgacatatttcatttaatttaaacttattaacttaattaggatatattctcttctttcaagattcttggtttttccttttctatctgtgtcagtattacatattgagaaagcacaaccatttgcacacactatcttttagggtgtgataaaaataccgcttatgatcagttttaatcagtaaactcatttgaacaattaaaagagattaataggccgggcatggtggctcacacctataatcccagcagtttgagaggctgaggtgggctgaacccttgagcccagcccaggagttcaagaccagcctgagtaacctggtgaaaccctgtctctacaaaaattacaaaaatgagccaggtgtagtggcatgcacctatagttccagctgctcgggaagctgagatgggaggattgtttgagcctgggaggtggaggttgcagtgagctgagattgtgccgctgcagtccagcctggacaagagagcaataccctgtctcaacaacaacaacaacaaaagatgaacacataataaatgcatattagctacatacctgtttatgccaccttaacctgtctctagatgtaatgaatatttttgctttctgagaatatggtcattatctaggaatatgaaaactgcaaatttcggcatttaggaagttacgttttgtatttttatatttgttgtttggtaaattattgatggactcttaactgaaatgatgtaagagagcaatatattgtacctttatgtaatttttagttcgagacagagttcggacaaagatggagagggatatactggtggaagtaaatcatccatttattgtcaaattgcactatggtatgtaatctttttttattcttttatttattttaatccttatttattaatatggaattagagaattatgatttcctttcacacaatactataacaggaaataaatactttagacttagtatattcagctacgtaaaatgtatttgtctgaaataaatatcagtatttagttattagttatatgtccttcatttttctaagttgattaacttgtttaaagtaacaagatagtgtttttcacttacctgtctggtagtatttatgtactttgctaggctgtaacttcataatcttagactataaactattgtgataatttacgtaaaacattcagttgcttgttcataatgaccacacttatctgtttcctggttatattccagaggaccatgtagttatgttagtctacttgagttatttttttatacttaatgggagaggactaccagtaagaagcagtagatgtaatggaaagaacatgagctttggagttagacatttctgaatttgaatcatggctccattgctaatttaacatatggttgagatcaactctatctatttatctatcgtctatctacctctgatatagtgtactttgtcacttccatttttctatatttaaaaaagtagcagctgttgattttattcttagtgtaattttgacaaataatacattaaaatatatacccagttatcactattaaaatcccataagaaatttccacccattattcttttactccttgttatactaacagaataaaaaaagtaacatctaaagaccttcccgtgtctgtagtaggtccatcagtaaacacttattgtgtgcaaaaatactgtattaaattcagtgggctatagaagtgaaacactttttgacctcaagaaatttataacctatagagaacacagagatgtaaagaaattaattgcataaatcaaaatggaataagtactattaaggaggtataaatataataagatctgaaaagtagcagcaagtaatttcaataaagaagaggttgaggaagctgttgaaaaatgtgaaaagggggaaatcctggaaagtttaatagaggaagtggcatttttcttcagctttatagaatttgtagattttcagtaggtttgataggctaagtttaggcaattgcagtataacatgcttcataaattaggaaatttttggtggtagaagttctgtgtaaagccacattcaagggtgattatgttctgggaaggggtttatagctatttctacactggattaaacactgggtttaattttgagatgttatccattaatgaaccatgtgagtaaatactgtaaaattctaaacatatattaatttaaaagcatactaagctttttaagcctttgagattatcctgttactatattctaatttttaattagcctttcagactgaagggaaactgtacttaatactggattttctcaggggaggagatgttttcacaagattatccaaagaggtaagtatctgtacatttttactgtattttcttaaaacaaaactaagaaaaatcacttttgttcttctgctaattacatttttcctattgaaattttaaatggtaactactctgtttcattgcatagtatttggtaatgtgcctgttaatggcattttctatcatctcaatgtgctactcattattttcttaggcaaaagaacaatatgaataactggataaagtagatacgggcaagcctaggactttggctactcaagctacctaaacacctctgggagtagtgtttattcattttaaaatagctttatggaggtataattgacatacaataaactgtacatactaaagtatacaatttcataagtttttacatttgtatataactgtgaaaccatcattacaataagaataattagcatatctgtcattcccgaaagttttcttgtgtccctttgtgatgccgtcctcctgccctaaccctactgaatctgctttctgtcactatttggtttgtactttctaggagtttatataaatggaatcatatagtatattcattttttttgtctggcttaacataagtactttgagattcatctatgttgtatgtactaatagtttattcatttttattgatgggtagtattactgattgtatggatacatcatgatttgtttatcattcacttgttgatggatttttgggtcattgcagtttttgatattactaataaaactgctataaacattcatgtacaagtgattgtatcatatgttttcgtttctcatgggtaaatacctaagagtataatgactgggtcatgtgctaggtatatgtttaacttttaaagacactgccaaactgttttccaaagtgattatataaattttacattcccaccagcagcgtaagagagtttcagttgctccacatccttgccaacatttggtatggtcagtcttttaaaatttttgaacattagattagtttttagtggtatcccattgtggttttgaattacatcacactaatgactaatgacattgagcatcttttcatgagctcatttgccatctatcctcttggtaaaatgtctgtttaaatttttagcctgtttttttttattggattgtttgttttcttattgagttttgagagttcttcatatattttggatgtaagtccttgatcatatatgttatttggaaatattttcttctggtctatagtttgtcttttcattctcttaacagtgtattgaaaagcagaagttcttaattttggtgaagtctgatttataggattttttccctttattaattgtgtttttagtgtcctttgtaaggcatatctgcctaatcacagctaaagattttctcttatgttttctagaggttttgtagtttgcattttacatctagatttaggatccgttttgagttcattttagtatatggttacaggtatgtgcagaagtccttttcatttgttcacatagatagccagttgttatagcatcattattggaaaagtctgtcttttcttcactgaattaccttacatttttcttgaaaaccagttgatcatatatatgaaggcctatttctggatactctgttctgttcattgatccatttgtctatctttatgacaataccacactgtctgaattactgtacctttataatatgtcttgaagtgaggcagtgcgagtccttgagctttgcttttctttttgaaagtttggctcttctaggtcctttgcatttccatatcaattttaaaatcggcttttcactttgtacaaaaatgtcctgctgggatattggttggaattttattgaatgtataggtcaatttgcagataagttacattttagtaatattgagtcttttaattgatgaacgtggtatctttctcaatttatttaggtcatctttaatttctcgcagtattgttttcagtgtaattcttgcaattcttttgtcagatttacctctaagtaaatcctgtttttaatgttattgtaaatagaattgacttcctaatttcaattttaaattggatggaaacacaaccgattttttgtgtattgagcttgtttcccacagtctagccaaacttaatacttctagcagcttttagaaatagatttcaaaggttgagaaagttttctgtaactatttttgagagttttataaaaaatcaggaatcaatttcagatttattcaaatgctttttcttcatttattgagattataattttttttagtcttactgtggtgaattacatcgattgatagtcagatgttgaaccaaccttgcatttatggatgaaccccacttagtcttgatgtattaccctttttataaattgttggacgatatttgctgaatatttaagaatttttgcatctatttttatgagggatatttatgattttcttgtattattatgtctatttttggtattagagtaattctgactccatagaatgagttgggtgtagtttttgcctctttaactttctgggaaagtttatgtagattcaatgataaattgcccttaaatgattgatagaattcaccagtgaaggcatctgaacctgaagtttttcttataggaaagtatttaactccaaaatcaatttatgtaatagacttgggttatttatttttgagtgagctttgctaacttgtgtcttttaaggaatttgtctatttcatcaaagttatgaaatttattggcataaaaatgttcataatattcccttattgtcctttttgtagttgtggaattgttagtgatgtcactgctctcacttttgatgttggtaattcatgtttgctctgtttttgttctgcttagcttcactagagatttatcaattttattactctcaaagaaacagcttttggtttcattgatttttctgtattctatttctgttctattacattgttttctgctttaatcttttttactgcttattttatgtattagcttttctttttctggtttttaagatgggagctgaagttattgatttgaggcctttcttttctaataatgtaggtgtttagtgttataaattcccctctgaatactgctttagggtcatctcacaagttttgatatgcatcatttttattttctctcagttcagtgtactttatgatttcctttctatttctttgacccattgatatttagaagtctgcttaatttacaaatatttgggcattctttcacatatctttctattgttgatttctactgtaatgttcttgtggtcataaagcatactttgtatgactttaattcttttaaacttatatttgctttatgcttcagaatgtcgtgtatcttggtaaatgttccatgtccacttgagaagaatgtgtattctgctatttttgggtggtggaaggttctataaatgtggactggatcaagttgtttgatagtgatattaaattgcctatattctttctaattttctgcctattctacaaatttttgagacaaggattgacacctttttactttaattatgaatttgtatatttcttcttgaatttctgttagtttttgtttcatatatttcaaagttctgttattaggtacataaacctttaggtttattattccctcttgatgaatagaccccttcatcgttatgatatgacatgactgactttatccctggtaatgttcttttcttcaaaatcttatttgatactaacatggtcacaccaactattttctaaaaaagtagtgttagtttggtatatctttttccatattttaacttcagcctatgtgtgtctttatattttaatgtgtggtacttaaggaagcatagtttccttgttttttaatccaatctgagagtccacgtcttttaattgggatattagacctgttacatttaatatgattatttttgtagttctatttaaatctactatcttgctatttgggattttttttggtcccatttgttctttgttcttgtttccctcttctcctggcttctttttaattactttttttttttttacaatttcatttttatctctgctgttgtcttgtagatattatccataacactttgttgtgtaatttaatagttaccttaggatttgtagtataagttttaactcagaggtcaggaaaatatggtcttcaggctaaatttggccagttatttttctttgtaaataaagttttattggaacacagacatactcatttgtttaagtattgtctgtgccttattttgtacttcaatggaatcatacttgcatagtcatttttactggatagcaggcatggtggatttaattttcttacatgctagatacctttattttcatataaatagtatttagcttttttctaggatgcagttcattttcttgtaaacactttgatcattttgggccttgtgtttaagctttgtcatgtgggactagggaagtatttagtttaagattaatttttcctcagtatgaagcatgtcctttcttatactctaacaaagactctttgaattatgaggcttttcattctaacttttgggaagaggagctatctcagtcctttgtgagctgagtgggttctttcttttaatcttttaaggtttttcttttcctgggcttgggcttgggtagtttcctcagatccatgtttgactatatatatatatatgtatgttttttttttttttttttttgagatggagtctcagtctattgccccggctggagtgcagtagcgtgatctcagctcactgcaacctctgcctcccgggttcaagtgattctcctgcctcagcctcctgcatagctgggactacaggtgcacagttagcccggctaattcttgtatttttagtagagatggggtttcaccatgttggctaggctggtctcaagctcctgacctcaagtgatctgcctgccttggcctcccaaaatgctgggattacaggcatgagccccagtgcctggcccatgattgacaattttttattagatggcagacattgtgacatttagcttgttaaggtgatgaacatttttgtatttctgtaactattattgatctctgttctgagatataaaactagtttgattattataggtcttttaaagttcgttagattggactaacacagctttcagtcgggttaattttgctccactactgaagcaaaatccttctgattactttatctgatgtcctatgaattatgagattttctgctctggctgaaggcaacaggaactattcctggcccatcgtgagttcatcgactggttcctctaacccttcgttgttttcctggtttagggtaatgtcctcactcatatgtgctgagtatttctccactgaatacttgaaggagactgtctgcatatctacagagttctctttgtgcaattttctcttctctaatactccatcctgtgaattttgtcctccttctctaagactctctgttctggcttatctactcaggaagtctttcaggctccacctgacttagccctccctgtactaccacttggaaactctttccaggtagtaagctaaggcagttgtggggcatacgtcatttgtcccctgtctctcagtattcagtgtcctttgttgcctgatgtccaatgtcttaaaagtatcatttcatacgttttgtcctgtgttttagatttttcaggcatgagattaaatcctgtctttgtctgcttcatttttctttaagtagtagtctgggagtagcattttaattgggtgatagtagttcaatgtcttacttccagaatttacagtgtagaatgtgggagagctactgttgattcccagagctctggaacttgctgtgataaaggaacagtgtgtttctggtatgctagccagtgcttaagtggctaaggtctctggcttatatctgtctacctccagtgtagaatgtgccatgcgtgagcatagggtgtgttattggtatattattaactatagaacataattcaatatctgaaaaggattgtctaggacccccttctaaggagggtctttaaaggaccttgttaaaaactggtaccaaggcatagaaggacaacagtataataaggctccattattcatgatttcttgcgttagattttagaagtgtcacaaatatgtttgagaaattcttgtagtcatttctaattttcttttaaactcttcagacctccattctcgatgtgggactatctaaacccatactagcacagagatgcagtcagtaagttttgattcctaagtctggaatattaggtaagagcaagcattttaaatgcgttacccaaaagatgataaattagtacattgattgagcagccttttaaaaaatgattcaatggttacttctgtagacagcttattaataattagatgtttgttggttatacttcacttgtgtgtacaaaaaattgttaatccaaaatgaggacatgtttttataccttagaaaaagactttttaaatacatttttgtatgttttaaagaattttataatgtatgtcatttgtattttaaagggaaagatacctacccaaatttcccttcctcagaatgtacacacacacacacacacacacacacacacacacacacacaaatacacaggtaaacaaacagaactcttaagctgtgctctattttccttattcaagaaaatgtggtagaatgaatggctgattattgtaaagctgtcagttcttctaaaattaatttattggcttaacaaaattccaaataaagccacaatagggatttttgtttgtttgatgttgacagaattattttatagtttccttggaaaagtaaacaaatgcaaagaaataatatttttgtttgtttgttttttgagacagggtcttgctctgtccccaggctggagtgcagtggcactatcctggctcattgcagcctcaacctcccatgctcaggcaatcctcatgcctccgcctcccgaatagcttggactacagaagcgtgcgccactataccaggctatttttttttttcagtagagatgggatcttgctatgttgcctaggctggtctcaaactcctgggctccagtgatcctcacgcgttggcctccccaaattctcttgtttaaaacttggtaatgtgcataagcaaaacattatagtatcaacacaagattggaaacatactgtatagcccaaaaatatcctgttatagattacttaccctatatcattatgataaaggaagaatcacaaattgtggcaaagaggttgttcaataaacagtatttgagaaattgcttaaatatttccaaaaatcattttaaaatgccatctctaccatatacaaaaataaatcttacttacataaaaaagctaaatcttgaaaaacaattaccctataataaaatgcagctaaatattttgttgaccattggatggagaatgtctccttaaacattaaaataatgaaggaaaaatcatacagaaatcatcatacatttgactagcaaaaattaaggattactgaacatcaaaaactagcacaaaattggataaatatttgcagctaatatgatagaaaactgacatttttaacatttaagtgacttgtaccaattgacaaaacagtaagtcagtaatagaaaataggcacaaacattactgataattcacaaaaaagtagaaagactactaaacatgaataacaatgttcagcctaaaaatcaaaacttaaatttaaaaaataatgccattttcacacatcaaatttgaaaaggctttttacagggtatattccatgctatcaaggatgtggtaagacagatgctttcgtaaactgctgatgggagtggaaattactataacctttttggaaaataattttatcgtgtatgtcaggaatcttggactatttatgctccttgacccagcattccatttttaaaaaaaaattcttaattcattttgagaaaatgatcataaatatcagtaagatgtacatatgcatattgcagagttatttcaaatagagaaaaaatggaaataacctaaatgtcaagaaggggaatgtttaggtaaattacagtaaattggtatggttatattgtgcagctattgaaaactatttataaataagttttaaagacatagaatgcttatgatatagtattaactggggataaatgaggtcactaaatagtacttatacagtagtgtgatttcaactgtattaaaatacacatggacacattcacacacagatatgcacaaaaaatttgaaaagcagatgacaaaatattaaaagtgctttactcaagagtactggagtttaggataattttcattttgtagggggggcaatttatgagtaggacgattagtgggttggaaagcattattggtagtaaacctaaaatttgtttttagaaaagcaaacattgcatcacagcatgtggcagaagtggcttctgggaaagagttcattcttatggacaagtatccctgagttcatgtcttgggcctccttcttaactagctgtgttattttggacaagttacttaatcactctgagtttctttctccatttgcaaggtgttcctaatacctacctcatagagtttggaagctataataacataatgattgtacagtgcctggcacataattgctacttagtaatgttttatttgcatctatcatgaggctttctaccaagacaacctcatgacaaggtgataggcttgcataactgtaactaggcaaagtttttttctaatttagttttaaataatttttatttagggttttcttacgtattagctttcagagaacattcttattatccttggttagaagttgaataggcaacagttatttttattcttaataaaaagtcaagtgaaagtgatgtttttatatttttaccattataatattcaaattaatgctcccaaaacttagataataaacaatctttgtttttctattcatcattttaaaaagtagttagcatatcagaagttattaactactaattctctgcaattttactagtaacctggatctgcattgtgaaatgcagtggccgtaattctaaatgggcaagcaggttgtaagctattatgacatgtactttatgcaattttgagccttcttaataatccagaggtttctttatgaaaataacccatttttagaaaaatatttgattttgccaatccggagttcttgctaaaccttaaaaattacatctaatggctgattagtgtccagttctatgtgtaaatgaatactcactagtgaatgaatttagatgtaaacctataataatagtaccatatggttatatatatagttttttaaagtttacaaagctttttttccacataaactgatttgattcttacgacaaccttgttaggtatatacagcagatattattgtcctgtttcacagatttagaaactgaggcccaaagggctaagtgacatgtctgatattacactgccaattctgatttcaagttcattactcttcacttcatcatagcagccgtatttccagacacaaaatgattaatacagtgccattacactcgtatacattactaacagaagaggcttggatccaccccttaggtcagagatgagttcacaattgtgtgtaagataagggtactaatttaacaacttgtggcagtgtagtaaaagctttgggtttgtagagcccctaaaagtgaaatttctgagaaatgatactagacagtaaaagagagaatggtataaatagtggaattctcagagctaaaacaaaataacttctatatttcaaataccctaagggttactatattatagcagattactataattaatattggtattatgaggcaaagtcactttataaaaggtgaatcttgttctttgcatttcgtgtgagaagatatacaaacaacataatatttcagaaagatttacattaaaatggttaatatcgaagagacacattcttaaagagtttagtttcatttatatagaaatcttatttctctggtattgccaataattatgccagtcaaataagaattgactgtgcttattatcttattattttcctagatttgtctgtctactcattttgaaagaagtatttttaaatatactgaatattgaaagaaagatcaaatgataaatcattattttgttctaggttctgtttacagaggaagatgtgaaattctacctcgcagaactggcccttgctttggatcatctgcaccaattaggaattgtttatagagacctgaagccagaaaagtaaagctgtattcttttgttttcatgttgtctgtatttctggaactgctttttgtcaaactctgttagatcttttaaatattatagggaattctatatgaattatatgaaacttatttgtaaaaagtaatctttgtgagacgtacattgactttaagttaatcatttaaattttttacatatattaaatagatactgacctatcttagttttatgatttttatgtaaatgttttatgtaaaatattttattccatcttgttgaatgtgaattttgatttacagcattttgcttgatgaaataggacatatcaaattaacaggtatgcagattttcttattatacccttagccacttgattcatattttttattattttaatataaatttgtctaagcctgtttttttctctttctgaatttgatttgcaatttatttgtctctttagtgaattaaatgttacttgatggttagactgctctcaatatgttctaattgtaatattaaaaaattgtacattagatacacttatcctgaataagaaatgatcgttttctaggtaacaatacatcatcttagtttgagtctagacagaaagatgtatcacaagtagtcatcacatttgaaatatagtgacaaccttccctccaccttccctaactcctggcaaccacttatatgtcctacatctctataattttgttattttgagagtgatatagaaatagaatcatacagtagtcacactactgagcatttatcctagaaaaataaaaacctatgttcacataaaagcctgtatatgaatgttcataacaactctatttgtaatagcccaagactggaaattacctaaatgtccttcagaaggggaatggataaatgtaaattgtcatacttccatatgatgacatactactcagcagtaaaaaggaaagacctgttgagacatccagcaacttggatgaatctcaagggcattgtgctgagcgagccagtctaaaaacgttacatgtgttttcaatagatagtaaattctgtattttattatttgaatgcttgaacagagataagatgaagatgtagtctttacagataattttaaataggtacagtttaaaggtaagttgctttaaattcctgtgtatactgcatcaatgcaaaaataaaatatcaaacagttacctcaaaagctgacataaatatgaaaaagttcaaataaagtttagtgactcttattgattcacagcctgattatctttttcatgtacaaaagaaatgatgtgggttcgaagatatttaaagagaaaaaggaaaggaaaataacagtagggaacagtagagttttgagatttttatataatacataaattttattttatagcaattaaaaagataattaaaaattaattcctcttttcatttttgcttaataaagtatacctcttttgtcagaggtgagaaagagaggtgggaaaaagagtagaaaaaaacctatttttctctctctacattaaggttaattacgtaaataatgatgccaagtttatttgtatttagtgttctcattttaattctgcttttgatagattttagagacttaaactgtctgaggagggaagaatagataggatcaatattaatttataagcaagggacacaaagcatgtaacaggaagaaatgtgatttataattttttttaacaattcaatttattttccttgcaatggcacaaacatttcttttaaagaatgtgtacaataataaatattagataatttacagttttatgattataaccttaattcaaggttttataatgactacatttatggatttaccccgacagcattccaatgaggttggttaaagtatgaccactcacagcaagtattttaacccatttatgcctgaggctgcaatgttttgaatttttgcagtcagactttgatgatgaccttgagcagtagaatataaataactcccacatgcttagcgttccaataatggaacactaggcataaatgggtttaagagttcttttataatgtttcttttgatttattttatattctttatctttctctttaatttttcttgtttgctttctttctcctagggagggtgtttgctagcattctgttttccaaaaataattgcaggagaatggcaatacttttttttttttcactagtccactttaaaaattctccagacataaggaatggacctagtatctggaatgacaggctactctgaaattacaatggactatagaaagtttcctgttgtaactgtgctttgagtctgattggcctcgttctgagattatgattgttataacagatcagtagaatgagagtggatgagagtggatattcccaaagatctacatccttgctaatatattatttatattatatattattatattataaaacttaagtttttttcctgaaatcctgcatccttttcatagtctgtataaaatcccttgtatagtttatataaaaataactttataatatgacttgcctctttagaaaaattatttcatcataatatcccattaaaaagtacagctcgaatacagaatttatttgaactgtggttgctataatctgttgcattttttaaattttattttttttaagagacagagtcttactctgtcacccaggctggagggcagtggcaggatcacatcttatcacagcctttttttaaattattattattatcacagcctttaactcctgggctcaagccatcctcccaccccagccttctgggtagctgggacttcaggaacatgccaccacacccagctattttttgtgtgtgtggcgacggagtcttgctatattgcccaggctggtcttgaactcctggcctcaagcaatcatcctgccacggcttcccaaagtgttggggtaacaggtgtgagccactgtgcccagcctataatgtgttttctgagagacataattcagaagcaacttctacaaagagagggaagaaaaatatacagtggtcagagatgtaaatcacacagtacttgagctgagtatttaagagaaaaatgtatacttgagaaaataattctatgtagtttttatagttacttgcaaactaatcactgagattgcttttatttatagtaactattcatcaaattattatattctgagtgtgcttttaaagtttaaagtcttagaaaagagtcatttagattacaaagatgtgaaacactgaccctcaaacctaccccccaaaataacccttccattatgtagttgaattgtacattttcttttattttcaaattagtgatttggaaaaaccactgtataacatatggcttgtctaatcttgtagctagttttatagttgtacaattttcgtcagttgattgctgagcaaattatagttgttaaataacagtttagtcagaaatagtacggcaaaggaactctgtggattggcataaatctctgaaaggtaacagaagaatgagaaaattcttctctccccccttctctctggaatggccaaagaaactagtgttcttctccacaagctgtgttatattcagctgaactgacctcgaagactagatgttaattatatttacttttgtaaatggaatgtgcagagtttacaagtttcagtatacagagagaacatagtggcacagtgaaaagatttttaaaaattataccaaaagttaaaatacattaagctattatctaagaataagtgatttttttaaatgaaaatgttgagcatagttagaaaatcagttttgaggggacctcaaattaagtgttaatgttgatattttattgtaaacaagcaatatagggcttataaacttttttatttttaaaatgaaagtatactttacatatattaaaactcatccttctgatgtcttttggtgttgggactatcccgtgtcttgactgcatcaatgtcagtgtcctggctgtggttatagtactatagttttgcaagattttaccatgggaaacactggataaacaggatccctgtagtttttcatgtaacagcatgcgaatctgcaatcatctcaaaatataaagtttaatttttaaaaatcacttttgttgtatgtatagttctgtaagttttgacaaatgcgtacagccttgtaactatcaccaaaataaaaatatgaaacagttccataaccctcctcccccaaattctccccttataagtcagtcctccctttaccccaagccaactaccagtctgctttctgttcctgtatatatgctttttccataatgtaattatgaacagaggcatacagtatacagccttttgagactggcttcatttaattagcatagggcatttgagattcatccatattgttggctgtatcagtgagtgtttttttaaattgctaagtagtgtttcattttatggtggtaccatgatttgtttatccattcctcaatgaggaggcatttgagttatttcctgctttgggcaatgatgaataaagccactgtaaaacttcatgtataggttttctttttaccataaatttttatttcagttaagtaaatacataaggagtgagattagttgatcatatgttgagtatatttaactaagtgagaaaacttcaaactgatttccaaagtggttgtaatattttgtatacccacccacaatgtccagtggatctgtatcctcactagcattttgtattgttagtattttttattttagctattctaataggcattaggtagaattatcatggttttattttgtatttttctaataactaatgacactaagcacattttcatatatttatttctttgagaagtgtctattcagatcttttgcccattaaaaaattggattgtttgtttggttttcagcctagaatgttttatattttttaaaatttaaatttaaattttgtgggtacatacgtgtatatatttatgtgttgtatgagatattttgatacaggcatgcaatgcctaataatcacatcagggtaaatggggtatatacatcacctcaagcatttatcctttatattacaaacaatccaatcatatgcatttagttatttaaaaatgtataattaaattatttttgactatactcaccctgttgtgctagctaatattaagtcttatttattctaactattttttgcacccattatccatccctcatcccctaagtcccttcccccgtacccttcccagccactagtaaccatccttctactctatttccatgagttcaattattttaattttcatctcccataaataagtgagaacataagtttatttttctgtgcctggcttatttcagttaacacagtgacctccagttccactcacaatgcagatgacaggttcttactcttttttatggatgaatagtactctgttgtgtataagtgccacattttctttatccatttgtctgtcaatgggcatttagattgctttcttatcttggttattaaaaacagtgctgcagcaaacatgggagtacagatatctcttcattgtactgatttcctttcttttaggtttgtacccagcagtgggattaatggatcatatggtagctctgtttttagttttttttgaggaacctccaaactgttctccatagtggttgtgctgatttacattctcaccaacagcgtatgaggtttcctttttctctccatccttggcagcatttttttattgcctgacttttgaataaaggcctcattttagggagaaattatatctcattgtagttttgatttgcatttctctggtgatcaatgatgttgagcaccttttcatatgcctgtttgccatttgtatgctttcttttgaggattttttattcagatcttgtgctcattttttaattggattattagactttttcctgtagagttgtttgagctccttatgtattcttgttactaatcccttgtcagatgggcagtttgcaaatattttcccttattctgtgaattgtctcttcatgttattgattgtttcctttgccgtgcagaagctttttaacttgatgtgatccagtttgtccatatttgctttgatcacctgtgcttgtgggctattacttaagaaatctttgcccagtccaatatgctggcaagtttatccaatgttttcttgtagtagttttattgttcaaggtctcagatgtaaatttttaatccattttgtttttatttttgtatatagcaagagctagagatgtagtttcattcatctgtatatgcatatccagttttcctagcaccatttattgaacacaccgtcctttacccaatgtatgttcttggcaccttgtcaaaaaggaatttactgtagatacatggatttatctctgggttctctattctgttccctgttctatgtttctgtttttatgacagtaccatgttactatagctttgtagtataatttgaagttagataatgtgattcctccagttttgttctttttgtgtaggtagctttatctatactgggtattttgtggatccatataaattttaggacttttttttctatttctgtgtagaatctcattctactttgaaaggtattgtgtttaatctgtagattgctttgggtagtatggacaattttaacaatattgactctttcaattcatgaacttggaatatctttccattttttgtgtgtctttctcaatttcttgcctcattttagtacattcctttttacatgtctttaatttctttttatcagtgttttgtagtttcattgtagagatattttacttcttggttaattactatgtatttgattttatttttagctattgtaagtggcatcactttctggatttcattttcagattgttctctgtcggcctgtagaaatgctactgatttttgtgtgttcattttgtatcctgcaactttgctgagtttgtcagttctaatagtttcttggaggagtctttaggtttttccaaatgtaagataataccatcagtaaaaaaggataatttgacttcttcctttctcacttggatgccctttttctttctcttgtctgattgctctggttaggacttgcagtactatgttgattaacagtagtgaaagcaggcatcctcattgtgttccaggtctcaggggaaaggatgacagtttttcccaattcagtatgatactaactgtgggtgtgtcatatatggctttgattatgttgaagcatgttccttctatacccaattttctgagggtttttttttttatcatgaagagatgttgagtttcattgggtgctttttcaacatcagttgaaatcatcatgtggtttttgtccttctttttgttggtatgatgtgtcatattaagtgatttgcatatggtgaaccatccttgcatacctgggacaaatctcacttggtcatgatgaacagtctttttaatgtattattgaattcaatttgctagtatcttgttgaggatttttgcattattattcatcagagatattggcctgtagttttttgttgtcttatctttgtctggtttgggtatcagggtaatacttgcctcatagaatgactttgggagtattccctcctgctctattttttggaatattatgaacaggattggtattaattcttctttaaatgtttggaggagtgtagcactgaagccatcaggccctgggcagttcattactgggagactttttattatgatgttgatcttactaattgttattggcctcttcaggttttggatttcttggtaggttttacgtgacaaggaatttgtccattttctctagattttcccatttattggcatgtagttgctcatagtagccactaatgaccctttgaatttctgtggtatcagtcgtaatatttcctttttcatctctaattttaattattggggtcttctccctgtttttctttgatagtctggctaatggtttatcaattttgtttgttttttcaaaaagcaagtttttatttcattggtcttttatattattttcctcatttaaaattcatttatttctgctctgatatttattacttcttttctccgactaatttgggttcaattcacttgcttctctagttctttaagatacatcataaagttatttgaatttttttttgatgtaagcactttcagctataaatttccatcttagtactgctttcactgtattccataggattttttatgttgtatttctattatcattaatttcaagaaatttttaaatttctttcttaatttttttattgactctctggtcattcaggaacatattgttttatttctatgtgtttatatagtttacaaaattcctcttgttgttgacgtctagttgtattcagttgtgatcacagaagatgcttgatattatttcagatttttgaatatattaagacttgttttgtgatctaacatatggtctattcttgagaatgatccatgtgctgaggagaagaatgtgtattctgcagcctttggatgaaatgttctgtaagtatctattaggtccactttttctatggtgcagattaagttcgatatttcattgttgagttttctgtctgggagatctgtccaatgctgaaagtggggtgttgaagtcaccggctgttactgtattgagttctatatccctctttatctctaataatatttgctctgtatatatggttgctccagttttgggtccacagatatttataatttttctatcttcttgcaggattgacccctttatcattatataatgacctccatttcccttcttataatttttgccttcaaatctattttgtatgatataactactcctgctcctttgtggtttccattggcatggaatatcttttcccatttctttattttcagtctgtatgtatctttatgggtgaagtgtgtttcctgtaggcaacagatcattgtttcttgttttttcatggttcatccagtctgtgtattttgattgtttagtccatttacataaaatgttattattaataaatcgggacttactcatgccatttttttagttgttttctggtcttctcttccttgttttcttcctccctgccctccttttactgaaggtgattttctgtggtgctgtgatttagtttctttctctttatttatttatttatgtattttttgagatggagtcttgctctgtcgcccaggctggagtgaagtggtgcaatcttggctcactgcaagctccacctcccgggttcacgccattctcctgcctcagcctcccaagtagctgggactacaggcacctgccaccacacctggctaattttttggtatttttagtagagacaggtttcaccgtgttagccaggatggtctcaatctcttgaacttgtgatctgcccgcctcggcctcccaaagtgctgtgattacaggcatgatccaccacacccagcctttttctctttattttttgcatatctgttgtatgtttttcggtttgagaataccatgaggcttgccaatatgatctaatacctcttattttaaactgatgaaaacttaacactgattgtccaaacaaacatgcaaaaaaacctgataaaaacactacactttgaattcatccccccacttttcaagttttcattgcttctctttatgtcttattgtactatgttttgaaaagtttttgtagttattatttgattggttcatcatttagtctttctacttaagaggaatttataccccacaatgacagcataataatattccctgcatttttctttatgcttactattatagtgagttttgtactttcagatgatttcttcttgcttattacatacttttctttcagattgaagaactccctttagcatttcttataagacaggtctggtgttgatgaaatctctcagttattgtttgtctgggaagatctttatttctccatcatgcatgaaaaatatgttttgccagttataatagtataaagtaattttttttcagcactttaaatatgtcatgccactctctcctaacctgtaaagtttccactgaaaagtatgctgccaggtgtattggggctccattatgtcatttgtttattttctcttgctgcttttaggatactttatcaatgaacttttggagtttgattattaaatgccttgaggtagtcttctttggtttaaatctgttggatgttctataactttcttgcacttgtatgtttatttctttctctaggtttggggagttctctgatattatccctttgaatacacttcttatccgtctttctctgtctactttctattcaagtccagtagctcttagacttgcccctctttggctgttttctagatcttgtagccatgctttattcttttttttttttaattttgtctcctctgtgtattttcaaatagcttgtcttcaagctcacaaattattttcactccttgatcagttcttctattaagagactgtcatgcattctttgatatgtcaagttgcatttttcaactctagaatttctacttgattctttttaattattttaatctctttattaaagttatctgatagaattataaattccttctcttcgttatctttagtttcgttgagtttcctcaacagagctattttgaattatctatctgaaagcctgcatatctatctgtttctccagaattggttcctggttccttatttagttcatttggtgaggtcatgttttcctggatggtcttgctgcttgtagatgcttgggagtgtatgggcattgatgagttagatttttaatttggtcttggactccgggcttgtttgtacctgtccttcttgagaagactgtacaggtattcgctgggagttgaaccccaagcccaataatgctgtggttttataggctcctagaggtaccccattggtggccttggataaattctggaagaattctctgtattaccagtcagagaacctcattcttttccattattttctcccaaacaaatggagtctatctctgtgctgagccacctggaatggggatgtggttatgcaatcacccctatggccgccaccacacagactgcattgaatcaaaccggaagccagcacaacactgggtctcgaccttggctcactataactactacctgggtaaaacctattttcactcaaggccatagtgctctacagtcagcagatgttaaggccaaacaggtttgtgtccttcctttcagggcaatgagttcccccaggccccgggcaggaccaggcatgctgtctgggagccaaggattagagtcaaataccttagaaatttacctgaagttctattctactgcagctaagctggcactcagaccataatccagaatctttgttgcacttccctcccctctccacagggagaagggcctctccctatagccatcagcaccagcagcccatagcaggggttctgccaggccactggcaatgttcacttaaagcccagtggctcttcagtcaacaagtggtgaatgctgccaggctgggaactcgttcttcagagcactgggctcccatatgccccagggcagatccagaaattatatccaagagcctaggctcagactcaagagacctcaagagcctgcttattggcctaccccactgtggctgagctggtacctaaggtgcattacctttctgtctgcttttctcaaacagaaggaatctttttctgtagctaccacagctctcaatttgttggatcacacctgaagccagcatttctcagagcccaaggcccatggtgtactacctgggtatattgctgctggtggttgttcatggcccaagagctcgttaatcagcaggcaatgaatcctgccaggactgggttcttctcttcaagacagtgggtttccttttggcccaggatgtgtctagaaataccatccatgagctagggcctggaatgggaacctcacaactctgcccagtgtcctgtcctactatggctgagctgatatctaagatgcaagacaaagtcctctttactttttgttccttcctccttaagcagaaggaaggagtcacttttgttgctgtgctgtctggggttgggggaatggtggcacaagtactcccttagccaccctggctggtatctccctaggtcacatgccaccctagtccactggctctaattccagttcaacactagtagttgcctaggaattgcagggtttgcatcttagactgccttttaagtttacctaggaccccagagcactttggcccaagatggcgaggcttgctgaggagttcaaatttagaccactgggacaggcaattcccttctgtctagttcttgtccaaaagcttcctctggccgggtgcagtggctcatgcctataaacccagcactttgggagaccgaggtggatggatcatctgaggtcaggagttcgagaccagcctgaccaacatggtgaaaccctgcctctactaaaagcacgaaaaaaattagctgcccgtggtggcgggcgcctgtaatcctagctactcaggaggctgaggcaggagaatcacttgaacccgagaggcggaggttgcagtgagcaagactctgtctcaaaaaaaaaaaaaaaaaaaaaaaaaacttaaaaaaaataacaaaagcttcctccatatgtgggtgctggctgaggccagcatgactttgctgtctgctgtaacagggcagcactgaattcaatgtaaagccccccagttgctgagctttccctcccctatctgcaaagactctcctcactgcatggctgctgctggggcatggggaaggggtgtcatcagtgattcatgactgtctcttctaccctcttcagtgcctctttcagcaatatgaagttaaaactaagtactgtgattactcacccgatttttgattcttgtgatggtgcttttctgtgtgcagagagttgttaaactttggtgttcctgcagaagctacaaatggtataggcttctattttgccatcttgctccatcgtttcctcctgtagcttctttttcattttcttaataccattttctgaagaacagttttgtttattttgatgatgtccagtttgtcattatttttcttttatggattatacttttgatgtatctaagaaatccttgtctaacccaagatcacaaagaatttctattttcttttttgtaaattttatagttttaggttttacatggtttacgatccattttgagttaattatttatggtatgaggatgaatttttctatatctctatcctacttgttctggtaccatttgttgaaaagactgctttttctccatggaattgcttttgcacctttgcaagaaattaagtgacaatatagagccagttctgttacagtgtgacatatgtgatctaaatcactgcagtatgcataaatccatagtatataaaccacagaacttatggggaaaaggaggcaaggggcataactttaaaatgtttgatcagtgatacacaaaagaaaagaaagtaaaaagggtagcattgttctgtatgttctaaatgattaagaaatacataaagactgcaataaatacagttccttaccttgaaaaagacccgaagtttgcttgtggaagtgggtgtcagaaaagttacagtttgtaagttataatgatgtagtggaagttagattatctggaattggaaggaaaattttaacgctagaggtggacaggagtggctcataacaaatacagtgaattagctggtagatgtttgaggtgtgtgcatatgtgcattttatgtatttctactttcctaagctcagctgattcagttttctgtgctcatgtaacgcttcctgcagacaaaatcactgataagccagtgtgaaattcacgttatacttagattgtttgctaagatatcaattgcattagaacaaatttgcattttcaaaagaagcattatagcagaattaactgtatatgtcagtctatcattgtacactcttctgttccattgatatatatgtccaacctttttccggtaccccactatcttgattaccgtagctttatagtaagtgttgaagtcaggtgtagtgtaagtccaactgttttcttccttttcaaaattgtttggactattttattttcttgacttttttatacaaattgtagaatcagcttattgatttctacaaaagaaattttgctgggattttattgaagtttcattgaattggtatatcagtttggggagaattgataccttaacaacattaaatcgtccaattcatgaacatggtataacttcccatttatttaggtctactttgatttctttcatcagtgtttccagttttcagcatataattcttgcatatatattttatagctgtagatgtatatttatacctgtagatttcatgtttgatatttttggtggtattataaatggtactttttaaatttcgtcttccagttgttcattagtaataccaagaaataagattgatttttattattgacatctttagctccttgaaggctttgaacaaaaatggaagctcaccaaagccaaagcaaaatttgcttttttaatattttaaaaagtataagttactatttctgttttgtcttagtctattttaaaacttagatcaatacggcagctttgatttaattgttgttccctcacattttttgcactatattctagtgtagacctgaaagcatgtattttgaggatgagatttcatggaaggatctttaaatgaccttaaagagtattgttatgaaatacaggctgctttctcttattgatctcacaggaactttcttaagagtaactgtaggccatccataaataattgtaatgtttaatacattattttgtttcttttgacgttttctattttctgaagtttttcagacacattttttggtaatactgttattatagattttggactcagcaaggagtcagtagatcaagaaaagaaggcttactcattttgtggtacagtagagtatatggctcctgaagtagtaaataggagaggccattcccagagtgctgattggtggtcatatggtgttcttatggtaagtagcattatttgttttaattatgccaacctgtgattgatattacttataattttattttaattcaaattttgggatttagcaggtcagaaaggggttctgataatttataattaacaccaaaaaccttgtgattggctagctgttacacaataaaaattaagattttggtgttctgatttattttaaagttgctagtaaaaaggtaaatgacagcattgttattataaactattcatatttgatacttatgaaaaggtctgtcagattctatatttcagaatgatgagtttatcagttacatggtattttaattgaatgttacagaacttagcaccttaccatatagtgtcttattgtcagattgttccatgtattccctagaaaggttataaacctgttgagcagaggaatcatgtcttatactacttttcatctcccatagtatcaagcatagtgagcatttcagtagatgcttaatacagactgactaatttggtatgagattaagcatagaatttgcagtagtcagtaggatgatagataaatagctgttgaattagatataatttgctttggaggtctgatccattttgtccatttatcactgagtagcacatcaaatgacacatcagcatgttaagtgtctcttataagcctgacatattaggaggcctgtggggtaggaactgattctaagtaaaatatttggtctttttcaattggagtagctaaataaactctcaagaagtaccttaggagttcagggaagatttaatgtaagctgatgtaggcaaactgaaagagttggaacttaaattaagccctgaaggatggataaaatttaaatagttgtgtgtcaagaaggagagttattgtgatcatattagcaaagacttggagaaccagtttgactagagaagtggagaaagagggcaaaggtggaggaagtgaagctataaacctataagcatttatacatgattctctgggtaggaggaaacttttagattcttaaacaggctggaaattagagaaaattgtattttaggaggattagccttgtaagtggattgtaatggaagaaactagaaataggaagacaaaagagatctttaaagacaattgtaagattatagagatttcaaagtaagcagttgttaaccttggtagcaaattagatgacacagatcaaaaagaaatgaattgtcaaagataaaaatgcctataaaatttgtgagctttagggggttcaaagaatggtaagttaatagaagagatagctgaagaggacagctggttttggaagtgggaaaaaataatttttaacatgtagcataggttactgtaggttaatgactcaccactgggtggtgataaactaggaggggtagtggagagagaatcagaatcatttggagaccttaatacgtatatattaactacccaacgtgtagtttctttgagggaagatgtgagtcctatatacgatccttccttcattctctccctgaagttctcacctctactcctccagtctttggtggcgtaccattacttaagatttgggaaggaaagctggagtttgagagagattaagccttgaacagcagattttgggagacattaagatacagaagagacttgaatgtataagctcacttagattgtgagttcagataaatgagtagaagactaaggccaaagccattattgaatctaagtttgagatttaaatactggtgatacaaaaacttctctttaatgtaccgtaatgatttttagaggtagtctttttttataccattatttaatgttctagttagtcacattgcagtcagttctctttgagaaaatgttttccttcttctgtgtacaactaaggtgtatgtttcttttttaaagctattggcatgtgtgtgtgtggcagggggtggtggtggtggtgtgttctgctgcttctacttcttttttggctcctgttttgggggcagtggagggaatgagaatggttaaatctttcttgggcatgtatgaatttgaaatgcttttctgcttatttttccttttcctttattattaaacattctcttcaaaagagtatatttgttttaacagaaaatgcattgtgtgtattttcaaattgctttggctttgagaacatagccgaattagaactgaggaagaattggttggtttcatctgctacaggtcaccatcagttgagctatatatatagagagagagagtgggataccagaagtatatttctttaagtgacctagttttctggtcccattttgatctactatcatgtagatcttacaatttgatgattatatagaagtctcagtttttttctagagaatcttattatatattctaaggtgttctaattgtgtggtgtgttttgtttgtttgttttagtttgaaatgcttactggtactctgccatttcaaggtaaagacagaaatgagaccatgaatatgatattaaagtaagtgtaaacagtatgtttttcttttgggaaaaaaatctcttgctttcagttttgaaaaaattattgcggttttaaaataatacaagagtacctttttctcactgtcttgttcccttctaccatatacaggactgtacccttgtaaaagcacttttaaaagttctttttgagatttttcaaatcaaaaatgtggctaaatttaagtgaaactttctaaaatcctctgtttcagagcaaaacttggaatgcctcaatttcttagtgctgaagcacaaagtcttctaaggatgttattcaaaaggaatccagcaaatagattgggtattgcttattttcttagcttttatagaagtaactgcatgttttgggaggataattaaatctttatgttttcttgtttgatttgagcacattacttctatacgtagaatttttcattttgtgtgtgcatttcagacaaagtaaactctgttatgtccatgatttaaaactgaagtagtctagttaatgaaagcttctgttcaacctattattatgtatatattatgggttacttcttttgaaataataaacagattctcccttgtacaggtaatcttttatatatttgaagatagatattttgcccccccccaatattcttcaaagtaaatattatgtttctgtaactacttttttgacccctttctggtagcctttccctgtatccagatgcgatcctattatttatgcccttgtgtgaaaatttcagtgtcactttattcctttgtacctttcaatttggcatataaatatgaggtcataagataaattaggaatgaaaaaatatgtatttaagtttaagtattatgtttaatactatttgatttgatttgggagacactttattgtagttcaatatgaatgttagttattttttatcacagtaaaatgaaaggaccagctaataaaatgccagataagaaatcatgtatttttcttttcaaggaatgtatgaaatatgtgtattcctgtttacagatagcagtctggaagccagggagcaagactaaaaaaaatcataaaaataaaatatcatttcttttttttataggatcagaaggagttgaagaaatcaaaagacatctgttttttgcaaatattgactgggatgtaagttaagtagaaagcttgttattgaataacttcatatgaccaacacaggatattaaagatttattgcttttaatctaaataacaaatcagtggaatatttacatacatatttatttttctttttcttaaaaaaataatttagatctgggggtgcatttgtgggttttttccgtggatgtattgcataatggtgggtttggatttctaatgtacccatcaaccaaacagtgaccagtaggtaatttttcaaccctcaattcccttttaacttctccccttttggagtcctcggtgtctattatttccatctgtatgtccatgtgtatccattgtgtagctcccacttataagtgagaacatgggatatttgattttctgagttatttcacataggataataatctccacctccatccatgttgctgcaaagataatgattttattctttcttatggctgcatagtattccatggtgtctatatacctattttctttatccaattaaccattgatggacacttagattgattccatgactttgctattgtgaacagtgctgtgatagacatacaagtgcaggtgtcctttttatataatgatttattttcctttaggtacatacctattagtgggattgctgggtcaaatttttttgattattagttctttgagaaatcttcatatggtttttcataaaggttgtactaatttacattcccattaacagtgtataagtgtacctgttttctcccacatccatgccaacatctgttattttctaactttttaataaaagccctcctgactgatataaaatcatatttcattgtggttttaatttgcatttttctgatgattagtgatattgagcatttttccatgtttgttggctgcttgtatgtcttcttttgtaaaatgcgtgttcatatcctttgcccaccttttaataggtttgtttgtttgtttgtttacctgttgagttgtttgagtaccttgcagatctcgatattagtcttttgtcagaggcatagtttgcaaatattttatcccattctgtaggtcatctttttactctattgattatttatttggctgtgcaggaaattaattaagtccaatttgtctacttttgtttttgttgcatttgcttttgggatcttagtcataaattctttgtatagatcaatgtccagaagagttttgcctaggttttcttctaggacctttgtagtttcacatcttatgtttaagtcttcaaacaatcttgtgttaattattgtaaacagtgagagataggggtccagttttattcttcttcatgtggctaggcaattttcccaataccatgtgttgaatagagtgttctctcctcattgtttattgttgtagattttgtagaaaatcagatggctataggtatgcagctttatttctgggttctctgttttgttccattgatctatgtgtctatttttgtacaagtactctgctgttttagttactatagccttgtagtataatttgaaattaggaaatgtgatgcgtctggctttgttaattttacttagggttgctttggctatttaggctgttttttggttctgtgtaaacattaggattgttatttctaattctctgaaggatggcattcatagtttgataggaattgtattgaatgtggatattgctttggtcagtatgtttttttttgttgttgttttgtttgtttgtttgttttttgacagcgtctctctctgtcacccaggctggaatgcagtggcacaatcacagctcactgcagctttgacctcctagacttaagcaatcctcccacttcagcctcctgagcagctggggatacaggtgtatgccaccgtgtctggctaatttaaatttttttggtaaagatgaggtctcggtatgttgactaggatggtctggaacttctgggttcaagcgattctcctgcctcagcctcccaaagtgctgagattacaggcgtgaaccactgtgcctggactggtatggtcattttaatgatattgattctttcaattcatgagcatgagatgtttctccatttgtccatttgatctagtttctttcatctgtgttttatagttctccttgtagaactctttcacattcttggttaaatgtatttgtaggtattttactttttgcggctgttgttaatgagattgttaattagaactttcatttggttttcagttagaacatcactgttgtatagaaaagctactgatttttgtatgttgattttatttttttaaacattactgaatttgtttatcaagtctaggagtctgttggaggagtcttttctgttttctcagtatcatattatatcatcaatcaatagaggtactttgacttcctcttttccaatttggatgtattttatttctttttcttgcttgattgctctggctaggactttccagtactgtgttgactaggaatggtaaaagtggacatccttgacctgtttcagttcttgaaaggaatgctttcaactttcctgcatttggtatgatgttggagtatgttccttctatgcctattatatggagggtttttatcatcaagtgatgttgaattgtctcgaatgctttttccgcatctattgagatgatcacatgatttgtgtttataattttgtttatgtggtgatttacatttattgattagtaaatgttgatccatcctttcatccctagaataaaatccacttgatcatgattaactattttttgcatgtgctgttggatttggtttgctagtatttcgttgaggatttttgcatctatattcatcagggatattggctggcagttttcttttttggttgtgtccttacctgattttgctctcaggatgataccggttttgtagaattaattaggaaggattcccaactcctcaatttttgggaacagttttagtaagattggcaccagctcttctttgcatgtctggaaaaacttgtttctagacctgtgtggtcctggctttttttgatggaacatttttttattactggctcagtttcactgttcctttggtctgtttgtgttttctgtttcatcctggttcaatcttgggagactgtatgtttccaggaatttatccatcatctagcttttctagtttgtgtgtatatacatgttcatagtaatcactgatcatattttatatatatttccgtggtattcattgcaatgtgtgcccttttcaatttctgattgtacttgttagaatcttctctcttttcttcttggttaaaccatctagaggtctatcaactttgtttattatttcaaagaaccagcttctcattttgttgatcctttttttggtcgcaatctcattttattcagcgaaatgagacttagttattttttgtcttctgctagctttgggtttggtttttcttgttattctactttcgtgtagtttgacaataggtcattgatttgagatctttctgtgtttttgatttaggcatttaacactaaaaactttcctcttagcactgctttagctgtgtctcagacgttttgttatgttctgtctctattttcattcatttcaatttttttattcctgccttgattttgttgttcacccaaaagtcattcatgagcaacttgtttcatttccatgtacttgtgtagctttgagagttccccttggtattgatttctaattttattctactgtggtccaataggatacttgatgtgattttgattttcttgaaagtattgaaatttgctttatggtcaagcatatgttcagttttggagagtgttacatgggcggtggagaaaaatgtatatgctgtagttgttgattagaatattctgtaaacatccatttggtctagagtccagtttaagtccagagtttctttgttgacatttgccttgatgacctgtctagtgttgtcacttggtattgaagtcccccactattattgtattgccatcaatctcttttcttaggtctattagtagttgtttttgaatctgagcgctctggtgttggatgaatatatatttaggataattaaatcttttggtttaattgagcccttatcattatataatgcctttctttgccttttcttactgttgctggtttaaagtctgttttatctgatatgagaatggctacttcggctttgttgtgttttttatttgcatgctatatctttttccacccctttactttaagtctgtagatgtgcttagctattctgtgggtctctttttggcagcagatggttggtttttgtctgttacccaacttgctggtctacgtattttggatggaacatttaggcatttatgttgaaggttatattgatatatgaggttttaatcctgtcataatttgctagctagttgccttggattctcaattgtatgattgttttataggatctttgagctttctacttatgtgtgcctttatgatggtgagtgacatacttttgtttccatctttagaacgtctttgagcattttttatggggcctgtttaatggtgataagttcctctcgtgttttcttttcggttaatgactttatgtcttcttcattttatgaagcttattttggctggatataaaattcttggatggcactttttaaaggaagctaaaattaggtccccattctcttctggcttgttaaaatttctgctgagaagtccactgtcagtctgatgagatttcctttattggtgatttgatgcttttctctagcttcttttatgatttattctttcacattgaacttggatagtctggcgactatatccctcggtcatgtttgtattctgtagtgtcttctaggtgttctctgaattgcttatatctggatatctacgtctctaacaagattggggatattttcctgaattagttcctcaattatgctttccaaacttcttttcttttttctttctgcctataagttgcaagtttggtcactttacattatcccatatttctcaaagactttgttcattttttaaaaattatttattctttaattttatctgatttaattcaaaagaccagccctcatgctcaaaattatttcttatgcatggtctagtatgttattaaagttttcaattctgttttgaaattcctttcatctttttttcacttccagaatttaaatatatatatatatatatatttatttatcttttatctcctgaattgttttctgatttctttgtgtttgtttgtatgtgtttgactttattttggatttcattgagcttccttacaatccatattctgaatgttttatctgtcatttcaaagttttcatttgattaagattcattgctaggtaggtactgtgacgttttggggatgtcattacattctttttcttcgtagtcctgaagttcttatgttggttccttcttatctgtagaagctgtcacatattacttttgaatttattttcatgtggacgtggttcaccccctgccgccgcccctcttggggggtgtgactgtagagtaggttgggaagtagccagaagtcttttggctttgtttctataggcctgtgcacttcttagtcaggtttttatgttgggttttacagttcaatctatagggcagtaggtggcatttaccagtacaagccagctgtggcaaaagcagatcggtatgcacttgatatttgtttattgtgtggtgctctatgttgtttcagatgatgggctggacagtggaatgccaggttccctgacctttttgtcccatgaggggtgaggggaaagggagctaggcagagcttgattgtctggcttgcacacaaataccccaatgatgaactggggagccagccctgacaggcatggttgggggagtacctggtgagcagtgccaaggtctcagcagagagtaggaagactagcttcacatcctagacaggtagcaatatgttctatttatctatcacacccctgactcagagctcatggctctcagttcagatccacaactgccgtgtatctccagaccgcagtatagctgatagccacagaagacacttgtctatgggagttgtttctgggtggaacctcctgaatcagtccaatacacacagctttgtgactcacctgtttgccaatgggtaatgcttctgctttctgtagatatcacagaggggctccactttttagcacacacagttgggtattagctgtggcagtatcgtctggttgggtccctctgagctcagaccccaggttgaggcatggtcatgtactagcagtggtagacaggactaggcaaacttccaattccttatcccccagaatgcccaggagacagcatgtatgagtcctgtaagaagtggaccaggaccaggccagtggacttgttaagtctctagactcctgctgctggctatgatagggagtgatgggctggttcttggagtcacaggcagaactctcaagcagaggcagggagagtactctggaagtgggagtcctagggtacatcatgtgcctgtggaggctgggatctcagaagggcctacaaaacacttaggcagttgcagaatgctcaggcagttggtgcatcaatgcatattgcagacctgtggggctgggctctcgggagcgcccacagaatgctcagaatatagcaaaacactcaggtgattgggtccctatggctgatcacaggcctgtgggagctggtctcttaaaagggcccacaggattctcagattatcacagaacgctctggtggtgggattcccatggctgatcacatgctttggggtctgggctctcagaatggcctgcagaatgcttatgtggtcgaagtcccaaggcccatcacaggcctatggggggctaggctctcagaaggcctccaggtaacaggtgaaatagttgggagcagggccgctgggctgctggcctttcactggggaaggcagaccccttcagctgaagcaatgaaggttggcagctatggggtgcttggcccacctgcacttccctcctgaaggagcagcagtggatttcactttggtgggcacacaaaggtgcccagtttccccactgtctccttggcccaggagtggcaagtgtgatggtggtgttgggcgcagctattgagggagctgaagagcgggtcacgagcctctgggggctgggctctaagaagaacaccaggctgtggccagtatatacatgtaagggcagggcagctgtgctgtagacccaacactgaagaagggaggcctcatttagaagggaacagctgagaccggcagtctttgcagcgtgcattctgctcactcctctgtactgcagctgcagtatctgtctttgaggtgtgcacaagtaccagcctccctgctccctgcctagtgaggtggcagcagccggcgccaggctgctcagggatcaaaagtccatgggattccacatgggcttgagcagtacctctgcacagattccaggcagctccgtgtattaaactggaggcctaggggattcaagggggtttacctatggttagcattgaaagtctgtggcagaattgtggaatcctgggggtctctcactcaatcttcccttccctgcatcgaagatctcctggctccacaccagtctcagttcacctgacggcatggctttgttctcttctttctgtgattcccaattccatcacttctctgatgaattctagtgtggcctcttagaaaatctacttgaagtgtcaatatttattgctattttgattcctcaccatgagagaggcatccactagcccactagctgtttctagtaagccataatgaactaaaagtcatgtttacatatttaaatacataaaaattactcaaaagtaattccctatttatttttccccactgtgggactgtaagctttttaaaaaatagatttcaaaagaagtggcttttatatattattagaaattttctatgcctttcaaaagcatagattttagcaagatagattgttagacgactaaattttcaattatctgatacaagcctaaacagtacatttaacgtatattttttatgtgaaaacaatattttagatgaaatatgcactttttaaaaataattattgattttgaaagccttgagcattttagttattactgtgctgtatttgcatggttatattatcaatgagcattcaaaaacttctgatatccaaaattgagcattccttgtgaattcagcttgtggcatcattgctatgtttcttggcctcattacatcttgaacattatctctcaaaattttagaatttggttaacagttatctgaaactatgaaattgttagtcttctattatggactgtgaatattatgttctgctttgttttacaaagtataataatttttaaagtcttaatacgttttgtgttatctggttgtcccttctagaaattatataaaagagaagttcaacctcctttcaaacctgcttctggaaaaccagatgatactttttgttttgatcctgaatttactgcaaaaacacctaaaggtaatgcatgtttatctgtaaaatcagagattttatttaagtagatagtttaaaaataaactttcttgtgaagtaaaaactaaaagaatggtaaatttgcagagagttgatttgtccatgggaaatatggatcctatacaaaacattgcttgggcagctgtcaagtagatgttttaaatgggaaatatttatcaggaaggaaggaacttggtcaaagggtggcaagtttatttctttgttttataagtcctttggcatgctaatgatttacaggatggggtaggaggtaagacgtgagatatattactctgtcttttacagtttcatacatacactcaagaaatattgaatacctactatgtactaagcaatgaggatgcagtggtgaaaagaacagacaaaaaatactgactaaaatggagcatgtatacagatgggaggagacagataatatataataaatcaaatatataatatgttagtagcatttaatggtttcctttatagttaaagaaattctgacactttatatcatagaattctgtgtgtagttataattaaatgtaattatttggacattagattctcccggtttgccagccagtgcaaatgctcatcagctcttcaaaggattcagctttgttgcaacttctattgcagaagaatataaaatcactcctatcacaagtgcaaatgtattaccaattgttcaggtaaattccacttaatcacattattcagttttgggataattatttctctgttattgctgatgtatttttaatcgttttttttaaagtgatttttaacttctttgtttatctattggtataaatcatgaattaaatatgtccctgtgtgttttggaaaagagcagacagtggtaacttttaattatatttatgtggcctcttttgaataaaatagaagggtgatgtataagctagttgctttattatgttgcctgtgggtatcaagaaaggattggttcacgccatttataaatgtcgataaaacttctgtagaatgaattattctagtttaaggcttaagatgaatgtatatttaggacaaaagacagaaaatgtgaaaatatttcattctccttagtattttactttttaaaaataaacagataaatggaaatgctgcacaatttggtgaagtatatgaattgaaggaggatattggtgttggctcctactctgtttgcaagcgatgcatacatgcaactaccaacatggaatttgcagtgaaggtattgtctctgagcctagctttcttgtttgttttatgcatgtaatttaaattatgagtcaactgacaaacattttcagtggagtaaatgaagataggaaagataagatgatgagaaagaactctgagacttaatattttcatttttgaatttcttttcaagcctttgttatgtaggaacagtgtatttcatgaatttttgcatttgaataattttgttatacatgtattatgttaagattgagcagttgtgataattacaatcacgtcatcattggtacaatttcagtgaaataggatataatgttaaaattttatttttttctgttaagttttttacttatgtttttaaatgttaaaataatatgaggtaagtattgttcttgagtttttaagcatttcttcaaaaatataaccaaatggaaaatactcattttagacaaaataatttgtttgcctcagatatttttcttttttcgtatttcttagatcattgacaaaagtaagcgagacccttcagaagagattgaaatattgatgcgctatggacaacatcccaacattattactttgaaggatgtaggtatttctaaatctaggtatcagctaagctaatgaagttatgaattggaactttatttttcttattgaaacttttaatgtctttagcatatttattttcatgcctcaaaaatctcaggactcttaaattgccactttaattcttttttttcccatgattctatacttctagtcttatagaatatcaaaaataactatgatgattggtaaatgagaatttcctcattacatgaatatattctggctggttaactagttaacattgtagctaacaccaattattattctgtagtgctatcaatacagaagatacaggagactggggatctgccaaattcttgaatattttactttcttgattgtatagtaattggctatagcattatggattgtttctggagtgaaatagtcttagtctatagttttgtaaaatgaatgtataactcattaatatgttactgaatgctcacttttcacataaggagataatgttcttttctctttatacaagacagaaaagtcaactatcaccttgatcaatttgtgtcagaaaatgatatgcactttgatttgtaatctttaaactactgtggtgtgtgtgtgtgtgtgtttatcatacatgccctgagtgaaaagtgtataaatacttttcttatttgttttccttggtatactattggaattcaaccagtatttaagataccaaatcagataatgaactgagggaagggtataaaaaataaataatgtatttactttcagaaagaagcttacgtttggggaagagaagtagctataatacagtaaaaaaattgagatagaggtcatttgagaggcacagagttctattttttaatttctttctttgatgaactgcaacctataacttcaaaaggcaataaaattatatgtttaacatggtttcatgggactgtgtaggtgactgtttagccaattcaacctttagtacagcctgtactgcattaagagtaaaggaataatgtcttttttgtttctaatactagagggaagtctagtagaaaggtaaatataattaaaatctaatttaagaattatagaattgtaattataaataataacattttaaaactgcgttcttttgtaggtctttgatgatggtagatatgtttaccttgttacggatttaatgaaaggaggagagttacttgaccgtattctcaaacaaaaatgtttctcggaacgggaggctagtgatatactatatgtaataagtaagacagttgactatcttcattgtcaaggagtaagttgtagttaacttttttctttctattctgggacctatatcttcatgaaaatataggagatgatgccacctaaaataagttgcttttattcacatatatttctatagagaatatcctaatagaactcacaatgtagtgaagtttatggatttaggaaataattattttctctctgccaattggccctgatttaatagcatattcatttaaaaagcggtttgaattatataaatgtacctaactttgaccttgaaattatgaaacttagacttaaaagtcaaacataaaagaattaaccaaagacatacagtgactattattcaagttacctaacatataaagttcagattacctaacatgtatcttgaaccttgccagctccttccctaaatacagtaagaaatgtgtattctaaacatttcaagtattactatattaaattttgtggtgaaggggcaggcttgttattttgcttcctgtcctttcataaagaaggcaaatgaactgagttacaaaaaaagggaacttttaatatatataactgtataaaaaaacaaagtagggagtaatttaaaaatttaagtttgaatcagttgtaatagtagatatgtaccaacttaataaagaaatttaaaatggcaatttagaattgacataatgaaaactctgcatgagcccctcttataattagtaaaaatagtactcaattttgtgtgtggtttaataaagtgtgttttcagtattttttaatatataatgctaaacagtagctttttataatttaattatttgattgtctttaccaaccctctctaaaataaagcatgaagagaatttttgagaaaagcatcataggcgtctcttctttattattgtatgaaccttcaggttcttacttcaattaactaaattaataagacacatattgagcatttctacaggccaggcattgtaaaaagtgttgtggattcaaacatgagtgagacctggtccttgtccttaaagagctttagcatagtaaactctgattaaagataaggataacttacatgaaatagagagtactagtagggaacttatcacatgggggaatttcagcatttttttttaccagtcctacattttatttgtttcttctttattgtttctatttttctgctgagacttccctttttttgctgagagtttaatgcattgaaagcatgttttgttttactttgttgaggatcattatgatagtcactttaaaatccttatctgttttagcatggttcatcttggaatcagaatttattgattttcttaagactgtgttctattttcttggttctttgtataacagataatttgggattgtatcctaagcattataataatcagttatagagaatttcatatttcattatttttctccaatagttattattgtttcatttgtttaagtgggtaattttcttggctgggcttgaactgaaaactagttttggggccagcagctctggtttttgttcatattatttgtctttatacaaattattttgatcctgttccacacatttttgatacaaggatcagttctggctctttcctttataatattcctccactcagcattcagagttttccagcatctcctttctggtcctctaagccagaaggtctgagggtttttccacatgtctttaccaccactgtgtaccatagggattgcacttaaacccaggctaaaagccacagatatggaaacctccttgtatggtttcctgtgttccctccttcaacaaagtgtgtactcctcatcagagtatatctgcttctgttcattctccagtgccttcagatagttgatttttgtattatatccaggttttatggttgtttacttcaaggaggggggtgttgagtagggtcttagtccattatggactaagtctaaacttagtttagtttgtaagtcaggaactgttcttatatgttatgtataatgctaagtgatataaattatggcatgtccaaaagtggatctgagaattgagcttagatatattgagattagaaatggttaacaaaactagtggcttttgagacaatagaaactggaacatctatctgacagacataatttcctgtgtatattttaggctattaagcttgagcaaaatgtcattttcaaaattttgtaatttggaaagtttctttttctcagatatttagttaattatatagatataatgttttatttctcagatatttagttaattatatagatataatgcttttttcctttatattcaaggttgttcatcgtgatcttaaacctagtaatattttatacatggatgaatcagccagtgcagattcaatcaggatatgtgattttgggtttgcaaaacaacttcgaggagaaaatggacttctcttaactccatgctacactgcaaactttgttgcacctgaggtattccttaaaaacttcaccatttgcctttcattcttttcctttagttgaatctatatgatgccatatgaatgttatggtaggcagataaaagctgttaccttgcccttttcagttttaaagttctttaaatcagggaattctcatagcccctaatataaatatttacactggtaactagcatctaaaaccaaccagaatgtaataatcaggctattctattctgttgggttatatactaatttcagaatcatatcattttagagttgggaggtagggaaaagatagccacagcttcaatatattatgtattctaaatgatgaaactggctcgaggtacttcaatgacttattcaagacccagcattcacttaataacaaagctaggatagattcatcaggtatgttaaattgtactgaacattaggtaatttatatagattagcattataaataacatataagggcagtgctatcctcgattgtctttaggatcttctaactcccggagaaaatagccacacaggggtttttgtaagccgtggggatttttcttggaaaataatatttatatctaaatattagggtgaagaatcaggaaaggtgatgctcttagggagaagagtcaggaaaggggaggctgtggtacagatttcacatttgggaagctgtaacattcattctaaccatggggatgagagctttgacagtagcaatttccatagcagtgaaagtgaagttggggaaaaatgaatcaagagatggcattggccatataagttctttataagctttctttgtcaagtcagtagctgttcttatatgttatggataatactgacatgtaattacatattcaatgaaaatcaaaatacctaaaactcttcagatttgaaaaaattgatgtgtcaatcaaaggagaagcagagtaaaataatttgagaagaattcctatcttctatatattttatgattttatttttatatcaaatactagctttttcagggaaatattgcacatcagatatattaacaataagccacttgtattttaatgtaaatcagtctttattttaaagtagctactataaaaataactttaataatgaaaatagaaatatagaaaatactatactgatattgaagaacaggcagaacatctgttttataagctttttatgataagagatttgactaagaaattatgtatgtatccggagaatgtgatttaatctatattttaataaaattcataaaataatgtacaagtttttatctgttacaacatattgtactgccctaaggtgtatgagataatagcctgcacataaaaaaattagcctttttaggacatttcagtggttttataggaggacagaatatttgagatagtgctcttctgttatgtatagcatatatggtcatcataaaggtcctcataaatattctaacagatttatacagtagacataagagcagagtatagaataaccattttaatagccaaatatttaatattgacttaatcttgtggacctaaactacttagtattatggcaacttctcactggaaaaatgttaaggtgaatatgtaccatatgaacatatcaacaacagacattaaaatgaaaaatatataatatgtccgtgtttagattaatggttaggtaaacagttttagattaatggttaggtaatcaaacccagagagtagaatacagcccattgatgtttttttcccaacagacgtacactgtagtaagacacttttaccattgtaatagtgaaggttttcaggtgggcatctggagaccacttctctctaactagtgcttgaactacctttgactagtatttgtattttcaaaatactttgaaagttttatttttgagtgatgaagcccatgctaggaatcaataatatatttgtaaattgttattaatgaaccacagagagtatttttagtgaggtttgtttctgaaatttcagacgctttcaaaaggtattagtaaaatataaaaatgcattcttttttaaactagaattggaaagattcaaatatcgatacagacatttcaataaaaaatggcatttatgggggtatacattcattatttcacaatgcataaagtttttgctaagagctacactgattattgcaattttagcttaacaataaggagaaaatattcttttagtttttagattatgcatcagtttgatcaacctcttggggtttattaggtagatgtgcttttctactatacctatgttgcaaaaagtgaatatatctctaaaatattttattcttcgataaaggcaaatccactacgttatattattcaactgttattataagcctactgaggttaaagaagttcctgaattaatggggcttacaaaaaagttgagggatactgtgtctgtagctttttataaactaattttgaagttaactctcctttgctctttctctgggctgtcatatagtttgccccaaacgttaattgatttgagcaacaggaatgaggagctctgtgtatgttactttctgctgacttgggaagccaaatgagagagagacttatgccactgactactttaattgtgacttctatctttgtatttaacttgatacaagtaaaaaggtgtgaaacataacctctgtactctttcattcatcctaccgctctgccttgtttcttttatgattatatgctcactagggcattgagactagctctagaaaaaaagtggactgtttcttctttacctaaaacaagtttttcatgtttggccctaattaagcaatatgacacctcctgcgcaagtctgtcctcataactaggaaggttataggagtatatggtacctacagctgacttgattaattctcacctgtccaagtagtttctgtgcagtggccctcaaccttagctatgcattggatcacttgggaaactttaaaaagcttcctaagggaatcagggaaaacatggtgatgattggaactgtaagatgtgatgagaaaaatgaagagataagggaataaaatcactagaagtctatgttttactagagtatgccaagttaacatttaccatgatatgagtggtttagctataccttgttatttgttaagtactttatagttccctacttgtattatttaaataagccaattatgcttttatatcctctttaccctcttaattttgcacatatgtgtgtttttatgaatacactaagactaggaaaaaagcatatttcaaattttaagtttgttatatactctttactcatgtttggcagttgtcaaagccaaacactaacatttttagccttttatagatgagaaattcatgctggtggttatataatccccagtgttaatgcattaataatgtttctattttgttcctatgcctagaggctttagcaatgggcttgtttttctaaatggaaagaaaatggcctcatttcatctctttatttttctaacgaactctattaacttcgtagctaaatctcacatgaatacatagcaaatgtattatttgatttttattaacactggccccaaaaaacagcttcctaaattattgtatgaaaaataatgaagccattcagtatttttcaaatgtgtacttgtggtccagaaatatactgctgttttcaagacattgaccttgtatggtaaaatataaattcattctgcttttgtgagattatatattatcttgccatatcttgtgtagtaaagaggcagttatcatagatattaaaaacacaggcttaaataagacttcgtgttctactacttactagttttgtaatgttgggtaagttacttaacattcacttcagtttcctcatcttcaaaattggaatattgtctacctcacagaatcagtgacagaattaaaagccttagtactgtatatagtatacattaaaatctctgtacatgttaatcttaagtttagatttctctatatctggtttcctaagaggaggagagaaaactcattgaggtgtcaggggttatgtctttactacaagtgcataaaatgattgaagtttttatattatgaacttttgtgagattattagaagtaaaacaacttctttgggagaaaattgagtttatattgagtaacaccatataatgagtatttctttcttaggttcttatgcaacagggatatgatgctgcttgtgatatctggagtttaggagtccttttttacacaatgttggctgggtaagaaacatatattcaaaagcattaacattgttatttgaatttcatctgtatatcttcatctagaaagagaatagattaaaataactttaaggaaaatgacatttacatttcaaaatattaatattgattgttacacataaaattaaaaaacataaagcaaggtgtagaatgaggcactactaatttatgacataataattttaaaaggcagttgtcacccagaactaaaacttttaaaaataaagtgcatttctattttaatcttgaaaatatttatttaatcataaaataattgcatgtttcaccaacaacttgcccactgaacttgttgatattgagttgattttttttaagcacttgaattctaactattctggcactagagatttagtaagattgtgcaaatctaagtagtaacaatactaaactgcagaaacacacacaactaccttcccttgaaaggataaagaaaagctcaaaacaaatgtaataacaattggactatgttaatgttgatactttacaaataatacaagtactattttactctcaataatgagtagcacttttatttagaagtgaaaacttatggatgtagaaattattccaagagggttgtgatagagaaaatggccaagtttagcaagtaattataatgtacttagacatatattttaaagagtgcatttagtatagtttaaatatgcttgacataccaagagctctataaatatgttataaaaatggtgactctaaattgtttaatggcttggtgttaattacacatctcagtacatttcacatacttttaggtattacaaatttatgcaaaatatttagttatagacaacagtaaatactgaagttaagggtagctaaagtttgcatgtcaagcacaattaatctacatgattgatctacataataaatgtaatggctatggagttttgtgtcgatatacatatttaaccaaccaagttttcatagatattaataaaaagtaatgataaccgtttccactgatgataatgatgaaatgttaatgaataaattgaaacaagacaataaatacattatgaatatatgaaattaacattttctttttactaggtatatttgtttgctaacctatgccttgaatatgtgtgttttagtgttgtctagttttaagactgaagctttacatgaaaagccacctttattttattttacattgattagctaatcagagacaaaagaaataattgatgtaaacaagattctccattttattaaacattttattaaggttaaatttatggtttgaaatataaaatttgactttaacagtgaaaggaaatatgagatgatattcttattcactgaagtaaatatttgcatgtagtaaatatatgtatctctttcattgtttgggtatgttaccctctagtgtctattaaaatctattttattaatagctacactccatttgctaatggccccaatgatactcctgaagagatactgctgcgtataggcaatggaaaattctctttgagtggtggaaactgggacaatatttcagacggagcaaaggtataaattataatgttttgttttgttgcattcatgttaaattgctcaaatccctaagttatagcttagtgtgtgtttgcagagtatctaatggtgtcatatgtaagtgttttgaattaacacatctatgagtgaattgaacagtgtacaaaggataacttttggttttaccatgtttctgggccaccttcctttctgatcaatttacatatgtgcagggaatattaactagttcctgtattaaccaaaaccaaaaatgtaaattttggaaacagggtatatttaaagccaactaaagtttatttctagatttaccatgtatttccatatcattttcttttagctcagaaattgacttgattatgtagacaatttttataatgctaacttattcttagagaatgaatggactatgtatctgtggcacctggatattcattcctgatggcttagagtggtccagtagaaatataatgcaaggcaaatacactttaaaaatttctagtagccacattaaattttttaagtgaaattaaatttaataatatgtgttgtttaatctaatatatccaaaatactattcaatataaaaatttagatattctttactttttcttactgagtcttcaaaatgtactatatattttatacttaaagccgtgtgaatatggactacccacattttaatacttaatagccacatgtggccagtggctgccatattagtgaagtttcagcataactggccttatttatttgggggaaaaaatagtcaacccaggcttgaaaactaatttcttacatttataatttttaaattttaagttttaaatgaaaactatcacttgtcaccatgtggcttttaggtcccagaagcttttaagctacaatttgaagtagaacacattttatgacaaaagtaattgcttatagttaacatgtgctaattatctcaagaacactagccagtttgtagaatacattgcaaagtaactccagcatcctgattattgtcaggataaataaactggggatttgtccctaacttttaaacattagaaaaacagacaagggattgttcttggatcttaaccttagtatgagtatgctgtgtgctatagtagaaggaacacttgaatggaagccaggaaaactgagcgctaatctttctttggttccttataactccaaaattcatttctatgcaatttggtttatttgtaatttgcaaaatttccccatgtaatgtgattagaactactagtattattcattaaatatgtattttctagtcatccagtttatgcttcagaaaatacattttaccaaggatcaattttaatgtttaaagccatgcatatgaatctcctgtgttccttttaaaaatatctgctgagatcaggctgttgacagttcctggttggctcacagttacccatgtcagttatctccgttaacatttccaagaatctttgtagaacaagttctccacttgcaaggtcttttatttatttatgtatttatttatttatttatttttattttttttgagacggagtcttgctctgttgcccaggctggagtgcagtggcgcgatctcggctcactgcaagctctgcctcctgggttcacgccattctcctgcctcagcctcccaagtagctgggactacaggtgcccgccaccacgcctggctaatgttttttattttttttagtacagatggggtttcatcgtgtttgcaaggatggtctcgatctcctgacctcgtgatccgcctgcctcggcctcccaaagtgctaggattacaggcgtgagccaccacaccaggcctccacttgcaaaatctttaaagtagaactcttttttttttttttttttttttccctttaaggcagttagcccattgccaaaaggtttactgacttaaggctggaaatgtctttctgagctataattccaaggacttctccacagctaagtgagatgcctcataccagtattaggtgattctttgtgtggacagaacagagcattttcatcttgtgtttaaagcaatttgttggcttcagctcctcaccactttctataccagtctcctattactaacccagtaatccctatgcaaaacaaacaaacaaacaaacaaacagaaaaagaaaacgctgatggtgattcctgtaggtttaagggcttaaatctcaaactttgtgttaggagtaacgggagtgtgctgaaagggcaagcaatacaacaagtcataccaaaaagccacattgttctctcctaagccccaaccccactccactcctgtggccagtggtccaaacagaaaataactggagaaattgaggaggtcaaaggattagggaactaagcgttatgtgaattcactagcaagatgtacagaatgcttgtgtttacattgttttttatggaattagcagaataaaactgatctattttaaaaatgcaaaaaaaaatctgctgaaatagatggggacttaacattgccaattgtgctcaaattttcagttttctgtgttccatgtattttatgtttaggcctatctggtgtttaagtaataatacatattaaagcttggttttattcttacgtctcttttagcctggtaatctggaaaacttctcatagcataagatattagggatagtgttgacatatctcataaggtctcttagataatccctttttatggatcgggtgactttagttctgtctttgaaacttttgtaggagctatgagaaccttttaagaaactatctcatgtaccttttaacagttctactgcttacatttgttttaaactaggcatgaaagtaaacttgttcagtttcccataacctcttaaactagtacactttagttgtgagaactgtgacatttgctgcccatcagacatagattatttattgcctcttcatttgtctcttttaaaaagattaacttgtacccagtgaacaatctctcaccatctcctcacccaccactccccatcctctggtaaccactattctactgtctatttctatgagaacaatgttttagattccacatataagtgacatcatgtgatatttgtctttctgtgcctggctgtttcacttaacacaatgtcctccaggttaatccacgttgccacaaatgataggatttcattcttctttatggctgaatggtgtttcattgtgtatatacacactatattttctttatcaattcatatgttgatagacacttaggttgattccatatcttggctattgtgaataatgcttcagtgaacctgagcatacagatagctctcctacatactgattccatttcctttggatatatatccagcagtgggattgctggataatatggtagaattttttgaggaaccactatactgttttccataatggctgtgctaatttgcattctcaccagcagtgtgtaagggttcccatttctccacatctttgtcaacacttatttttttatttttttgatagtagccactctgattggagtgaggtgatgtctcattgcattttttttttttgatacggagtctcactctgtcgccaggctgcagtgcagtggcgtgacctcggctcactgcaacctctgcctcccaggttcaagcgattctcccacctcagcctcccgagtagctggcattacaggcaggtgccaccacacccagctaatttttgtatattcagtagagatgatctttcatcatgttggccaggatggtctcgatctcttgacctcgtgatctgcccacctcagcctcccaaagtgctaggattacacgcttgagccatggtgcccagccctcactgcagttttgatttccattttccagatggttagttattttgagcacctgttcatgtagcacctggtgctttattttgtctatttggtgaggtcatggttccctgaatattttttttttgaaaagtataatttttttataattttaagttctgggaaacatgtgcaggatgtgcagatttgttacataggtaaacgtgtgccatggtggtttgctgcacctatcaacccatcacctaggtaataagccctgcatgcattagctatttatcctgatgctcactctcctcctgcccctcccccaacaggaccaggtcctgattgtgtgtggtttccttccctgtgtccatgtgttctcattgttcagctcccacttatgagtgagaacatggagtgtttggttttctgtccctgcgttagtttgctgaggataatagcttccagctccatccatgtccctgcaacagacatgatcttttttatggctgcatagtattccatggtgtatatttacctcattttctttagtctatcattgatgggcatatgggttgattccatgtctttgctattgtgaatactgctgcagtgaacatacacgtgcatgtatcttcataatagaatgatttatattcctttgggtatataccctgtaatgggattgctgggtcaaatggtatttatggttctagatcctttaggaatcaccacactgtcttctacaatggtcgaactaatttatgttcccatcaacagtgtaaaagcattcctgtttctccacagcctcaccagcatctgttgtttcttctacttttcctctactgttttcccattactatttggcatgtactttattagagcatgtactacattctgcctggtcatatattcacttgggtatatttttgttcttcacacttaggaagccttaaataacatacttattttgcattttctatcacatctagcataatgagtactcaattggatactttttcataagctaatcatttcttctggtatatcttgcattgttaatttacacagcaagtgtttttgatacattttatacacatgttgtcctattgtgagtgactataatttgcagtaagcagtgagattttacataaatatttatttttcattttggcttgaagtcgcttttaatagaagatgcttttttcatgtgtttacaggttatagatttatagagctagaaggcaagtagtccgtcacctcatcattttgcagataaataaaccaaggcatatagaatttaacttacacattaatgagtggcagagttggaactgattttcagtttagtactcttttcaatccactcacattagggaaaacttcctagaggaaaaaaggatttattaccttgtttcatgtagattaaatggaggaatgagaggtctgtaaccctttaacaattgaaggctcgaaggaaagctattattgttgacccactttgtttctgtagtaatggacatgaggtatacagaaccttattacctgatatgttttggctgtgtccaaactcaaaatctcatcttgaattgtaatccccataatccccatgtgtcaagggagacaacagatggaggtaactgaatcatgggggcaatttcccccatgttgttcgcgtgatagtgaatgagttatcatgagatctgatagttttataagtgtttggtagttcccccgcgtccattccccttcctgacttccttgcttgcgctcctccttctaccgtgattgtaagtttcctgagactgtcccagccatgcaaaactgtgagtcaattaaacctctttatacatttcccagtcttgggtctgtcttcatagcagtgtgagaatggactaatacattatcctttacatgatctgtactacagagcagttttacagttaaaataatatattgaagttcatctagaactcatagtagtaccacagttttcaaatgcctcatgccaaattatactagtgaaagactacactatctaagccaagctaggaaggtacattagcaaatgcaataatcagaaatcaacccttgttttttctgaaagataattatttattattatttgtgatcagagcaggtgttgctgaagtatatatggaagttattcatttatactgaggatatatgaattgttacacatttgaccaattaaacatgcttacagaatgagtaataaaaaagttttacctcagatacatgattacattgaaaatagaagtgatgaattattaatcagtattttccaaactttaatttggaattaaatttgagtactgtgagaataggaaaattgctatgctcctatattcatgtatgttcaaagtatttagagttttgctggcttttatccatttaaaatggacattaaaaataagaaacctgagtacattgagaggggccaagatggccaactataagtaggtgcagtccacagctttcatggagagcagtgaaaatggtgtgttaattcagcaccttcaactgaaatatacagtttcgtgtattgggactgactaggcagacaactcgacccatggaggagaaagaatagcagggtggggtgatgacacacctcagagtggcacggagacaaaggaacccccagccaaggcaagtggtgagcgattgtgtgaccccacctagagaaccgtgcttctcccatggatctttgcaacccatggatcagtagattccctcatgagcccatgccaccagggcctttggttcaatatacagagctgtgaggagtctcagtggggcagctgctgaggcacatacagagacccaagacccaagagttttgcatactctggctccaggaattccagcaaggcagaagacccatctatacttctatcctaggaagggacctgaatccagggagccaggcagcatcattctgtgggccccacttccatggcacctcacaagttaagacccactggcttggaattccagccaaccagcagcaacaggctggaaatggtctgagatggaccaagttccagggggaggggtgaccaccaactctctggtttggttgggtcagtcattctaacctgccagctccgaggagtccaggcagtccagacgaggaggagtccctgatgaagcagcacagcagctgtgccagattgtggccagactgcttctttgagtgggaccccaatccatctctcctcactggacagatcctttctggaggacttcagcacttcagccagggttatatagagagaaatttgatgtttccctaggatggagcccctggtgggaggagtggcaccagttactgcagttcagtcaacagccattctagcctgcctgctctgggaagtccaggcagtccagaggaggaagagtcccccacaacccagcacagctgctgtgccagatcatagccatactgcttctataactgggaccctgatctatccctgttcactgggcggggcctccctatgggaatttcagccactccagccaaggttattgggacagaactctgatttctccctgaggtagggcccccggggaaggggtagccaacatctctgcaggttgttcgactcagtctttccagcctgctgtctctggggaggccaagcagtcaggacaaggaagggttcccccaatgcagcacacctgctctaccaaaaagcagccagactgtttcttaaagtgggatcctgatcccatccctcctgactgggtgagacctcccaacagggatctccagacacctcctacaggagcatacaggtcagcaacaggtcagtatccccctgggacagaactcccagagtaaggagcagattgtcatctttgctatttcacagccttcaccagtgatacttccaggtaagggaaaatcaatgaaagctagggtgtggagcagtcccccagcaaaccacagcagctctatggaaaagtggcttgacttttaaaagaacaacaaacagaaaacaacacaaaacaacagcaccaacgaaaaagacaccacaaaaacctcactcaaaggtcagcaacctcaaagatcaaaggtaggtaagctcacaaagatgagaaataatgcaaaaatgctgaaaactcaaaaaggcagaggacctcttccccttcaaatgactgagatggctgaattgacagaagtgggcttcagaaggtgggtaataacaggctttgttgagctacaggagcatgttgtaacccaatgcaaagaagctatgaatcatgataacacaatagctggtaacaggagctgacaactagaatagccagtttagagaggagcataagtgacctgatggagctgaaaaacacaagaacttcacaacgtaatcacaagtatcagtagcagaatagattaagcagaggaaaaaatctcagagcttgaagactatctttctgaaataagacaggcaaacaagaatagagaaaaaagaatgaaaacaaacaaaacttcagagaaatatgggactatgtaaaaagaccaagcctccaactgattggggtaccggaaagagatggggagaatggaaccaagttggaaaacacacttcaggatattatccaggagaacttccccaacctagcaagaaagaccaacattcaaattcaagaaatccagagaaccccagtaagatactccaccagaagatcaaccacaaggtacataatcatcagattctccaaggttgaaatgaaagaaaaaatgttaagggtagccagagagaaaaggccaggtcacctacaaaaggaaactcattagactaacagctgacctctctgtggaaaccctacaagccaaaagagcttgggggccaatattttaacattcttaaagaaaagaatttccaacccagaatttcatacctgggcaaaataaacttcacaagcgaaggagaaataaggtctctttcagatgagtaaatgcttagggaattcttcacgaccaggcctgccttgcaagagctcccgaaggaagcactaaatatgaaaaggaaaaactggtaccagccactacaaaaacacactgaagtacacagaccagttacattatgaaacaaccacataaacaagtatgcaaaataaccaactagcatcataatgacaagaccaaattcacacataacaatattaaccttaaatgtaaataggctaaattcctcaattaaaagacacagaatgccaagctggataaacagtcaagacccaatggtatgcagtctttaagagacccatctcacctgcgaagacacacagagacgcagaataaaaggatggaggaaaatttaccaagcaaatagaaaacagaaaaaagcaggggttgcaatcctagtttctgacaaaatacactttaaaaaatcaaaagacaaagaagggcattacatgatggtaaagggctcaagtcaataaggagaactaactatcataaatatatatgcacccaataaaggagcatccagattcataaagcaagttattgagagacttataaagagacttagactcccacacaataatagtgggcgactttagaactcctgtgacaatattatacagattatcaagacagacaattaacaaaaaccttcaggacttgaagatttttgtatcaagaggacctgataggtatctttggaactctccaccaaaaatcaacagaatatgcattcttctcattgctaggcggcatttactttaaaattgatcacgcaattggaaataaaacactcctcagcaaatgcaaaaaaaaagaaaaactgaaatcataacaaacagtcctctgaccacagcacaatcaaattagaactcaagattaagatatttactcaaaaccacacaactacacggaaattgaagaaactactcctgaatgactcctggataaataatgaaattaaggcagaaatcaagaagttctttgaaactaatgaaaacaaagagacaatgtaccagaatctctgagactcagctaaaacagtgttgagggaaatttatagcacaaaatgtccacatcaaaaagttagaaagatctctaattaacaacctaacatcacaacagaaagaactagagaaccaagggcaaacaaacacaaagctatgagaatacaggaaataaccaaaatcagagctgaactgaaggatatagagacacaaaaaaccctcaaaaagtcaacaaaaccaggggctgatttttaaaaaacaaaattaataaaatagacaactagctagacagaagagagaagaatcaaatagacaatagacacaatcagaaattataagggggatatcaccactgatcccacaaaaataaaaacaaccaccagagaataaacacctctatgcacataaactagaatatctagaataatggataaattcctggaacatgcaccctcctgtgacaaccaggaagaaattgaatccctgaatagaccaattgtgagttatgaaattgaggcagtaatagcctaccaaccaaaaaagcccaggaccagatggattcacagctgaattctaccagaggtacaaagaagagctggtaccattccttctgaaaatattccaaaaaatcgaaaaggagggactcctccctaactcattttatgatgctagattcatcctgatatcaaaacctgacagagatgtaacaacaacaaataactccagttcagtatccctgttgaacatcaacacaaaaatcctcagtaaaaactggcaaactgaattcagcagcacatcaaaaagcttatccaccacaatcaagttggtttcatccccaggatgtaaggttggttcaacatatgaaaatcaataaatgtgattcatcacatagacagaactaaagacaaaaaccacaattatctcaataatgcagaaaattcaacatcacttcatgttaaaaatgctcaataaactgggtactaaaggagcatgcctcaaaatgttaagaaaataatatgagaaacctacagccaatatcatactgaatgggcaaaagctggaagaattccccttgaaaacaggcacaagacaaagatgccctctctcaccactcctattcagcatataattggaatttctggccagggcaatcaggcaagagaacaaaataaagggtattcaaatagagaagtcaaattttctttctttgcagatgacttgatcttatatctagaaaaccccattgtctcagcccaaaagcttcttaagctgatgcaacttcagcaaatctcaggatacaaaatcaatttgcaaagattgctagcatttctatgccccaacaacaggcaagcggagaggcaaatcataaatgaactcccattcacaattgctacaaaaagaataaaacacctacgaatacagctaacaagggaagtgaaggacctcttcaaggagaactacaaaccactgctcatggaaattaggacacaaacaaatggaaaaacgtcccatgctcattgttaggaagaatcgatatcatgaaagtggccatactgaccaaagcaatttatagattaaagggtattcccactaaactaccattgacattcttcacagaattagaagaaactattttaaaatttatatagaaccaaaaaatagcccaaatatctaagacaatcctaagcaaaaagtacaaagctggaggcatcacactacttgacttcaaactatactacaaggctacagtaaccaaaacagcttgatacttgtacgaaaacagacacatagaccaatggaacagaatagaaactcagaaataagactacacatctacaaccatctgatctttgacaaacctgagaaaaacaagcaatggggaaaggattccttatttaataaagggtgctggggaactggctagccatatgcagaaaattgaaactggaccccttccatacaccttatacaaaaattaactctagatggattaaagacttaaatgttaaacccaaaactataaacaccctagaagaaaatctaggcaataccattcaggacatagaaacaggcaaagatttcaatgacaaaaatgacaaaagcagttgcaacaaaagcaaacaattgacaaatgggatctaattaaactaaagagcttcttcacagcaaaagaaactatcatcagagtgaacagagagactacagagtgggagaaaaatttttcaatctatccatctgacaaaggtctagtatttacaatctgcaaggaacttaaacaaatttacaagaaaacattcaaaagttgtcaaaggagaagaacagacacttctcaaaagaagacatttatgtagccagaaatataggtaaaaaagctcaacatcactgatcactagagaaatgcaaatcaaaaccacaatgagataccatttcacaccagttagaatggcaattattaaaaagtcaagaaacaacaaatgctggcaaggctgtgcataaataggaacacttttacattgttggttggaatgtaaatttgttcaaccattatggaagacagcgtggtgtttcctcagtgaactagaatgagaaataccatttgacccagcaatcccattactgggtatatacccaagggaatgtaaataattctcttataaagatacatgcgtacatatgttcactgcagcactatttataatagcaaagtcatggaatcaacccacatgcccatcaatgatagactggataaagaaaatgtggtacatatataccatggaatactatgcagccatgaaaaggatgttctttgcagggacatggatatagctggaagccattatttttagcaaactaactcaggaacagaaagccagacactgagtattctcacgtataagggagctgaacaatgagaacccatggacacatgtcggagaacaacacacactgggccctgttatggggatcaggggaggcagagcctcaggataaatagctaatgcatgctgggcttaatacctaagtgacgtgttaatctgtgtagcaaaccaccatggcagacgtttacctatgtaacaaacctgcgcatcatgcatgtttttttcttgtaaaattgtttaagttccttgtagattctggatattaaccctttgtcagaaggatagattccaaaaattttctcccattctgtaggttgcctgttcactctgatgatagtttattttgctatgcaaaagctctttagtttaattagatcccatttgtcagttttggcttttgttgccattgctttttgtgtttttgtcatgaagtagttgcccatgcctatgtcctgaatgatattgcctaggttttcttctagggtttttatggttttaggttttacgtttaagtctttaatccatcttgagttaatttttgtgtaaggtgtgaggaagggatccagtttcagttttctgcatatggctagccagattttttaatgccatttattaaatagggaatcctttcctctttgcttgttcttgtcaggattgtcaaaaaccagatggtcgtagatgtgtggcattatgtctgaggcccctgttctgtttcgttggcctatatatctgttttggtaccagtaccatgctgttttggttactgtagccttgtagtatagtttgaagtcaggtagcgtgatgcctccagctttgttctttttgcttaggattgtcttggctatacaggctcttttttgcttccatatgcaatttaaagtaattttttctaattctgtgaagaaagtcaatggtagcttgatgggaatagcattgaatctataaattactttgggcactatggccattttcacggtattgattcttcctatccatgagcatggaatgtttttccatttgattgtgtcctctcttatttccttgagcagtggtttatagttctccttgaagaggtccttcacatcgcttgtaagttctattcctatgtattttattccctttgtagcaaatgtgaataggagttcccccattatttgtctctctttttgtctgttattggtgtataggaatgcttgtgatttttgcacactgattttgtatcctgaggcgttgctgaagttgcttatcagcttaaggagatttttggctgagatgatggggttttctaaatatacaataatgtcatctgcaaacagaaacaatttgacttcctctcctcctatttgaataccctttatttctttctcttgcctgattgccctggccataaattccaatactgtgttgaataggagtggtgagagagggcattattgtcttgtgccggttttcaaagggaatgcttccagcttttgcccattcagtatgatattggctatgggttcctcataaatagctcttactattttgaaatatgttccatcaatacctagtttattgagagtttttagcatgaacgggtgttgaattttgttgaaggccttttctggatctgttaagataatcatgtggtttttgtcattggttctgtttatgtgatggattacatttattgattggcatatgttgaaccaaccttacatcccagggatgaagctgtctttttcgtggtggataagctttttgatgtgctgctggatttggtttgccagtatttattgaggatttttgcatctgtgttcatctgggatatgttgttgtttctctggaaggtttgctctcaggatgatactggcctcataaaaagattagggaggagtccctatttttctattatttggaacagtttcagaaggaatggcagcagctcctctttgcatctctggtagaagttggctgtgaatttgtcttgtcctggactttttttggttggtaggctataattactgcctcgagtttagaatttgttattggtatattcaggcattcgacttcttcatggtttcatcttgggagggtgtatgtttccaggaatttctctatttcttctagattttctagtttatttgtgtagagttgtttatagtattctctgatggttgtttgtatttctgtgagatcaatgattatatcccctttgtcattttttattgtgtctatttgattcttctctcttttcctctttaatagtgtggctagcagtctattttgttattcttttcaaaaaaccagctcctggattcattgattttttgaagggttttcgtgtctctgtctccttcagttctgctcttagttatttcttgtcttctgctagcttttgaatctgtttgctcttgtttctctagttcttttaattgtgatgttagattgtcgattttagatatttcctgctttctcctgtgggcatttagtgctataaatttccctgtaaacactgctttagctgtatcccagagattctggtacattgtgtatttgttctcattcatttcaaagaacttctttatttctgccttaatttcattatttacccagtagccattcgggagcacgtttttccctttccatgtagttgtgtggttttgagtgagttccttaatcctgagttccagtttgattgcactgttttcctagagactgtttgttatgatttccattcttttgcatttgctaaggagtgttttactttcaattatgtggtcaattttagaataagtgtgatgtggtggtgagaagaatgtatattctgttaatttggggtggagagttctgtagatatctattaggtccgcttggtccagagctgagttcaagtcctgaatatccttgttaattttctgtcgtgttgatctaatattgacagtggggtgttaaagtctcccactattattgtgtgggagtctaagtctctttgtaggtttctaagaacttgctttatgaatctgggtgctcctgtattgagtgcatatatatctaggatggttagctctttttgttgcattgatccctttaccattccctttacatctttgatgtcggtgaccttcggatggggtttttgtctggacatcctttttgttgatgttgatgctattcctttctgtttgttagttttccttctaacactcaggcccctctgctgcaggtctgcctgagtttgctggaggtccactccagaccttgtttgcctgggtatcaccagcagaggctgcagaacagcaaagattgctgcctgttcctttctctggaagcttcatcccagaggggcacctgccagatgccagccagagctctcctgtatgaggtgttaggggcacctgccagatgccagccagagctctcctgtatgaggtgtttgttgacccctgctgagaggtgtctcccagtctggaggcacgggggccagggacctacttgaggaggcagtctgtcccttagtagagctcgagcgctgcgctgggagatccactgctctcttcagagtaggcaggtaggaacgtttaagtctgctgaagctgcatccacagctgccccttcctccaggtgctctgtcccagggagatggagttttatctataagcccctgactgtggctgctgcctttctttcagagatgctctgcttagagaggaggaatctagagaggcagtctggctgcagcagctttgccaagctgcagtgggctctgcccagtttcactttccgggtggctttgtttacgctgtcaggggaaaactgcctactcaagcctcaatgatggtggacgcccctccccccaccaagctcaagtgtccaagtttgacttcagactgctgtgctggcagcgagaatttcaagccagtggatcttagcttgctgggctctgtgggggtgggatctgctgagctagacctcttggcttcctggcttcagccccctttccaggggagtgaatagttctgtcttgctggcgttccaggcatcactgaggtatgaaaaagaaaaaaaaaaaaactgcagctagctcagtgtctgcccaaaaacggctgcccagttttgtgcttgaaacctagggccctggtggtttaggcactcaagggaatctcctggtctgtgggttgcaaagacggtgggaaaattatagtatctggtctggaatgcatcgtttcttatggctcagtccctcacggcttctcttggataggggagggagttccctgaccccttgcgcttcctgagtgaggcgatgccccaccctccttcagctcgccctctgtgggctgtacccactgtctaaccagtcccaatgagatcaactacgggtacctcagttggaaatgcagaaatcacccaccttttgtgttgatctagctgggagctgcagactggagctgttcccattcggccatcttatcagccctcaagaacttatatgctagactgttgagtttttttatttttttttaagcaactatagtggggtagaattgataacaaatgaaatgcatgtatagtatatagtgttatgacatatatacacacccatgaaaccatcatgaaaatcaagataatgaacatatccattaccactaaacatttcctcatgctgttttgtaattccctccctcctgccctatatcttcaatttataatcttccacccacctcattctctggcacacacagttgtgctgtctgccactacgggtttgtatatttttttaaatgtttataaattaaatcataagtctgcagtcttttgatctcttttatttaataataattttgagatttatctatgttgttgaagtacagcaatagttaattctattttattgctggataatatttctttttatggatacatcattgttcatctgttcacctgttgatggacatttgtgttgttttgtgttgttaaccaataaagctgctatgaatatctatgtgccagtggttttatgtgtgtatatcttttttttttgtataaatacctatgagcagaatgattgagtcatgtgtttaatgttttaagaaaccactcaattgttttacaaattatctgagctattttacattttcaccattagcatatgaaaattttagttcttccacatctttggcaacactggatatgatcctttttaaaaaaattagctattttaaaaggtgtgtgatactatttgaaagtagttttaattcgcatttccctagtgactaatgatgttcagcatttttcatgtgtttatttgccatccatttatcttttacctttccctttttcactgggtttttgagtgttaaatgcaagttctctgtcagatctatgctttgcaattattttctttttatggattgtctcttaattctcttaagtgtctttcaaagaagtcatttctaatttgataaagattattctttttctttcgtatatcatgtttttgatattagagctaataaacttttgccttatgcaagatttcaaatacagaggtttcccaacttatgatggtttgactgactgatgattttttgacttaggatagagtgaaatcaatatacattcaatagaaactctactttgaacctttgaattgttatcttttcctgggctagcaatgtgcagtaaaatactctttggcaatgctaggtagttgcagccagccacagctcccagtcagctatgtgatcatgagggttaacaatcagtagtctgtatttttcaaggtgtaaccccttaataaatcagagcatctgtattttcccctgtgttttcttttagaagttgtatcattttaggttttatctttaggtcaatgttcttttttgattcaacatatatatatatggtacaacatatggtcaaagttcatttttacatgtgaatatgcagttgttccagtaccactttttgaaaataatatcctttcttcaccaaattgcatttgtatttatatcaattgtccaaatatccatggttctgtttctacacttcctattctgtttcactgatctacttgtctgtctttacactattaccatactatcttcgttactgttgctttataattaaattttgaaatcaggtaccatgagtgtttcgacttttttctttttcagagttttttgagtttttttgttgttgttttgctattttgggtgctttgcattgccatatgaattttataatcagcttctcaatttctatcagaaaaagaagcctgctaggattttgaatgagatggtgatggagctatagatcaatttgggaagaattgacattttaacagcattgacttttcttatctataaacacagtatctctttccagttaggtctttactttctctcaacaatgttttatacgtttaaatatacaggtcttaaacatcttttatcatatcttttccccaaatattttgtttttaatattaatggcattaaaattttattttcttttgttgctagtatgcagaaatgtgattatattgatcttgtatgttagaagcttgctaacctcacttatgaattctagtagcttttttctagattctatcagtttttctacatagatggtaatagtttttataaggacgttttagtgcttgatttctaatatggatgtcttttttcttaattttcaatggctagaacttccagtatagtgttgtagaagtggaaagaatgcacattcttgttttgttcctgatcttgagtgtaagcatttatttttcaccattaaggatgatgttagctgtgggttttatatggatgcttctaattaggtaaaagttccattttgtttctggtttgatgaaaatttgtattggagatgaatgttggattttgtctaagccttttcctatgtctgctgtgataatcacatgattttctttattgttgttacattgatatacatttgaattacgtttattttcaaatgctaagccaaccttctattcccaggttaagtaccactcgatcataatagactatatatagctaaatttgatttgctaaaattttccttacaatttttgcatctgtgttcatgagggatgtggatgtactattttattgtaagtatctttgtctgattttgatattggtgtaatagtggtcttaaatgagttgagaaacattccctcctcttcaaatttctgaaaaaagttatttagaattagaatttcttattcttaaatgtttgttgggattcatcagtgaagccatctggacctacagttttctttgttggaaatatttaactataaattcaatttcttaaaaaggtatatggctatttcggttagttgtttcttcttgagtgagcactggcatttttttatttcaaagaatttgtccattgtcaaacacattggcacaaggtttttcttataatattttctcactaactggttaatatctgctattaaccactgtaatctatactgatgtctattttctcatttcagctatcatggatttgtgtatttctctttgcagttataatggtttttgcttcatgtattttgacgctttgtttttacatgaataaacatttataattttcttctcttgattacttatcccctctgttatgaaatgaccttctttatgacgtgtaatatttgccattatttttgaaagatatgttcacacaatataaaattattggttgactttttttcagtatgttaaatatgttgctccatagtcttcccatttgaattttttttttctgacaggaaatctgttgttatccttgtttcacatgtaacatgtccttttttctctagctacttttaaggttttctctgtgtcttgggttttgagtgatttgggtttgatgtgccttgatgaaattttcttgatttttcttgtacttgggttttattgaccttattgtatctgtgaatttatggttttcatcaaatttggacatttgggggccattatttatttaaatattcttttctgctcccaattttcttctgggacttcaactgcatatatatattagaccatgtgaagttgtctcacagctcactcaaactgctcttttgttgttctatactctcgtgtttctttttggaaagtatctattgtgtatatttgatttagtttcattatttattatctcacagtttttatctctagaagtttgatttgaggttgtttgtttgtttgtttgtttgtttgttttgcgacggagtctcgctctgtctcccaggctggagtgcagtggcgcaatcttggctcactgcaatctccgcctcccaggttcaagtaattctcctgcctcaacctcctgagtagctaggattcaggggtgcaccaccacgcccagctaatttttgtatatttagtagagacggggtttcacaatgttggccaggctggtctcgagctcctgacctcatgatccacccgccttggcctcccaaagtgctgcgattacaggcgtgagccaccatgcccggccttgagtctttttttatatcttctctatctctacttaatttctgaacatcaggaatacaattataataactatttaacatccttgtctgctaattctgacatctgtgtcagttctgggtcagttttgattgattcttttctttctcctcatttttgtgctttttgcctgcccactaatttttgagaagataccaaatacggtgaattttaccttggtgggtgctagatacttttaaattcctataagtattctattttcttaatgtcaaatttgtttttcttaagatttatttttaattaacacataaaaattgtacatatttatgaggtatagtgtgatgtttcaatacatgtgtatattgtatgatgttcaaatcaggataattaacttatccatcaccttaaacatttagcatttctttgtgatgaaaacattcaaaatcctcttttttagctgttttgaaatgtataatacagtatcgttatttatatttactctactctgcaacagagcaccagaacttacttctcctatctgtctataattttgtacccatttgctaatctctccccatccctccctcaaccctaacctcctcattccctagtaaccactgttctactctgtacctctatgagatcagcttttgtaaattccacaaatgagtggatcaggtggtatttgtatttctgtgtctggcttattttctttaacataatgtcttccaggtttatctatggtgccacaaatgacagcatttcttttggccaatagtattccattgtttatatttaccgcattttataatccactatctgttggtgaacacataggttgcttttatatcttgtctattgtgaatcgtgctgcagtaaatataggagtgcagatatcttttcaatatactgttctcattttgtttgggtaaatagccagtagtgggactgctggatcatatgttagtactgtttttaattttttgaggaaatgccatgcagttttccatagtggctgtactagtttacattcccaccaatagtatagcgcatttcccatttctttacatccatggcagcatttgtcatttttgtactagccattctaattggggtgagataatatcccattatggttttgatttgtatttctgtgataattagtgatattgagcatttttccatatacctattggatatttgtatttattcttttgaaaaaaaaaagtctatttagattttttgaccactttttaatcaaattatgtatttttttgctattatgttgtttgaatgttttatatattctggatattaatcccttttcagatatgtactttgcaaatattttctactattctgtagattgtttctttcctctgttgattatttcctttcttgggcagtaactttttagtttgaggtaatcccatttgtctatttttgcttttcttgcctgtatttttgaattcttgcccaaaaattcttatccaaaccaacgtcatgaagcatttcccccatgttttcttctagtagttttatagtttcaggtcttacatttaagtattcaatccattttgtgttgatttttatatatggtaagagatgttggtctagttttattcttctgcatatagatgtccagttttattggcactatctattgaagagaccatcctttacccaatgtgtgttcctgctgcctttattgaaaatcagttggttgtaagagcatcaatacatttctgtgttgtctgttctgttccattggtctatatgtttatttttatgctgactccatcctgttttggttactatagctttatagaatactttgaagtcagtgttatgcctccagctttgttctttttgctcaaaatgactttggctatttggagtctttcgtgcttccatacgaattttataattttttctatgtctgtgaagaatgccattgataccttgatagagattgcattgaatctgtagattgggtagtatagacatatcaacaatattaattattgctattcatgaaaatggaatatttttccatttatttgtatcctcttcagtttcttccatcagtgttttatagttttcattgtagagatcattcacatccttggttaaatttattcctctctatttttttgcagctcctaaatggaattgctttctcaatttctttttcagactgttcactcttgtcatatagaaatgctagtgatttgtgtatgttgcttttgtatcctgcaactttactgcactttgtttattctaacaatttttggtggcatctttagtgttttctataaataagataatgtcatctgcaaatagtaacagtattatttccttctttccaatttgcatgccccttatttcttttgcccaattgttctggctaggacttccagaactatgctgaataaaagtggtaaaagtggacatatttgtcttattccatatcatacaggaaaagctttcaaccttcccctattcagtatgatattagttgtgggtttgtcatacgtgacctttattaagttgaggtatgtttcttctatacctaatttgtttaatgttttttatcattaatggatgttgaattttatcaaatgctttttctgcattttaaatttatcatatggcgtctgtccttgattctgttgatgtgatgtattatgtgttaatttgcatgtgttgaaccatccttttattcctgagataaattatacttgattatgttaaatgatgtttttaatgtgctgttgaactcagtttgctggggatttttgcatctatgttctttaaggctattggcctatagtttttgttgttgtgttcttgtctggttagaacagttgggttattctggcttcatacaatgagtttggcaggattctatcctctttaatcctttggaattgtttgaaaaggattggtatttgctcttaagaattcagcagtgaaggcatcagatgatgggattttcattgatgggagactttttgtaactgatttaatcctgttactttctattggtctgttcagattttctattttgtccattcaattttggtagattttatatatccaggaacgtatcaatttcttctaggtgttccaatttgttggcatatagttgtttgtaatagtctcatagttctttctatatctgtggtatcagttgtaatgtcttcttttccattggattttatttatttcaatcatctctcttttatcttagtcccattaaaggtttgtcaattgtgtttatcttttcataaaaccaactctttttcatttatcattttttttattctctattttgtttatttcttctctgatgtttattatttattttcttctaattttaggtttagtttgctcttcattttcctataagtatgcttgaactttattctgggcagcagttaagttactggaaacagtttcatacttttgggtcctccttttaaatttttttaggcaggaccagactagtgtttagtctagggctactcactatttaggcaaggtgcttctaaatactctgtaatatacttcatcaattatgatgttttccattttggcgggtggaaacaggcaatacaccccacctgtgtgagtcctgattttcatccagtgctttcagatagttctttccaaggtcttgggttgtttccccacgtctttatcagtagtaatctgctagataatttagtgagaccttttatagagccttgtaattctctctctctgctcctcacacccctctcataccctgtcctgctaactatgtctgccttggtctcccctgagtctcagttctatctcctcagttcaaacccacaaggcttcacctgagtttcccttacctgcacagcagctagaaaactctctcaggttggtaagctgaagcaatcacaatgctaacctgattgtttcccactattaggaatcactttcctttattgcctaacatctgatatcctgaaaactgttgacatattatatattttatgtttttttttaattggttgtttcgggcagataggtacatccagtccttggtatttcatcttgaccagaagaagaaatccttggactattgaaattttttcatttttccaataaccatttaggtttgtttcattgaaatcattgttatcttctttttagtattaattgtccatttatttttgcacatgtttccccagtatggcttttgattggttgccctagtcccttacatggtttcaaccaattacgttattcttcagaatctgatagtactacttagagtctttagtttcactgcttctgtgattgatgaaacgtttgcatacgatgattttgtttctatattagaacagtttttttttggttaattttactttaagttctggaatacttgtgcagaacgtgcaggtttgttatataggtatacatgtgccatggtggtttgctgcacctagcaacccgtcattcagtttttaagccccgcaagcattaggtatctgtcctaatgctctccctccccttctccctaacccccgacaggccgcagtttttaatgttcccctccctgtatccatgtgttctcattgtttaactcccacttatgagtgagaatacctggtgttttgttttctgttcctgcgttagtttgctgacaatgatggtttccatcttcatccatgtgcctgcaaaggacatgatggctgcataatattccatgttgtatatgtgccatttattcttcatccagtctgtcatcgatgggcatttgggttggttccaagtctgtgctattgtaaatagcgctgcaataaacataaatgtgcatgtgtctttatacaagaatgatttataatatttgggtatatacccagtaatgggattgctgggtcaaatggtatttctggttctagatccttgaggaattgccacactgtcttccacaatggttgaactaatttacactcccaccaacagtgtaaaagcattcctatttctccacatcctcgccagcatctgttgtttcctgactttttaatgatcaccactctaactggcatgagatgttatctcattgtgttttcaatttgcatttctctaatgagcagtgatgatgagctttttttcatatgtttttggccgtataaatgttttcttttgagaagtgtctgttcatatcctttgcccactttttgatggagttgtttctttcttctaaatttgtttaagtttcttgtagattgtggatattagacctttgtcagatggatagattgcaaaaattttctccccttctgtaggttgcctgttcactccaatgatagtttcttttgctgtgcagaagctctttagtttaattagatcccatttgtccaatttggcttctgttgcaagtgcttttggtgttttagtcataaagtctttgcccattcctgtgtccttaatggtataatccaggttttcttgtagggcttttatggttttaggttttatatttaagtctttaatccaccttgagttaatttttatataaggtgtgaggaaagggtccagtttcagctttctacatatagctagccagttttcccagcaccatttattaaataggggatcctttccccattgcttgtttgtgtcaggtttgtcgaagatcaaatggttgtagatgtttggtgttattgctgaggcctctgttctgttccatcggtctatatattggttttggtaccagcaccatgctgtttcggttactgtagccttgtagtatagtttgaagtcaggtagcatgatgcctccagatttattctttatgcttatgattgttttggctatatgggctcttttttggttccatatgaaacttaaattagcattttctaattctgcaaagaaagtcagtggtagcttgatggggataacattgaatctataaattactttgggcactatggccattttcacgatattgactcttcctatccatgagcatggaatgtttttccatttgtttgtgtcctctcatttctttgagcagtggtttgtggctctccttgaagaggtccttcatgtcccttgttaagttgtattcctttattctctttatagcaattgtgaatgggaatttaactatgatttggctctctgcttgtgtattattggtgtgtaggaatatttgtgatttttgcacattggttttgtatcctgagactttgctgaagttgcttatcagcataaatagtttttaggctgagacgatagggttttctgaatatacaattatgtcatctgtgaacagagacaatttgacttcctctcttcctatttgaatacctttatttctttttcttgcctgattgccctggctggaacttccaatactatgatgaataggaatggtgagagagggcatccttgtcttgtgctggtattcaatgggaatgcttccagcttttgcccattcagtatgatattggctatgggtttgtcataaatagctctattttgagatatgtttcatcaatacctagtttatggcggtttttagcatgaagtagtgttgaattttattgaaggccttttctgcgtctattgagataatcgtggtttttgtcattggttctgtttatggaaagcatttagcccatttacattgaaaattaatattgttacgtgtgaatttgatcgtgtcatcatgatgctagctggttattttatacattagttgttgtagtttcctcatattgtcattggtctttatattttgatgtttttgcagtggctggtaccagtttttcctttccatatttaatgcttcctacaggagctcttataagcagacctggtggtgacaaaaatctcacagcatttgcttgtctggaaaggattttatttctcctttgcttatgaagtttagtttggctgtatatgaaattctgggttgaaaattcttttctttaagaatgttgaatattgaccctcactgtcttcaggcttgtagggtttctgcagagagatctactgttagtctgatgagcttccctttgtaggttaacccgacctttctctctggctgcccttaacattttttcctttgtttcaaacttggagtatctgatgattatgtgtcctgggattgctcttctcaaggagtatcttagtggtgttctctgtatttcctgaatttgaatgttggcatgtcttgctaggttggggaagttctcctggatgatatcttgaattgtgttttccaacttggttccattctccccatcactttcaggtacatcagtcaatcgtagatttgattcacatagacccacattttattggaggctttgttcgttccttttaattattttttctctaatcttcatgttttgtttcattaatttgatcttcaatctctgatatcatttcttctacttgatcaattcaactatccatacttgggtatgcttcacgaagttctcttgctgtttttcagctccatcaggtcatttatgttcctctctaaactggttattctagttagcagttcctttaaccttttatcaagtttcttgccttccttggattgggttagaacatgctcctttagctcagaggagtttgttattacccaccttctgaagcctacttctgtcaattcgtcaatatcattctctgtccagttttatgcccttgctggagaggagttgcgatcatttgggggaaaataggcattctattttttggaattttcagcgtttttgtgctggtttttcctcatttttgtggatttatctgccttcgatgttagaggctaatgacttttatatggagtttttgtgtgggagttatttttgttgatgttgaagttgttgctttctgtgtgttagtttttcttctaacagtctggcccctattctgcaagtctgctgcagctttctggaggtccactccagcccctgttcaccttggtatcaccagtggaggctgcagaacagcaaagattgcttcctactccttcctgtggaagctccgtcccagagcggcaccagcctgatgccagctggtgctctcctgtatggtgtgtctgttgacccctgttgggaggtctctcccagtcaagaggcacaggggtcagggacccactttaggaggcagtctgtcccttagcagagctagagcactgtgctgggagaatcgttgtcaggatcagctgctctcttcagagctggcgggcaggaaagtttaagtctgctgaagctgcgcccacagttgccccttccccgaggtgctctgtcccagggaaatggggttttatctataagcccctgactggggctgctgcctttcagacacgccctgcccagagagaaggaatctagagaggcagtctggtcacagccgctttgctgcgccatggtaaattctgcccagtccaaacctcccggcccccttagcattgtcaggggaagaccgcctactcaagcctcagtaatggtggacacccccccgcccccgccgccaccaagttcagttgtcgcagtttgacttcagactgctgtgttggcagtgagaatttcaagccagtggttcttagcttgctggactccgtgggagtgggacccactgagcaagaccacttggctccctggcttcggcctcacttccagaggagtgaatggttctgtctcgctgggattccaggcaccactgggttacgaaaaaaaaaaaaaaaactgcagctagctcactgtctgcccaaattgtctcccagttttatccttgaaacccaggaccctggtggtgtaggcacaccagggaatctcctgatttgtggattgcaaaaaccatgggaaaagtgtagtatctgggctgggtagcacagtccctcacagctttgcttggctgagggagagaggtcccctggctccttgcacttctcatgttgggcgatgccccaccctacttctgctcaccctccgtgagttggacccactggctaaccagtcccagtgagacgaactgggtacctcagttgtaaatggagaaatcacccaccttctgctttggtctcactgggagctgctgaccggagctgttattcggccatcttgccagattctccgaaaatgcttattaaggagtttacttctcatttctctaaaagatacttgggagagacagtttatttaaaagtattacatctcttaatatattgaaaacttttcctgtgaggttatattaaatttacaacttttttaattgcaacagagataacataagcaaacgtacacttactttgatgaaaaacttaaaatgcactggggactactatgatagaagccacttgctaacattggatttatggtttgctttcttaatatgttatatagtaagccaccatttattgaatggattaactacatgggaccctcccttctatgttttatctgtgtacacatacccttacaccttggggcccagcttaacagcaaagttcaatagctcctgtctcctgctgaacatttagcatattccaggataacttctgccattttacattttcctcctccgtcactaatcacacatccaattccacagatttggcatcttccgatttaaaagtggtgtataaggatttagtatatattgaaaaatatctctcaagattctcttgagagttaactgtgtagagtatagattttactttgcataaatgtaggtgaaataaacagattgcaaggctattgatgctgtcacattttcagcaaatgtaaaattatgtatgtatacatgtatatatatatacacacacattttttttaatttaggatttgctttcccatatgcttcatatggacccacatcagcggtatactgctgaacaaatattaaagcactcatggataactcacagagaccagttgccaaatgatcagccaaagagaaatgatgtgtcacatgttgttaaggtaaaacaaacatacttttatgtgccttcgagatatcagatacctaataaacccattgtgtgccacttgctgatgacacttaaataattccttaaaatatctttattttttaaagatagatttttttaaattaacaaccatgtacttttcttagcttactttactactgcctttcagatgaaacaataaaaatttcttgattgtacctgccgtgcttttgagagatgctggaattaggattgggctaaatgcaagtttcatattgaaataaaagattgctgttagttcttaatttataactgttcgtgaaaccattcctatatccatatacattgaatcaaggttttaaatgttagtgttttaacaattttttgtaaaaagcatagttattgaaatgaaaaacatacaagtaatattgattcatcagtgcacagatctaatatattcaattattttcctaggaaaacattttgttctagatatatgtctcatgatatcatgtgaaagttttttttttttttaactccagaatttgtacttagtttgtggcattttctcattactttagggagcaatggttgcaacatactctgccctgactcacaagacctttcaaccagtcctagagcctgtagctgcttcaagcttagcccagcgacggagcatgaaaaagcgaacatcaactggcctgtaagatttgtggtgttcctaggccaaactggatgaagatgaaattaaatgtgtggcttttttcctattcttatcaaaggcatcgttgtctgctaaattacttgaatattaagtaatattaaatccccatttttaggggaagtgagatttaaaaaaccattcacaggtccacaatattcatactatgtgtttgcagtagtgttcaagtgtttatttaagcatataattggtgtccaccaggtcctcacaacttctctgcacacaagcttctaaaattcctttcaaataaagttactttaatattt

SEQ ID NO: 2 - DNA, 304486 bp, Homo sapiens
  promoter (1)..(3000): SITE
tctgagcaaatgtcctgcttcatttctagaatctatgctggttcccttatggttgcggttcatctttcaagaattccagccatttgtagatgcatagcctggagaaggtaaagagaatgagctgggtagtctgaacctgggttccagcatgctgtcttacacatccctgtcagccaatctaccaagttctccacccctcaggttcatcttgtcacagtttttggtgtcaacaaggttggttgacattagtttctcatgaaatcttgactagtcagtagctctgttttattgaaaaacacacttctatttactatcaaacacttattcctaccccattacaaattacaaatacagattttgacagagaaaattatgtcttaaatggtagacaataaatacatgcctgctcaataaatcataattaaactagtcatataaaaaatgcatacagccagatacctaaatagatagtatactctcaatggctgattacaggttattttccaacttttaaatctttgagccagagagaaagtctattacttattcaaaaataaagttttaaaatctgtctgacaaatcagtagagttctactttgtgtgttcaaaagtgctgtattcaataaatacatcaatgtggtggacaaaggaatgttttgcctaaattccccttaagtgaaggatttgttactgcagccacagggactacagctgacagaggcctttagctgttgttacctgtaagaattgccagagctgcctgtcttaagttcgtgcccctcctaggaacagcccatgctcaatgattgatgtggatgtaaaggactgactatctcagaccaactcatggtcattctgaaggatcaccctagctagtgagctccataaggggctgacaaaggctgcaattggcttgcatcacagcttagcttctacctctgcctgctcctgcttcctgtatccctcccacccacagatgttggtccttagtcaaatactgcaggctaaaccctcaagaaatggcattacatataaagaaaacccttaggaattcatgcaatgcagtatttacagaaatagcattattattcatctataatttttaccttgcacgcttccaaaataaatttgaggcagctaacaatcatcatcatatttaactagcagaaacaaatgtacaaacacaaactttatgtataacaggaaggctattttaagtgattaaagttcaatatttcaaataaaaattttaactgatactaaccaatgacaaataaacgatatctcagtaaaaatatgttctggcttttgcatctatgtgagcacacatataagaaagtcaaaagtagaaaatatttatgcatatagttgtgggggtgacgatggtggcagtgggtaggttgttagggctgtgtgcagctttgagaaggaagagatgagtatgatacacaaggacatgggactactgagggagaaaagtaatcatccatttatcctgtggtttctgttttgatatggccagtacaatattaaaggtactaagtgaatggaaaacaagcaaggggaaatgataactactatactattagctagaaaactgtctaaggtcattgagtataataaggaacggagagatacaaatccttattttgtaacctgctatctagttatgggatcagattccatatccactactagctcataagggtactcttgtggcagctgaagacagacaagagcgtggcttcttcatttccaagggtgaaaactcaggactacttcgtgaatgtctttcattcactagagtcttgataatgctggagacttcaagagtctgggagggcagtaggcaactgggaagtttaagttgaaccacaaatatcgttgcaaacttcagcccaaacatcactgaacacaaaggcctgacatctcttggtacaaaaatgcgctctatgtttgtcataatctgtcggctttcttaactaggcaagcccaagcttgctattagatgtacaataaacactttgatgcccagtctgagtttttgtcccagctcaggaaaggcaaggggacagccaaggcagactggttcagacttgaccggggcagctcatccagcaactggcttactagccagctagcaaagtgagcatgaaaaggtccaggaatccttcttcccctgggccttaaattcgaaatcagacagcagccaaggagaagctgggtggtcacagcctagccccacaaacactccttccaggtaaggctcagcaaccccaggctggaggagtctctgtggacaccagagaccattgctaggctggcggattgaaggatggccactgagaggtcacagataaggggttctctagatgtttgctactcggagtggtgggagggccagcagcagcagcatcacctgcgaggttgttaggcttgcagaatctcaggacccagccaattgaatctgaatctgcattttaattcacaggcacatgaaagtttgagaatcaccggtccaggtggtggcaaagaggccagcccactgttctgcttctcagcccctcctcctccccacctctgctagcctcttaagcaaaactcaatcctgcaatggtcctgaacaacatttctgcaaatcccgagtgttgggggagagggcgtgcaggagctgcccccgccggggacgactgggagtaaacgtgattggattggtcgcctgctccagctcccggctcctggctcctgcaccctgagtgtagacacgccccccgaccctccccctcacctccacccccggccgccgctacagcctgcctcccgccggctgccacgaaaacccggagcagctgcgtacgctcatggacagtcctccgaggggcgaagccgggcagctgggcatgctcagtagctgggggaggtttgggtggagagtagaaagctgtggctctgcctctcaccccctcccgctggcccccgccccccttgcccctacccagccagtagtagttccccagcgtgcgcccggggagaccgggaacatggcgctgggagcgctgtagcagctgagaaggggctgaggcaccgccgcttcgctgacagccggccaccaggtaggcaggcgcgcctgtcgggactgcctgggtgggtatcggcgagaggagaggcgcctgcggggatgcgcgccgctggtaaccggagcatctctctttgccgggagaggatgcgaagaggaccactgtgacagcgcgatcctgcatctcctaggagatgcaaaggctagggacctgctgcctggagggtagggataggggaccgcccaccctcaacccatgcgctactatccagcctgggctctctaccaaccaaataaaggatggttccattttgctgggcgggagggcgtggaggagaaccccggattccagccctgagagcgtgctggtaccccgaactgctcgcaagcccagcaccagttctttgtgtgtatacagcagcgcacgccctgggggtgccacggcgctgcttttcccgagaagtgttggacatattgccagagcaggattcccgcatggtttttgtcttttggcaggtgtggactggaggatgcaatagctgagcctttatgaggctgtgttttcctttgttatttgtgtgtgtgtgtgtgtgcgcgcgtgtgtgtgtgcgcgcgcgcctggtgggttgtaatggagagacagagagagaacttctgcttgtgggcacccttgtgagcagggcgtggaagaggatcttggttccaaactcttgtcaaaattgtgtgccatgtcagcctcctagagggtaaacttgagaagtccagggatcatcactgctcagtttggaggataagcgcgatcgcccttcttgaaaccatgtgtgtgtatatggagctttcaactcgggctgagttaatttgtcaacattagatatcaattaagggaacctgaaacgctgaggctatttatggtggtctttttatggccagaataataaacaaaaagaaaagttggcacaaagccagaggaaagtgtcattgttaatagttgatgacagcttgaaagactgaacacacttttaggaaaatcaggaaacctggcattctacggagttctaccaagacatagccacagggtcttaggtaactcacaaaaatcttctagtgcaaccgcttctcttctggggcttggaggctgtagacactctttctcttacctgatatagatggacaagatgctttttagaagtccctcctacactagaattctatgaaattgacaccttataataacagctgtgccaccttagatttgtgtatctgttatattcaacagcacaacaactcaatcttggggagaaagataaccaggttagttagggagaaaggtaaatttaaagcatgggtttattttttgctttataatttaaaggatgggcatcttagcatatgaagtttggagacagtttcctctgcactcacactatttatttttatagtcttgttaggtggcgagaatgagtcagtatcatcaaaatgaagtgggggaatacataaggaaaatgcatttaagtaaattaaagccataaatgtccaatctgataataccttgacttggaatacatatgtgcaaggcagatgcactgctaaatggaagcaatataagccaatggcaagttccttggtaacgctggccttagactaaaaacctagtgttcttctgtgagtgggtagagattgtactaggtctctgcagcttgttattttattaataaatgtgagtttaattgaaaattgatatctaagcattgaatcaattgatatctaagcattgaaaatctagtctggcagtgcctctccactctgtcaggacctgctgaaatgctgtaggtatgggaggcaggtgtttgaactctgactcttctatgtttagctttgtgattctgggaaaattatttaactttcctaagcctcagtttcctcacttatagaacagtcctcggaatagtaagacttatctagatgagtagatgtacggattaatcataatttaaaccaccaggaaaatggtggtgactgttatgagtaagagggaaatggatatttactgaacctatgctatgtgccctatataatgattcaggctcaaacatgttaattcccttaaatttcccaactatattagtttcctagggcagttataacaagttaccaaaaactgggtggcttaaaaccacagaaatttattctctcatggttctggaggctagaggtccaaaatcaagatgttgtcagggccatgcaccctccaaaacctctaagggaggatccttgcttgccttttccagcttctggtagccccaggcattccctggtttgtggcagcataactccaatctctgcctctgtattcacatggcatcttcctctctgtctctgtccctgtgtccaaatttcccttttcttatacggacatcattcatattggatttaggacctgttccttattcaatatgacctcaaattaaccaggttacatttgcaaatgcctgatttctaaacgtcatattcacaggttctggatgggcatgaattcttgggagacactcttcagcccagtacaccaacatccctatgaactgtgtttgatgacaacattaacattatgttagcataagaggtaaagatagagtagcatggattcagaaaatactttaaagattatttaagggaactaacctatttgaggctttatatacacaaccatggtagggaagagcagggttcaaaagtacctcatttatgcatgtcacttacctgggagccaagacaacccaggacctggatcttctagagtatctcaaacatactgtgcccccagttgatttgttgatgttgccttccatccttcttgctaaccttccaaacaaatgcaaaaaaaccaaaatgcattaaataacaaaaacaataatagcaacaaaggcaagctaacaactttctagtccttcttaacctggtaaatggcaccactatccatcgagttgctttaaccagaaacccaagaatcacctttgacacctcctttcccttgaccctccacatttaaaaccatcatgcagtcttataaatgctattttctctatagatctctaaatacatcttgataaattatttctatccccaccaccaccactatcaaactgcaagagatgataatctcttacctggaccactagaataacttcctaacaagacccctggttccttgcctggctcttctaatccttcatccagagcagagaggataatgtcagttccctacataaaataccacccacacacctgggacagattccagagactcctagagcctctgatacttggccagcctcatcttgtgcatctcttcctcttgatgctgtcacactgaccctctgtctgtttctggaacatccccaaccatcttccctctatattttaccagtctgggtcatactcatcccttgggcttccttgatctctcttccccaccgacccaactctctcatcatcctttcacttttattccacagcacttattccagtttgcaaatgcctgtttgtttggctatgtctgtaagctctataatggccatttattttatgtcttatatgcagaaggtgctcaatgaacttttggtgaatgagtgaaggaacgaatactctgcctcatagcattttcctgtgccaacttcaagatctgatttttagaaggtttttcttttctagtatagaaagagcaatttagaaccttattctgacttcaaaaggagagtattcttggctttaacaaatgcatcttttaagaactgatttaggctagaagttaagaattgagtcaaaatatttttttggcaactgaggtttttgtaaaaatacatcaatcagtcaataaatattctgcaaatatttattgcatgtgtatatttgccatgctacacccttggaagacaatggtgagctagactttcagtcttctggaaattaatggtaaacaataaataataatcattatgtataataataaataaataatacatttaattatttgttgagcttctgttatatgccagtgtctcttcaattcctgtccataatcatcactataaccctctaatctttcccaataacacagaaccccacacttactaggagcacaacaaatattttgtgaattaataatttgttggtattattattctgatctgttgaaataggaaatgtgtgcttatctggggaaaaaggtatctgcacatatatagaagagcatgcaattgctttctcaccatatctaggtgggcactgttttattttaaaaatcatcctggacattatctctggaggtaaattaggagagaaattgctctttataaggcgacaagttcctggtgtgtttctgtcaaaatcctgaattgttttatttcgtttaattgtctaaatttgtcctgtttaatccaagtgagaagtgacttaatgtctctcattcagagccctttttgtagttttgaatagtaagactgagtgcccaagggaaatgaatgtagttcaacagaggaacacatgttatgttttagatttctaaagtggcttcaatttcagatttttatattagtctcataaaagggtgatttttaaaaataaacagtaaaatagcatttaatgcctatgtttaagtaaagtttttaatgtaagtaaccaaaaattagacagtgtgccttgattttgtttaaaagtgatggatacacaatgggatcaattaaagcctaaaggaactagtacaaaatttcagtaatgtttttggttataaaatacatgttcaaaaattgatggccacccaaaatgccagtgttttactaactaaaaagtagaatggaaaatgattctattcatgaattcactaatatataaattgagtgggaaaaacaggaaacatacataaaatactcgtgaataataagaaatatataaaagtctttgtggaaagctatacaattttactcaaaccaataaactgaggagcaaacaaagaaagacataccttatgctttggtagaaagatgaatattatgaaacggttatttttcccagatcaattatatagttttattacattgggatttgggagaaccttacaaaatgattctaacatccatctggaagaatcaccatataaaaataaaagaaaacttttcaaaagaatagtagtggtggtgggggaggtgagtggtggtggtaagatgggaggtggtggtacatacaggtggtgtggagagtggagttctaggggtggtaaaggtggggggaagtttgatggtggagacgaatacatgaaggtggtgggatgaggtggtatggatgggggtggtgggtggtagatgtgggtggttggggggtgaaggtaggtagatggaagtggtagtggtaatgggcgccattgttgaaacaaaagtaaatttgatcattggctacaatcattatagcaatgtagtactggtgtaagaattgattaatagctcagtggaatagaagagcctggaaacagatttaggtcattcttagaaccagaaatatgacgaagaaggcattatgaaacagtgaggagtatttgacaaaacgtcatggaaattggctaagtgtatagaaagcattcaactaagaacctctgtttacatgaccctccaaatgaaattccacataaattgataagtttatatgcaaggtggaaaacaaagctagaataaaatatttaactaattttaggggaagtactttttaaatataaaatcaatgggaagaatcacaaagcaaaataaagatttgaatatattttaaactctcatatgcagcaatgcatgcattttaaacagaacagaatcatggaaaacaatattgacaccaaacatgacatgacagttcatgatagccctgaggaactcttgcaaatcagtgagtaaacaatagcaccccaatagaattgtccaaatatctgaatgcataattcaaaaggaaaaaataaaagaccaatgcacacgtttaataaatgttattactattaatcacagaaatgcaaattaaaataacaatgaggtggtgtgttttatgtattaaaaaagtaaaaaatacaaaaaataaacttaatactggcaaggatgcagtgagaaggacaatctctttcattacttgtggtgggagggttacataatacaagcttcctacagagaagtttaaaaatacttatcaaaatgtctgccaggcacggtggctcatgcctataacaccagcactttgggaggccaaggcaggtgaatcacttgaggccaggagtttgagaccaccctggccaacatgggaaaaccccatctctactaaaatacaaaaaaatagctgggtgtggtggcacactcctgtaatctcaacttcttgggaggctgaggcaagagaatcacttgaacctgagaggtggagtttgcagtaagccgagatcgcgttactgcactccaggcttggtgacagagtgatactctgtcaccattaaaaaaaagaaagttgtcaaaacttttaaaagtttaaattatttgacacactattcctcttgttttttattttatgttttcttttgtctttggtaaggaagtagatgcaggtaaaattgtgtattataatatatttcatagtaataaaaattaaaaagaacctatataactagaacttgggagcttggtaaaggtatttaaggtcccaagataacagactattatataggtattaaaattaattattttgaagattatataatgacgtgaaacaaacttcaccacaattttaagtggaaaaaaacaaaatgcactactacatatattgtattgttaatatgtgttttgtgaaatgaaaacatcatgcaggtatatttaaaaaaacagaacaaaattaaaaagttaggcaatctctgggtggtggaaatatggatggcttttattttgttacacttttccaaattttaaaaaatgtagtaggccgggcacggtggctcacacctgtaatctcaccactttgggaactcgaggtgggtggatcacttgagctcaggagtttgacaccaggctgagcaacatggcaaaaccccatcactacaaaaaatacaaaagttagctgagcgtggtggcgcacgcctctgatttcagctactcaggaggctgaggtggaaggaatgtctgagcccatgaagtggaggttgcagtgagccaagatcgcaccactgcactccagcctgggcgacagagtgaggccctgccttggaaaaaaaaattagtcaatgatgtgttgttttatgaatcagaattaaaaaatccactattttacaaataagcagtgatatacatttaggttgtaattattcatttatgtaacaaacattcattgattatctcttgtgtcaggggctctgccaagtgtatcagcagtgactcttagattgcatgcaaatgaaagaaactctgcctaactccagcaaaaggaatttactagaaggatattaggtagctaaaagaattgccaggaatgtggcgggggatggggaatcccaaacacacgcacgcgcgcacacacacacacaccccccaaaacagcttggaagcagatgaagctagtcagttccaggtctagatatcaggcactttggaaacagactccagccaagaatgacttgttcagagcttgaccacagtccactggctgctatgcttggggttcttcccccacacccagcccactgggcatgggataggagggggggtgccttgattagtaggcccactacccctttccaatgaaagaaacacaagagatgtgatgggtgctaaaaagccaaaaccaaaccaaaccaaaccaaaaaaagacaacagaaatgcccattaccctaggacttaggggtaaacagggttgaataccactcatccctctctttgcatcccataggaaacatttgctatgacatgggagtttaacttaaggttcatggtctaatgttcactgtgatgtgcatcattgaaggtgcaatgtgggtgccccacacagaaggtacattttcctcttggagccagtagtgacatgttgggaagagtgtggattttcagcctgatccttatataccggcaggaattttgaaagaaaggtcccattttctcaggtaagagaaaatcctagaaaggtataatctgaaagaaacaatgaagcagaagcaagaggtataatatggaaatagatcaggcaccaacacaggtctagccttcttttctgaatgatcttacattcagcaagcccaacagtcatgtttgagtttagttcaccatgaataccagcctcttggttcaattaactttggttgataaagttattattaactaaatattaaaagatcatacttagatcagtgcatattcttactgcagttctcatgataaataggaaatgtctgtggctgtccaggtaacttaacaatcatgttgttgcttttataactctttataaacttttctgtgaagaaagatgacttgtaagtggaagagaaaaaagaggtaattgttcttcacttgtgcttaaaacaggacagagaaaataatcttaaattagcaagacaggtccctgtcctggtcactgtatattagagggctctgctactctgcactccaacttctcagcacctggacagcactgctcctctgcaacaaagaccccttgagctctttgcctccttctgagccttgctctgcatagctgggaccttggaattcactgctcatggggctcctgggcccagtgggggttgaaaaaagactgttggtgggaggtgtaaatggagcttggacttgtggactttgtagtctccacacaagtgctggaggccatggtagaaagagaaagggatggggaggggagcaaagactgggtctccttgctttgctatgttctggaatttaagtactctaaatttaaatctggcttctacgttgttatgaaggcatatttgttaagccaggaagatataacacatttaaattaacatttttttagcttgatttatagtatttaaatattgagatctgtaacatgtgggactccttttgtactcttgtctctcaaatgttaggatcaggcctgctcaggtaactgttttagaaaggtaacactcaattgttgagacacacatgcataattagctaatcatgcaacggcaatgtagattataagaaaaatagattatactttttgtgactatagctaacatattgaacaagatgtgttaagcccacatccaagtattttacctatgtatattaattagtcttcataagaacaccataaaataggtcactcaaggcaaaatatcatgcagctagatagagtcaagaagctctggatcatctgaatccaaaggccattgtggtatacaacaattcaatggataagaaaagttttatctgaggtccttgaacctccgataatttaaaagtcactagattaatcaacccactttgactctccaatactctcaaatcctcagattcagcatttctgttcgtgcagatttcctcatgaaaagggaaagtaaatgaatggactgccttgcagtgtttggtattatgattaactgaatgttccaaaccaaaaatcaatatttggttgtttgtataagaagcacatttaacttacatttttaaacagatgtccagttgtgggatttccttagctaattttcaatggaattgccattgtgtcattgatgataaaggtaaattggccataaggtgtacctttgtttctctaaagcactggttcatacccctactactcttctattaccctattttccaactgaaccaagaagactttctgcaatcaatattggttccatcacttttgataacaacttacataatgggggaaggaaaggaagaagggagagaaggggaaagagaaaaacagagagagagagagaaactcccacctttggttccaaatataagatctctgttagacacaacagttattagcttcatttccaattaaaattcaagtttggcatactatttaagaaattctttagctattttctagaggaaacttcatgtgtttgcttctaatggtcactggattcatgcaaatggtgtttgtataataatgattttttagctaattgtttttagctgtcagctgtgttgcccccttattttatagctacattacagtttactgacccaggaagtgaactttctggaagaatagaaaattcacactttatttgtttaaaagaaagattcttcctctaaagtaggaacagttgctatgtgggtatgatttaacttctaaaaataaaaacactgacggaggagggggatttaaaaaaattttacagaaacaacttagaatttttctcatcctggtggtttttaggacaatgttctctcttctctaggtattagagatagagaaacagacaaacaaaaataatttgccaataatatttgaataatagttattcacgccttgagttaagtagtggtttaaccctttttgagggttaagataacagctacctacctgcaggttgtgtgctatctctttcagattccaagtagagtctactcttctcctaccctcacagccaagcagtatctaaattgcaacttggctgggcgcggtggctcacgcctgtaatcccagcactctgggaggccgaggtgggtggattacctgggtcaagagttcgagaccagcctggccaacatggtgaaaccctgtctctactaaaaatacaaaaaaacattagctgggcatggtggcgcacgcctgtgatcccagctacttgggatgctgaggcaagagaatcgcttgaacccgggaggcagatgttgcagtgagctgagatcacgccactgcactccagcctgggcgacaaagcaagactctgtctctaaataaataagtaaataaataaattgcaacctaaaaaacattttttgcatgtgcccccttccctgtctccactgccactcccctttgcatctctcaattagatcttctgtcagcctcctatctgttctctttcattcttacctccaaaatgatcttctgttatctgagtatcacaccttcattgaaaactttctatacagctaccacattcccccaccctccccctcccccaaaatcctcccacatggttctggattgttcctctcaaacagtcatcacaatgttttgtagatttttttttagcttccttcttgcactaaactgggaactcttcgaaagcaggcatcttgtttcttcacctttgtatccccagcacctggctttatgcctacacatgtcaggtctttaataaatgtctatggatgaatggatattacccaggctaaaaaatttgaatgcaaatgtgaaaaggcggtgctatgatttaaaaggccactcaaggcaaaatatcacgcagttagatagattcaagaagctgagtcaaaccctgtatcatctgaatccaaggcaatcaatgctgtggtatacagcaactcagcagataagaaaagttctgtttggagtcctaaacaaaaattggctaatctaaaatcagctaatctaaaagttatctcatccattttctttaaaaacacacaaactagtttcacactaatactggttttctattttctgtgaataaaaaagaggtagagagaaagattaaaccctctacctgtttctggttggagaaagaaaaaaaattccaatattggacatgggtataaggtagagattctaaacacatggaaaagagagaaagtggtgaagcaaactataagctgagagccggcctccccacaatagagtaaataaactgagcttcttgaaaaacattgagaataattatttgattgctagatgcagcttaacacctactgctctattgtcccagacctggtatgaatatgcctaattggtggtgtgattcctgctcaattatcccttgatttgtgtagtgaaagctggggttttatatggttctgcccaagtcttcggcaccaatgtttccatagcaacagatgcactataaaatgaggaagaaatcgaaagtttgggagagaaataaatcaaagcgagtgatcccagcaagaggttccccagggtgagtcccaagggcttttcaggcttctagtcagatatatttgcggcagaattctcggagatagaagattctaggatgataaatgccagggattttttaaagcaatatatggatactatctatgacagcagttaccttgtgtgcttcagtttggatttctaaggcaagttcctgtgttgggatattaggagtctacactggtcgaaagaatcacttaccagttagagcctactaacccctaggtaacaggatatgaaaactggagatttttactatctttgaaattgaattgagctatccttctaaagctgaaaggtgtagaacactgtgaattctattaggtactaaatatttgactatgtcaatttagagcagtgtctaattttgtggtatagatgtgttctgtttaacacagacagtgtgaaatttcccagaaaaaaaaaaatctagatttctgacttctcttattcaaaaaatcacacacacaaatacagacacatacacttagttgcaaaatttgtcaacactgaccatgtttcttcatggcaatcatttgctgtagctgagtaacagctgcctttaacatggatcagtcatggagtcttcactttgtcacagtccccatcactccctactgcctgtaatcaggccattttaattgtttatgttaccctcttggtcattaaagtcttgaatgtataacctttgaatttgagtgaatataattcaaattataacctttgaattgctctatatttttggatcattttatcctcatatattgtgtcttcttaattttgcttcccaaaaatatggaccaacttatttaataatttaaatataacgcaaatgacattttagaaagatgcattttaaaaattttatcctagaaaaaaaatttagaagtaagactttatgctggtatgaattttatttaaaagtatagagtttagagatatttggtaaaaacatttgcataaataggctgctgcaactactttttcaaaatttctttcaacactggacaatttttttaaaatttctgttgtgttttatttgaactaaactgaaatggaaaagaaatcatatggtcttaagcttgctggaggtagggattatcaaaacgcaaaaggatggtatcatatttcatatgatattatcatctggaaaatataaaactttcacaatgcaactgtgctcacagcagtgaagtagaaccttataaatgatatgcattctgtagaatggctggaaaaatctcaaaatcaatattggaatcacccagggcaactaagaataacaatatcactgtaccttactaaaaaataataaaaaacaagtgagatagaaaattccagagttgtccctagagtttttatttgcttgggatagaaggatgatagcagtagcttcccatttcttttcagacctcaattttattttaaacacagagcacagtccacccactgaaaagaattttcttttctacaaagaccagggtgtgcaatgactgtgagtatcactgtaaaacagaagaacatttggctctaagttactcacttaactagtaggatcaaaacaagataactggatctgtctattaggctagtgtctgtttatgtctgctttctcctgtaaagactagattaaaaaataggaaacagatgttacaatgaacattcccaacgagtcactcacccattcagcagctgcaccttgagtactattctgcttacttcaggtggcatgtaccatgaggaggcatatcaatgaatatagaagataaaaatgttcttctgtgaacatagaagataaaaaaagaaggagagacagacaggaaacagcataataaataaatgtatagaatgccagaaggtcatcagtgctttataaaatagagtgggataagggaaatcaaaagggctgtgatgggtggcagtgaagggcgtgattttaaataggatggtcagaataggtataattgagaaagtgacatttgaaggaggagaggaaattgccgtgtagatacacatgggacagcatcccaggtagggatggggaccatttttgcagtgcactccaaattgcatatgatttgtctcctgcaaagaccctcaagttttttcatttggaagtagaattttagtgctggttcaaaccctaacctcattctaatgctgctaaggacaccattactccagtcctgccaattcttctctagataaaccataactgctttttggaaatttttttttaagaggcagggcctcactttgttgcccaggctggtcttgaactcctggctttaaacaatcctcctgccttggcctcccaaagtgctggaattataggcataagccaccacacacggccttccttttggagatttaatattctttctccattttggtttaattcaaatgaaacacaacagggatttttttgaaactgtctaatatttaaaaagtatggaattatggcacatactaggtatttgttcagttttttgtttggttatagaaagaaaagatgtgcctcagagttaaaacaatgattgagcagtaatcaagttctttgaacatgtattatatgccagatattgtgttatatgttttgtaaacactatctcatttaattcttgcagtaggcttataaattaggtgttatcattattcccattttatggctagtaaatagcagattgtgctgccttgctctattgattaagattaatcttctacaagcacaaaaaaaaggtaaaaagtattttctcaagttctaagtgtaattcctttaacttcctctattttagttaggatttcccccccagaaacagaccttgaggcaaggattcgagagcaagtagtttacttggaacgttcatggaataccgttaagagagagggcaagtaacacatggaaggggagtttattagtctgttctcacactgctaataaagacatacccaagactgggtaatttataaaggaaagaggtttaatggactcacagttctacatggctgggaaggccacgcaatcatggtggatgacgaaggaagagcaaacgatcgtcttacatggcggaaggcaagattaattgtgcaggggagctcatttataaaaccatcagatctgtgagacttattcactaccaagagaacagtatgggggaaccacccccatgattcagttatctccacctagccccacccttgacacgtgaggattattataattcaaggtgagatttgggcggggacacagccaaaccatatcagaaactgtcagtccattcctgctgctataacaaaatacattagactgggtagtttttaaataatagaaatttatttctcatagttctagaggcttggaagtccaagatcaaggcaccagcagattcagtgtctggaaagggcttctcctctgttcaagatggcatcttgttgctatgtcctccagagggaaaaggatgaatgctgtgtcctcacatggcagaagggtaaaaaagggataaacttgcttccttaagcttttcttataaggacactaatctcatctatgacagtggagctctgatggcctgatcacctgctaaagactcttagtatttttaccttcaggtttaagttccaacatgtgaatttcagagggacatgtacatctaaatcatagcagaaaggctgccaaaattagagtattaaaatagaggaactgggaaatggtataaaactcatgcctccaaatcagtccacccaaaggggagggaactgagatatttatacaccaactcccatcagtcattagttgaaggctgttcccagagaatgttagtttctctgtacttccaaactcccatgatatggaaaagcccttagaaacacagatactgactgttggaagtcagtaggcatatgggtggatactaataaggcttgctacatctatacctgttgcctcccttgtcatgtgcctgccaaacttcagagatcctttagaagttgttgtaagaaaagaaaatttatcttctgctagtcaattgtgagctattgattgggcaatttattagtgtagacaagaacattccatgtacagtgggtaaaagagcattgagccagagtgttaagttgccctcaaagtcatttccaaatgaacaagatgatataactataggtgttcaattgtacataattaagcagggagctgttattataaacgcatctaatgttgaaaaatgaaacataagtaaggctacaactgaccttgtaatgaaacagaaagataaaatattcctaagagtaaatcatttgactgtgtaatgtaatgtaattgcttttttttttttttttttgagacagatctcactctgttgttacccaggctggagtgcagtggcaggatctcagctcactgcaacctctgcctcctgggttcaggcaatccttcagcctcagcctcccaagaagctgtgactacagagatgcaccaccacacctggctaattttttgtatttttagtagagatggggttcaccatattgcccaagctggtctcgaactcctgagctcaggcaatctgcctgcctcagcctcccaaagtgttgggattataggcgtaagccaccacaccgggcctgcattcttgtagggtactggtggaccagttgataagctgtatcttccatggcattatttggaactctatatccccaaaaatgttgttcatgcccatgatcatttttaggaatagctatcttgaaacttgggaaagctggagaatatataatacaaaatgattttgagtctgtcttgcaagaaaccccaaaccaaaaataattttgcatggagaaagaagagaatgtaacaagtatgctgagagcagaaacgcaagtgttagtcctctgtatgtgtggataaacagtaaatgtcagtgaattttaactgagtgataaagtgtatttgatcaggaaaaagtgaagaatggatgttgggcagttagcagcctgaagtaggatctcaaaaataacccaggtggctttctcgatgagaatgtatcagttgaggtcgtttaggatagtagagaaggtgttttaaaattgtggagcaagagggggatccaaagttaggtcatatcagtgattgccaataggatactcaggttaggcaggctacaaagtattgtgttctgaaaataatcacagttaaccttggatttgagatatggatgggttttaagaaactcatcatctttttttgtttgtttgacagagtctcgctctgttgcccaggctggagtgcagtggcatgatcttggctcactgcaacttccgcctcctgtgttcaagcaattctcccacctcagcctcctgagtagctgggattacaggtccccaccaccatgcccagctaaattttgtatttttagtagagatggggtttcaccatgttggccaggctggtctcgaactcctgacctcagatgatccgcctgcctcggcctcccaaaatgctggattacaggtgtaagccatcgcacctggccattatcattggtatggacaggaaacctgttttacacaccagagggcaccatgggttctagagtttcccacttggagtaacagaaaagtgttcaaaatttctggtgttcctaaaagaataaagagaagaaatttggggagagctatcagggctggaggaagagtggtgcttaggggctttgcatcaccaacattgagccttcctttaccaccttgggttactctaattgttgaacctgagtgttacagttctgctcttccctggttctcatttcagggctccccattcagttcctttatattcagggcttccgtgttacacagcttcagaagacaacattcacatctgaggtgattccacttttgttgtgcctgccttggtccatactttgtaaaatgggtccccttgcttcttaattatgtgtgttttgttctccttctttgtcccattggactggaagaggcaggaaggaagaggctaaattttatctagttaagttagagcatctggcatagcaccttccacgtacagttgctcaataaatttttactaaacaaatgaattcaataagaattctgtgacctactgagacttacatctcctttaatccaaatagcagccccatgggttacatattgccttcatttctccttggaggaggtgaggagtagatcaatgaatggacacagtgagtaaaaacaactctttcaataaaaataggttgtggcccaatttaattagactctaaaatgaaataggagaggccttctgcatataggggaaatctgagttttataaaatgcatttaaagaaaataagctttccttctacttatttctaactctcaaattcattttcatgacagttgatattatgaggaggaaataatgaatagattttctatagattaaacttgtctttgcagctggatgttatgggaagcagggcgtatcttcaacaggattcttttgtttgaaaggggcagaaacccaactcaaagtggatcaagaaaactaggaatttgttgacctgtatagcagaagcatccagaaatacagttggatcgggagctcaaaaacattatcctggtgccatttcttccccatctttcagctctgagtgtttctgtatagttttctttcttgggcaagctatcttatttttctggcaacatggacagcccccctacagctcttgggtttagatcctactgatctcaaaatccagcaaaaaatactctctagcccaatagttctaacaaaagtctcaggattgaatttcagtagttctaatataacatttgtgggtcatgtgccagtaccggttccttgtctaattactgttgcctggggaatgtgtggcttttgtattcccacctgggtccaaacccacccctagagtcagaaatcagagtggagttactttctcccaaaccaccttgacagagaacgggggtagagaggatctgcaaggattatcaggaggccattatcagaagcatagcaaagaatgtggtcaggcaaaaaaaaaaatgttttttaaatgttcattagtgtaggtaaaagtaaaattgagcaatataattatagtctccacaaagatttctgacccagtgaaatttctctggtcacaggttcgttggccctaatttactggactgattgcatttgaaaatatctatcttttcctggaaatgaactataagttaccaaagacaccatacagagactctttagccctatcctcttgtgatcttagctcttcatctttgtgtcctattttgccattgtactgctgcttctattttgaaaccttggtaagaaatagatttgtgttggtttactgatttagtctgaaattaaaatgaaaaatcatttgcatattcagaaccttacttaatgggagggaaatcggtgataaaattgcatcctaaacatttctcagggaaatgaacttatagtgagttagtgaggacatattgtcattacaaggtaacatgaaatatatgcacccaagccctcagcttatcctgatgctttctttctttggctttctcatgcagagtatattttctctcagtgatttaagtgatttggtattattttgtcctatatagcttcatgttttattagatcaattcaacttttggacaagacaaattggaaagtaactttgatagctactatatgttatttggctgatattatttagttgaaatgatgtccaatacccatgtttctttatatttgcagtccttcataccagcctgtgttctatttttacagtgatacagctatcattttgtttggttttagatgttcatgcattctagagaaagtggaaaacttagaagcctaattaatgactgtcttctggacctctgagaccatgtttctagtgttttccgtggaatattatcaggtatagactttatgataattttcatccattcagagattaaatattaaattgctttaggaatattacggtctatgtgcatctgttcagatatatttgctttccttggaatgctttgaattcttggtttttgcagattatcagtctccaccaagattcatccttgcgagatactgttcccatgttaatggagggtgtttcagtgatattcgttaaagcagagtaaaaaatactttatttagggccatcatgataggtagagggaccgagcaatgagatttcacagtgagggacagagatggagcttaactctgaacacagcatggaccagtagtaatttatattgccaaggagcagagtaggagtggatggaaaattaccaagaggaaacatctgggtaaggaggatggctaaactgacctaatgggattcttgcttaagacagtccagcatgatcagacatacctgaggaataatggaagaggaggaagaatctgatcagatatggagagtgatcagatgtcaaggtggaaggattttgctaaactgacttagcagggtattttgttaaaactggattttaaaaggaagtgaacagataccaagggtgtttactctgaatttgcatttcttcttttgtgtttctttttaaaaaatccttctttgattttacaacagaaggagttgagtatttgttttagataaataaaagctggattttatgtggcaaagatagctgatgacccctagctgctctgtgttcctatgaagaaagtcccactgccagagattggccatgcctggccttcaccaggtgtgacctcattaactgagaattgatagctttggaatttgacataaatcatagagtgtgtgcaaaggcagtttatgtaatgggtaagagattggattatggacttggcaagcatggattgtagaccctgtccagaccactagttgtgaaaattggtcaaatttattagtttctctaagcactgatttcctaagcattaaaaaaatattattaggtaccttgtttgggctgctgtgggttgtcatcactccatccaaaatgtgactctgatttactttctacatatttttttcaccatctgaaattatctcgttcatttatctgtttattttctgttatctttcttcttcaccagtaagatatctttccccggagattaaggatcttgcctattttgtttggcctgctatctccagggcctggcacatattaagagatcactattgtgtgtgttgaaggaagaaatgaatgaataaatcgggcttagcagagaagtaggcacttagtagtgctcaatgaatgtacattatgacatttgctggtacctgaagctcttctatgtgtagacttgaaagtctttcatttggatagatttttaaaaaaattatacgtatttaaaatcatggaaactgagagcttgaaattgtagttttgagactccaattcagccaaaaactgttataatgttgagtgttcttatttacataattctttatttttaatttttattatttattcatgtagttagttttagagacaggatctcactatgttgcccagcctggactcaaactcctaagctcaagtgatcctcctgcctcagccttccttgtagctgggactataggtgtgcaccaccatgcctggcttgtataattcttaaactcatattatactataagtttcctcctgtaaaataaataccaggtctcagcagttagtagcagacacaagattccaggtgtcccatattctctataccttgcatcatctgcatagtaatggttacagtagaaaaaatagaactgataccagtagtaatagtcatggctgctgtactagtcagggttctctggagaaaggagatttattataaggtatggtattggctcacataattatggaagcagagaagtcccatgatctgctatctgcaagctaaagacccagtcaagccagttgtggggttcaaaagcctgagaaccagagagaaagtatggattccaatctgagtcagaatacctgagaaccaggagtgcagagaacaggagataagactgatgtccaagctcaagcagttaagcagagagcagattcaaccttcctctgcctttttgttccattcaggccctcaatggattggataatgcccactcatgttggggaaggcaattcactttacccaggctgctgattcaaatacaaatttcttctggaaacactccgatggacatacccaggaataacatttaaccagatatgtgggcattctgcgatccagtcaagtcgacacatgatgttaaccatcacagcagttaacctttatttcgctattaatgggacagataatatactcataataaccctatgaagttagtacaattgtaattcctgtatttaattttaatatttaattttttaaatttcccaggaacacagattcaagttgcccagaatatatgctccctataatcacatatttcagaggacaaaactgaggctttagaggccacactgccactttctgaacccagtcaagtctttctccagagcctgccctcataaccctgaagtactgaccccagagttccaatctcctataattcagaactttcatgaaatcagactgactttagacaccaatgactcagaactagggagttctattgtagtgtgactttggagctctctgaatggtgcatgtaatttctgttttacagagcaaccttcattatgagaagctgtgtgtctggcctgaaggaagggaacactagagaaggggctggttggtttgagttatgtgacagtctggcaggaactgtcatttggcatgtgaaatagaagcatattggattttattttcccctgagtaggttggtgggattaaatttaattaccattttaataagcagaagaaaggaacaatcccaaatagtagaaacaaaaagaattgctatggtagaccactaatcattagaaagacttatttttgaatcatgagggaccacagtgagaaaagcagaaaattcagaacacagagaattataaccgacgggaccattgcaaaattatacctgaacgacagtttgacaagtgaaagtaattactgttccatgtcagagctggggagggtctgtcttaggggatggagcaatgaggatggcccaaacctgagtgtatggatttgtagagctgcttcagaagggagggatggcccagtaatgtcaactgaggcctaatcatgactttcttcccaagaaactccaaggactacacacatgcatttcagttttcactctgtgttcctgaggaatgatggcaggcattcatattcatattttcaatgcagatattaaaccagatttatatggtgctatggtttggatatggtttgtttggccttgccaagtctcttgttgaaatttgatcctcagtgttggaggtgggcctggtaggaagtgtttggatcatgggggcggatccttcatgaattttttggtgccattctcgaaggagagagtgaattctcactcttatttcctgcaagaactagttgttgaaaagagcctggcatctcctcctttctcttttgcttcccctcttgccacatgatctctgcacacagtagcgcctctttgccttccaccataagtggaagctgcctgaagccctcaacagaagcaggtgcaggtgctatgcttcttgtacagcctgcagaaccatgagccaaataaaccttttttctttataaattacccagcctcaggtatacttttatagcaacacaaacagactaagacatatggctagcaatatagatagcaaaagcgtcacatgacttccaggtgtccaccctcgtgtcattgctgttgcctgcacaaatgctcaaaatatggcatttggggattctgaatccaaaaatgattcaggatagtcagattatgaatggacagatactttagtaagaatttagctgatttgtttttcttatatatttattttttatgtatgtattttatgaaagagtggcatatgatataaagctctgactcagtaaatctaaaagaacacttttacaaggtaaactaaaaactctaatagttaacagtgttgtgatattttaattggcaactcttttttctttctcagtgattcctaaaataatggtgcatcttgcaattcttggtgtcttggtcaattaaattcttggtcaatgaaatatggcaatattaaccacaatcattttttcaagacatgctagacacagtgttaagcccttcatgtgcattatatgatttattttcattctcatttcactgatgatgaaactgactcaaacagctaaaaaacctcacaaaaatgcccataatttcataacaattcctatgtagaatctttcaacggcctcactttggatctaaataaaatgtaaactccttgccttgtttatagtacccttttctaacatagtcctgcctactcttcccacttcatctcataccattctctgcctggccatcctgttgaagtccactgcaattgcacttgcctagaatgcacttgaccaagaacctgatggacctggctcattcttgtttttcagatctcagcttatatgcttcctattcagaaaggtgtttctgacattcatcctaaaatagccagttaatctctatcctatcatatgggttcatttttatttaaagtaattttttgaaggttaacatatacacaaaaaaggacacacattttaaggctataactggatgaattttcaccaagtgagcacacctttgtacaagcacccagaagcccttgctggatttccttctagttgctcactacacccaagggcaaatgccatcctcagctttgtcggcatcgattaaaattggaatcataaagtcctcatttgtgcctggcttctttcattcactatgttattagtaagattcatccgtgaggttttgtgtagttgcagttcacttattctcattgctatatagcattccactgtatgaatacaccacagttttttctgctcttggtggacattggctggttttccatttagggctattacaaacactgctgctatgaacgttactgaacattttttggtgaacatcatgtgaattaattttaactgtattacttaaaactatgtggtatgtatttgcctatttaaactttttctgtctagctttacccccatcacaatgttactcctaagatggtaaagaccttgtttatattttttcttattttatttttgagctttattgaggtatacctgtcttacaatatacacccatggaattgttactgtggttaagctaatgaacatatctgtcacccccaaaagtttgtccttgctccatagcagtccctctctaccacttcttcccgtagctcctatatctcctaccctaggtccctattgctctgttttctgtgatgttcattagtttggattttctagaattttttatatacgtgaaatcatacataaggtactcttattttgctttcactcatcacagttattttgggattcatctatattgttgcttatatcaatagtttatttcttttcatttgtgagtggcattccattgtatggttatactatatagtttgctatagattttttagctttaagttactattaaacttttttatttttattttttatggatacctaatagttgtacatatttgtggggtacttgtgaagttttgatacaagcatacaatgtgtaatatcaaatcagggtaactgggatatccgtcacatcaagcatttattatttctttacattaggaacattccaatttcactcttctagtaatttgaaatatacaataaattattgttaaatgtagtcaccctattgtgctactgagcattagatcttattccttctaactatctaatttgtttatccattcatctgttgatgtgcatttgagttgattctaacttttgcctattagaaatgatgctactatggacatttatgtatgaactttcatatatatccctctacttcccttgggtgtacatctaggagtgtaatgggtgggtcatatggtaggtacatgtttaactctataggagactatcaacctgttttctgtctgagagttccaattgttccaacactttatagtaagtctttttaaaatttttgctaatctgataggagtggtgatatctcattgtggttttatttcacatttccccaatgactaatgatgttgaacatcatttttatatgttacttgccatttgtacatcttctctggtgatattgtctcttcaaattttgtgttgattttaaacatcgttttttttttgttattgagcttatgaaattcaaaaataaatattctagatataaggcctttttaaggtatgttattcaaatattttctcccagactgtggcttgacatttcatttttataatagtcttttattttaatgctgtcttcctaggaactagaatagtgattggcacatgttggtgctcaaaaatattttcttgaaaagacctagtgaaaggtgatcccctaggaaatatcttagattctaaaacaggtttgagcaaccccattacccctaatattatcaatatacattacacaactaggtatgtatatgatgtaggtgctataattcattttctttatgagaataaggaaactgaggtggatggagataaactcaccttaatcaaagtcaccaagagagaagccagtgtttgaagaaggcagtctgactccatagtttttggagggtgtggtttgcagaagggatgtagaatggttagaaggagcatagtaacttcttccattgtccaactttgcattgtttcagtggcggggtgagcacatgttattcttaaattttggaagtgtgctttaaagataatatttgaaaagaatacatgtattcaagtgctggactacataatttgaatatatagatacatacaagattgactgagtttctagatgtatagaccagctaggttgaatgcagccattcttgttcagttccgtgggctgtccagtctcaggcagtcatgcaacctttaagagagtgaaagtagttggcctctggtcatagtgccacttctcccttctgttctcactctgtattattagactaatatcccctgcatggtagaatttgtcctggactgttctttattagtaaaaaccaaaacaaaccaaaagctttcttttttacccctgaattctaggaacacatatttctttttggggtgggggagagagagagacagagagaatgttttagtagcatttaggagttactgggcggcctttgggaagtggcatccataggagaactgtgagacagtatacaatgtcacgatgataagtacatttcaagaaatcaaatttgatcatattttggggcagattaatttataaggaggggatcattggccagtactttgtaaacaatctggagctcattttgctcaaattaaatattccatagtcattaaatagatattcaagtagtgcttaggcagcacagaatatatgaaaatctgcagcaaatcatactatccacataaaccattatactgcttctattgctgatgtggctgccagatgtctcttttgtgagctggattccatagtgtttttcatatctctagagttatttgaatagttacttcttgaatatctagcaactgtatgtgcatatgtatgtatgtatatatgtatgcatgtgtatgtatatatgtctatgtgtctatgtgtatgtgtgtatatatacgtggtgaaacttaactttgcaagtcttttattcatttttgtgagatatggtgaaatgtgatcttgtacatttttattaatgcaggttgggaaacagattctccattagggtaatagcagagagggtacattttaggtacattttgcaattagacaattttaacagctactggtcaaagtattatttaaatttggcatcacacttaagaaatataaactgttgtgtctaaattaagcctagttaaacactgcccttgatacattctttcttgcttccttttctttggagacagggtcttgctgtgtcacccaggcgggagtgcagtgttgtgatcacggctcactgcagccttccacttcctgggctcaagggatcctcccacgtcaggtgcacacttcctggctagttcttttaatcttttgcagaggcagggtctcactttgtttcccaggctgttctcaaactcctgggctcaagcaatcctcccacctcagcctcccaaagtaactggactacaggcatgagccacaacggctggctgtttgatacattcttggtttgccttgctatttgtattttcctaaaagcgcatgctggggtcacctttggtgctttcaaatttacaacctctggaccccattccatgtgattctggttttattgatctggtatgtagtgctgaaagccctccaggagcagaggaggttgggaaccactgctgcacaggtggaaggagcctggaagtgcatctgtgaccatggacttaactctgacaaaaataaggtggtaaaatgaaaggaatgagtctcgggaggatgtagcaaatgtaattttagcattggaagggagagcttcggcgttaggaaaatgcttacaaaatggaatgtcagaggaaagataaatagaatcctatggccagagtcttccaaagtttgacagcttaaaagaatggagaggcttcctcagagtaaaattctgaccttgcagccattcaagtagtattctccgaagagtggttctggtaaacctgttatgttaggtagttcataagcttttaaaaatcagtgacagctatgagtcaatataaagttggtttaaatgaaattgtaaataaataattgtacaggtgctgtgtggtagaaatcaggaaggtgggatgtgaatagtttaagaaatgtttgacaaacagccattaaacacaaagcatcttaaaaaaactataatgacagcacaaagagctcctgagctcagagttttgaaggagtggtgcttataaatacacccaattaagtccctgatgggaggaaaaaaatctagttatttgcctgagtgtgatcattcagttgtctcagctgttactttctaaaagcagatacttgtggcctcatggtatgttcttgttttctcctttttatttttttaaacaccaagttattcccaaaccctagaatcggagtcattatcatgatgagcctgccaaaaaaaaaaaaaaaaatttggattgcctcaacagaaaagtagtatcacttgttctctaactgaattgttcattttgcagggtcaagttacctttccagacagtctgatttgagagatctggtgtgaggccctgagatctatatttttaagtaagcttcatagtatatctagtgtatgctaaaggctcagaatgttggctgagaatttggagatgatgttgatgatgtttcccacactggcaagggcacccatctcccttccagaagtagcatctaaacaaaattataagcaatttcatcacataaaaaatagaataatataaagaacatcgacatacctaccacccagtgtaacaaatagcatattactaatatactagttgattcctgtatacttctccccagattgcattaccttctgtttttctagagataatcattcttctcattttttaaaacaacagatttactgagttatattcatattgcatacaattaaagtatacaattcaatgatattcacagagttgtacagccatcaccacaatcaattttagagccttctcatcaactcagaaagaaactcagtcaccactagaaagcatcccctattttcccctaccccaccaaactccaagccttatactcctaccagtttactttctaactctatgaatttgcctgttctgaacatttcatataaatggaatcatacagtacttattcctttgcgatcgcttctttcacttagcataatgttttcaaggttcatccatgttctagcatggatcagtatttcatccctttctgtggctgaataatttttgtttagccattcttcagttgaaggacatttgaattgtttctgctttttggacattatgaatactgcttctttcataaacagtcatgtacaagtttgtgtgtgaacatacattttcaattctcttgggtacttacctagaagtgcaattaccaagccatatggtaactcagttgaggaacggccaaactgttttcaaagaagccactccctgggtgttgatacttcttattttcacatacttttttgtacttctccttgtgttagtatttctaaacagtatgtagaaatgttatgtattctttcaaacaatgtaaggaatttcattttatattatttataacctgcaccttactttgcttatcattgtgtttctaagttcatccctgggtacacacaaagtgctaatacattcagtttcactggtggtacaatattcagttgtctgaacacacctcaacttgtctattttctgtcttgtctctttgtccgttttactgtcagagtaaataataccatctctgttaatacaaattcccaaaatccacaataaaatcttttgtatcattcatgtgaagttcaaagttaatgcttctggctggggaactctcctgcatgtgctaactcgggatctcagatttctccatcttgcatgattgtcattttctaggtcctcccctgagtttctagtggatcatccagctgtctactaagctagaaggcagggttgcactggatttattattattattattattattattatttattttttgctggaagggagtcgttactgttgcccactttgcattgaccaaaacctagccacaaggcccccaaattaactgcaagggaacctggaaaacatagtcttgctttgtattcaggaagggaatggggaatttttagtatccctgccacattgacttgtaagtttatttcaactttttgcagctgtaaacaatgccactaaactttcaattttcttgttcatgtgggtctcagtttctctagagtatgtatgtattagtgggattgggtcacagggtgtgttaatgttgcattttattaggtactttcaagtttttctcaaaggtatttatgctaatttaagctccaccctgccgtatgggtgcttcgctttcttgccaacactgagtatttgccaaacctttaaatgtggggcaccagctgttaaaagggagatgacaaatggcatggcatttgaaatggaggaaactaggatactaaaaatatagaatagtgtatgtggtagacagaataatgactcttcaaatatgtctacatctcaatccccagaaccacagaacacattcctttatatgacaaaaaggatttgcagatatgattaagttaaggatcttgagatgcggagattatcttatattaactgagtaggcccaacatgatcacacgggagtccatatgagaaggaggcaataaggtcaaatcagaggagatgtgatgacagaagctgaggttggagtgatgtgctctgaagatgagggaagaggtcacaagccaaggaatgtgggtagactgtagaggctgaagaaggcaaggaaatggattctgtccttgggccctcagaaagaaccagctctgccaatacattgattttcatgcataagacccattctgtacttctgacatccagaactataaaataataaatttatgttgtcttaagtcataagtttgtggtgatttattatagtagcgataggaaattaatataatatgaataataatagctagcatttattcagtacacagtcatgctaggttctcttatctgtggctcatttaatggataacacactccttagtgtcaccattttagagctgaggatgctgacttagaattggagaggacttagtgaaaagagaaggtagctttgcaaagagctatctctttgcaggacaaactcattttcgatgggaaagcccacacagtgacagagctctaaaatacagtgagttacagaaataagggatatagatataggaggtagggtctttacgttctgtccctggttctgggagggttctcaaagaggtgacttttgacatagggcctgaaagatacaaatgctctgccttgtgtcttgaaagaaaaagaaaattcaagatgcaggaactggagcaaagctgaggcctggaaagttatttttaccacatcattctctgataatattataattaaataccatatctactggctgggtacagtggctcatgccagtaatcccagcactttgggaggcagaggcgggcaggtcacttgaggccaggagttcgagaccagcctggccaacatggtgaaaccccatctctgctaaaaatacaaaataaataaataaataaataaataaaattagctgggcatgttgttgcatgcctgtagtcccagctgcttgggaggctgaggcacaagaattgcttgaacccaggaggcagaggttgcagggggctgagatcatgccactgcactccagcctaggcaacagagtgaaaccctgtctcaaaaacagatgtaaaatcttagctcctcatccatctctctcccttagctattaaaccattagtgttttctttggtagtgctactaccacaactgaataaaaaaggatttgcgtgagaactgcacctgattttgattgttagactattgaaagagttctgcaaccaacaccactatctaattttagaacattttctataatccccaaaagaaattctatgcccattatcagaaactttccactctctcctcccccaggcccctgcaaaaactaatcttctttgtgtctccatggatttgcttatttgggatacttcttatatgtgtaatttatatctaatataaatggagtatgtggtcttttgtgactggcttctttcacatagcacaatactttgaaagtccatccatttgcagcatgtatcagtattttgttcctttttatggtggaacaatattccattgtgtgaatataccacatcttgtttatccattcatcattgatgaacatgtgagttgttttcactttttggcttttatgaataatgcttattctatgaccgctttgtataagtttttgtgtgaacgtaagttttcagttttcttgagtatatacctttaagtggaattgctatgtcgtaaggtagttttatgtttaattttttgaggaactgtcctgttttctaaagtgactgtactttagactctcattttacattcacctgagagttttgatttctccacatccttgtcaacacttgttattatctgactattcgattctagccatcctagtgagtgtgaagtggtatctcattgtggttttgatttgcatttcactgatgactaatgatgcgaaacatctttttatgtgcttattggccatttgtatttttttgggaaatatgtctattcacattctgtgtccatttttaaattgagttaattgtgttttattgttgagttgtaagagttttttatattctggatactagaccattatcagatataatattagcattattttatcccattttgtgggtcaccttttcacactgttttttgattgctgtttttgtttgtttgttgttttcttttaatagagacaaggtcgcactgtgttgcccaggctggtcctaaactcctggcctcaagcaatccttccacctcagcctcccaaagtgctgggattacagacatgagccatcacacctggcccacactctggatagtgtcctttgcatgaaagttttaaattttgatgaagccaaagttatcatttttcttcggttgcttgcgctttaggtgttataactgaggaaccactgccaaatttgatgtcatgcagatttacccttatgttttcttctaagagttatatagctttagcttttacatttaggtctttgatctatttttagttaaattttgtatattgtatgaggtggtatggtcaaatttattcttttgtatgtggatatttaattgtctcagcaccatttgttgaaaatatgattttttcccatgtattgtcctggcacctttgtcataaatcaactgaccatagatgtatgaatttttcttgaactcttagttttattccattgatgtatatgtctatcctctgtgcctgatttgaaagattacttgaagtggttgcttgatgcatctacatggctgatcttggagagtcagtaagtttgaaaagacacttgaaaatctgtattttatcataagcactaccatgtgactctggggtcaatgttgagaaactatgacagcacctctcagacttcaaggaacatacagatcactagagcgcttgttaaaatgtaaattctgatagagcaagtctgaattagggcctgagattctacatttctaacaagttctcaggtaatgatgatgatccacagactacactgttggtggcaaagggctagaaggtctcccaccacagcatgatcccatgccttccaggtaggcctctggaggaatcactaggcttaaatgaatatttgaattttctttcccatgttaattgttgcctttatttacttttatgccactgagaaaaccaacttctccagctcatttatggtgccttacaaacctgatttgaggggatgggtttttgaaaaggctttaggattttatttctggatttacagaaaaagacatggagttaggagtgcttgatggaaaagtgaatttaatggattctgagtaattttcctacttatttaaattctacttgtttccagaagaggtgtattagcaggttatttttctcttacaatgatactttaatgcagatatataattgcatcatgtttgcctaaaaattggtttagataaatcacaagtgccaggttacatcagcttttccaacatccacttgatctacatttatttcaaaggtttgtttagaaggctgacatgttcttagactcttctttttctgctgtttgctttcagcaagtcattccaattatttgcatataaaatatctccattttcacatagggacccttatttgtccctatattctctcaggaaaactgctatttctcaagtactttggtagaggattgccctttatagttcgcaaatgtgtgcttggatgtgcccttgcttatccttccatgctttctgggcttggcgtagtaagtggtcattgattttctgttcaaggggttttgtagtttcaggcaactgcagctgttaattctggatgtcaatttctagtaaacctacatttataaaatgagctactcttgtgcagtgtttgctttcataacactagccgtgttctgagttacaagcacttgttttttttcttgtttttgtccttaattgttttctaaacatttccggaagcttgggtgttcttcaaataatagtgtcttcaggttagttgtgattttctgacttgaaatatttttagtgcagcaccttagtggctctgtacttagctagtatttgaagcatggtttggagctttttttttctttaatttgtactaaatagtgtttttgctattagtaattcatatatatatatatatatatatacacatacatatacaatagtacagaagggaataaaatgaaaattaaaccccgtttctagttctattccttctgttggttgccacttagctttaaccaatattttggcatctctgtttattgaccgatgcagtctcttcttcaaccactcctaaaaatgggggatgcagaacaatcacatctcctaactttcccacccctgtccaatgcccagtttttctctacttcctgagattatatttttcttccttgtgattttaaagtatgtgcttacgttctttttcaaagactttcagaattattttgaatgggaaacattcatattcaaaacaataaaaaatcaaaacacaaaaatgtatgcagcaagtctctttacctctctatgtaagaagttaatgcctccaacagataatcaatggtaattatttctggggttccctttaagatgctttcaatgcataggaaagcagatgtgtgcgcacactacttcccttctcccatatttataaaatgtctgcgtttatcattgttcatatgcccttccaatcatcttgccgtcactgtctcttcttccaatcactgcaatgaccactagcccattaagacctccagccattgtctcgtgagtacaacctagctaacctcacatgatgcctaagccatatgccctttgcctcctgacccagattgtccttgtttctagccaaagcatgggactccatgagatcccacttattgcccacaaatgcacaacccagaagtatgggaagttcatgccctatcgggctaatctgtgacccaagggaaacaaaagccggttaaaaaaattcttgctcttttacttccccagactgactgtcctaagaagcattttttatggattctcagaagatggttctttgtgatcaagcaaaatcagccaaccttactggtggccagcaccataaagcatttttgtattggctctgtttgcttccttgttttagtccccatgtctctcactcttgcttcctgaaactgtactcccagataaagtagcaatacatgaagcgccatgcccttttcctagttacttttatatgtggaattaattttaatgtatctcaagctatttgggattgagataatttaactcatgctggcttttcttactagaatgaattaataaatttttatatactgaaatgttgaattacaaatcttgccaatatcacatttaattaaatgaatttgaaatcatgatggagatggagatagaaagaaaattttagtttttcttcgtgacagaaatagtttacctggtaatattatatattctataaatataaaagtctattaaagtctattaaaagctccatatgaacagaagatttatatgtataagtgtatgtgtgtgtgtatatctataatataatgtatatatatgtttatatatatatacaagtctcacagttccataagaacagaatatttctaagcaaagcaaatttctagacaaacttcttagatagtttattagtcacttgggaaaaaaaatctataaaacagtttcataagaaccagatttaaatatatttatatctatataaatattttgacattgaaggaattagctgtgtgtgctatgcctaatattttcccccatggctagtaaatatttgtagatagtgtcccttttctccttgtaggttacagtgaaggggaagagtgacccactaaattaccccagccatgacctgaaaaagttacatgggaggttttcatcatttgttttctcatagggacaacagtaatattgaagtaggaaacctctatgtcttctattgtttactgattccatatcttactgtaaaataaaagcatgtgagagtgaactggagatctagatatcccctttaacttgcctgaactgttgttagaagacttttcaaactctcctgcagtctttgaattttcagaagatgcctttatatttatttctttcactagttagctctcggtggtgtctgtggcatcttagctctcaatggtgtctgtccgtctggagctgggaatgtgatggagaatctctaactccttagttgggggagatttgtagatgagagaggccagttagtcatgtttgtgatgttgggttaatctctttaggaaaagagcaggctggcagcaggttgatgtttgcaactttgatttacttcttggaaagtagattgtggaggtaacaaaggttgttaggccataatcagtcatttcgtcagccatggagaagttggaagtaaatctaccaaaacagtcacaatgaactgcattattgtaaattcaaacagtagtaccagtgttacacattaatttttctacttactggtaatatttatcattctgtcaaaacttgtcctgctgtaagaatattgagaactaaaaagtctctaagtacattcttcttgtcatcccacaggcatgaagttcaaatagatgtcaaatgccacaataaatggtcataaatttaaaagattttttgatagtggttggctaataaagtcaaagtttaacaaacaaaagtgacctttatgcttgaagctaaatgagaatatcaaatcatttggttggaaaaacaggctgtgatagaaatttcaatagaagaaacagataaaatgagttatctctatgaaaatgaagcttagaatcctcaaagcgtcatgaatctcaaagttagagaagaaggtgaagatcgttgttctagtgctcagctaatatcattcaaccttggctaagaagcctaaaggattagcgaagacagttctattttattaggaagggtttttatagattgtaataaaacttgtcttcttataaattctacacttgaatcttttagaaaattatgatgaagaagactacactatctttcatttgtcaactatacagatatttctttttctttctttcatttttttttttttttttttttttttttggagacagagtctcactctattacaggctggagtgcagtggtgtggtcttggctcacaacaacttccacttcctgggctcaagcaatcctcccacctcagcctccagagtagctgtgactataggcgcacaccatcacactcagctaatgtaaacatttttttttgtaacagatatttctttatgggtgtcatgctgctctctcatttctttattctcttcctttcacaaaagtctatattctaggccaaatattcccaaaacattcaattatacctcatgttcagtggctgcagaaaggcacattatcattagagtagaattttagattataaatctctgtctcacactgtggtatccagaactgaatataagacccatagaaatcttcccaggacagaagtgaatggaaatatcactgatattcaaccacacacacattcttattaatggagctataatcataccatagacttgttgcatgttccagttaaaactcttctggtccttttcttacatgttgttgagaagtagctctatccttttcatgtacatttcattttagggacataagttcaataaaagataagcaaacaaataacaacacaaaaacaacccaaagatacgattgtcacctattaagtggacagcagtgaaaaaagttgttgggtaagcatacattacagtgggcctcttttgccctatggatgactttgtaaagtgtttggaaggccattgtgcaatatgtattacagtttaaaattgcacaccttttattcaagaatcatactttttagaatgtattttttctaaaagtggctgagatagatatctataaaggatgacttcactacttatctagtttttattccatggatcatcagtataatcatggtcttgcaaatactttttaattctttcctttcccttctttgcatttatgcaaagctctccctagatttatgattctcaaacgtttaagatccatcagaatcacctgaatggtgaatcttgttaaaaaatattgctgtgtcctatacccaggtattctgatattagtagatctggggtggggcccaagaattaccatttttagtaagttaccagctgaggctgatgctgctggtccagtgactgtgctttgagaaccatcatccttgatgaacccagccactcatgattgttgcttgcaagagtcacttggcagattgatctcactacaaattcacgagccccaaatttaaatgtacataaaacactacctggaaattctaggaagctactttttccattgtctgcaaataattttatatcttgcctttcctcttcaaacttacctgttcttctcacatgcactaactgcacaggctttaagctgatgacttgcctcatgcttttttgagaaaacagaaaccaccagagggaagattcccgacatcttcttatcacaaatctgtaaatcagctggtgtctgaatcctgttttctccttctgccttttctttcctggagaaagtcaaaggccaaccctgtactttgaatcctgtacagtctaacctctacattcagtagtttgcatgtttattcccctctcaccatactgaattattcccattaatacacaaatttgctctaggatttcccatctttacattacacacgtagcaatagctatgaaccattttttttaccccataactcatacagttactctactttttctgcaaccttgcaaagtcaaacttctcctaagaattgtcttcctatattttctattccctaataactggtaaacccattccaatcttgttttggcccccacattgccactgaagtagattttcctttggactcccaacccctcagtctaacagagttcttagtcctcctttgtgtaacatatgccacagctaatagttctactggcttcttttccagaacagccttctctaaatggtctctacaggttggggtacttctcttctctcaacctacttttcccatgtagtcttattcatctctctctggatgatatagaaccattcatatactgatgaatcctttattttatatctagcacaaacctttatgctgagatccagaccctaatatacacaggtgcctaattggcatctccacttggttgtccaaaaggcaacccaaaactgacatatccaaagtggaactctaaattttctcataagtcagttcatctcctctccttctcccattttattttattttattttttcttttttttttcttgtgctcttatttttatatacgaatattttttattatactttaagttctagggtacatgtgcacaacgggcaagtttgttacctatgcatacatgtgccgtgttggtgtgctgcacccattaactcgtcatttacattaggtatgtctcctaatgctatccctccgccctccccccaccccacaacaggccccagtgtgtgatgttccccttcctgtgtccaagtgtcctcattgttcaattcccgcctatgaatgaggacatgtggtgtttggttttttgtccttgcgatagtttgctgagaatgatggtttccagcttcatccatgtccctacaaaggacatgaactcatcattttttatggcattctactcatctgacaaggggctaatatccacaatctacaaagaacacaaacaaatttacaagaaaaaaacaaacaaccccatcaaaaagtgggtgaaggatatgaacagacacttctcaaaagaagacatttatgcagccaacagacacatgaaaaaatgctcatcatcactggccatcagagaaacgcaaatcaaaaccacaatgagataccatctcacaccagttagaatggtgatcattaaaaagtcaggaaacaacaggtgctggagaagatgtggagaaataggaacacttttacactgttggtgggactgtaaactagttcaaccattgtggaagacagtgtggcgattcctcagggatctagaactagaaataccatttgacccagccatcccattactgggtatatgcccaaaggaatataaatcatgctccttctctgattttagaaaaaggtgctacctagttgctaacagtctggaaggagtcatttgacaattccccatctcatctgctatatcgaatctatcaccaaattttgactgtagtctttgatcttccacacatttccatttccactgccccaactctagaccaagccccaccactgtcacttgcctgtttcctgtgataaccccaacaactttccttgctggcctttcttcaatccttttttcctgcagcagtaagagtgatcgttcaaaaatgcaaatctgatggtcaccttcacttagtaatttcctactacatttggaataaaatttaaacttttcatttagcatcttacaagatctattaatcttccacctctccagcctagccttgggctacatgctatatacaatagccatgtcccatgtcttccaatttcctggaagcaaagtctctcttggggccatttgtgtctgttgttccctttgctttgatgcctcccatctctttgcagcctggctttttttcatcttttagttcccaaattatcagttacatcctcagagggaccttcctacccatccaatagaagcctcttctgttgttccctctgttcttctccctcatattgcaaattattattgaaatgatatgttcatttttgcttttttgcttatctgtctcttctagtagatctaagagttcattaactgtctgaggttagagatcttagctgttttgttaatgtctaaattcccagtctggctcattgtatggcaaatggtatttgttgggggactgaataatatttattagagcattcctgttaacagtgtaaaatcaggctatagtgtaaaggtccatcaatttaatgattaagttataggacgatccattgtaaagaaaaaagtaaatttatatatgctgatatggacatatatttaataaatattaagtggaaaaaagcaaattgctaaacctcatatagaataataccatttttcttaagtatatttccatatttatatctgcatgtttctttctctctcttcctctctccctctgtgagtttccaggatttctactgggactttttccaggtaatggcaatacagagtgacaagaatttattttattttactctttctgaactattggatttttaaatttttataagttccatgtattccttttataacatcctccttcaaaagcatgaagaagatttaaagtgtaatacaaaacattacgtatgttcattcactccacaaattttattcaacatctactatataccatgtactgctagaagctgcacatatagtgagatccatttattgtatataattgggtgtcaacaaaagataattttatgagggtttgtttctggcagtggtgagattatggttggatttcattgcaccttctcattttcaaatttttgatggtaaacataactttgaaattacaaaagttatttttaaagacatttaaatatttacatggaaaaatgctcaatcattttagaacaatttacttggacaggtgaatacttttcaatttaatggaaaaataattgtataagtgccttttgtatagatgcaattattaaaatatgtgtagcacatatagcttagaaagtttataaataggtagttgtttccagtaggaaacaggatactcaatctttcttttctttgcctatagactgatttcttcaacatggcctactaatgtgttcctcttggaaaatgagtacctaggagagattcaaggttcttgttaatgagttaatatttgtaacatatgcctgagggtatttatggaaaccatgtattggatccagaagggcaaaactctccgggtggtggaagctgagttgtaagcagtgtgtgttgagaggaggcctaggggatacagactgtcatagcttcatgagaaaaaaaaattaaaatgtgctgtaggaaagggaatgagagtgagaagaaaagccatccaggggatctcatatactgaagatatggtttttctttcgtcgaatgtatgatacttggaggaacaaagaaagataatatcagatttgagaactgttgccttaaaaaaaagataaaatgctgactttctgcaaatatctttatgaccaactttccaaaaacagggaaactcaatgtgcctatgttttttcctctggtttcttaattccttgatgttctcatttccagtgacctttttagaccctcccaaggtcacatgttggatgctattgtaaccagaagtaccaccatgtccaacatcacagtgcatatgtcttgctctctaactaaatgttctaactcatctgcttattttctcctataagcagcatttctccttcatcaagaccaccagtctgctaacccacattttctctctattctttagccttgtcctattagtctttattgcttatgcagagagattcaatgatttatatccactttccagctcctccatcaattcttgccttttttgcttgttttaaattttttaaatttttctttaagttctgagctacatgtgcagaatgtgcaggtttgttacataggtatatatgtgccatagtggtttgctgtacctatcaacctgtcatctaggttttaagccccacatgcattaggtatctgttctaatgctctccctccccttgcccccaccccccaacatgacccagtgtgtgttgttcccctccctatgtccatgtgttctcattgttcaactcccacttatgaatgagaacatgcggtgtttgctcttctgttcctgtgttagttcgctgagaatgatggcttccagcttcatccatgtccctgcaaaggacatgatcttattcatttttatggctgcatagtattccatggtgtatatgtgccacattttctttatccagtctatctttgatgggcatctggattggttccaagtctttgctattgtaaatagtgctgcagtaaacatatgtgtgcatgtgtctttatagtaaaatgatttataatcctttgggtatatactcagtaatgggatttctgggtcaaatggtatttccggttctagatctttgaggaattgccacactgtcttccacaatggttggactaatttacactcatcaacagtgtaaaagtgttcctatttctccataacctcaccaacatctattgtttcctgactttttaataatcatcattctgactggcatgagatggcatcttattatgattttgatttgcatttatctaatgatcagtgatgatgagtttttttttcatatgtttattggccacataaatgttttcttttgagacaggtctgttcaaacaaccccatcaaaaagtgggcaaaggatatgaattcttgtctttttctccctctggaacttgtttcacaattctagaacactctgagtcagctctctttccaccttaactgaccaacctataggtaacctattggtataggtagtctgcccagtaggctactttgtaacagcatacattgatactatttacaaaattttatctttttaaaacatgtatgtgtttaccaatttgttttatacttctgctatgaaacagtgagattcataaagtcaataatatagttcatatctccaggaagttagcacagtatcttttgcagcgtggatgcttaaatgatgggtgaataaatggataaagtcagctccaaagaaagaactgagaggtagagggagtgaccagtgtattgatgtgatacataaactcttctgcccacatgggaaaaaaatggcatcctggttaggctgtgcaattgatgagtcgctatcagttattagaaaaaaacctttcccacctaagttttcaaagtattctgctgaggcagtagagcgaacttgacttagtcccttttctctaaacgagcatcatctcagttgcctgcctgtctgcctgcctgttcgctttctgccagaacgtttgattgattgagtcatatccttgcttcattcctagaaggattccaggtgccaacttccacttgatccgaggttaaatatatctcctgtaacctaggaatctctcagtcttgctttctgctgctggagaatactaccataacatgtagtaaaatataaatggaatatcagtcatctctgaaatggtaatatattctccagctttctattcaaatatttttctttgagcttctttctttggagtctgttgttagatattctcaaaaggattgtctccacttaagtttcttgaaaattaaattgtagatgtgttcagagatgtgtctgccataagttactgtcccacttaaaaagttcctggcaatttttggatatttcctcatgtgaaaataactcctgatactgtcatggactggggagtataattcctgcaaacagcacttctgaaaagcacttggctcttggctctgttgtagaacgattatgacttcttgacataaaatgtgtttttaatctagaggccagggccagagatttgctcaccatgattaccaaacttctcccgcaacccttagacatggtatcttagattgtatgactttctgcttctgtttttctttagtgagagatggttacttggaatgatcctttaaatattcaacacatcttcattgatttcccctgctaatgtgaagatgttaaaaacattttcttacattgttagctcatttatttatttatctgcctaataaaaaagtatagcatgcactgcatgcaggggggatcaaaatagtcattgagtcacccctcaaggtgcttacagtctggtaggggagaaaggcaggcaataaataagaacaatgatatggcagaaattctcagatcaggagggaagagggtgttctatctgtgaaggaggtacaggcagaagaaattccataaatgaggtttcacatggatttagttctcaaagacaaagtcttgcaaagacttgggaggaagccatttcagctggagtcagcagtttgaatgaagccctggaggtgtgacccaatcaacagggaggtgtccctgggactgttgcctctatgaggctcatgggtcacccaccggatgcctttttaaaggagtccttctttgctttatcaccagcccattggccttagctataaagcagatttatgcaaagtaattataatagagcagtttggtttgagctatattttctaaaaggattggtgaatcaccagtgcctgaattatttactgctctcattgagagtcaggtccaaatccagcctgatgcacaggtgggaaaagaaggactcagtggtcagagaaaagtcatcagagagatgaaggggccagcacttggaagtcttatataggtcaagcagtaaagacattgtcgggtgaggggtggatgtggcacacggcacacacctgccctgttggcacttgtcagaaaggcaggaatcaaagttgtgcccaaggtcctgctttgggtgactgggtgagtggtgggtcttttcctacaacttcaaatggaagagaaggtggaacatatttgtgaggacagacaaggagttcaattgtagacagacatgtggactgtgaggtgcccaaagacaccctgctaggtctttccaggaagcagggattttggagtgtatgcccagggcttaaaggtgatatctagcctaaaacttaacatttctaagtcgtctacatgtgagttgtgactaaaggtggtgaatgaatatgctacacaatgagaagagaaaggtgagagaaatgacgctgaatattggactttactgccatgtaaggagaaggcaaatgaaacagggactgaaagctgatgtctatgaagtaggagcagaaacaggagaaggaaggtcatggaagccaaggaatgtaacccattcccaacacccattccccgtgatgtggagaggccacggaagatgaggaccagatctcattacaagagtgtatggtacagagtgagatatctccacaaatgcctagtacctttcccctttcatcactttttattcaagatcaaagtataaaataaccaaattcaactgggtgcagtggctcacacctgccatcccagcactttgggaggccgagatgggaagatcacttgaggccagaagtttgataccaacctaggaaacctgaagataccctgtctacaaaaaataaaaaattaggtgttgtgttgtacacctgtagtcccagctactaaggagggtgaggtgggaggatcacttgagcccaggagttggatgctgcagtgagctatgaccatgccactgtactacagcctggtcaacagagttagaccctgtctctaaaataataaaaatcaaattcaactctttataagttggctttttttaagagatcattctacctttgtgtaatgtccccagcccaaactttctatcctcgcattcccctgggccttggaatggttttccctctcccctcttttcctcttttcatgtgttatctcaaaatttaacttttctggggcccctttatactgtttgctctctgacaagcaaacaaatctgattaatttgtttacataagatgctataccttaattatggcatagtgtgtcttcagcaccaataaggtttgtattgtaaccttgattctgaaagtcttgagataaagcagtagattgcaggaacttcaaacacaatagcaggacccgtagaggaagttttaggggtacagcttcctttgcctctgcataaagcaggctctttgggctgtcatggaaaagctgtttgggaaatggggtagatgatgctggagaatgaagctccttaaactaaattctgccagctccctcctcaatgtcttcaagcatttgctcattcatttacttgcagcttgggcgtaaaaaggcccctcctttcctgacctgcatcagcatctttttgcaatcccctgtggttagaattaggggaaaatgagccacttcattttgttggtatcattcctggggcaatttttgactgatgccaacccatatggtgatcaagagtgactttgtaaccatcactggaggttaagggagaagggatgctttatttcagcttctcctagaatgtcagcaagaaattcaggagacagtttgtctgcccccaaactcctggtctttcccatcttccccagaccatcatatgtggaaccagggaaaaggatccaggtctggaaccagcaatgtccattaagtgttgtggacccttcctggtctgaaataggtctagcttgtagtgctcttgaggtcgcctctaatactgactcttaataatggaaaaagtaaacactgtatacaagcaatgtttttagaaaaattagaaaagaatggcttaatggaaactttttttttcattgaaataattgagagtttaagtgagtttttaaagttaaagtataaatgctgacaccttgaagagccgagcctagtttttcactttgaattgcgagggcatctggtgtgtgttctgaaagggagagcagtttgatagccatctgtccccaagaaaaattcagatttggaagtaagatgaaacaagaatgacacctgcttaagccaagagttgtgaataagacataacttctgctacatacagtattttttaaagaagaaagaaaacccaccaattagagatcttacagaaatgttcagatgtacttgagtcaccaagggcttgcctttctggcagccagtacaaacttctacaaaggattgatttaccccaaatggcatactgagaacttcccatagagctcagctaggaaccctcttcacaccttccagtggagggagggactcccatattccaatagttgtatctttgcaagtaaacccagacatctttgtggatagtgactggtctttattcacctgcatagccttagtgtgtttgatggttgttgaaagctctaagacctgaaattcaccaacatgtctgtgttcccagctgcttggctcaaggtaagttttacccccctgggtttaaatactagtctctctgcttcttggaggtgggggtaaaactctaggattaagctgaatcttggaacccctttgttatccaagacaagaagaataggaagagggtctttctgtgaccaatagagttatagataaaattgtgtccacatatccagtttaaacaaacatgaaagtacaacagtggcttggatatgaccagaaaatacaaaaagacaaggcactgtatgtgatgctgttttttgcaaggtgataaccaggtgtatctctgctcttcatctgccaaacaataccttttatagaagagattttaccttccagttctaacactgtagggcttctctcctcttttcatcatattccctctttggaatttaatacagttgaagagggttagctgtttagaatctgggtggtaacggggatggatttcatcctgacctcaatttaaagcttatctggatctgtaatgacttgagtatgtgccctgacaagtctttggaaacctgtcagaatgaggtatcaataaagagaccaggcatctagtgtgaccaaacactcatcaatgataggttcttgccttgttactggagatacatttgctgtgcttccctggttgagatgtctgtgtatttcttagtttaatgtgtctaggattttctgcacattacattgaagctgttactatagtcatctatgaccatcattggtataaaatcacattgaacaaagtcaatgctttctttaatttgtttctgtttagagaaaacaaagaaaatattgtggctttttccccctatcaacagcactagcttttgtttattcaattgtatatgggtactcacacataccccccccaacacacacagacacatgggctggagacagctaatttttagctattattaaaattaaattatgtaaacatacaattaaataagttttgttataaaacaaagaaataaacactcaaaagtcaccacttcctcattattttactgcattttactattatctgtgattgtgtggttatttatatctattgcacctgtatgagggaaacactgtaccatggtgtctactgcatatctattcccaaatctgcacttagcagtatcaagaatttttttttggaaagccagtagttaaacatttaccagcacaccactgtaaatgtatacacatacacacacacacacacacacacacacacacacacacatccttttcatagccatgcagagtaagcgtcagtttttctattttatagatgaaaaactgaggcttagagagtttaacttacccaagagaccactcccatgctttagctctcctactctaaaaatataaaataagaagcccagggaatgcttaaggatgactgaagctgtattgggatataatttaaaagcagaggtttgctagaagtgagagcaagagaaccatgtggaaatctagacaagcactccaggcacagggtgccaccagtgcaaagacctggaggtgggattgttttgctgcttctgaggatagtaagggtactactggagctggaatagaaagagggagagggacaataggaagaagtgagacaagtagggcatgatcactaactacctttatgaaaccaggcaagtaacttgacccctctatgctttatttctcttatctgcagaatgtttcacttcatgctcatggtaagagtatatgtgaatttactgtgctttagttaaattattgttactaaagctaaaccatcactgttgccttactagtgcttaaatataattttaaggaagaataattttttcttgccatcttcagcagctgcatgaggagtgattcagaaatacttaaatttatcccttgcaagtacctattagcctattttaaatctatttaaaatccacagtgcaatgcagtttaaattcatctgggattaataggacttgcctttagctatattactgatggcatgatagaatctcaacaggaaacttttgtgacatttttcaaatgtccagtctgatggattatacaagactaaaaaagtattgaaacacattctgtattttgaaaacccaagagttcgattttaacagtcttcacttatccataaaacagattaccatgtcctcttatccacatatcaattatataggtgtatcttagagtatatttaaataacggtatgacataaaatcatgagcttctgtggctttgataactttcagtttcttcttaaatgatgtgttcactttatcaaaattgatcaggctgtacacttaggttttatgaacttttctgtacatgtgctatactgcatttttaataaattttttcttaaaagagtgagtagggctttgagaaaggggaaagtgtttgctctcctggatcaaggaagcaaggtacaagatgagatgaggccagtgttagaatgaacatggcatgtgtgtgtcacagccaggacacaaatcatccacgacatgtaaatgtttgttgagcacttcctgggggccagtatggaagatactgtgaactgggtaccctaaaatcattcacagcctacttcttccttgaaagctcaaatactcaacttcccagcctcccttccagctgggcatgggaatgtgaagttgttgaatgtgcttctggaaaagcctcttaaaaagggacactcaatgaatataccccttaattatttcatcttttgctttttgctgtcttcatcctgctggaatcaggctgtgattcccggaagcaggtcagccgtctttcaaccatgacatgacaagcatcaggacaaaggcttatgtactaagaatggaagatggacagatgggaagcgtgaagccccctagagcagtgcctctgattggttgggtgtctgactgataggcaggtggggagggtcaaggatatgtgggtcagtctattctaggacactacttttctgaccttgaggctgcccaaaaaattgccctttgggaattttgacatcatctacagacatgccactttaacaattatttgtttgttttgacatactggacatattcttgcaagggacaacacatgatgaaattaatcaaagttttgttggttgccaactttttcaaagttactagcagaaggtcttcacaaatacaaatactttcctctcctatgccttagacatttttttcccctctaagatggtaatcttatgtcatcctcatctttgagcaattctctgtaaaatgaaacctttgcaacatttccatatctagagataccactttatcttcacttggagacttgataattttccaaggagatacgctcagagggaaatacaaagagtggaaaagaatttgttaacttgaaaagttggcaaaacagatgtgctgtgattctttcagttgagctagcacatttcatcatagccattttgaaatataacattaaaattctgtgctcaatgtcccttatcaccaaaccttttgtttctgtttgcattttgttgttgtaatcatacacgtcataaatgagtatactaaggcaagtgataatgtgtataataaaatgtagtcaatactaatttgccacctttggggagaaaaatgacttccatctgcatgataactatggaataaatattctacatccttacctaaaataatgtattaatgtcaagaattcttacgatgagcataggctatagaccagaaaaatagccaggggttatgatatttgattaaagttttaacttgatacaagttaaaacttgtatcttggcttttaaaagccaaaagatacactgaaaattgaattgaatctagtataatatgacggcttactgaaattattggaaaggaggtactatattaaaataaaggaaatgtcttcctttgagactaaactgccattttgttcaatttgatacacactttatccacctatttctttatttgcctgcaaagagtaagagcttaggtaaggcagactgcgaaattcaaagcaacagtgtggctacatgtgttgttttttttttctttttttacttcttctcttgttttttgcatgtgatcctgcctttcacttcacagagaatacgatccatctatgtctgatgtcttcccttcctcctgcaatgaggttcctatattaatactatcaaacctcctgcaaaatttgaggcttggatctcatacattcccacctgatataggacttggcttctccaactgaaccctacattttacttgtatattcaaaacatttctatcttggcttcttgtcctcaatccacaaatactatttacaaagaagatttttctctgcttttatcttcaggtttctgtctcccagtataatcatatccagccccatggctcttactggggcctctgaaatgtttatcaccaaatctgtgtctgcagttccaacctcttccctgagcatcaaagctgtatttccaactttcgctggatgcttctatctgggtaatatcttaagctgaatttattcaaatatatatattcaaaatggaattcatttgacatcttcctcttatctttgaagctactgtcattcccaatcctatatctcagcagtggcaatcctgtatcttagcagtggaagctgaacaagttccctcataatattgtagaagtacaaacagagcatcaagaatagtccttttaattaaagtttaagccagtaaaaacctattttatctattctgagattcccattatttggttatatcattgtagattattcagaggacactggcatctgctacttaattttattttttttaacatttcatgagacttaaattacagtctcatggtagaattgtagttaattgatgtaccttatttcttgccaagattctgttttaaacaatttgattaatgtattgaatggcatgaatacaaaaattgggttagttaaaataatttttacagatggaagatttttagcgtggttaaatctattaaatggggaggtaagagctgttttggccttcagaatctttgagaacatcatgttaaatgagacaaccactgtttctaactcttctttcttaacttactgagagttacaacattgtattgcaaattataaatgggatcatagaattaaacaatttagacttaaagtatcacatacttttagcctgtgtaataaatatcactagacattatttgaacacaatgttccgtaacatatcttcctcggagaaacaaaacagacacctttcctgagggtgaaggtaaccttcttgagatttatttaagcaaaaatagaatcgaagatgttgagatagagaagttcttagagttccctgcctccttggttagaatgatgaaacagaaattcagaatatcacacggtgcttagaggcagaaatggaaaaaaagaatctggtgttcaaattcatacttcaggtaggaggcaagcagctatctggaccttaaatgattctcttgattgattgataaatagatagatacgcagcaatactttagttggatacattatttggtgcctcttatgtcagtactctgatgttttccattttcacagtttggaaataagaaaaagtagacaaacttggctgggtgcggtggctcactcctgtaatcccagcactttgggaggctagggtgggtgcatcaccggaggtcgggagttcaagaccagcctgaccaagatggagaaaccccatctctactaaaaatacaaaattagccgattatggtggcacatggcatatgcctgtaatcctagctacttgggaggctgaagcaggaaaatcgcttgaacctaggagatggaggttgtggtgagccgacatcatgccattacactccagcctgggcaacaagagcgaaactccgtcttaaaaaaaaagaaaaaaaaaaaagaaagaaagagaaaaaagaaaagaaaaagcagacaaccatgcctactctctctttagctttgaaaatgtttgacgatggtggtgggtggggcctcctctgggaccctatagtcatcacagcagttaacacattataattcaatttctgattctttgtcagacttcctactagactgaaagttccttttaagagcaggagctgttttcttttctattactgtatctacaatgcctgatgcaaagcaagttcccagaaaagacgagtttaatgaatgaaagggagaatggagtcagaagtcagagcacccagggacccctctcctgtcatccggttttaaccctgtgtaggaggtaaggaatgatactcccagataaacactgcctgccacccacatgtcacggattccaaggtacacatatttttatattttaatatccctgacatcaaaatctaccttacaatcaatagcatgtcgtagtaagatactggcatgactcacagttgatggcttattggatccagtgaagagtattatttttgtaggtaataagctcttacataaatgagaacaggtaagaatcgttggatgggctgggttggagtatagtggtgggaggggtttgcagtaataaacaggatggcccaggtgggcatcattattaaggacatgagattcaagaaagactagaaagagctgagggagggaacatgtagatatctgggggaagaatgttccaggaagatggaatagatgacagtgcaaaacacaagtgcacctggcacccaaggaacatgtcaggaagccactgtggctggaacaaggggaggtaatagagacaatgtcagagagagaaagggaggtggggagtagatcatataaggctctgaaggttattgcaaagacagattttactctgagggacatgtaggggcaaagcagagttctgagcagaaagttgaaatgacttgattcacttaaaaattattcggactgctgcgctgagcacaaactatataggggaaaggggagaaactaagagacctgttccaatacatttatattgatgcaggagagagtgggtggtggctcaggacagatgttaacagtggaggaagtgagaagcagttggaatctggatattttatgaagatagagccaacaggattccctgaggcatgggatgttggtacgggagaggggaaaggggtcaaggatagtgccaaggttcttggcacgagaactaaaaggaagaagttgccatcccctcaatggagacagctgtgggtgaagttagctcaggagggaagatcaggaattcaggtttaggcatgctgagtttgagttgtctgttagacaggcgagtgctccaagtgatgtctagaaggctgctggatattagagtctcagtgtgggaaaagatgtctggtcttaatactagttaaccacagcatcccagtttacacaaatatggtgtgtgatatcttcttatcttattcgcctctccaggctccagaccaggcaaacacaacatccttttaacttacctttctcacagtggccttctatgggtgagctgtcacattgagacctgacccacataagctttcctaaaatttctccatttctttataccttattttagacaaatggtccatcctatcagcagtaaattcatgaacaattatgcatacctttctctcaggcctacccaattctgttcctaaaatctacaattgaggcagtgatggaacccattagatgtatagtcaattaagatttaatactctaaaaagaagaggcttcatttgtcaaatgctatacagttgtattgataaatatcagtgatagctgccaatataaaagaaaccatatattatccatttgaatatttttctctattcctgaggatgctgtattaggcactgtttatttcatgaaataaagagacattcaatctgagacctctgtcagtaaaggaaaaataatcatcataatacctagataagtaaaggtactagcacattagagttttgatgatatatgagaggataaaataataacttatgtataattataaattttgaagtttatgacatgaatatagaacacactcaaagcaagttaaatctctgtctacttaacattcaaatggttataccatgtgaaaagatttactcatagcaaactactattcaatgtttgaatggtttagagtagcagcttccaaggggggtgtgtactatatgagaaaatgggtatgggaggaaaataactaagatgtattgttttatgtatttatatatatatttctatgagtatagaaatcttacatttaaaatcttgattagtcataatacatgtatacatttttaaataaaaatagatatattgatggtatatgcctagtcttattgggatgatgatgtgcatggtataaacatctcagcatcttccaagacatggataacattttgcttttgattaagaaaatatgattttagattcaaactagaaatggagttagtaaaagaatgaataaactttgctttgaaaaatcaaagctcaagaagcaaaagatgagacattttttccttggtaaattttggcctaactttccaaattgcaccctattatgtcatatttatttctgattttagctatcattttcttcctctgatttagtctttcacagaaataaaccagaggtgaaaagggttctagaagtgttatttattgtgtattttttatctaattttgccccatgctaataaaagctcacttagagctaccttcttaccacaaatctagaaaagttatttttggtccttggacccctagtctccaaagcatgcagaaaatgtgtctggtgatgtcaatagcttcatgggagattttttaaagaaatgttgttattgagagttgggagcctagcttaaatatggtaaagcatgcattttactataggatacgtgtattagaattgatatggggtacaaatcattattatacataatttgttcaagatttatgctttttgtgctttgatctattagtaaaaagcatatgattgtaacaattatcttggttaagttgcattaatatatttgccctttgctgctaattggaaacaagggcctttagtagaaaatcttctactaacaatgctattaagtgttttaagttgagttgtaataatagttctaaggggctataattaattttgcctatagtattaaatatagtataaacataattagaaggaattatgtaaagatggctactgagctctctatctcccttcctccccaactagtgccaagttcttagaaactgacagaaaatttataatcagttaatttaatgtagtatcaaccaaaatgctaatataattttatgaagcatcaaatctgatgctaggatgtatctgaaaaaaaactcctcagaaaaaaagccaagaaaaatgtgaaaaaaagaacgatactgaaaaagtctagcccagatttcagaatgtactgtcataattttaaaagtgaactataaatttaattatattttagatatatatatatattagacaaatgtccattatatcagcagtaaattcatgaagagccatgcacagctttctcccaggcctacccagttctgttcctaaaatttagaatttagatagtaatggaacccacttagatacatagtcaataaatatttaatactctaaaaagaagaggtttcctttgtcaaatgctaaacagttatattgataaatatcagtgatagctgccaatgtaaaagaaagccatatcttgtccatttgaatatttttctctattcctgaggatgctgtactaggcactgttcatttcatgaaaaaaagagacgctaaatctgagagctccatcagtaaaggaaaaataatcatcataatacctagaaaagtaaaggaacaagaaaataactatcccaaaggccagaagaaagattacgtgtagggaagggcaagaggttttacatgggaatggatattctgagggtttagaggggctcaaaatgttctctacctcaacttacgttgcggtttcctgagagttcactttataattatttattaaactatgcatgtatactgagcacccttttctgtgggtgaaaagggcatagaaaaagagtatggtctagccacaggaatagaccaacatatcactggaaagaattggtagtccaggaatcaactcatgtttatatgaaaacttaggatcagtagcatttccaattactatggagagggtgaactatcaaatgggacaactcactgcctcattctatataccaccataaaagttaggttaaggagctaaatgtaatctaaaatctcccaaagtattggcctgtcgtggtgactcacacctgtaatcccagtcctttgggaggccaatgtaggtggattggttgatgccaggagtctgagaccagtgtgggtaatacgatgagacccagtctctacaaacaattaaaaaattagccaggcatggtgatgtgcacctgtagtcctagccacatgggaggccgaggtgagagaatcacttgagcccaggggtttgaggctacaatgagttgtaatcatgcaattacactccagcctgagcaacagagtgagatcctatccttaaaacaacaacaacataaaaacctctaaatgtaaaaatattaaaatatagcattggagaacttactcttcatcacggagaagttatacacacgaacatgggagatattgtgaagactcaagacccaaatcaatgaattaaaaataacaataaacacaagtagtgttcttttggttaatattttaactaatagttaaataattaaacactagaacacatttgtgaagggaactgtattctacttataagagtaccagccaggcatttcctcattctcatgcccatagagcttcacgtaaacccacccaagtgcttagcctataccatcccttcttgtgatgcaggaaatatcattcttgtctaatccctaatttgtgcctctgtttgcacatcctcaccttgttcattctcccctttttaatgggatttttcccatcctcacacatgcataaaagagcaggagcaccactgtcctcatcgatgaccctggcatagcccatgctagattccccactagttttgttctcctttctagctaatatttcaaaaaaatatatgtatataatgatcttccttatttctctttagttattctttcttttactctaattggtccttgatttctaccgccctgctataggtagttgtataagtctcttcttatgcttctaatagagacatactcatgactgggtaatttataaaggaaagagattttattgactcacagttccacatggctggggaggcctcataatcatggtggaaagtgaatgaggagcaaagtcacatcttacatgctggcattgcctgccaggggaactgccctgtattaaaccatcagatcttgtgagacttattcactatcatagagcagcactggaaaaacctgcccccttgattcaattacctcccaccgggttcttcccacaacatgtggggattgtgggagtacaattcaagaggagatttgggtggggacacagccaaaccatatcaccagtcttaccaaaggaaccagctatctacctccatgctgccatccaatgcacagttctcttttctcatcctttatatctcgagtgaaatgttcttgaactcctgagctcaagcaatcctcccatctcagcctagcctcctgagtggctggaactacagggatgagacacggtagctggcttcaagtgaaatgttaaattctcagagatgccttctcatacctaaaattgcactaatacctttccatttctttcaatcctttgttttcttcctggcacatctcatataattagctcatagtgaatatatctttatttatctatttaattgctatgtagcaaattaccaccatgtttcttggcctcaaaagcatttcttatcccatagtttctatggatcagaaattcaagagcaacttagctgggtgtctctggctcaagttctcttatgaggttgtagtcaaggattgatcatggctacggtctcatctgaagatttaactggggcttgggggttggtgggaggtctgcttttaggctcattcacatgggtcttgacaggaggtctcagtgcctcacttcatgggcctctccatagggctgcttgagtgtacttacaacatggctgctgatttccccatcacaagtgatccaagagaaagtcacatgtgttagaccattcctagaaatgatataccacttctgacttattctgtttgtaaccaacaagtcactaagtccagttcacacctaggaggagggaaattaagttctacttagtgaaaacaggagcatcatataatttgtggatataacttaaagccaccatactccccattagaatataagctcccacagtgcaaggaatgggttactgcttacacttacatcattagtgcttagaacagcaggcactttgataaacatctgtttaatgagtcaatagatgaatgtctcttactcttgggcagcaaagatatagactatggagttatctagacctggtatgaaattctgactctaccttttactagttctgaactttggacatattagtttctatgagcctcagtttcctcatctgtcacctgtgctatattcctgcttcacaagaatgctatgaggattaatgaaattgcatatataaagcacttcttcaagcattcagcacaagataagcattcaaattctagtttctctgattttttccatcttttcactttccccaaaactttcaagaatcactatttctttactacatgataccaaaattttctatttaactttctcataatgaattaatcatatgttcaattatttacatgtcgaagaaaaatcaatttatcttagatttaaagaattttttcattagtgcataatttctattagatcttcccaagagaattctaatatgtccctatggaggggaaattaacaaatgggccaattggaaggaagattccttagcataagaggtacaactgcaatttagattcactattctgtctaaaagaaaagtatgttaccccataaatagatacatacttgttcaagatttatgctttttgtgctttgatctactagtaaaaagcatatgattgtaataattaccttggttaaggtgcattaatacatttgccctttgctgctaattgaaaatgagggtcttttgtagaacatacctactgtgtacccacaaaaataaaaataaaataaaaaataaaactgtgataatatttagtacttgaaggaaagtcacaaggctcatctgtagttgcaccattttttcttgtggaaaatgacagcagacagaatgaatttgggaaattattttgtggagggaaatgaggccatgggaccctgtgggtggaatgaacagtactgctaaacagcttttgttgaatcagaaactgagcgaagaatttaccaagctgccctgaaacttggtatcattatgagtgagtctaccgacgcagctcttttttttccccttaaaaaatcaggtgtaatttacattcagtgaaaggcatagatctaaattttatagccctatgacacatatataaatatgtataacccacaccccaaccaagatagaacatttctatcactctaaaatgatccttatgtctctttccagtcatttgctaccacccccaatgtaaccactgatctgacttttgtcatctttagttagtatagcctcttctagaaagtacatactgctgtgcttattagtatatgcctctgagatccattgttgtatgtgagtagttttttgtttgttcgttcgtttgtttttttgaccgagtctcgctctgccgcccaggctggagtgcggtggcgccatcttggctcactgcaagctccgcctcctgggttcacgctattctcctgcctcaacctcccgagtagctgggactacaggcgcccaccatcacacctggctatttttctttcttttttttttaatttgttagtagagacggggtttcaccatgttagccaggatggtctcgatctcctgacctcgtgatctgcccaccttggtcgtccgtcagctcctctagtgagtagttcttttttgctttgctgagtagtatcccaatgtatgactatatcagagtttgtttattcattcatctgatgaagaacatgtggattatatccaatttttagctatcatgaataaagctgccatgaataatcctgtacattttttctttgggatcaatactgaagaatataattgctagataataaagctgatgcttaattttataagaaaaagccaagctttaaaaaattggttgttctatttttatactcccatcagcaatgtatgaaaagtccagcggctccccaactccagggatggtaatggctgtgttttcactgttaggcattctggtaggtgtgagcgagtatccacaatttgctctatcacagttttgtgcatgttcgtgttttctccaatcatagctttcattctttactttcagaagagaagtgttgggactgtatctgttcctcctttcccccaagctctttcagtagggagtgttcccatggagtgaggtgtcacactgaatgaagtgttcaactgcatcaggtcttcagcaccctaggtagtcccagattgacactttcttaccatggcgatgtaagaaagcatttaccatagtaagcgggtctgaccccatatcctgctttgctttgtggatctcccatctcctcctcattctggcccttgttctgaaatataaccttttgaaacataagtttccctcactgtccagtgctctgtccagtatgagaaaacagcaatagggaacagttactgcaaagcatgaactcaggactcagactgcctgagcacacatcgtaactctgctgctgaccagtactttaccttagactcacctcaagccttgttttcttcacctgcaaaatgggacaactacagtaacaaccttgtagtgttgttgtgagaattaaataataaaatatacaaggcacttagccatagttatgccaaggctccagctctgccgtctattatgcgaacatctactctgatgcctacggacttgactttttaccactctctgcccccacttgtcctgtgcttttatcttcttttctttctcttctctttctcatatacttcctagccacatcaagtcaatgtgtaacttggctttgggctttcctttttcacttatgaactagttataagaagggtgacttccagaaaaatagaagtgaaatttaccaaactcatgcactgtctaagaattgaacaatgagaacacttggacacaggaaggggaacatcacacaccagggcctgtcgtggggtcgggggaagggggagggatagcattaggagatatacctaatataaatgatgagttaatgggtgcagcacatcaacatggcacatgtatacatatgtaacaaacctgcacattgtgcacatgtaccctagaacttaaagtataatgataattaaaaaaaagaacttaaagagaaccaatgcaagcaaaagccacttctctttctgtgattattcaagtggagatggtacacttgcatgagctttgggacatgttgtacttctttaggcaaagccagcttgtcttccagagagatcttcttggatggccttctacaccctgttgaattcatcatagccctggtttgattttgaagagtgagaagtttgacttctcatcatgaggatttatctctcttagtttgagtttcctcaaaagcagatctcctgaaaaccttcaaggagagacaatgcctcagtttcccatggaggggtgaggaagttggaatatttttcaaccaacatgtgcccttcattggttgagggtcactcctggagtgttaactccctgacacatccagccttccctgaacattctgcctgactattgggccaagaagaatagggatgccttgaaataggaggaaagtctctgctggaggacccaggggatatgcatggcactgacaacatctacttcattatttcgcaaatgtaaattcacattaaccacatccaccaaacacagggagtaatgcatgaaaagcaatcatgctgatctattaatattctatgagaaatacaaccaccagaaattccaactatgtgtgctttgacagtggcagcttctttatggtcataaccaaatgggaagccagattatcttcacaaggagactggggcgtttagagccggtgacatgagcttcctggctggtgtcattgacctctgaccaatggcatacatcccctagcggggatattccttcacagggatggtctagagactcctgcaaaaccctctttcttggatttgactctgttttttctttttatagacaccttgctgatcatccttctcctaatagtgcactttcttatctgtaagaattatatttataatcatccaattatacctcccccagctcatcttattttagttctgagggtcaccactaatctcttggtcatagctccaggtggctgctctcatccattacctttggattctttatcatttcttcactagaggttaaatttaaccttagttttcggaaatttgttgctgttaactcccatagctgcctgcagacattttcttgataaaataagaaagtgagtacaagaagtgggtaataatttatttttgttttttattgttttgagatagggtctcactatgtcacccaggctggaatgcagtggtgcgatctctgttcactgtatcttcggcctctcgggttcaagtaatcctcccacctcagcctcctgagtagctgggaccatgggcatgtaccagcacacctggctaatttttgtattttttgtggagatagagttttgccatgttgggcaggctggtctcaaactcctgctcaagtgatccgccctcccccggactcccaaagtgttgatattacaggcatgagccactgttccttgatggcaatagtttattttaaaaatatttgaattcattaggccaagtgtggtggctcatgcctgtaataccagcactttgagaggctgatacaggaggattacttgaggccaggagttcaagaccagcctggacaaaagagtgaaacgcccatttctacaaaaaaaaaaaaaaaaaattaaaaaattgactgaatgtggtggcacacacctgtagtcccagctccttgggaggctgagtgggagggtcccttgagcccaggagtttgaggctgcagtgagcagtgattacaccagtgcattctagcctgggtgacagagcaagaccctatctcagaaaaaaaaaaaaatttttttaaattatagatctgctctttcttgacactccgctatagtagaaagaaaactggagtatgttaggttgaatgacatgaagttggcaagattcaaccatttttgacatataataggtcaatttcatatagttcagcctacataaagacaataatgttctctatcaaaaagtgagtcaaaatatcttatctctgttagggctatgtggcttttaagcagatcctcccacctctttgggtcttagttactcgtctgtaaaatgggtttgactagataaattataggagctttcccacaagttttatgaagattggtcattgctatggctgctataagaagtcttgtttcaaggatgttttttcttgttgtgacatcttgctttcttatattcatgttctcttacgtttatacccaggagtctcctctgtccttttctaatctttcttgctggacagttttttccttaagacagctctatctgagttgtttgtccctatggcagccatgaacaatagagtaactatctttgctgtttatctatccttgacaacttgatgtccaacttggcactgaggataacttcttgagggctcatatttcctttagggcctcaactccaacgattcttcaaccccactgtgacctgaagtccatgcatcctaccccctttctgttgtctctcactctcttcatgtcccaattttctactgtttcagctcacattttactctgcattactctaattactgtcttgcattttctaggcacacattgctcctttccccttcctttgtaatcacttggcaaaagtctaacccagattacattcaactcttcatgcccatcttgcctacactcacccagctaaacacagatggagtgaaacacaccattgtgctggctcgtttcactttacagccatgactacaaaccacgcatgggtccttagagctgccgagtagtcagattatgtttctggagcccctttactctcctgttcttttaggtaaccattttataccttatcttctccctcattttcaccttcagctgattttcttgtttcttgtttctttaagaaaatagaagccatcagaatgagacatccccatgtccccatcatcgcactgtcaaccgcacatctgcactcatctgcgctcatgttctctgcctacatttctttttcttagaatgaactgcctgtgcttcctgcagagtccaagccctcctcttaggaagtcgaccgtcctctttcgcctgcccaaggacatcactctggcaactcttccccctctcctgcattatctgttggcctttctgtgtcatacacataagctctattttttccccatctaaaaaagaaactcttttgactctaggcctctccagctaccaccctacttttctactgccctttagaaaaaactcctcaaaagaattattctcacatttctgcaggataacatcacacattctcctagttctatattcttctactagctaatttctttgtaccatatttagtttttaaagagctcctttctctataaatatttcatttcaatgccagaggtcatatcagatggagaatggctggttggtggtttaacactgattaaaagaagaggaaaagcagccaaatatttagagaaaaggtgaagccattccatcccatcacctttgttggagcatttcaatttgctctctaccttagatgtgctctctagcccatagctgagctaaaggtatattaagtctgtctaaccgggcttgaagggaagctggaagaccatttgttcttttaaatctctttagtttgcaattgtcagattcaattttgaggggaagagttcagagcatcattgaaagtaaaaaagatgggggaaagtgatagccacatgataaattatatcaaagtcaaagtcatcgccctatggcaatttttgcaaagaagtatttttagcatggatagctaaagggtagagcatgtcacagagacacctgtgtagttatgcagactggcagggatgtagaggaactattagaacccctgccttgtgtatcaatggcatattttatccatgggcagaattaacagataatattcgctatactgattaggagagaagacagcttcatctccacacagctggaaatatgtcctgcttcaatgtcaatttgtttatgctgtgggccaagtcatgcccccatatgtctgtattttcacatctctaacagatattaattgcactagatttgctgtgctgtacttttgattagtcagaagttcggcctagtaagttagaactctgtgttctcactgtgttaaagggatgcttgtcattatattgttttgaaattgtttaaaagatacagttagggaggcatagtccttgatcttttaaaatccttttaaagaaatgaacggggcaattagatagcaaaactgaaaacttatgggcttttattttttaatttttaattactatgggaacataatagtcatatattctttcattctttttttccctcccttcttgtttaagttaggccagagtccagttttctttatggttttttttttttctttctttcccttttttttttttttttttttgcgatggagtctcgctctgttgctctgttgcccaggctggagtgcagtggtgtgaacttggttcactgcaacctcagccgcctggattcaagcgattctcttgcctcagccttcccagtagctgggactacagccgcgcgccaccatacccgtataatttttgtatttttagtagagacagggttatgccatgttggccaggctggttgagaactcctcatctcaagtgatccacccaccttggcctcccaaagtgctgggattacaggtgtgagccaccgcgcctggcccctttactttttatttattctttctaatttcaacttttattatagatacagggggtacatatacaggtttgttccatgagtatattgcagcaggtatgggcatagtacccaataggtggtttttcaacccacagcctcctccctccctcccccatctatagtctacagtgtctatcgttcctatgtttatgtccatgtgtgctcgatgtttagctcccacttatgagaaagtgtgagatttggttttctaaaccctgcacgtgtacccctgtgtctaaaataaaagttgaaattagattaaaaaaaagtgaagaccctggtataacttaaacaggaagaaaaaaagaattaaagattatattactattacgtgcccatagtaatcaaaaattaaaaaaataaaagttgtccataggttttcagttttggtgtctaattgccctgtttctataagaggattttaaaagatcaaggactatgcttccctaactgtatcttttaaacattaagtcacttagggtaatggcctgcagctgcattcatgctgctgcaaagacatgatttcattctttttttatggttgcatagtattccctggtgcagatgtaccacattttctttatccagtccaccattgatgggcacctaggttgattccatgtcgttgttatcatggaagtgtttagaacaaaactagttgaaaaaatatcccctagaatcccaggtacaaggagaaggggacaaacccccaataccactgacctgcatgggctgaatgtggcagcaagaagcagagggaggcaagggggtgactgaagaaccagagaaggtggtaaccccaaactatccaagaggtactctacagaaagcttaaatattgcctggaaatgggcacttttatggattgcccactctgctaggctcagttttagatactagagaggccacattaaatgaaatgggaaaggtctctactcttacagagcctatgtcctagaaaagaaacatagacaaataatagaatttcagacacatagatgtgctaaaacaaaggaaggcaatagaaggaacccatgtggctgggtgaggaactctgcttttaatgaggaggtgacatttaagcagacaggtgagtggaccttagcggacctgatttaataaatagaatgatgaggataagttcattgattatttgatccaggattgaacattattcacttaaataggttaattattctcctttatgatctcatctttcaaaattttcctccatgcaatatcaaaggctgcctgtatccaaaatcaaaatgagtaattttgatgacataactgcatgtggcctgataaaaatcatccttattaaaatgaattctggaaggggtcccaagattggtgcagtggttcagccttgctgccaagaatccagctccccgcagtctgtcaatatataccctcaggtctgtgtattttggccgaacgtgggggccatggtcaggcatcagcatcttgtgggagcatatcctaatgttggaaggaaaacaaagtttgtctcttttctctcttgtctcttctctctcttaaaaaaaaaaacattttaatgaagaattgtgcacatattgttaggcatatatagcttgatatgttttcacaactgaatgcatccagcattcaaatcaaaaagcagaatatcattagtacctaagaagtctcccatatgccccttttcaatcactaccttctccaaatgtaattagttgcatgattttaagtttctaacatcttagattagtttcacctattttaaccttttgaactttaaatggaacttcacactatgtactgtctcatgttggcttcttttactcattatgtttgtgggattcacagccatttttgtgtgtaaattatttttattactccatagtattccattgtccattctgcttgaaaatatatatatatatatatatatatatattctgcttgaatatatatatatatatattctgcttgaatatatatatacatatatattctgcttgaatatatatatacatatatattctgcttgaatatatatatacatatatattctgcttgaatatatatatacatatatattctgcttgaatatatatatatattcaagtaagtagaatatatatattcaagtaagtagaatatatatatattcaagtagaatatatatatattcaattagaatatatatatattcaattagaacatatatatatatttttttctttttttttcctttttgagatggagtcttgctctgtcgccaggctggagtatagtggcgcaatctcagctcactgcaacctctgccttccgggttcaagtgattcttctgccccagcctcccgagtagctgggactacgtgctcgcaccaccacacccagctaaattttgtatttttagtagaaacggggtttcaccattttggccaggatggtcttgatctcttgacctcgtgatctgcctgcctgggcctcccaaagtgctgggattacaggcgtgagccaccacgcccagcccattctacttggttatcattttaaacagtaaccagtttggggttatgagtagtgctgcagtgagcattctagtgcatgtctcttggtgatcatatatacacatttcttttgactttttgaagtcctgtatctaatttcctctcaagtcttcttgggcaacattaggtcacatgcccttcctatttgcagtagaacctgggaaagaaaatatctaagaatgtgctgttaaatgttcaacaactagctcttagcaggagggttgggcagggagaagcagggagagggcggcttgatgggaggagggttgatttgcagcatctgcagtttcttggtgtgaatactcccatcatggccagttttaagctaccagtgttatatcaaccaccttgcaaaaacagctctcatagaattagtgttagccagctccagaatgccacagaaaatagctaacattttcactttttataattggaatttggctctgccagaaaaaaaaaatggaggctagggagggataggaaatgttgttccataggcaatcagtatttttgcaccaattattgagttactactatgaataaattaataaactactatcctaacccaaagatgattattataaattttagattattggtgaaaatgaaaactggcttttagatactattaggataggctagatttatactgtggttaaaaaaaatccccaaattctagcatttcacccataggagtttgtttttcactcatgcaaactccactacaggtcaagaaactctccagtgcagctattcttcatgcaatgatttagcaacccggacagcatcatgttatggctcattatttcaatactcagtctccatggtcaccgctggagcaaaggagacaggtgagggttatacatgtgcttgtaataccttcagtgcaaaagtgatttatcaattccactcataggtcattgcctaaagctaggtactgggtccctcccaactgcaaggacaattggtaaatctgtgggtatatgatgtactgtaaatgtctctgccacagagaccatccactcagaaccatctgttattttttaaagatcctaaaatgggcctttaaaaatatttagttctatgcatttccaccctaacatatatccagttactctagaaaaataaccacaactgtactcgaagttatccaccagtaatgcattatctcagaaaatagaagaaactgtttcttaagcaataacaagatcaatcctaatgaagactcttttctatatatgagttaattggcttttatattggactataatctccagtataggtaatgccttcagccataatgcttattttaccatcaccgtagtaataattgatgtaggtaagaatcatcaacagacgctaaaaccactgggctaaagggtgctgagcaagagaatatttgcataggcttcaaagtgatatacccacatttattacttacaaaggggaaaaggtagcattaaaatggagagatttggcagatattgcctcttccaattgatcaaatttaatattgccaataatggggcaaactaaaaccatgtgccccatacgatacactgagaaagggacctaatggtatctacacagtatgcctaccaaaactgttaactatgaacataatcttaagaaaaaactcaggcaaatcctaatttagagctgtcttgaaaaatctcagctgtactcttttaaaatgtcaatgtcatggaagccaacacagtgtccttgatgaaaggagatcaagaatgatgactaaatgcagtgtgtgattctggttcagatcctggaaaatgaaactataaaggacattattggtttaaggtggagattttgattatagactatatatttgataataatattgtatcaatgttaaatttcccaaatgcaattagtatgtcatagttatacaggagaatgggtttgtcttatgagaaatgtgttgattagtggtgaagtgtcatacctgcaggtacattcaggtgattcagaaaaaaaggatacgcatatatttatatgtgtatgtgtgtttaagtatatgtgtaattatatgtacgtataaacacaatatacacacatacagagaaagcaaatgtggcaaaatgttaatctttgaagaatctaggtgaaggttgtctgggcattaattcttccactcttgcaacttttcttcagtttgaaatgtttcaaaataaaaaaaacgggaaaatactgtttgttccatttttcctgcagttttgcaactcaattcactcctgtcatatttgctaaaacatcagtcttattgggaccaacttcattctgttgcttaatctgcacggtttagtcatcagatgagagttgggcttcaagattgtttttttttgttttgtttttcatttaacagcttattggtttatttttgatatgtgtctgtatcagttaggataagttaggttttttaaaaactacttttttgcagaatggttgacatgtaagaagctatacatatttaatgtgtacaactcagtgaatttggggataagtatacacctgtgaagccactgccaccattaaagctgtaggcatatccatcacctcccaaagtttcctctcaccctgcccctcttattataatttcatgtgtgtgcatgtgtgtatgtgtgataagaacactgagttctaccgtcttggtacaatatagtattgttaactataggtactatgctatatagtagatctacagaacctatttatcttgtataagtaaaactttgaaccctctaaccattgcttccccatttccccttcccctcagccccaggcaatcagcattctaccttctgcttccatgattatgcctattttagatttcacgtataggtgagatcatacagtatttgtatttctgtctctggcttatttcacttagcataatgtcctctagattcatccatgttgttgcaaatagcaggatttgtttctttttttaaggctgaaaatatttcattatgcatatacaccacagtttaaaatccattttcctgttcatgggcatttaagttgtttccgtttcttggctattatgtgtaatgccacagtgaacaagggagcacaagtgtctcttcaacatcctgatttgaatttcttgggataaatacctagaagtgggattactggaccacataattctatttttaatttcttgagaactttccatactgttttccataatggatgtactaatttaccttcccataaatagtgtaccagagtcccattttctccatatctttaccaacacttattatttgttgtctttttgataataaccattctaacagtcatgaggtgatatctcattgtgattttgatttgcttttctctgatgattagagatgtcaggtgccttttaatatagttggccatttgtttgtcttctgtggagatatatctgttcagatccttcctgtgcccatttaaattaggtatttgttttattttatttgctattgagttgtatgagtttgttatagattttggatattaacaccttatcagatacaagatttgcaaatatgttctactattccataggttgtcttttcattttgttaattgtttcctttgctatgcagaagcattttaatttgatgtagtcccatttgtttatttttgcttctgtttcctatgctttgggtgctatatccaaaaatttactgccaagaccaatgtcaaagaattttttctctgtgttcttctaggagttttacatcttcaagtgttacatttaagtctttaatccattttaagttgatttttgtgtgtggtgtaagagaagggcccaattttattattttacatgtgaatattcagttttcccatcactgtttattgaagaggctgttcattcccctttttgtattcttgctgcccttgtcaacaattatttgactgtatatgtgtggggtttatgtcttggttttttattctgttcaattggtctatgtttattttattttcctgtgaagaagaattctcaacaattttttatatggctactctacccttaaggaggtggagtatgacttccaatccattttaaaaaaaaattcttgtgtgtatttaaaatatgcaacatgttacaagagatgtatagatgataaaatgattacaatagtgaagcaaattaatatatacttcatctcacatagttacccacttatgtgtctatttttatgccagtgccatgctgttttgattactatagctttgtaatataattttaaattgggaagtatggtgcctccagccttcttgttcttgctcaaaatcggtttggctattcagggtctttgtggtttcatatgaattttaacactgctttttctacttctgatgttaaaaattgccattggaattttgatagaattttgatgcaaaattttgaatctatagattgtgcagggtagaatgaacattttaccaatatgaattatttcaatccatgaacaccgaatatccttccatttatttagatattctttaatttcatcagtgttctacagttttttgtgtacagatatttcaagggcagggattcttaataatcaccacctgattctaaacgggcctgtaacacaggaaagagcccacccagaagcagagatgtccttctgtgtgtttggattgcacattcagttaatacagcatcaggcctggaagatgatatttatttttctttgaaaaacagtgggatcaggaaaagcaaagctaaagataactttctccttttaaacttctgttgcgttaggttagcctgtggtattagagtcaaacatatttgtctactctgaaatgacactagtgatactttatatgcttatgctctaaggaaaactacttagcatattctgtagtgttaaatgaatgtactgccttcttagaacaggccctgtaattttgtgataagagtatcaatgtttcactttgggaggctgaagcaggcagaacacttgaggtcaggagtttgagaccagtttggccaacatagtgaaaccttgtctctactaaaaatacaaaaaaattagctgagcatggtgatgcatgcctgtagttccagctactcaggaggctgaggcacgagaatcactgcaacctggaaggtggagattgcagtgagccgagatcgtaccacggcactccagcctgagtgacaaagtaatactctgtctaaaaaaaaaaaaaaaaagaaaagaaaagaaaaaagataaatgtcagatgaattgatatagatgatgaacttcaaaaagaagctaaactcaaatatacatttttttttttttttctcaaccatcaaaaacttgaaattctctgttgagaacttctgggatcttttctgaccattctgtcaggagcagcccagtagatttgtagaattaatgttgacacttcacaccttttatgagtccctatccaaatgggagaacggaagatttgtatcagacagttttgctaagaattgaaaacaagagccattttttggctgttaaccagaatgaattgttgatcaagtagatcatttagccctcaatactggaactcttgatgaatagcatggagaactaaattataaagtttcaagattttttcactattttgtcattgtcctaaaagtctggaatgagctttttaagtttctgtctctgtttttggctctaatttttgacatcccccacaaaacaaagtagtacttcagttattgaaagccattatgattttcagtaaatatgtctcatgttgctataacactctggtggtcacagctacccaataacatcacatgggaaggacagctacatggttcaatttaattacaaatatttacacactttcaatataagccctggaaaacattagcgtagggtgacccctcccagtaggatagcagctttttgcagctcaaagctgtattctttcctaaaaggttttatttgactaaatggctgacaggcggatattgaatagagaatgagatggttggtatatgttacgacttgaatgtaaccctagacacagagtttaaaatagtctgctggtgtcttatagggacttgttccccatgacccaacattcctgagaccaggcaacattctcttcctgcctccagggagtcatttcgtagctgggtttgggtaaacgttgtgggtttcaaaaaagccccatgcatgggaccaggagagcctcctttttcgaccttgtaccacaggcatcaccaactactcaagatgtaactttgttcagggcagggctactcaaagtgaggatccctttccttcatctgggagcttgatagagatgcaaattcatgggctcctcacctactgactctgaatcactggggtgtggccaggaatctgtgttttaacaatctctcccttaaattctgtgaaatatcacaattttagatttcccataggtgtgaggcaacaagcagacattagctgaatacttctcaaaagatgatcgtgatcatctgttcaccttgccaaatagaagtttcccataatgcaaacagaggaacacttatctataggatatgataagaaacaggttctgacaaatgtaaatatgctacaaagtaattttattaaaacatggtgttatcagatgtttagcagtgaaaagagatagtaaaattaaaaagcccagctacattctgcatcatcttatataacttgctaccctttcaattaatacttttcctggatttgttatcttgtaggacgtagtgttaagtgaaatcttccttatttgattgtgttctcatattgaatgcttgcttgctttctcagtaagagttcctaggagccaagcaaatattacattagtgcataattcctccagggactgtgttgtggctggagtcacaaggggttctgtcccctttggagagcagacctggaactacactcagtgggctctgaaacctgagccccagagcctgttttgttggtaaaaactttgctgcctttcttttccttagacatggcctccaggcagaagacaaagtagacaatttataaagaagggagcctgtggatgtcacagaggcatttcttgtccttactggggcaaggacataaatagaacacattttatttctataagctgctatgttcattttagttcccacacatttagtggtgaactgataaatatttaacaacctagactccaagggggaaagccttgaattttagccttggcaagttttcaaaatctaaataccatggccaatttcaagctaccaatgcaatgtcacaaagtcatggtagtggggctgaaacaagaggctgcaatcagctatcactgttctatgcgagctggcttcagcataccactatgcaaagtactcccttccaggtactcgctgagatagaagatgggaattgagctagcccatggtaggcatggataggaaggagacatcactgttgttgcacgggcttggcatgtggggagatctgttggtgttcagtacatgagaaaacattcatgtgttcgatgccactcccttctatgctaatgttttggccacatgtgttggggaggtaaaatgctggattctatacttctgttaatagtatgatgagttcaccacaaagcataaggctagaaaggacatccgtacctcacattttcagataaatgacttagcaacactattattccctgctaaggctgcctcaagagcaaaagcaggcatgcacacaattctttgttgccccagaaagtatcgcatacatttgctattgtaggaaagtataaaaaagcttgcaatccaaataaggctatgaagtagctcccaagggcagcataacttctgaagcaaactccttctgcaaagaatcctgttatttatataccaagagcaacaaacatccatatttacttttgtctgtgcccgaattcaacatttgattgttcttgtcactgtgaggcttggcgtctaaaacactgtaatgacaggttacgtggtgtgagataataaagagtggatgcattttatccagggaactgattctactactatcaaagtattttgaaagaagaggaggagaattcaagtaaaagtttaaaaatagaacagcatgaaggagatcagtatacatgtgcataaaaatgttcttggaagacctagaatatggaacatttcattagtgtattcatcagtacttggttcttgaagtcaggccattttatgagggagaaaatgttgaagcacttgacctaaggattcaagtccatgctgaaatatgacaaaaccctccactgaatctcagaacctctgaactctggtatttcttttaaaaatccagagcttcccaaacaaaatcgaagaagagaacagagctttaaaatcttattaaagactggactgagtggaaaccaatgaaatatctagagtggggttaaagtctgtatttgatttgatgaaatctaacttttatattttctttttatattcatcatattttattttcaacctatcttggataagcatgaaaataggtctgatagtcataaggctatcactacacttcagctttaagaagttgttaggtttatgaggacttgtaataaaactccttaatgaattaaaagatgtggaatatatgtaatagtttttttttcatattgtcttaatttgggttgtaccacaagtagaccctgagaaaggattctaacacaagtatagtttattgaggaagtaaccccaggcaatacctaggagaggagaaatgagacagggtagggaaagaattcggtgaaaagtatgttttcaaatgagttgccattatggacaactggtgctcaatcctgctggggaactctaggagacattgtagaacatgcccctgaattgtcccaagtgaaaggcaagaaagctggtgtaaccagtagctctctttctgccatcagttaaaaactgcttccagagtcatgaactatttgacactacggactttccctgcactagggctaggtgtgttcccatagtgagataaaggccctctgggtaataagttacagatgtttgcagtaaccggcctttgctgtgtagagagaggacatgccttagttcagcactgtccaaaaaagtttaatgtgagacactaacatgagctatatacattgttttaattttttcaggagccaattacgatgtaaaaagacacaagagaaattaattttaataatatatttcttaacccagtacatccaaaataccattttaacatgtagataacgtaaaaagtgttaatgagttatttcactttttgtcccagaactttgagatttggtgtgtattttacaacgcatttcatatttcaacaaactagccacttttcaagtgctcagcagctgccgggatgggaggctgggggtggtgtgtggctactgtatcaaacagtgcagttgtaggggaatgcaccagtgtttgcattgctatttactggatttggggcatgtgtcccaattaccctctgagctacactgaaggccagctccacccaatgcatcagagaaccagatacgtagatacatttttggctcaactaatgctttactttctcaactagattgtgctcctctgatttctcaagacataaaacctggaaaaggttatcttccacatattttatagaatattaatattagtattttgaaaactaacattttcaatgtcttcacaaatgcattgctgctgtttttattagtatgagtttgggatttaattagcacaaattcagtcctcacttattctctttttttctcccttcacccagcaaatacttactgaacacttcctgtgggccagcacaggccctgtggcccctcagtgatgaaacaaaaccctatcttcatggagctatgtttcagtgatggggaaggcagacagtaaacagaataccatatccatatataagagccatgaagaacaggttcttggcaaattgtttctgcaatgggttgacaggtttctgcaatgggttgccatcttactttaatcagatgccatatggaaaagcaagtaagatggatagtggttattggttttaaaagaggatttctcactttttggtgcatttcatctcagtgttcctttcattagagccttgatttggattcttgctttgaactcatttacaggagactttttaggaactgaactgaactataaaatgagttgaactctgtctagttaggagggttgtgtacacacactgccgtgttttgcatctctgcatgcctaatttaagggcagctcacacctcttccccacaggctgtcagatgcatggctgaatcatggtttgtgtcatcttcatggcataaactttacagacctttgaaaactagctgccagcagggcaggtcaaagactgtgtctttgcatatctccagtggagccctatgaggcatttatttgagttgctaatgtgagtgaggctgtgatctctctattaattaagtctgttttattcattgggtgaaatgtgaaagcatttaataatttaaagacttgatgttttcaaagtgtatttgacttttgaggttcaagacactaaaaaaaggttaacttgaagcaatggcacttgcaaagattttactgcagctgattgcatatgcaggatatttctggagtcatgtattatgccagaaagattatttcagtaggatttaactgcctgagtataataagtagagtgttttattacataataattgccttgaatgggtgtgggtatttcatttggctattgaaatcatgctaatgtaactgtaattgaaaagattacatttactttttaaacatcttcgttaacacatcacatttggtcagctgatgcaatccttaaaatcatatgttttattttcacagatttaatttcctgagatatattgcctcttgaaactttttaaagacatttcccgatagacaaaggtagttgagttcctttctatacaacttattgaaactgcctttgcaaaaattataacagggagaaaattgtgacagtgaaagaaatctgacctaaccaactccatcttgcttataacctccaaactgtccttgttcattcctgggcacagccctaactttgggaggaacttagtttatagtctaactttgaaagaaacatgataacaatgcttttctgaaacaaaaccccttcttgcctatgaaccagactgcctttgtaggactaacaaattaacgacaagattagaaattatggtttaggagtcatgcagctagagaccacaagattctaaacctccccaattgctcctagggataaaacatctctattgtcaaacctaagactggtgctcaagatatttttcagaccttgcactcaatggatcacctggcaccacccaaatcagtaaattacttcatctggtctcatggccccacccaggaatggactcaacagaagaggacagttttgatgccctgtaatttcatctctgacccgaccaatcagtactctccactccctggccccctacccaccaaatcatccttaagaaaccccagtctccaaattctgggggagaccaatttaagtagcaataaggtgaattaaagtttttctttattgcaattcccctgtcttgataaatccacagttgtattaatatctgggcagtgggcaaaatgaactcactgggcagttacattatttcctaattttcagtggttaccaacaaagtaaaaactgatgctaactatcttaagcaaaatgaaactttctggaaagatatgggaaggggagaattgattggagtcttaagtatcaaactttgcaaaaaggcatcttaggtaggagcccttggatgcagacctgagatggagattctttgtgcaaatgacttattgagggaatgctctctggagaaagctgtaaaagagtgagggaaactggataggcaggaggagaagccaagcacaaaataagggctcaggtgaagtctagcctcagcttgatccatgaggagctctggagcttaaatgccatcagagatattctggtattctgctttgtctctctggtcttcctccagtgttctttcttatacaacagtctacagatgagcacctatttgggattgtgttttcagtaggctttggcaaaacataagaaatatattttgctctttgttagctttttgattaggatttccctttatgggctatcacatttcatacaatgatgggattaggagagggatatgagtatgagtgattgtgtgtgtatgtataatgtccttcttggtaacataagaagtggaaaattgtatgttaattggccccaagaattaagaaaaaaatactcctctgtgaaactctatatacactcctgcattcttacttggcattattaatgtgaagcttaaagaataggaatttatcatatagttcatggtcatagctgttaatatagacccaacctcaaacaatacattatactatatgctcgagtagcatggccttttttgatgataataaatcttaatcaaaaaaacaaatgcaaactttcttgagtagggtattgcacttttataagttacatagtgtaaacatacttgattctttactttcccactgtaacaatagattattgctatgtaacacacaacttcaaaatttagcaatttagaacaagcatttatgtcacttattggtctccaggtcgtctggggaattattgtgcatctaggctgagctaggttgctgccagctggttggctcatgtgtgtgaggtctgctggagatcagcttggggttggctggtctgggttgccctcttctaggatggctggaacttctaggataactgaggccactttctgtgtagtctgtcatattgcagaagacttgttctcatgccagtgacagggttccaagtgagggtggaagctgcaagccttcttgaagcctagcctcagaacttttggcgtgcttctgcctccttcaattggtcaaagcaagtcacatagccaagcctagattcaagtcatggggacaacagctccacctcttgatgagaggggttgtgaagtcacattgcaagggcttggatacaaggaaggatggcaaaatgtggtcatttttacagtctgcctcacgctgattccccatattaaatcagtaattcttacatattttatcattcaatttccttagtcttatttttaaattttttaaattgataagtacatatttatgggctatgtgtgatattttgatgcatatatacaatgtgtaatgatcaaatcagagtaataggatatataccacctcaaaaatttatcatatcgttaaatctacctcttaccactgctaatccaagccaccattatctctcccctggtagaccacagtaactccgtaacttatctccctacttccactcttggccatccctaatccgttcttcacacagcaggcatgatgattttttgaaaatataaataagttttttcctcttgctcttagaataaatctatgctcctttctgaggtctaagagttgccacagtgatctgccccttgcccagttctagccttacctgtgtccttagaagtcctggaacctatcaaaccccttctgtttcaaggactttgtgattgcttgaccctgcctctggcttgtggcttctgcaacttttcatatgatttatttcttttcatacaaccacatcttccttagtcacctatgtcaagcaggactcccctgattactttccgctgtgtagctttgtgtacttccttcataatactgttcagtttgttaatttacacgttattgccaatctcttccaccagatgtgtgcttcataaagtcaaagacaatatctatctcacgaggtttggtcatttacaaaaaagaaaaacacagcgtctgattatgcacatcattgcacatattagttgctaagtaaatgtttgttgatgtcttacagagcagactccctacatgaattttttactaaaaacaaaacaggaatccttgtaacctttccttttaaaatgggttttttatttccatattttcatagcaacatacccagttacacatcataagtattaatcatgacaaatgatcttttgttatggaaatagaattgatatggtccaaggaattaaaggtagatgcctttgatgctgtgtatatagtgatgactcttacaaaagaaatgtattagtccattgcttacttgatacaacgtaagcaaacattttcagaaaggggtctttgctagataaaggcctcagtgttcagagcctcgttttccaaatggagaagaaagtactataaaattgaattttattcatgcttcagagagaaatgaaatctctccatcatgaaaaatgggtatcaggtgcttttgatttccttttatcagctgctaatcagaagagatgaggagagagaagcaagctaatgaagaaaccagtcgtttcaaagacagttgtattgtaggaatctttgtgattgttaattagatgatggccttgggaaatacattgtgagggagagaaatggaggaggaatttgaatattgcaaagcctctcccgagatggaaagcatctaaatgtgtttgtgttaaaatattttgagtcataagactacattttgaataagacatagtgtttcaaagacactagaggaccttttacctcatagattctgtatacattagtaatgatttatatctatacagtagtaattattcatataatctcttagcatgactaatttggaaacacattgcagtgcacatttgtaattttgggaagctaccaaatatctacaatctcttttctagctgggagaacttgttttgggagactgcagaagccaaaaatgccatacactgtcaactttcttggtagctagggcacagacatgtggcctgggcttgggaaatcagataaacctactctagactataatgtaaagaagtggagcaggagagaatccattttattggtggaggtggtatcaattgggaccatagggatggtaggaactatcatcagtactcatggtggttacagtgtaagcgatcatggcccttaatagcacatatgtatcctcattgggcaggttggggtggaattctagtcatagaatctactgctgctgtagcttcttttggtccttccttattctttaggcaacactgtgagctatctgttatcctttcaataaatttcataaattactttattcttccagaaagcaaggtaacttttggttgcttatacataaaaaccatgactaactcactccaacagaaaatgctaaccattttgagtaccatataatgagtagttgtatttcgtcaaatacatagtgctttatacaattatgttgcccagtaccacccaaatattgagaggatttatttagaaggatttaaaagttttgttgcattggctttccaattcttcttttattaatttaaaaaatcattactataattttaaaattagagcctccttcgtcaatgttatgcttcatgctgtctgtttaaaatgatgtcacttttatcttaaagcagagttgtgttaaatagaattagactatttaacaatatttctgttgagtctgatcatactgcttgtacgtgttaggaggtgtcagggaggcaaacatttgctgccaccaagagagtagtaatttccctctggtagaatgtttaggccataaatgaaaaagggtgatgcactcaataactgaagatctatgtggctcaacaacaatttatttacttaaaatattttattggatatttactaaacagccaggatctataaaacactgagaagtcagaactgaataatgtttggataatttgtcttaatgaaatgcagcttttaagagctgataacgtaaggcagtgacgctcaaaatctggcctgaggacgtgtgccatctgcaaactgagtgttactggtcagcaaggagatatggaacttgggctaccatgtaaatcattcaacaccctgtccagtccagttaaactgtagtccagctgatgcttctctcacataagactttcctgatgaaggaagcaatgaattgatttacatccttgtgcggattccttgtcttgtcatggatcagaacaaacagtttgtatactggctactttttgtagcaataatttagtgggtaataaatattataaaaggcaggaaatacatgtgattacatcaatctatggaacagcaataatatgacttcccattaacattcatttcaaaacaagcatttaaattagtacttatttcagaaccctcatttatcgtctgctttccatgagtgagccctggacaggtgctatgtacaaaacagttttctgtattttgtcagatcttaaataatcattgtcatcaggatttttcaacctcataagtgggaaaaggcataaaaccaattgtatacattcaaatatcagatatgttttataagaattatagattattagaaagttctgtctctatattattttaaatgaatactggataagttgaaggttatatttttatctgaattgtgtgtgtgtgaattatgtatgtgagttggtagctctcaatggatatttcaaaaattaattactgccaagtgagaactagtaagtatttgaaataataatgtcaatgataataacaatagtaaagtaagatactgagtttttttgtgcacagctatggggttatttcttaattgctttcatattattcatacaaataaagcaccagatctaaaaaactaaaatatgtttttcaatcacaataaatttaaactaaagataaccaaactaacctgcggtaatggaggccagaagaggagttaagtttggaggatactggcaggaacagggcacgagagaaacttctgaggtgctggaaatgttctagggctgtgctgttcaacatggcagccactagccactgtggctatttgaaattgaattaattaaataagtttctcattcacattaatgagatttgaggtgctcataacactgtggctaatggctaatgaattagacagtgcataattatgtttttttatggaacatttccagcattgcagaaaattctattggatggcacagctttagatcttggtctgagttgttatgtgggtgtccaaatatgccaatattcattgagccatacacttaagatttgtgcactttactgtagtatgttgtatatattgtagtaagccatatgtcaatttgaagagtacatttttaaaaagttagagatttggatcagatagttgtagatatgtggtgttatttctgagggctctgttctgttccattgatctatatctctgttttggtactagtaccatgctgttttggttactgtagccttgtagtatagtttgaagtcaggtagcatgatgcctccagctttgttcttttggcttaggactgacttggcgatgcatgctcttttttggttccatatgaactttaaagtagtttttttccaattctgtgaaggaagtcattggtagcttgatggggatggcattgaatctataaattaccttgggcagtatggccattttcatgatattgattcttcctacccatgagcatggaatgttcttccatttatttgtatcctcttttatttcattgagcagtggtttgtagttctccttgaagaggtccttcacgtcccttgtaagttggattcctaggtattttattctctttgaagcaattgtgaatgggagttcactcatgatttggctctctgtttatctgttattggtgtataagaatacttgtgatttttgtgcattgattttgtatcctgggactttgctgaagttgcttatcagcttaaggagattttgggctgagacgatggggttttctagatatacaatcatgtcatctgcaaacagggacaatttgacttcctcttttcctaattgaataccctttatttccttctcctgcctaattgccctggccagaacttccaacactatgttgaataggagtggtgagagagggcatccctgtcttgtgccagttttcaaagggaatgcttccagtttttgcccattcagtatgatattggctgtgggtttttcatagatagctcttattattttgaaatacgtcccatcaatacctaatttattgagagtttttagcatgaaggtttgttgaattttgtcaaaggccttttctgcatctattgagataatcatgtggtttttgtctttggctctgtttatatgctggattacatttattgatttgcgtatattgaaccagccttgcatcccagggatgaagcacacttgatcgtggtggataagctttttgatgtgctgctggattcggtttgccagtattttactgaggatttttgcatcaatgttcatcaaggatattggtctaagattctcttttttggttgtgtctctgcccggctttggtatcaggatgatgctggcctcataaaatgagttagggaggattccctctttttctattgactggaatagtttcagaaggaatggtaccagttcctccttgtgcctctggtagaattcggctgtgaatccatctggtcctggactctttttagttggtaagctattgattattgccacaatttcagctcctgttattggtctattcagagagtcaacttcttcctggtttagtcttgggagggtgtacgtgtcgaggaatttatccatttcttctagattttctagtttttttgcgtagaggtgtttgtagtattctctgatggtaatttgtattcctgtgggatcggtggtgatatcccctttaccattttttattgcgtctatttgattcttctctcttttctgctttgttaatcttgctagtggtctatcagttttgttgatcctttcaaaaaaccagctcctggattcatcaattttttgaagggtttttcgtgtctctatttccttcagttctgctctgattttagttatttcttgcattctgctagcttttgaatgtgtttgctcttgcttttggtactgttggcttttctggttcagctcaaggaatgtctgtgcggttcctgtggcagccactagccactgtggctatttgaaattgaattaattaaataagtttctcattcacattaatgagatttgaggtgctcataacactgtggctaatggctaatgaattagtgcataattatctttttttatggaacatttccagcattgcagaaaattctattggatggcacggctttagatcttggtctgagttgttgttacatgggtgtccaaatatgccaatattcattgagccatacacttaagatttgtgcactttactgtagtatgttgtatatattgtagtaagccatatgtcaatttgaagaatacatttttaaaaagttagagatttggtactgttggcttttctggttcagctcgaggaatgtctgtgcggtttctggacaagaaagcctgggcagggatgtaggtgatagagaaaagatgcgttccaagacctgcactgccatgatggaggcagccctcaatgggaaagcgccttaagagacaggtgtccagggtgtgtggggcagggcagggctatggctgtatgaagtctatcagccaaaagggaagtgtctgggaagtttgtcctttggataacaaataaaaggctatctcagtctttggaagatcattaccagataagagaagattttattctctgtatgggagatgagaaagttttgggaacattttttattaacattgggttttctctgagtcattttttaaaactttcttctctttgatgcatatcctctcttgattactgatttttaggggagaaaaagctgaggccacaagtactgagaaaacaatatatattccaccaaactggattcatatttctctttttttttagttgttacattggcattcccccttcttttattatctcatttttcttttcagatttagaggatttaggcagaattaactcatttttctgttcttcattcatttaaaacgttactacataaaatgtgatccatggagtggcagcattggtatcaccaaaagcatgttaaaatgcagatttttgggtcctaccccagacctacaaagctagaacctgcctttcaccaaggcattcatgtacacattacaggtaaagaggcactagtgtagaagtgtaagatcaacgcatgaaaataatatagagtacccagaatattgcaacactaataaattaggttaatcattttgtaacccattcattagccaagatgagttataggtattaaattgaccacctattatttgcttaatttaatagctgtaaaggattttttcctttttgtggcccttttcatttcacaaactgagtcaccaatgtcacagttctacaagcttgttctaagaggatgaactgtgacattggtgatatgtcgaatgtgattcatatattaaaagcaaaaattgctttttgatgtggtgtcactcacagcaaaaatgacattaaaaaaagataaatgcaccattttcagcttttgactaaatgatggcaatttagtaagaaataacttgatactgacaactttcaattaatcgctaagattgaaagaagaaaaagaatgaatgagccccaatgtcaggtaattaatattacttatagttagctttgtaatatatgttctgtttacttttgactggggatggcttcatctcaaatcaaatactaagaacatactctgaaaatgggacttggaaagagtgaagtgtctgtctactgtcctctctctatccccattctcctgttgttaaaattttttcagcagtttataccatgggcaacctaaaccactcatttcccaagatgcttctcagtatagttaaaatgagctgattttgctgaaaccacagagcaggaaccaaatcatagtagcatgaaaaacaaaacttgaaatttgagagtgatgtggaaacaaaacaagaaaccatgctgagggctgtgggtgtgtcaaccattaccccaaaggcaagaggagataggctgaagtcacagcatgcatgaccttgagctggtgtggaacatcattcaagctggtggttgaccacacacatctcattcatgtgaagttctgctgtgatgctgtttcttcagggagataatcccaagggtctgccaattgtctattcaatccacaatcccccatttagtcaataattgctgatgtttattccaagctcttactttgttaatattttaactcatttatccacttactaaacttattttaaaggtctgtgaagactgggaatgccaatgtcatcagaaaagtctttgtttataatgagcttaaatgatctagctcttacattaggagcacaagccagttttgccagttgaatgagctattaggcataaattatttgttctagaagaagccatattgcttacttgggtttttgtttatacttaatgaactgatgtttcaggactttgtattaatctttaggctctgtattaatttcctgtgaccactgtaaccaattacctcaaacatggtggcttaaaacagttgacatctattctctcagtctgggggccagaaatctgaagttattatcaatgacctaaaaataaggtatctacagggacccacttcctccagaggccataggagaccatctgttccttgcttcttccagcttctggtggattccagcattctttggcttgtggccacatcctgcagtcttcaaggccagcatcttcaaatccctctcttcacattgctatctcctctgtgtgtcatttcaccctgtctctctgttgtaaggatatatatgatggtatttagggtacacctggataatccaggattatctccacatctcaagatcctgagcttaatcacacttgcaaagtttttgccatgtaaggtaaaacttatgcgttccaggaatgaggacgtggatatcttttgaggggccattttcagctcaccacagacttcaaagaattagggcatttcccttgaaatatttacttactctttaagaatagatattgtttctagcacattgttgttccacaaagcatcatggaccctacccaacatagaaatgtcactgataactagcaaaaaatatgagcaaatcataagcacaatctcagtattccaacgtctatttctgaaggggccttgcacctctgatacacttcctaaagatccccttttctgcactgggatgcattttggtttaggagtcactgacaggcattacctaagagtctgatacaagttagttcagtttaagccatttagatttagggcttttcagggttgtttttgtttgtttgtttgttttgttttgttttgtttcgtttgtttttttttggcttgtttgcttgttttgagacagagtcttgctctatctgtcacccatgctggagtgcagtggcacaataatggctcacttcagattcaacctcctgggctcaagtgattctcccatctcagcttcccaggtagcttgggctacaggtgttcaagaccatgcctggctaatttttttattattttcgtagagtcgatgtctccctgtgttacccaggctggtctcaaactcctgggctcaagcgatcctcctgcctcagcttcccaaggcgctgggataacaggcataagccaccatgcctggccagttttttatactttattagtcacacaggtagggagcctacatgactaaagtccattctccagcagactatgtctcataaattccagctaattacctgaagcaatcaaggcagaccaggaataacctgctaaaaccttagagaagtttctgggttatcgtcctgttagtaaatcccatcacagtgtttacaagatctgttcttgtctaggtcaatgcatcctcagggacatcccaaggagggaaagaacaagtgacagatccactgttaattctttttcctacacgtgaattatgttttaggcatgtttgaggaatgtttgcttatgactgtacccagatattggtggaacttttgaggccttaccatttggagatcaagtatgtagataaatccagataaccaattttttttaggtccatcctgaaatgatagagagagagttttctgcaacaggaatatagggaaaacagcagaaatgaaagaggactatgtaacagcaaaggttgtgagtggaatgtctgcagtttgaacagaaattggtatatttgcttatactgaagatgcctaagggacttcagctaagagagtgatcatcagtgtgtataatttatttataggatatttagatgcttttattgcacaagattaaaacaattaaagcccacttccatctgattaatgagaaagtgacttgggtgaaataatggggctcttatttttattacaggccgatctgcttcagtttcatagttaacctttgtatggagacaataataaaagtaagacaattttctttcactttttatttggcttttgcatagaagaagcagtgtacacttggcacctgatatttacagtgccatctgtctgttgattttcttgctgtgcacagattcacagtaacattggggtggggggaggatccttatttgaatcttctctaagaaggtcagctctgtgtggcacctggatacccttgggtggtgagggttggatagcacatgaccttattcagctctccctgctctgtgccttaagaaaatctgttcaccttgggaccaccgttagcgtccaacctccatttatctttctaaggtagggtttaaagtaaggacacaaaggcaagatccctgagtagctacttccaagggtagcctgatatttttccaagtattgctttcaagacaggaaagagggaggagtttttttttttctctgctagatcattgggatccttcttagtgttgcttgaagaagtggatgtatcaaagattccatgcaagacctcgagagatccccaggtcttaactaagttagttaccttatcacttccctaacaaagacctttgcccttagcaactgtcctcatgatagagcctaacattttctgaagcacgttagggtttttaaatcagtgttacagatgttgtcacattttctcctcagaataaccctctgaagtagtctggacaggtattaatctttaatttatagatgaaaaaattgaggctaaaaggggtcaagtagcttgccaaagtcatatcattcttaaccaagagagtggagacttgagctcattcctccgaaagcttaatccaaaagcaatattgagctcattcctccgaaagcttattccaaaagcgttattgctaaccgtgctagacatacttagattcctcccgggactgtgtgtataatgtaattgagtgagattggctgggataagaaaaataacggtgggctcaagggtaatctaaaggagtgtgcacaattcattaaggtattggattgtcttgttcccttcttatttcccttcttcttgtgtttcttccctcttttcttcctttctctctctattctctgtaagcaaacggaagctttactgggtctcatggagccataaatttgtcactcctgtctttcaactttgcagacaatttaaaaagtaaagtgcacaaatcttgtaaattataattgtccagcagatgactttgcatagataaggttaacttagaggcaattgatggggtagagtttgacagtgcattctgtgataggtatgtctaaggtgacagagaaaagattttgatgggtcagcccaagactttgtgttggttcagttgcagaaggctgagctgtatggcacctgagagaccatctagttaaaccctttcatttccaatcatttcagagagcatgtgtgccctatcagttatctactgctgcagtcatgctgcataacaaacaactacaaaacctcagaggcatattgccatgaatgtttattgctcattgatcttgaatcagctcttatgatcttggcttagctaggttcacttgcatttctgtacttggctggctgttggctgattgaggctggcctccgctcaaaagactggggcaactcgaatctgctctgtctctcatcttccagcaggctagccaaggtacgttctcaagttcagggcagtggtgcaagagcacaagagaaaacacataggtctctcaagatgtagtcttagaggacaaatacgttgctcagtctgttggccaaagcaagtcacacggggtagagaagtataactaccttttggggagccactgcaatgttacatggcaaaaggtataggtaaaaggagggtgaatgatggaggcattaccaaatctacaggttacctagcaggttagggatagcaaaaagacagagccaggcctcttattccacctccacacaaatgcctttcacatcatttctagagaataccaatttgtcttagacatgccccactccccaaataagcccctggtatttagagccttcacattgctgccaggctcccttctaatccttcggttgtgcgagcaccttgttctctgccagcagtcaactaatccctgccaaggtattaacacttaattaaggtgtttgctgttaaagtccagtagtggctccgacatttttccaccaaagaacgcttttaattccttttccctatagactccacatttggaaatttctttgtaccagagttcttaataattatttctccctctaaaatatgaaatatgtgcaactgccaaatatagcttctggttggcatgagatactgaactgagcatgaaaagctagacagaagcaaagactttgactgcttcagtgacgacctgtgttttcatgggtcgatgtccaagtttgattaggccttttgaccctcacttttatcttaactgattccaaattgagaatcatggtgagaagatatgcttctatcttgagcattttcttgtaactggacttttctgagaacaaaacatacttcccttctatcctccagtcagtcttgtattctctcaaattatgcttggaaaattattgattattcaaaaaagtttgattattcagtcacactcaggtcactaatctgacccctgagaaaaagttggggcagaggtacaggttcatatcgaaaattgatgtcatttagcttttggaaaagactatttaaaaatcaaaagaaagtatagcataaattaaaaatggcactgtcccttgggcaggtaatttagtgtacctgtttctgaatttctggtgatttgaaattcaactcccataacataaactaaggatgatattagaagagccagcaatttcagtatagtaatatatttcacaaacatgagttgcactccaaatgcatgcaagtttcttgttatctctgtataacaagagagattaaatacagagatcattttacgcagctggaatgtttggttatttctatagctctagccaagtagagaagtcagtgagcacttagaaaatggtttcaagctgtgatttaaaaaaaaaaaaaagaaagcaaagaaagaacttgtgttgattcaactgcagaaagaaaaatgtttcactgtggtgtggcatcatgatatttgagagactttgcatatattacagagtattgtaaatactggataaagtacctgaagcatttgtgaaaggaagaataatccctgtgtgctttttaaatttatcattcacttattaaatacagtagaggagttagagcagttgtgtctagaagaactttcagcattaatggaaatgtttatatctgcacagaccagacagtagccattagcctcacgtggttatcaagcacttgaaatgttactattgcagctaaaaaactgaatgtttactctccttaaaaaaatgaacagcattattgcattgtaactgacacacacacacacacacacacacaaactgcccatatttaaagtatgcaatttgatacatgttgacatatgtatacaaccatgaagtcatcaatttaatcaagatagcaaacatctccatcactctgaagttttattacagccttttaaaatttctctcatgccttttcctgtctgcccaacctctgtccccaggaaaccactgatcttctttctgacactgtatgttaagttgcatttcctagaattttatataaatgaaatcgttcagtatgtttttttcctctggcttccttaactcagcataattatgctaaggttcatacatgttgtagcatgtgtcaatattttattctatttattgttaaacagtactctatagtatgaatatagcacaagttgtttatacattcacctgttgatggacatttgtgtggtttccactttttggctattacaaataaggttattataaatgtgtatataagcctttgtatgggaatatgcttttatttctctggtgtaaatacctaggcatgaaatggctggatcatatagtaggcatatgtttaacattattaaaagctgccaaactgttttccaaagtgggttatacaattttccattcccaccgagaatgtgtaagagttccagttcctccacattgtttttttgtttgtttgtttgtttgttttttgtttttgagaccgagtctcactctctgtaaccaggctggagtacagttgtgcgatctcggctcactgcaacctccacctcccaggttcaagcaattctcctgcctcagcctcccaagtagctgggactacaggcacgtgccaccacacccagctaatttttgtatttttagtaaagatggggtttcaccatgttggccagggtggtctcgatctcttgacctcatgatctgcccacctcggcctcccaaagtgctgggattataggcatgagccaccgtgcccagccccagttcctccacattcttactaacacttggtatggttaatcttttaaattatagcaattctaataagaacacagtgatactctattttgttttcattttacattttcctaatgactaatgatatggaaaatattttatgtgtatagttgtcatctctaaatattcatgaagggctattcattaaaggcttttcaaaatcttttgcccattttttagttgggttgcttgcttttttttaattattactaattactactgcaattaaaacattttctctgtatttattctatcagttattgagatagcattaaatcttcaataataattgcatatttgtctagttctccttgtagttctgtcagcttttgctttatgtattttgaaactctgtttttaggcatataaacatttaggattgttcagtcttgatgaattctctcttttattgttatgaaatttatccttggtaatattgtttgctctgaaatctatgttttatgatattaataagcattccaactttcttttggttagttctagcctatccttttccattgtttaattcaatttatttgtgtctatctatacactgtgttccttagaggcagcacagagttgagtcttgcttttttatccaatctgatatctctgtcttttaatggggtgtatagaccatttacatttaatgtgattattgatatggttcagtttatgtccatcaccttgctatgtcttttctatttgttccatcaggcttttttttttgtctcaaaatatctttatttcactttcaatgttgtaatatatttttgttggacatagaactctagattgatgggttttttttctttcagtaaagatttagctctactgtcttcctatttgcactgtttctaataagaaatctgatggcaactttatttttctctgttcatatattttcccccactggcttcttctgagactttcttattatcactgtttttgagcaatataattatgatgtgttatagtgtcattttcttcatgtttcttgtgcctggagttgtgaacataaaataacatggagaccagacttcagaattctctgaacagacaaagccaatttggccacataagcgaaacttaaagttatttcttgtaaacacttctgatcatcgtaaaccaatcttaagttgccttctaaagatggtataagatgagcactttcaaccaatcctctgtgatcttgaaaccttcatgataataacccatcattttaaaggttaaacaacttccccatttttgctgtgtaagccagcctgtaacatcatgcctctgagcttcctcttatgactgggcttgaggtcacttagttcatgaactattattttatatgcacaataaacttaaaaaatttttaatttgatctgattttatttttgacaggattcactgggcttgttaaatctttagatacattattttcattaagtttggaaatttattcaaccattatatcttcaaatgcctcttttgtcccttcctttgtatcctcttcactagaaaatccactgacacttaaattaggctacttgaagttgttccacaggttggtcatatgttgttcatttattttagatcatttccctatgtgtttcattttaaattgtttctattggcctggcgcagttgctcatgcctgtaatcccagagttttgggatgccgaggtgggtggatcagctgaggtcgggagttcaagaccaggctgaccaacatagagaaaccccatctctactaaaaatacaaaattagctgggggtgagggtgcatgcctgtaatcccagctactcgggaggctgaggcaggagaatcacttgaacctgggagttggaagttgtggtgagccaagatcatgccattgcactccagcctaggaaacaagagcaaaactccatctcaaaaaaaaaaaaaaaatagattgtttctattgctatgtctcaaagttgtatttttagtagagacgggtttttgtcatgttggctgggctggtcttgaactactcctgaccttaggtgatccacctacctcagcctcccaaagtgctgggattacaggcatgatccactgcgcctggccagtggtggatatttttatagtcctatcaatattcttgagctttgttttgagacacagttaagttacatggaagctgttttttgttgtttttgtttctattttttgtttttttagtcttgcttgaagatttcttcagtgcaactagggcagcgctcaatctggggtaattattcccatctattttggcaagaccttcctgagtactttacctaacgtccacggatcttcggcctttccagtcttgctgatgaaaacagaaaccattcctggccttgtgtgaaggttggccatggtaacctctaaacgttttaggtggttctttcctcagcctcaagtagtttcctcacatgcttgaattgatcactactcagtcgaatacttgagggagacattgtacaaatctctgattctctggaattttttattttttcctttcactcctgcctcactttccccctaccccagtctctctccagctctcccctctggtactccgtctggaaaactccagctccttttgtctctttggtctcttagctcagtcaacaaaactcagggaactgatcttttcctgggttccccctccctgcaccacatcctggaaattctttcttaggccttgatttttttaatatgaaaaaatgaaagtgtctacaaccaattggtagggttattttaaagaatgagatctgatctttaaagcacatagctagtaaatattcaataaatgaaaaatccttttcctgtacatagcattatgtattctgtatactttcctcaagaataattttgacaaatgacagttcattcaccacttctgagtcagccacagggaaacctgtttctcccttttgttgttctggggctcttgtatcccatagcatcttctctcaccagtatggttgtcctagactggttacctgtctcttccttcgtttctgcaaaacacctctaatgctaggggaaatatttgctagacgactttattaagatttacagagagtagttattacacacaatgagagaaaacttttagatcagtgttggttattaacatttctgaaagcatcagacatttttagctacagggaaattctcactggctgtgtgaattattagcaggtcctggggtttttctctgactcccaccccgtggcctgtgtgtatgtgtgtgtgtgtgtgtgtgtgcatgtgcgcgtgtgtgtctatcagtctttttctctcttgctttcttcttgtatctttctgtgtacatatttctcttttgtttctccttctattccctaatgtcatttctgtatctctgtctgttagtctgtctctccctgtatgtatcccttctcttatcattccctttttgatttatttaatttcccactcaccattaccaaattcattttactcttccatggaggccatcctagctctttgactttttccaaacagacctttggcctgcttttgcatatactgacactcaatatagagcttcttgtaaacaaagacccaaacatttccccaggttttcaactttcctaatacagaaagctgtcaaaaactttgagattacaacttttcttttagcacatctgagatcaacttcaagcccacagtagagttaacaggataaattcacctattaccacaggaaatctaacagaagcttcctcaagacaccttcttctctatgatggtctttggaaggatttctacttcacattaaaaggagagcatcctcatgttaatgacccaaccttaggaagccatttaaaatgtgttaaaaccagtgtaggaactctccacacagatcattgtgtttggactttatttttaatttttatttttacttttgtaccgtagtggtatgcttctgagcagagaatcaggtctggagagatgggtagagaaacttcctgaattcttgccagtctttcaaatgaccagccatcatggcaggaactaagacgagtaatgttgtttaaaaagagaaagaaagagccaggtgcggtgtctcacacctctaatcccagcactttgggaggatgaggcgggtggatcacttgaggtcgggagttcaagaccagtctggccaatatggtgaaacccagtctctactaaaaacacaaaaattagttgggtgtggtggcacctgcttgtaatctcagctacttaggaggctgaggcaggagaactgcttgaacctgggaggcagaggttgcagtgaaccaagattgtgccactgcactccaggctaggtgacagagcaagactctgtcaaaagatagaaagagagaaaaagagagagagagaaagagagagagaagaaagaaagaaagaaagaaagaaagaaagagagattggtaactatgtgaggtgttgaatatgctaattagcttgattatggtaattaattcacaatgtatacataaattatcaattatacctcaataaagctgaataaaagtaatgtattatctcatctcaacatcaatattcactgccttcaaagaatttatcccagtgtgtaattatgcatgcatttgtttaattgattacaaactatcttccttactaaactattacttctgtgaggtcaaaagaccatgtctgatttatgcataattcactatccagtccctagccagtatctgcccttttaaacacctgataaatatttaaatctagttctgagaaagtaggttcttccagacacaaattagtgtccagtaaaatacaaacaaagtgttatgtttatcagctagttccaacagtattggtaagagagagaagttgatgtcttttccagattatgctactgttttagggttttaaataatcaaagatgataattggtgtctacgcaagcaccatttcttctcaaataagggagaattacaaatcttagagctaactggaagaaagcatgtccagacagaggtcttactattttggggattttgttggggtatgattgaagaggatggagtgagagaaagagcaaaacaggagacagatattgacagaccaatccaagattagagaacccttgaactccctatagcccttgggtagacagttgtgtaggaaagggaagtgtgtgggtatgtatgtaccaagtggaattatcagagctattggaggaatcagatctacaagagagctcacatgagtttattttatcacaaagacctggcagtaataattatgctaatttaaacctggaaggatgtagactaaaatgtcaacaatgtcttgagtactggaattctgagtgattttttttctactttgtgtttttatgcattttcccaatttttctataatgaacacatgcataatatatcatcagaaagttttaaaaatgcataactttttccatttatgtttggtacaaactatttgtttcataatctcctggaatgcaaatgaaaatttaccctagaaagcacactttaagtattactgagttatcaggtaatttggcagccttaatgcttgaaatattttattgatgttagagtacttaagctgttaaaactttttttttaacttaaactttcagaagagtgagttttttccatttttgatgcagtactaatttttggctagaagttttacagcttgacatgccacctgacctctgtaccttctatttgacctaatgtatctatagatcatttaaatgagctcatttgactcaagagaaaatccttacatgtcaattttccatgtaaatgaattctgttttaaagactaatactttcattggaagctgattataaaactgtgctattaattttgacattttatattcacttcagactttctatattattatgttttattacccacgctgcagaatataaataaatgtgttttattatttgcttgggattttaactaaggcttaatggattttattctggcatactttaatgtccccacaattcgtctacttgcaaagtactttagttacaaatgagccaatttccattaacatacttcctctacactgcaaagtatctgctttagaacaggcatattttaaaactaaaataaaaccccaactagttggtgtcagcttttgtcttgctagtataatttctatcaacatgttttagaaattttattaatctgttatatttgaaatgtcattgttttcctctctggttattgctattccaaagatcctttaaccatcacagtaaatcacagagttcctctagacttgaatgcccaatacaggagccactaaccacatgtggctactgagaacttgaaatgtggctactccaaattgatttgtgctgaaagcataaaacacatactggattttaaagacctattatgaaaaaaatgagtgaaaatgtgttactgatacatgctgaaatggtgatattttggttatattttgttaaataaaatgcattattaaaattaaactcacctctttatttttccttttaatgcagctactggaaaatttaaaattatatttgtgactcacatttgattataatgtgattataaaattgttctattaattttgacattttatattcacttcagaccttctatattattatgttttatggttcacattatatttctattagacagtgatactttagagcttgtttcttcaccaaggggatatgctcaaccaaaatgtgttggtgaatgaagcccccagtccctacatacatcaagccttgtcaagataatatccacccaagctttctcccactatgtcagtgttcactggacctcactgatgtttcttgctgaatatggcagcacaggaattttctcttatctaacctctggatttgcttttgggaaacgtttcctgttaaccattggcaaatgtgaatcccagtcatttttcattagataaggctgagtccttatataatagatcaagggttagcacagcaaaccagcatacaagaatttgaaatcagcaatcagagcttagtgaaacagaaaatcacttgcttgggctaatagtggttgcttgtgtctgcaatttagtggtgtatgtgtatgccttcaatccacaggagtcagcatgacatgtaagaacacaccgatatcatcatagcttctccagtttgttgtcaagagctttctaccttttacaaagagtcttcagagcctgagacactcgtaagcgtgcatatattcttgcttttttggtaaaggcttgtgacgcagaagaaatgataaatacctaaattgaaagcaagggcttgacattcctaagtttcttatgtaactgagagaattgtttaaaagcagaaggttccacagaagtgtactttttaaaaatttatctgaaaacatagcagtatagtagcactaataagttgaaacatcctcttgtcatggtaattttattgatgaaagctggcatttagtttaaaaatagtaaatataaaaacatcaatttgaattgtactactttttgttagagatataaaatgttaatagctcttaagtattttaatttataggttgtcacttctaatatattttaatacacacttcttatatatttaataaaaataatcttaaatattttaaaatctgctatcatataaataactaattattttacatgtctatttttatgactgtttttaggagaacatgatttagcatttgtttgaaaaattctaattctcttttttttttttcgagacaattctctctctctctctctctttttttttttttttttccctgttgcccaggcttgagtgcagtggcatggtcacagctcactgcaacctccacctcccaggttcaagcaatcctcccacctcagccaccagagtagctgggactacaggcacacatcaccacacctggctggtggtatttttgtatttcttgtagagacagagttttgccatgttgggcatgagtacaagaatatgtaaaaaaaacaaaaaaacaaaacacagcaaatctaccacaggtttcttctatctttgagctctacctgcagtggggatagatatatgagttctttctcacttcatggtccaagaggactgctgaaactccagccatcacatcaattttctaggcaagaatcagtagaagcagggaagatcaaatagggctcattcagcctgccgtctcattctaatgagctttccctaaagtctcactaaaacgcttgtgcttgcattccattgaccacctgtgggtacaagagatactgagaaatatggtcttttatataatacctcgtgcctatataaaatcaggattctgatgctatggaagtggatattgagacaacaaccagcagtatcatacacattaatcaaaggtaccaactttcagtttgctctgattgttggccaccattatcagacatcctaacttttcaaaagaaactacaaacagaagttttatgtgaaatctcctgtattttaaatgttagcatctcattcaaataaattttaaatttttattatgaattttttttttttgagatggagtttctctctgtcacccaggctggagtgcagtggcacaattttggctcactgcaagctccaccccctgggttcacaccattctcctgcctcagcctcccaagtagctgggactacaggtgcccaccaccatgcccagctaatttttttgtatttttatgaaataattttaggcctacagaaaacttgcaaaaaaatggtataaatatttccatatgttcttcatccttaatgttaacatattgtataaccataatacaattacccaaaattggaattttaaattggcacaatactataaatgaagaattggcattattttgtagtaagccagataggaactattttaggtgtctgtcactacgacttaattccgctattgtagtgagaaattagtcatagacaatgcgtaaacaaatgaaggtatctgtgttccaataaaactttatttacaaaaaaaaaaaacagatctaggaaggatttggccatgggctgtagtttgccaactcctgctgtaaactgaacttatagaccttgtttgatttcatcagttttctcattaacatcctttctctgttcagtccaagatccaaaatgtatttagttgttgtctccttagtgtcttgcaaactgagttatttttccttcctcttttgtgaatttgactcttttgaagtgtactattcagctattttgtaaaatatccctcaatttggattagtctgaaggcttcttgtgattagattgaggttatctataatacagatgtgtgcacatgcatgggttagtacacatatgtatatttcctaactctgagagaacctacagttaatgacaccccagtagcaaacagcgcacctcttattttagtttatttgtgcactataacaaagtacccgggattgggtgacttagaaacaataaaatttatttctcgtgattctggagtctgggaaatccaagatcaaagcaccaccaggttcagtgtctggtggaggatccttcctcatcgatggcaccatctcggtgtcttcatgtggtggaaggaatggaagggcaaaagcagctctctgaagcctcttttatggccgtagatcctgtccatgagggcagggccctcattgcctaatcatctcccaagggccccacttgttaatattatcaccttgggggttaagttccaacatataaatttttggggggacacatacactcaaactatagcacctagcacccagatcttggtggtttctaaacaccattctccaataaaagaaaatgagtctccttgaagaagtgattgagtccatggctggggcagggaaactatgagaactgctcagaatttttgtgttgccaaaaaggaaggtagcacacaaaaataataagaacatgtacacaggacacggaaaccaacatgcaggagccccaagtggccagaactggaaaaattggggcaaccaaatataaaatcattttgcattatatgcaagagttttgcattataacccttagaataaaataaatatccacatgtacatggtgatataaatgaataaataattgaataaataaataaataaatgggggagaagggacagctctccttgactgaagaattctaattaataaacatagaaggaatgagagaaacagaaaattaccatttggcaaatagcatagtaacaattgtggcagacacaatctactgacagatactaaaaatattgggacaaagtttgtagagagcaggatgtttgcataatcttaaatgtcaccctcaagatatttattgattgcaaagggaaaaatatagtaactttttatggtgtgacagaccccaccttaaccaaatgaagaaggttaacttcaccagtaataacacatgacatcataaacctcccacttcaaacactgatttggttaatcagtgaaaagattttgcccttgggtcatcagtttgcaacctttaccgtatagtacacttttgatagtatttaatttgctacagctttgccattacaaaagtaatacagaaataatttatgttttcctacctcttaaaaaataatgtatttttttgccaattagaaaaatgttgaagtatgactcatttacagtaggttaaatgaagtatacacgtttgatattccctgtattcaaatgctaagattggtaggatttctagaaagaaaacaaaggccatacaacttcaggaactgcactagatttaaaaattatgaagattctaattggcaaagcctttggaccaggcattccaaactcataacttccacttttactattctgttgaattaatcatatcccagagcacacctctttggtgtctagcatacatagttgatcatcctttttggtcgctaatacttttttttcttttactttaagttctgagatacatgtgcagaatgtgcaggtttgttacatagctatacatgtgccatggtagtttgctgcacccatcaacccgtcatctaggttttaagccctgcgtgcattaggtatttgttctaatgttctccctcccctttggtcactaatacttttaaatacttggtgaaaaatccttgtgcctttccccttcagccatataatcaaagttctatttatacttttatttgcttaaaaaaaatccctgtactcaattttgacctgcatttagttaacatcattttcacttagaatcacttattcttatcaggatttcagtttattgaacacaacacccaaggtagatgaattccagaagttaaatcgctgtgtgcaagccatcacaaatacagacagagctgctgaggctattactttttgactgcgatgtttctaagtcaattgaactgtgattgtcctttatatatatttcagttgacttcttaaatttaaacttctgagcaatcatattcaagaaaagtagccaatggcccaccaaacaggatccataagcctaaaaagacatgagtttggaagctgtgtctgtggattcaaacttaggtgagaatttgtttagaatgggaaacttatttatgacaaataaccaagttaaaaagctgacaatgaaaacatcacaaaatcagagagacaaggtacctttattaagaaactgcccaccccacctctataataccttgactcctacaaatatgggctgtaaatgttttgattatctctctacctttaaatataaaaattattttgaaacatcattctatagagaaaatagaaatataacacagcctttcctctaggatgtcaaattaattttttttcttattactgaaattttaaaaaatgccttttggagtcataatttgttatttgtaatggcacatactttttttagcattatcaaactttggaaaatctttattgcatttcttgaatataacatatcagtgcatttaaaatttactcatataatgactagttttaatactctttaaactgattaaatacatcaccagttgctcatcaatgtcttccttgttctgactacgacgagtttgtgttctctccatacttgttactattttgtgtcaaatctgtaagaatttaaatatttccccatgttcatgattcacttttcttcattaattaaatttatcgaataagaaccaaaacattacttctattcaaaattttccttctaataaattgttgctgttggtgtgtgttgaaaattcttttgccgtaaggggcttcttgatataagaataatttttattgatttaatcatttaattgttttttcatgttccattcatttatttcttagcttttcttccattacatcgtaaataaatttaaagatgaccaaaaatggtacagtcttatttttatgcaatcaaagcagaaaagtttaatttctctcttgttttaataataaccttcaaaacatctttccttattgcagttaaatgatatattcccaccttgacaattttctaaaccatatttctgtaaatttttgcaaatactttgtagcatcctcagcacatagttgcacccatgttgcagggcttatgaagctttataaaaggcagacttcctccaggcacatacatctagtgttgcagggcacagttatatgcaatatgccctctcatcctgcatgtttgtttgtgtgtgtgtgttccattttattttaaaaattttttaaatttttaatttttgtgggtatgtagttagtgtatatatttatgggttatgtcagatattttgatacaggcatgcaatgtgtaataatcacatcagggtaaatgggatgtccatcacctcaagcatttatcctttgtgtttcgaacaatccagttatactcttagttattttaacatatgcaattaaaatttttttttactataattatgctactgtgctagcaaatactaggtctttttcattctttcatcctgaatctttgtgctatgatgccggttgagtcagtacatggagatgctggagaactcctggaagccattcctacagtggaatgactagcgaagatgaactataatggaaattgagtgcaaaacacatacatatatcccattaattacaaaccaaatgcattgccaacaacctccctcttagctggagcccccaaataaccatagctaccccaactccaagaaacacctagaaagaaatgtgatactgaaagtcattgttaaaaattcttatttgatacattttgtaaagatatatgaccatgtgaacatatttctagggcttttcccaagcccataatgtccacgaacaagtgagtggccttgagcttttggcttcattggcttcatgcaacatacacctctgttccttaatgtgcaagagaatatcgaaaaccaagtcaatgaggacactgatgacaaataagtagctgaaggttggcaattcaaagaaaacgagtgccttgaaaacttacacttgagagaagtttctcaaatttgacaataactcttaaaaatatcatactttggtactaaaaacataaataaatctccgtaaactacaaattaaaaaaaatttttttggatcagccaagctgggccttttttggaatgtgcttgtggaagtaaataacctcgattttgtggtttagtcattcatttatgcttcagtttctgttacaaattttctgctacatttgtaatgcgcaagcaatctagaatggacgggcagtgttctgttgcccacagactaagattggctgacatgaggtgctattgcattactcatatttgggtgaaaaggaaacatcctgagtcaggtctctattgccatgaaaatgctacataataagcagccacaaatttatttatacatttatttagctcacaaacttgtgaggttcagctgatctagactgggcttgctcatgcatcgtcaggcaactgcagtgggtggactagatagctcgggtgatttggctggatttgcacatacttctcaggtgaccgttccctaatctaggctgcctttgcccagggtgaccagggccacatggcttctcttcttatgtgtctcattctgcacctgccagctggacagatttcatcatgatggcagagatgcaagaacagttcgaaatgcacaagtatttgaaaatgtggagatgtcagtaatgtcccatggagcaaatcaaatcacatggctaaacctagagccagagagggagtgacctacaatgttacctggtaaaaagcacttatacagtgaaagatgaagaattgaggccgtattttatttttgtaatctatcatggcatctggaactgaacccttggtttttctaaccaacacatttaaatctgattctctccagttcagtaaatagaactcctatccatcttgatgctcaggccaaaatactttcccttgcacctcaaagccatcaataagtcaactaaagtttcaaaatacatctagaattgaaccacgtctcactgttcccactactgctcctacccttgtgcaaacccgctgccattcttcacctgtgcaaacttgatagcctcttaactaatattcccactttcagtttttccctcctgcagtgtttccaccaattccaaggaaacttttaaaatgtaagttagatcatgcacccgctgctccacgctcgtcaatggcttcctcgttttattggactttattgtaagaccaataaaatcttccaacagacaccaggcacggtggctcacacctgtaatcccagcactttgggagaatgagggggggaggatcacttgaggtcaggggttcaagaccagcctggctgggaaaccctgtctccactaaacatacaaaaattagctgggcatggtggcacacgcctgtagtcccagctactcgggaagctgtggcaagagaatcagttgggcctgggaggtggaggttgcaatgagtcgagatggcgccgctgcactccagcctgggcgatagagcaagccttctaaaaaaaaaaaaaatcttccattggattctattgggacttacaataaaatccaaaatatttactgtagcctgaaaagccatgcgtggtctggtcactggctacttaccttgcctttttaaaaattaatttatttatttatttatttgagacggagtctcgctctgtccccccaggctggagtgcagtggcgcgatcgcggctcactgcaagctccgcctcccgggttcatgccattctcctgcctcagcctcccgagtagctgggactacaggcgcccgccaccatgcccagctaattttttgtagttttagtagagacggggtttcaccatgttagccaggatggtctcgatctcctgacctcgtgattacacgaggtgtaatcacacgtgattacaggcgtcagccacgacgcccggccttgccttgtttttataccactcttttctcactgactctgcttcagccaaacaagtcttcactgttatttataaacattgataacctcaggacatttgtacatgctaactccctggcctggatgacattttgtacatgctaactccctggcctggatgactgttgaccctgctgttgtctataacactcactcactcactcagttcattcaagtttcccttcgaatgaacttcttaacctctttctctagtaattttctgtcactctcattctagaccagctctttttctttcttcatggtacatgttaatagtggatattataaagtatatttgaattttaaaaaatagcctaaaggaataaagttctagaagcattacagctagaatgaaaatatttccaaaacatgatatatgggagaagtgacagcacaggaaaatcctaccactgaaatattaaagtctattgcttctggacattttaaatgtatagtttttgtgatagctaaatattacctttacctggaaagcttttagtcatgctctattttctgaactatatttcccagttaatattagaagtacattaaacacacacacacacacaaaaactatgaatggctttgatagcaagctttgtgctcactggctacattctttttttttttttttttttttttttttttgagatggagtctcgctctgtcacccaggctaggagtgcagtggtgcgttcttaactcactgcaacctcctcctcccgggttcaagcgattttcctgcctcagcctcccgagtagctgagattacaggtgcgagccaccatacttgggctaagttatttttgtatttttagtagagacggggtttcaccatgttggccaggctggtctcaaactcctgacctcagctgatccgcccacctcggcctaccaaagtgctgaaattacaggcgtgagccaccacacctggccaacattcttgctcctatatagctgtggagccttcagggagcactccagcatgctaggaatatctggatcactgttaagtgatgccaagcccgagtccttcgattgagccacgggaagaggagcccatataatggtgggaaaccctttatatccagggtttaatccttcgctgtgccacagagagacgtctttggcagtctccagaagaccaggtaccaatttttacaaaatgttttattttgcatatttatcgagataaaatttatgaaacaattaactcgtttaaaatgaacaacttagtgacatttagtacattcaaaaagttgtgcaactaccacctctatctaattccaaaacatttccattgctccaaaataaaaccctgtacccattaagcagttactcctcattttcctgttcccaagtctctggtaaccacaaatctgctgtctgtctctgtggatctacctattctggatagttcatataagtacacttataaaatatttgactttttgtgcctgccttctttcccctgacatgtttttgaggttcatccacactagcatgtatgagtactttattcttttttctgttgaataacatccgactgattggatatactgcatgttatttatccaatcacctcttgatggtcatttggctgttacaacaccttttggctattatgaatagtatcattatgaacattcctatactacatttctgttcagtgcctgttttcagttttttggggtatatacctgggagtggaattgctattatcacagtggcaacataattttttaatgtgtaaaataaaatacctaggattatttttaaaaggcaataatgttgatacagagctatcaaaacattaaataaaataaatgtgtgttatggtgatatatatttcttgattttgcagtataagaagatctaacagcaagtctactaactgctgtgcattcaaagtatacatgatatttccaggcatctgcaacacttgtaatatgaaagggtttgtgtttttttggtcatagctccttccagtattattgggatttattgactgttaaagactaataaaaataaaaataactttttttctatccagattcatggaacccttgaagtctatctaattgagctcagggacagttacgacccccagttaagaatccctgttttaaagaaggagagtgatggtgaaaaactcacttcctagtagcacatttatctctgtctaactctctcaccactcttctcccatctcatcttttgtcttaggaaatctaaactcagcagaatcagcttggacaggtgcagattcattgaagtgaaggttagtgaattcacacactccaaagacattacaatggagttgtaaactgttatcttttaccattttaggtgtcagattaaatttaagttaatttccatataacttaaacacaggaattaaaatagcttttctaaacctaatgttgtgtagtaccttacagtatctaaagttctcacacatacatcattgtatttaatcctctgaaaattgtgtaagtaggtgttatagaggaagtacaccctgttattaccttggttttaggatgaggaagcccagaggcagaacaatgtagtcaattaagtttacaaggcaaacacaatgggctagaacctgggtcatctagttaatacaagctgtaagtgctttttctattggagcagaaaatcaatatatctaaacataattgttcacttggcttttcagtgtaaagaagagttactccaaaaataaaagccaacacattataaattgctctctaaatggggaaaaaaaagcagtaatgtgataatagtatagttcacagtcctagaaactcaattcttctattgtctcaaaggaaatgtctcaaaaaggccagaaactcaaaccagatattttcctgtaggaaaaggtgaagatgttttcaaaaactgagttttattattacctgaaaagaaaaataaaatagtgctcaatctgtatttggagaaatccccaaacagaatattgttctgtatgctctgagaattttctctagagaggatttccttcactggacaaatgcataaataatttctgccaaaatgcagtcccgtatgtcatagttttcttgttttgaatgaaagaaaatccatttgtgagtgggttaggttttctatgcaccaaaagttacttattcaatttttagagtttctattatgttgctttctcctctggacacagaacataccttattccaacagtgaagggaaagaaaaaatgctgagctctggctataaatattttatcatgtatagagacaaagatcacaggggggcagatgaggagcaggaaacaaatgtgatgttgttttcaaatcctcagaggaggtttatattaaactgtctctgccagcttgttttctgcctggcttttacactgtcaaacccggtaatggaatgattttgtgtacttggagttcacctactggatgaggaatgcagcatctaactgaaagtgttaaaattatccgtgttataaagtatacacaaagaaaaattggcagagtgatgaagacatatttctaagaaagatgatccatattaattttttttggaaaggttctatcctagtaaagacaataggttaaaaaatatgttgtatgttgagttttctcaattcagtttattatttttctcctcttaatctaatgtctcagagttgttggaaggttttaatctaatgggatctagcatctttcatgattttgcaacccattggagtttatttggctagaactcaatattaatgaggacaaggtcttgagcccattctccataaagggaggacaaaatgctattttcctaacataatggcacagaccatgtatcaaacctgctactcacaaaattgcatctctgggtacaaggaaaagtggatgaaagggtgagtaatacaacagccgttctaaattgcagatctgatggaacccttccagtctctaagttaccttgaaatgtatttgcataaggggctgccatccttgagtgtgaggaggggaaaaaagcatgcagagactgtttatactagtagagctgatagtaaatcttccttccgttttatataccatagaattttagagtttaaaatgtttaaagtttaaattttagagtttttaaactctaaaattctatggtatataaaacagaagaaggctcattaactctgagatatgaatatattattgatattggtcacttaccaatgtgcctaatagtatactcatatctcagagtcaggtgccatttgtaatgcagttgttatatgttttgaatgcatatcatgaatgagcatctgatttccaacatccccaatataggagtaaaatgtccagagaatctatttaaatgaagtacaaaatggcggccgagcgctgtggctcacacctgtaatgccagcactttgggaggccgaggcgggtgtatcacctgaggtcaggagtttgagaccagcctcgccaacatggcggaaccctgtctctactaaaaatacaaaaattagccagtcatggtggcgggcacctgtaatcccagccactcgggaggctgaggtaggagaatcacttgaacctgggaggcagaggttgcaatgagccaagatggtgccactgcactccagcctgggcaacagaatgagacttggtccttggtctcaaaaacaaaacaaaacaagaaaacaaaacgaaatggcataaacttatgtgaaaacttagttatgcttagctaggacatttaggattgtttgtttgcttgtttgtttctttatcaaattattcgcagatatcaggtcactgatttttggcttgaagagctaaattcggtgactaaaatgctgcttggaatggttttgtacttggtaaaaaatgcacgctaatttttttactattcattattggccaggtactctgcataggccattttatagaaaatggtattcatctccatctttcagatgagtaaataatctcagggaataataaattgcctcaaatcgtgcagctagtaaaaggtggagccagtctgtggatacgaagccaactccttcctcttgaaggttggaaatgcaaattctaaatttaattgacatctgtaccttttgttaaaatatgcattatcaacatgttcagaatggtatagctgttatctgaacatagtctactaaacattcagcattcagatttctttcttttttttttttttgagataagttctggctccgtcacccaggctggaatacggtggcatgatctcagctcactgcaacctctacctcccaggttcaagccatccccataccccagccttccaagtagctggggctacaggcctgcaccaccacacccagctcatttttgtatttttctgtggagacggggtttcgccatgttgctcaggctggtctcaaaactcatgagctcaagtgatcagcttgcctctgccttccaaaatgctaggattacagggtaagccaccacacctggcccccagattgctttttaaaagtcagtaatggtaatatggctttagcagagatatatgggtaaagtcttcttaatggcatattttaagagaaagaaatatgagaggagagttttagtgatgatttgagaatgtcttaaagtaattttatgatatctaatatttacttgattaatttggctatttgcagcataaatagaaaattaaaagtagtacgaatccttagaaaataaactcaaaatggttaattatatagaaaatgtaatataatgatgctaacaaaatacagaaatttacaaaatgcatttagttttatttttctggatcaaagtattacttctgtcaaatatattgaattatcttattattatgcagtagacccacatgattatatcaattttaaaactgtattccatgacaggtgacttgtctgctgatactctgaactgtttgaatagggtatcaaaagatgcagaaaagaaattacatctacttatattttagcctattttgaactaattgaaaagcagctgtcagaactgtccttctagaatttattgttgcctgtgtaaggcattgctttttactaagtcctagtgatcctagcaataatagtttaaaacatagagcatgctttttttttttttaatttttttttttttattatactctaagttttagggtatgcttttgagaagatattttttttcagctactgcatgttctttctttgattttgatttttatgtacatattttttattacactttaagttctagggtacatctgcacaatgtgcaggtttgttacatatgcatacatgtgccatgttggtgtactgcatccattaactcatcatttacattaggtatatctcctaatgctatccctcccccctcccccaacccctcaacaggccccggtgtatgatgttccccttcctatgtccaagtgttctcattgttcaattcccacctatgagtgagaacatatggtgtttggttttttctccttgtgatagtttgctgagaatgatggtttccagcttcatccatgtccctacaaaggacatgaactcatccttttttatggctgcatagtattccatggtgtatatgtgccacattttcttaattctgtctatcattgttggacatttgagttggttccaagtctttgctattgtgaatagtgccgcaataaacatacgtgtgcatgtgtctttatagcagcatgatttataatcctttgggtatatacccagtaatgggatggctgagtcaaatggtatttctagttctagatccctgaggaatcaccacactgacttccacaatggttgcactagtttacagtcccaccaacagtgtaaaagtgttcctatttctccacatcctctccagcacctgttgtttcctgactttttaatgatcgccattctaactggtgtgagaaggtatctcattgtggtttcgatttgcatttctctgatggccagtgatgatgagcattttttcatgtgtctgttggctgcataaatgtcttcttttgagaagtgtctgttcatatcctttgcccacttgttgatggggttgtttgtttttttcttgtaaatttgtttgagttctttgtagattgtggatattagccctttgtcaaatgagtagattgcaaaatttttctccgattctgtaggttgcctgttcactctgatggtagtttcttttgctgtgcagaagatctttagtttaattagaccccatttgtcaattttggcttttgttgccattgcttttggtgttttagacatgaagtccttgcccatgcctatgtcctgaatggtattgcctaggttttcttctagggtttttatggttttaggtctaacatttaagtccttaatccatcttgaattaatttttgtataaggtgtaaggaagggatctagttccagctttctacatatgcctagccagttttcccagcaccatttattaaatagggactcctttccccatttcttgtttttgtaaggtttgtcaaagatcagatggttgtagttgtgtggtattatttctgagggttctgttctgttccattggtctatatctgttttggtaccagtaccatgctgttttggttactatagccttgtagtagaagataattttagcaacaaattacttcctatgaagtacaagtaaatcaatgttaagggtaaccattttaaaatattgagaggtaaacttaattggaaaatcttcacaacacctttaaaacataaattcatattctaagggggtcttaaacatttttcaaatgcactggaaaatagccatgtaaagtgtgatctctccattgtacctctcacagagtagttgcatttttacagaaaatacaaggaactaagtatggaccttttgtacctgcagataactggtgcatataatgaaataaaagctgttatttcttatctcatccaaggagaaaaatggaaaaattttctaggaatttaggttgacctataatagacaaggcgaactattaacctacaattattttcagggaaattttctttgaacttctctgaaaattggaggagcaataattaatatcatgcagaagaaaatacccgggctctgggatttcattttttatagatatgtcataattgtacctattttggggatacatgtgatgttttgatacatgtatacaatgtgaaaataaagtcaaggtaattgagatatccatcacctcaaacatttaccttttctttgtgttgggaacattacaattcttctcttctagttattttgaaatatacagtaaattacggttaagtgtaattttcctatggcactcccaaatattagaacttattccttctatctaactatatttttgtactcattaatcaacttctcttcatttctcttgctccccactttcctttccagtctctggaaaccattattctaccatctacctccgtgaggtctacatttttttagctcccatgtgtgagtgagaacattttctttgcctggcttgtttcacttaacataataaccttcaattccatccgtattgctgccagtgacagaaattaatttctttttgtgactgaatagtatttgattgtatacatataccacattttctttatctattctttcagtgatggaaacttaagttgattccatatcttggccattatgaatagcactgaaataaacatggatgtggagatatctattcaatacattgatttcctttctgttgatatatactcagcagtgggattgctggattgtatggtagttctatttttagttttctgagaaacctccatgctgttttccgtaattgctgtattactttacattcccaccaacagcatacaagtatttccctctctctgcatccttgccagcatttgttatttttttttctttttgataatagccattctaactggagtgtgataatatctcattgtggttttgatttgcatttccctgatgattagtgatgttgagcattttttcaaacacctgttggacatttgtatgtcttcttttgagaaatgtctatttgggtcttttgtccatttttaattggattattatttgctattgagttgtttgagtttctaatatatcctggttattaacctcttgtcagatggatggtttgcaaatattttctctcatactgtagattgtctcttaactttgtcgattgtttcctttgctgttcagaagttttttagcttgatgtaatcccatttgtctacttttggtttttttgcctattcttttgaggtcttaccccaaaaatctttgcctagaccaatgtcttgtaatgttttctcaatgttttcttctagtagtttcatagtttaggttttacatttaagtctttaatcaattttaagttgatttttgcatatagtgagagagaagggtttaatttcattcttctgtatatgtatatcaggttttcctagcatcatttattaaacagactgtcctttcctcaatgtatgttcttggagcctttggcaaaattagttgcctgtaagtgcgtggatttatttcagtgacctttattctgttctattcatcggcatgcctgttttcatgctagtaacatgttggtttggttactatagctttgtagtataaactgaaatcaagtactttaatgcctccagttttattctttctgctcagcattgctttagttattcggagtcttttgtgtttctgcatgaattttaggattcctttttctatttctgcaaagaatgttattaatattttgataagattgctggaatttgtctttttaataggacagcagagaatataagaaataatatttgctttcatttcctgggtctttcatgaagaatataaaatctgtatgtagtccttttctaccctatctataatatttattatttaatcatttaaatggcaaggatatgctgaactataaacaaggttgggaatttgtataatccgcatataactgtgattcagccaatatgtatatagttgattctcattatttgtgattttatatttacagattcaccttctcatatcttttacatttttgtgcttaatgttggtgtttccatggtttgaaatggccccatagtgtagggctgacatgctgtctcatgttcctaagcacaataggctgtcacgtgccttatggagaaaacacgtgtgttagaaaatctttgtggaggcataagctacagtgctgttgattgtgagttccatcttaataaatcaacaatatgtataaacagtgacacacataaaacaaagttatgtattgattatttgacaaaaatattggagccacagagttgtggaaacctaaccctctatttctcctagtaacagtgctttggtatttgctaatttagtgtttgtggtgacttcataaatcataactactgcaaatatcaagaattgactgtattgaaaatgtacgattaccaggtagtgggctaggtcccagagattcaggaaccaaaggcacagttcctggccccacaaggttcaccatctattggagagatggatttgtaatagaataattattcacagtggagaaaatgctgtggtacagaagcagccaaggtactaagaggagtctaatcctacctgaggtgtcctggtaataatatacagatccgtagctgcatgcctgagaaaatgatagaaacagtaaaaaagaatgcactgcatgtgggagaatgtaaagtaaatgtggctatatgtttatttttaagcacagcataatgttagtggggttctgagtcattttggtactatattgttacagaaatacactgtggtgaaatgcttccacctcttgctaaaatgaacactgaggaaaaatgaagaagactgacaagcaccagcgaaaagttgcagaatagaaatagccacactcctctggagtctttaattcatccacagccatcatataaaggtaggggaataaattaccttatggttttgtccttttaaaatgtattttaaattaaacaatactaaaaacacagaaacatgcatgtattgattcaacaatattctctgggtgactgctataggcctgcaaactgttgtaggcactggaaactaaaagggcaagcttatggccttacagaagctgacattctagtggaaagtgtaagtaaataaatgaaaaggaaactgacaaccattgataatgtttatgaacacgataagcaggttaacgagataagggatgagggactactttagtttgagtgtcagagcagaacacttagagagtttgagctgagaactgagtgatagaaaaaaatccagccaagtagggtttgaagatcataccgtccatgtgtcatgaaccaggatccatgtggatatattcaaggtttggaaaccaggccagtatcactttaagagaggggctttggagaagcaggatgtagaagatgaagctggagagctgtttagatgtgggaaagtgtcttcattttattttaataaagatggaagttattggaaagtcattagcaggtgaatgaaagaatttatattttttaaaggccattcttctttattatagccaattagtatgtgtgggggtcgctactaccccccacacatgggtactaccccccacgcagggtgctcttacccacataaaagatactgctgacttggcttagggatgtggcattggagatggatgaatttgaaagatattttggcatagatgaccacccaagctccttatgaagctccaaatataattctaatttccatatgcagggtatgctgtctatttgcgttttaatacacaatggtcagattgtaatttattctactatttcaaagaactgcttatgatgtaatatgtctgtggcctgcttctgaaacttagagggtgttgatgtcaaattgtgaaaataaagaaaattctgcccaaaatctcatattctaggtgggttatcttgtaacactcggagacttgagcattccagatgtgtttgtagctcttctcctgcttcctggatggctcctgacttctaagaacattgttgctcaagccactgagaggcagcctgtgggaagggtgtccttctagatgagaaacaagagttttttaaggactagtgacttagtcaagcataacttcaacactaaaacttttatactgggttattcatagatggaatgttgaagggaaaattaaaatccaatttcaaattggtgctttaaaaagatgagaatatatggattcaagcaatggttaccagagaaacttatcctctgtgcagtgagggtttcacatcttgtattatgtaaccattgtgcaatgtccatagggtagggaagagaggaaaagaaatgtatgcaatgaccttgtaggtccaaatttgcaagaagccaacagctactgttggagggtggacaaccctcaagtctgtgctcagaagagtttttcatgatagctcaactgaacgatattttaagatctcagcaaagtgcagcttgctttccaatattcataactgtctgaaattgttctaaacttgtatggtgtaggaattcttccttcctccaccccagatccttctgtaaagctttttgtcgttagaaaacagacccagtgcagcaattatacagtgctggataattaaaaagtcacatgagactctacaagttcacaggagggccaaggctcaagcttactgaggggtattttgtaccacacagctcaggtagctaaaagcaagctttgttaagaagtaaacaggtgcaaaccatcaatcagtacaattttgataaacagcatggacaagctggtacaggatgctggtggggttttagacttttcacagcaccttataaatcactagctagtacatttcacctctgacatggccaaaatcaataccatgtttccaggtgcccatcaacataagcaaagagctaccagtgcacaagttaaccctttcctcatatacttagagaaatgagtcaggatgaaaacaaagaagacctaaaaatgacatacgccattctgtgattagtcttgtcatccactggactagataaaactctttgattagctggagagatcattaaaaggctaggcctttcaaatggaagtcaaagttccttaagaaatgatgggattctgaagcaacaagactaacatgaactagaaagagtaatggtgccttaggttgatttccccagaagcagagtgaaacaaaaaaaatggagtggtgtttattcagaggtgatgccagcatacgtttgtagggtagtgtggatgtgagcagaggaaggaaggaagacagaaaaggtgccttattgtttctgtgatcaagaggggtacaatcacattaggtaactttgggatacagcatagaacataacttggatttatcccaatcaagggcatggaaattagggtatttagctatcttctcccatatgtctcgactaagggctgctcccttaggtggaggtgtggagtgtcgactccctggtatttgtaacgtgccatacatgtagaccaagaaaactccagcagccagaggaagtcctcaggcagagtcccagatgtttgcttttgaaagccaccatcaggtcaacttgcacaatgatgataactgccaatgagatgaggatagagcacctacagtttctgctacaagaagtgacaacaaagatgatggtgatgatcatgatcatatgaatagagctatcatttattgagcaactagaagccaagtcctatattaaatgctttacatgcattttatgatttaattcttatatgttggatatcccctctgtcaggtgggagatatttatacatgattatatatatataatcaccacaatcttgcagaatgtatgttattccggtctttaatttagagatgaccacacaagactcatcaaggttaaccaaattatctgaagtaacataggaaatacagttgagattcaaacccgagtttgtctgcctccaaaagggtcccttggcttaggcaacagtgtaatccttaattaactaattaatcattgacatttttctgacactcatctattaaaaagtcagcaggcactatttagattctgagatacaaaacaaaaattgctcctgttcccaaggagttctatgtttatggaagagaaagaccaaaagactgtagtattgtaagggctaaactagaagtatgttgtagctattgaacaggatcagtaaaaagagtatcacttgttgctgtataacaaatagccccataactcagtgacttaatataataagcatgtgttattgatcaggagcctgtgggtgagttggggacttctactgatttggactaagcttggctgatcttagctaggtttgctctgtagtctgctgacaggttggctggggctgaatagtctcagatgcttcactcccatgtctggtgcttgactggatgtggctagggcaactcacttctccagcaggctagccaagacttatttatggcaactgagccatgtggatcacttattcaagagaataagtgtaaggatgcacggctgcttaagacctggcctcagaacaatcacttctacaacatatacatcaaagaaattcacagaatatctcagatttaagggatgtggaaatagactgtacctcctggttagaggagttcaaagtcacattgcaaaagacattgataaaggaaaaagaagaactagattgcaaacaatctaccgggaagaaaacatctgaataaaagattacagagtaagtaggtattccctagccaaataagattggaagagaatttaaaacaagaaggggctgggggccatgcctataatcccagcactttaagaggctgaggtagaaggatcacttgggtccaggagtttgagaccagcctgggcaaatagatgctatctctacaaaaaataaattaactgagcatggtggcactgcctgtgatcccagctgctttggaggctgaggtgggaggatcacttgagcctgggaaataggctgtgatcataccactgcactccaggctagacaacagagcaagaccctatctcaaaagaaaagaaaaaagaaagaaaagaacagaaaagaaaagacagcaggatagtatgtatttaaagacataggaatatagtgtttcataatgctgtcaggttacttgggaatggtgagggagagagaggaggattaaagacaagaaatatgtaaaatgtaccagaattcaagaaaataaaagttaatttgcagacagaataatatgtttatgaaaaagtgacagacaaatgggaagatgaacaaagttaggtcaatggatgaatgtatggttgattggacagtcaggtgtggtaatagggttaccaaaattgttaagattatctcttagaaaaaaatttgtaggttgggcttggtgactgacacctgtaatcccagcactttaggaggctgaagcaggtggattgcttgagctcaagagttcgagatcagcctgggcaacatggcgaaaccccatctctacaaaacaaacaacaacagcaaaaaaaaaaaagaaaaaacaaaaattagccaggaatggtggcatgcacctgtggttccagctactggggaggctgaggtgggaggatcacttgagcctgggaggcagaggtttcagtgagccaggattgcactgctgcactctagcctgtgaaacagagcaacacaccctgtcttaaaaagaaaaaaaaaaaaaaaaagagtttgtagaatgtgtatttatttcaacaagaaaggaaggaggaaatacctttgtaagtgattgaagaaaacaatgtggttgaaagcagacctgaaggaaataacttttcagcatgtgactgtgtttgtggcttcagggagctattgtgatgatgcagattgcaggatagaaactctttctatgggacaacttttataagcgtgctaagcatagtgagtaggtttttgtgaaaacgaatgaagagtttgacttatggtcacaagcaaattccgaggctctagattctcacaaggccaagtctctgtgaagactgctgatgttagagatatatttatatagagatttgtgcatgtgtgtgtttccactgcagtatgtatgtgagatggtaattttcaattcatggatttctccactctgagactgagttgtgtcaattgaatagcagctctattctacagttttccctagccactttcattgtactcctgggtctgtgttgcaagtgtggagctcgcccaaatcaccaggtgcagcagattttactgtgaagtttaacaggctaatattagcatctcaagctcctcagaggcagaatggaaacttttgtagttcattttcacagctaagtaatactacactgtgttaatatatcacaatctatctgttcttttatagattgatatttctgttgtttccattatctgctattatgaaaaatggtagtatgatcattttttatatctctcctggtgcctgtgcaagagtgtttcaaggaagtatatgtagaagtggattcctaggatataagatgtataggtaaccaaccatctcggtttgcctagaactgaggagttttccaggatgcaagatttttggtgctaaaaccaggacagtctcgatcaaaatggtataagtggtcaccctgaatacacatatgatcgattttacaacataatacatattaacctctttcccacagaattggtagcaatttacactcccaacaagtggtatatgactattccctttgaacaccattctcctcatgcctggtgttcttgatatcttaatttttgccaatcaattgagtgtgaaatggaatcttgctgtaatttgattttccctggttactaatgaggtttggcatcttttcatgtttagttttccctttgtgtgttctgttctgtgaaatgccttccatatctttcatcttttttccattttttttttaattagacacatctcatttatctcttcagattttcttgatttttccagtacttaggttagggtctctcaacagcagcagtgatggcattttggactgcataattcccatgggatactgtcctgtgccttgcagcacatttagcagcacgcttggcctttacccactaggtgccaatagcacccccaagtaatgaaaataaaaaatgcctctagacataaaactacctcttgccaaatgtttccccagggaacaagattgtacctggttgagagcgtcttgctcctgtgggaaagagaaggtagcagggcaggtaaattccatttccatttctctctctgacgatctctctctctcaaggcctatacagcatgtctctgtccagcatgtttagaggtgttgcgtgtcacatagcagatgctttgctggaccactggctgtatttcttcatggtaatgcagtggtcacagcagtaacgtctcactttgtcttgctgctcacccttccctgcttcatttcctatctcttctctttcacacctttggatttcatattccaattaaaacctcagcgcttagtgcatgtctcaggttcagtgtcctagggaaactgaactaaaacaagtagttgcagacaagataccctacagataggattttggaatttgattactgacctgtctgaaagcaatagggatcatatatttagtggtaaattgggataaaaataaaccctggcaggcaggcagcagcattacaattacttaagctctcatctgcagtaaattgggaaatggcttaagtggaaggtcaggtgttaggaggcctactagctgcaacacaaagtggctattagagcactgataattatgcagattgtgaagtagatggctacttttgacctgcggaaagaaaatgttaagctgaggatggttcattgccaacttaaggcatgctctgaaagccagaggacatcatctatggctactttaaaggagactaatctctggtcactgcagggcagactgtgttgtaacacaggcccaggatctgactataaggctggtagggctgccaaggagacaggatacacagctttgtaaaatcttctgtttcaaactcagggccatgttcggaataaggagacgtgggatgagttcatttggatggatgagcttgaggaccttaaacttctaagttcccttaaaccaccctggaagacatgcgtcatccttctttttctgacagagaagaacagctttccttgcccaacagttatgtaggaatctcacctgaagcaggtgccttatgagataattcttgcatgtcttaagatctttctctgccactgctcattgcctccatgctgataactagggtgtagactcagcagagaagtatcagccctctccagggggaaatagcctatccactgaacaattgaagtaccttgaaacccacccgggagtggaccttgtattagagtggagggggggatatatgttgtgctgaactcttattgacttcactggggatggcaccaagttcaagaggcagaagaagagacccagagccagaaacaagacattgggtctcatgaaggtcttacatacaggggagagagtccagtggtggtgagctagaccggagaaccacaacacttgcaaaaagcatacagtttatgtagcattttcacttagcaccctcccccaataacttccacctggcaaccttttaacccaaaacacagggcctcaatcccctgcacagcccacattccacatgacaggatggggcccagatgttcctcatagaaaaggaatagctctctgggttggccactcctggattccctagctcggaattcccaactgcattcaggtgcaccatacagggtcattcttagcatatgcttaagtagttgttatcaggtacattcaccatacaacataatgctagatcagagggtattcattgatatagagacactcactcataagtcaagattcagttgtttgtcaaggacacttggagctgatcccgatagttttctggggtggttcttcaaatcctgtatgttctgatggcctacagcaaatgaggaaatgccacaagttctttgacagtctcaataaagggtctgacggctcagagaggagggatgttagaatgcatgttaaaaaggttttaggtacaatctatcacttgactatttctaactaagaaggcccagaggacactcccttcacaaagacaataagggatcctgaaaaggagtgctgacacctttgagaaggtggtctgtaaaggctgccatctatgggctgggattgacatttgaagaagctgtccaggagttaatcttcctaatattgctgggatcatgaggcccagaacaacagaggctaagtagcagtgcttagtgttagaggtcaggtggccttaactactataatgcatagcaaaattaaagtggcagtcagggtgccttgagccacagggatctaacagtggctaagagatcctggaaatctttgggacaaaataactgggcagctaatagatctgcagtttggataacaggggagaacaactgaagggttataaaccatggaatatcacgggccctttataggtttccacatctaagtcagttcagagatggcaaaggaaccatggcagtcagctcatgttcatgtttcatgtttattccgtttttaattttttgtacattttacacatcgtgattgcatatctacctgctaattctaatacatgatgtacaatcggtacagtaaaccagttgattgttatttggtcatctggggctctgggtgaccaaagctcctcaggtcatgggtaatatttgagtattgatgcaagcatgcaggaatccttagggcctctactggggatgttttcccatggagagtattgctttgtgttctctaggagtcagaaacattggctgtcttctgggattccacgctagggactgactctcaggcttggctgctctgcctcccattgcccagggcctaggctttgtctttagagcagtgttgttatcatcatattcctttagagctacccagtcattttcattagtttaattcctaaatcatttctctggtgtcatttctctgtcatttatttttgagggcaaaggtggagggaagctcggatatttcatttactaactactgagcctagcaaaacttattacttctttttaaattttttttgagacagggtctctctgttgtgcaggctggagtgcagtgatacagtcatggcttacagcagcttcaagctcctgggctcaagcagtcctcctatctcaacctcccaagtagctgggactacaggtgcaggccaccacacctgactaattttttatatttttagtacagacgaggtcttgctatgttgccctggctgctctggaactcctgagcttaagtgatcctcctgcctcagcctcccaaagtgttgggattacagacatgagctaccataccaggccatcaaaattgacctttttataaatgtatccaggatatatttgcattttagcatagggatcctttaaattacctagactgcatactaccaaaaatgaaataactttgtctctctacttgcctgaaaatgacttagagcaattagcaactgcccagtgcaccaattataatggcattgagtttggtccaggaagtctttaattgaatgaatcagaggggtattcttatctcttgtgcagtatctttttcacatggttaacaggtattgatcagaagactgggcctgtgggcatagatcctgagtgacgtttctctagtcagaggcaagctcagctggtcccctctccacacattccttatttatggaacgctcacattcaacatgtactgagctgtccctcttatttagtcattttttagaaacatgatagtaagtggtacatttcaggaatattttcacacgtttcaaagtttgacatgcacaaagacaaacttttaacagttcagaacactttcatttttggaactgctatctctatatatatagagagaaatgtgaatgactttgcttgactcgtcaaagggcggagcagggttctcatgcctgtggagaccacacggtgtaaggcaaagtaggcagggggtgagaactttttaaagagggaaacgtaggcctgtgtaaatgcaatggctcctacctatctccaatctatcattgctctatgggaatgtgggccagatattccagttttttaaaggaagcttgaaagctagagttttctaatataaaacctcctaatttttaaacattgctttattaaaaaacaaaaatatgcaaacaacaagtgacctgattttctgggggaacaaaacatgtttggggcgccctatgtgctgaatctagcctgcagacaagttcaaatatgcagcatgtagcttgagatggatgagtttcaaaatgcaaatcctatctgcactggcaattggattattttgtgtcaaggaaaatgacttttctgtcttaaggtatttttgcttttgattgcttcttttattgcttgggattttcttctccctggtataacagcctagtggatactttatcatctaaagctacatataagcctatactatgatccagcaatagcccccctaagtgtatacccaaggaaaactagtgcatgatggatctattcaaagtaacacaaaactggaaacaagccagaagttcaacaacagaagaatgagaaatcttggtttattcattcagtggaatgctgtgcaataataagaacacacttttgttttgagatagagtctcactctgtcacccaggctggagtgcagtggcacagtcacagctcactgcagcctcaaccttccaggctcaagcaatcccctcatctctgactccctagtatctgggaccacaggtgctttccatcacgtccagctaattcttttatttttgtagagactagttctcactatgttgtccaggctggtctcaaactcctgggctcaagctgtcctcccacctcagcctcccaaagtgctaggattacaggtgtgagccgctgtgcctggcagaacatgctgttgatacacacaacaacatggatgaatcacagacatgttgagcaaaagaggctggactccccaaaaatgtatgtatgtgtgattctatttataaggagttcaaaaactggcaaaattaatctttggtgttagaactcaaaaaactagtttgatatgctgagttaactgtctccttttgcagctcaggggtaactgcttagtaatttgaaggaggttcttaagtatccccatttgccataggcatgaggggaccgggtgcttgaaggtccacccttccaacccttcaccaactgagacatctccatcacaaatgcatgcatgtagagaggaaggcactctgattccccatgtgacacgatatttcacttttttttccaagtcctccaggttcccacttgtgaaatacaagttccttatctatcacagagatgtagagttttttttattcctaccaagcttgaaggagtggctctttgtggagcattacttatgtatatgttgatctcacatcttccccagagcatcctagtctctgcaaataaaattaaaaaaaaaaaaaaaaaaaacaagaagcaaaataacccttctcttttccctcataagcttaatatttttttttcaagtttcaaaaggaatagactgaggggaagaaaatatacacccaaggctatttctctactactaggaactggtgtttccatacagattgtttctcatacctgggggatgagtaagtgcagccttgaaaggaagcaattggaaagtacctaaaagaagctcccagcctattttatttggcatctgctctctagggcagaaagtgagacaaggagaatctgcagtaaaaaccaaaggagtaactgaagaaggggaaacattcactgagcccttaagctagaaagacctctaatattacaatcaggactgtgtatttcaaacaacctgtaaatagtggtggcttattgcagctgataggacacaaaagacagcaagcagagatctggttgtagtacaggggatgcagaggagggcattgtgaccgttctttaaggatttgttcctaattcaaagtttcctctatctccacattattcaaacatcactactcaaaatagaaaacatggaaataactcagaatctataaagtgcttcacttgcttatttatagtgcaggaaagataggaaaaattttaaaaatggttattttgttaacttccagttgttacatctctgtgttttaaaatcaagttatatctcattacaataagcttcatgggaaatttggcattttattccatggaattctcactggtcaaaatcaaaggtacttgacctaggtaggaatcaggtgtcttaaatcatgcttagctacatcacttatagacagcagtgcagtcctgagttcacttcttaacctctctgagctccgggtgaaacaaagtgaaatccatgctttgtctgttccttaagtttggtatgaaacaaagccaaaggcatcctaacaaagtgtccagtgtgaagtgaatgggaggtatgatcattcttagccctgtgacttagaaagcaaaccaaatggcatgtctgcaaacattccttagatgaagtgctttttgattcttgtaattaatgtttgttttttattgggaaatggatataagtatcattagttatgggaaagaagttaaagatcatgcattcatggggagaaaaggagctatacagttacaatagaaagcactgggatttatactatcctctctttctggctggccaaagagtataactccatggagttgtttttccttatgtctaaaaagaaataccagtatctaccaccctgaactacttaagaggagtgttgtaaacatcaaaggaaatcatgtgtgaaaaggctttaaaagttcaaataaagaaaaagagtatatagttgaggggttataccacttttccctcttattctcagaaaggtttctaagcattatattgaggattctgataagattaatcaagccctctcccacttgtaatgctatctgatttttttcttccttttacaaatctaaataagtaaagatagtatctagcaaaaggcattgtatgtgtcaaattcatcatatggtcaaacagtactaacatcatgggctaatgtcaattagcccatgtcaacattttattgtattaaaacattagtcaatgttttactaaatcaatgttggccataacttgggaaaatgtgtattgtccaaaccaagactggtttgagagtgaaaaggagagctgttaataattatgtcagcctaacaatagtcaactaagactgttcttggacaagaagtattgtctaaaagcttcctttttaaaataagtcataattatgcctaatatgttctacagactaataaaggtatggggaagattttttaatgagcaaactaaatgtattccatggttctagccttcaagtagccacaagtctatatggtaagagaaagattatgaaatacaaggatgcaaaattagtaaatgaaagacatcatgctagctaacaagtgttggattaagtggagtagaatcttggagatttagacacaaccataaagattcagacaaccttgagttgttggggagcttctctgagagggccaaagtctttaactgggtctagaagactggtgggagctaggtagatacagggaatagcatatttggagatatttggggcaggtaaaacaacatggagaaacacataaattgatgagtttccatggttgtgtgtagaggggagagagaagggctgtaatgaaatttcataaggataaaatctattgtttcaacaaatagttattcagcatctgttacatgatgggcagtgttctgacttatagaaataaaacagtgaactaaacatctttacccttatggagttcgcgttttaacaaaagagaacttcccttaaaatctgcaccttctctaaatgaatgtttagatggcaagaagtcttttgggggaaggaggaatgctgcttgaaaaacagaaaatggatgattcaggtcattttgtgctaaaataataagggatttccagcaactgagctaatttatcttcctaagccagaatctaagacatggagaaaggtttatcaagcatccatctatccattcattcattcaacaaatgtttattgagaacttactaggtgtctggctcttctgtgtgctggggatactgcaatgagaagtttggagaaagttccctgcctctgcagagatattcatctagtaaaactggatcttcccagattgtatattctaatccacaaaaatgaaaattaaaaaaataaaaactcttcaagtacttctttacatattcactttctacatacagagctaataagatgaaagtggttatttgcttttctccataatacaattttcttactctgattattactgtgtgggtccaagtctttgcttctgcaaatgtaatagatgcctgtggcaattgagaatcatcaggaaacagaaaaaagaaactttctcccacacgtctgtctcagagagtagtagaccatttgtcgtgccatgggcagtcctatttggaagatgtgagatgataacctgactatgaggttaaataaccccccaaaatgcttctgcttcagacaagacagtatatttacaatgttgtaatatagagtaaatgtcactgtataaaacaaaaatgtggatctgttattcctagtttgaaaactggggcacacatgagaatcacagaaacacagattgctagacccacccactagagagcctgattccaaaagaattttcatttctcccaatccccagtgatacctatgctgatggtccagaggttgcaccttgagaaccactgtgttcttctttcaatatgtggctgtcatatgaatataaaatggattgatctgtagataatgtaaattgtaatatttataaaggaggtcattgtattcatgtaactgtctatggctgggttacccagtatggtagccactggccacatggagctattgaactcttgaagtgtggctagtgccatatgttgaaataaaaatattttggatatcttacgttaaataaaatatattattaaaactaatttcacttgatatttaattttaatgtggctactaggaaatttaaagttaacatgtatgactcacatttcatttctactagataacactggtctatggagcttttctaaacatgtatatgctgagggttggtttttatagaacttagaagtttttttagaatttaaaagttgaatttttttttttagaatttgagagttaccagcagaggaaacgaaggtattgtgctttctttgtaaatagcttctcaacagtatcctttttattcagttgttttgagtagtagttaatgagataatattcactggaagattccttcaatagatatttgccaaccttcacatcttaattaatgtggctgttgggtcagtttctcccaaatttaccttagttccttggaagtgttaagaaatgctctccttcagaagcaagggttattaaaggtcactgcatttccctttttagaatccattcaagctccagttgcaatacacatagatcgatttcagatagatcgtaaatctaaaagcaggataatgtatttatgaacttggtgggagcaaagactttttcaataggacatagaaggtaataaatataatggaaaagatatgtaaattggactaaactaacaagtattcatcaaatacatctttgagcacgaaaaggaaaaccataaagtggaagaaaacatttcagtacgtttgttcgacatatccagaatttataaagaactccatcaaatgagtaagaaaaaggaagagaacctaatgtgaaaatcggcaagggacttgaacagatatttcacaaaagaggatatctacatggccaaggcacatgaaagaagctcaaactcactagggaaatgcaaaataaagccacaatgagatgccactgcacacccaccagaatgacaatcatgaaaggattgacaattggaaatgtgaataaggatgtggagcaactcttaagctattggcaggagtgtaaattgagcactttggaaagctgactggaacactctactaaagccaaatatactcatatcctttgacacagcaatgctactcttaggtatgtatatacccaataataatgcatcatacatgtcaccaaaagacacgtacttgaacattcaggcagcattatttttaaaagtcctgaattagaaacaacccaagtgtccatcaacagtaaaatgaacttacaaattatggtatagtcatgctatgatatactagacagcaataagaaagaacatggcctatcacgacatgaattaatctcacaaatacaaaatgttgaatcaaacacacttagaattgtatgatgtaatgattctattcatattaagtccaaaatcaagcagaccaatcaatctctggtgggagaagctaagataatgttttgcctttgatggagttggttacttgggcacagcttgaggaaggcttctagcattttcctccttggactgcactgtggtttcaggaatgtgttccctttgtgaaaagtcgttaagctgtacccttttctgtatacatgttatacctaaataaaatgtttaacaaaaagagagaagaggaaatacaaagtcataccccatgaccctcccagaaaaagtaactcacctctttaaaatacacacacacacacacacacacacaaacacacacacacacaaacacacacacggccatcactgtatctcactacttaaaatttaaaaataatatagactttcgttttatacttaaaactctagtttgcaattgaccttcagaaattgttttaatataataagattttttgcagctatcagtccaatttatttatcttaaattcctcattggacatatatcattgaaaatcaaaactagaattatagtgcaaagttaaagtgtaagtagtatattgttctatattcaattcaatgtcatctagctaatagagatcttatgtgattcaacttgtcctgaaaaaccttaaaaaaaaaaggaccttgatgataggcaggttctatagaccatgagatttgaataactctatatacagttaattccaaagatttatgacaatcataaatttatacaactgatatccaattatttacattagattgaagtgagaaactagatagaggtattattttaaaagtataaattgttcaacacagcttattcaaaagtgtttatgagtgcatggttatattccttacttgtaggcagctggtcgtaatgcgtatctttataaagtgttaatgagctttattatatgactcaaaatttatttgctcaacagcatttttggttttagcatgtaaatgtatgattatgatacagtaacacataatatatctgcagaactacattgaaattctacattactgacaagtttccactaatgtgaactatatggctctttgagttagaaatgtctgtaattactgttctttggctttattggtattacttatggggaaattgtgtagtgtatgtgcatgtgagagagagagagagggagagaaagggagggaggggaagatggaaatggacaggtaaacgagagatgaagagagagagaatggaagcagaagggaggagggagagaaactactacaatgaattaaacttttgtttctgcattgactttttgcatttggagaataaaaagccaaagccatttctccttgccatctgtgttaagagggtcttatcccactcttagattatcaagaaaaatgtcagatttacaaggagtgccacttttatcccctttctgaaagaggaatatcaattgccttcacaaaggatcaattgtattctcttatcataaatgacttatttgccatccacggaaccaggaaagttgccattcttctttgcttaaccttaagtcaattcaactgcaggacactcagaataaagtggaaatatattataatattgacccatgatttgctggtagaggagaaactagcattgctactgtggcagtcactttatatactttattttatttaattcttttagtaaactcataatatagactttttgttttgtgagttcgtgtgttggtggggagagagtaatgtatttatttatttaattttttttaatgaagaaactggagatggaagaaatccaggatttgtccatcaggctcacacagctgccaagctggtattgattggcccaggtgaactctcactcattctaggataacatccttcctcccagttttggaactttgtcatgaccatgcctaaatcttttaaaatgccaaaatttccaaagtttgtgaaggtatattttacaactatttgaaaaggcatcatgaatacttaagaactagggtgatgattaatttccaaattttcccctctaaactaattaaatgaatgatgttgaatcatctgttttaaaaaaatcactgagagttagaattcctttagtgttgaactccttaatttaaattatattatatagaagtttatccatggaataaaacataacctaactgccagttgaaataaaattgtggctgacccttccaaagttctgtggataaactcctatgcatacagcagcgaggatgaatacttaacactccggaatcccattctgagagcatatttgcattttcatacggaaggtgtgatgccaacaagtaatttcgaaagctaattacaaaagccatctttaaccttcttattgctttcacacaaagaatgtggctttaatatttcttggtgttaatgtacagttagaagcgatactgatgaaacaggtcctgatatctggctgttttcttttacatgaaaaattctgactatgctcaagtactctatcagagtgagcaattgcttcagtattaattctctggaaggaagaatgtataatcagcaaagtttgaacaattagtgatgtatgtaccactatgtacctgggcaggttgagtgatttctttgcacatggacttaaacaggatgctgcataataaggtaggctttgcaatcttagaagcttttagagggagggactaccagccccatttcaagataagggctgtgattgcctgtcaaatcctttggaaacaggaccaagtttgtaaatcaaaggtcaaaactaatgacctcaaataaaaatgtttataaaaataacttgcagactgaatatggggttttcttgataggaaataagtattgttctgtgaagaaatgggcaatattgtttagatataaagaatgtaagagggacttttattggagtacatttgtagttttcattgtaaataatagtaatagactatgatttgtgacttatttgtgaatattaatcattaaaattaggtgattattataaataaacgtgactagaaatccatgatgtcttcacaaatttttgtccagagcaaggaaaggacacctatgcaaaagagcctatctggaataaggttgtttactatttaaaccctaccaagttcaggctattcagtcaaggtggtacagactggatggtggttgaaaaatgaccgacagtctttttctttccgtagagtcttatgattttcgtgacagttgcctaaagctttttggatcacctttaattctaataacaacttttcttctttaccctcgctcttcctttcctcactctctcccaccaccgtctgagtgtcctcagtaatcaagtgctttcctcttttggtacagatgtctgttgggactctatgggttttatcctggtaactcagggacccctgcgtttgaaacacctacaagtgcatttcttcttaggcatgtctgagaatatgtttctaggaaactagcaggcagctgcaaggtgcacagatgttgttgccctgcctatatgcccttggctttctctggctgctgagcctgtgtgtggggacagacagaaatgctgaggaaggctgggtaccatggctcacacctgtaatcccagcactttgggaggccgaggcaggtggatcacctgaggtcaggagttcaagaccagcctggccaacatggtgaaaccccatctctattaaaaatacaaaaaattagctgggcatggtggtgggtacctgtaatcccagctactcgggaggctgaggcaggagaatcacttgagcccgggaagaggaggttgcagtgagcctaattgcgccattgcactccagccttggcaacaggagtgaaactccttctcaaaaaaaaaaaaaaaaaaaaagaaagaaagaaagaaagaaatgctgaggactacccctggaagaatctctctcaaacctctcaaaccagtgatggataagagctgatggacaaatgctgcagcttcctagatccagggtgggaagcaggggctggcagtggtatcaactctgaggtacatttaccttctctccagagaaggtccccagcaggattgaggcccagttgcccacggtactcactttctcattcactcaccttgcattgacagtcttcttttccctgaattatttccctacttccctaccattgcttcctgggactaattcccagataaactaccctgcactcaaatcctcatctcagagtcagtcttctgaggaaacccaatctaaagcacatatttcttctttcaaaaatgatatgatataccagaccaatgctgattttcatcaaaagctaattcttcctgggaattagcagtccatattgaagaacagcagccactacacaccccacatgggggcattttagatggtgtgtcgctgtcagcctgccctctgcacaactccactgggcattttgattcttcttcctgtgaccaccactctcctttgcctttctgaccagggacagttcggctgacagtttttctatcagctgacctaagtgagtcaattcctatttttatgatactgttgtctgactggctgagcaaagaagattgtttttgtgaggatggagaggtgtatgtttttttcaaatatctagagtacagcatcttgttatcctcaaagaagaactgatcttcatgtgagggtgtctgtatgtaaatgttcttatgagctctgatgcctgcaggcctgcttggcaattatgtgcctcagaatgacccagaagcccagatgtgttccagttatctccatacacactttaaataagcacatatgagtctacacatgaattcacagagatatctaagagagattagctgttaaactcttcttctcactctaataccaagatacttcaaaccccaaggtaatcattatggatatagaggatcagaaagccatttcagataaagacttagaatctgaataatctatttatctagttcaagttaccgaaatagcttcctccaagccaatttgcagatgtcccttgcttatcagcggtttcattgctgagttccaaatcattaaacacttgaagcatgaaggccagggaaacgagtaattattggtttggtgaagttgcaagggaggttagggaacccaagagagagttaggctgccggatgtagacacaaaggaagaagaggggtgaaggaggagaaataattaagagtctgcttgcaatcaattgagtgtccatctgctattgaagtcatcgtaacaccaaatgcaggtaattggtatttggctgagtagacaattaggctggccacagaatgaaaggaagccaaagactggtgagtttatcctgcatccttatttcctcctctttctgaagcagagtttgagaactgagaagtggcagacagaacctcccacggtttctgcagaggggagtttgcagagcagtgacacctcctgactcagaaaacaaatgaagcagtacctgaggagagaaatgttcaactacagacgttaattatacaaacaaaggcaataagggctggcggttctttgggaacaatttgaaatatcacttctcttatataaacgaagttgttattgtgatgggactattttctaatgattcaggatgctcatttagaatcatatgactgagtccacattaatgtcactgcatctgcagtgatgaaactacgttggtgcagtggtactttatattaattgaggcttctatcacttggtaatctgagaatcatcactgagtaggactaactagaaaaatgtaagttagaatttatatagcacacgtttttgccagtcaccgtcctaaattgtttcatatgacaacacatttaacaactccagattttttttgctagtggggaaactgaagcatagagctgcacaggtgaagtgttggagtgcccctgactcactcctcaccccatcctgtgttcatgctatgtgagcttcaacctcttggtgtcctgggaggaattaccaatgtttcctggtctctgcttgtccctggctttgcttggaggataagaggaaggggccatcactgggcagacaaggatgctatcagcagcaggaaagaagccccaagcaggggagccttgtgcccaccaaggacagatgggcagtcagcaggagaaccagggacctgagtcagggttggttatcaactgagtggacatacaggtgttttccctctgttcttataggccttggggtatttcaggtctcctttgaagaagccgctcgagtttaaagagattctctgtcctctcctaaataacacaaagtgcgtggtgttggggaggcaggcagaggggtgggaagtagaatttgtttgaagcaagacatttcctgctggcttctcatgggactctcagaagaagtctagcttggaaataggaaggaagcaaagtctgccctctgtttaaatctgcattttcaccctgtacctgcccttggtttccaccatggctgttctctggccagttttagactatttccgtctccccaaagggctctggcctctctctcaccttggtgtctgcttatatatttagcacatccttatcccagtccctgcccccaccatcgccaatccatccaaccctttccccacatctcagagccttcctggacccagcattcaattctggaatggataatccctacacacatacacacagatgcacacacataacacacacacacacacacacacacacacctccctctcttgacttctcttcatctaagcactttttataccccagtgcaattagatcattgctttttcaggtaattgtttctctgagtgctcatggggacaaagattccctattctgtgcatctatgtgtgtcctcagtgctgggcacatggtggacacagaataaatgttaattgaatggacaaatgaactcaccctagtgcctcaaaaaaaagtgtttggccgtatctgaagccatgatgtgcgggtttcaaaatcaccatgtatttctatgtctggccccatgcttttgaaaaacatgaaacctggagaaactctgacccagttaatacccaaagacgcccctgtatgatgagcatcatgtaaagtaatatttttggtaggcagtgaaggaaatcagaattcaaaatgatatggtaaaattggagttttagaataaaatccagtaaaaataaagagcacaagggaccatgattgggcaggggaaatactttacaagttagacagaaaatgataaactacaagttaaagtgtcatttgaagatatccaggagggatgatgtgactcaagcttcctaaaaatgagaatcttggcactgtatctaaaaagaaaaatacgcagacttgatatatgcagtagatgctctctgagcgccacccatagacattgagcttttccatttgctctcagctgccagtatgggcagccttggggctgaggcctcttttttccatcctgagcaaagctatacagcctgggcataagataggctggaggtttcagtgaactaacacagtccccaagagcagctctctgccagttaatctctcaggagggggcggctcctttgcctcttaggtgagataatttggaaatacttgctttatacctattctcagagttccctagcagaactgagcctcagtcacacactgagtcactggctcaccagctcacccttaattagctttcttctctttcacatattacatctttgcttccctaacaatggtccctgtacttcccaaataaactccttatactcgatgcttgcctcaggatctgcatgaggggcaactttaaacttcaagaatgtatcagtcaggacacagataagaaagggaatccatcccaggtggttcagacgaaggggtctcaatgcaagggttacctgcagaggtatggaagcaaacaaaagatgttgaggcctccagggactagcagcaacaggaagcctttactgttcctaggtcgaaggggataggggatgcaagtgttttcacaggagctgtaatcatagaaggatgcagctacttccagtgtgccaaagtgggctgcaggggcgcacacctgtaatcccagcagtttgggagaggcaggaggattgcctgatcccaggaagtcaaggctgcagtgagctatgagcatgctactgcactcctgcctggggtgacagagcaagagcctgtctcaaaataaaacaaaacaaaaaagataccaaacaggaagggaaaaggaaggaaataccctggcctgtgtctcctcctgccctcacatcatctacctgtgcctcccagtgactgaacccactcagacaccagtcaccaagggagtctctgtcaaatggtccacatgggtcagcttctgaggcacagagaagggcagatactggatttggcaaagcacacaaagaataaatagcccacatggttttcctgtttattacactggtgacttggctcctgctgagagcacatcagattctagattctattactgaagaatttagaaagcctttttttgccactacaaagatgatttaaaaattaaggcattgagacccattaagaaaattatttaaggcttatttagctcaaagaaaagcagtctgaaggaccaattaatattgttcattgcatgtagcaacactttgctaatcaagaagttgggctccaacatgataatttttttatcagaaacttccaggccagggatgcaggcagaatggggctgtacttcataacacagagggtgtcattgacatgacatgttcagtagataagtactagcagaagtagtaccagctgatattgagcacatgctatgttctagacactgtgctatgagtaccatatcacctcttgaaattctgagaacatcccaattatacagatactgttatcccctttgtattagttttctggggctactataacaaagtaccacaaactgggtgacttaaacaacagaaatttatgaaatttattgtttccaagttctggagctggaagttcaagatcaaggtgtcagcaaggttggttccttctgagagctgtgagggaaaaaatctgttccacatatttctcctaaattctagttgttttctggcaatcttttactctccttgtcctgtggatctctttcttcatgattatttgtggttctccctgtgtatatccgtgtccatattttccttttctataaggccaccagacatattggatcaaggcccaacctaataacctcatctaaactaattacatctataatgactctatttccaaataaagtcacattctgagaccttggaagttaggacttcaaaatatgaactttagaagaacccactcagacaccagtcaccaagggggaaaactgtaagatgagaaaactgagtcagcaaaaattgaagtgacataaggtttacccagttagaaaatgcttaaaaagatactcagtgtgctagagaacatgtgtcacaagcctgagatcagtgctatggaaagacaagggaagtgctggattcagatttttagagatgttagaatcaagctatattcaagaaggaaagagattaaatgaagggctggaaggagagagtgcatttatagtcctgagaagttggggctggcaagtggtcctaggagaaggaaaggagaaaatctggttagatcctctatacagcagcctcctctgagaatgaaggacatttgccaaggcaggcatactccccacttccaccccaaagatccccagtggacatcagatctgccttccagccagcttttagtcttaagaaaaccttctacctatttattagaattcctggaaaagaggaggaaaagctattaagaggcttaaaaatactaatgcctaactgaagaaacattcagcaggaatgagtgtaatcagtttggtcattagtttttttgattgaagtggagaatagaaaaaaaatgcatttatagaaagcaacagaggccatgcaggatgcagtgcaaacatctaaatgtccccgggaggaaggaaaagaggatcatcatgttacattagcaaaccagtatgtccacatgcttcataataaagaaaactggcaagttatcaatagcttttttctgcacgtttctaaaccttgaatactgcccttccttgaagtctttcatggcttgactttcatatcttcaatgccctctgatttaatctgagcaggcacatcactataacaaacagcttcaaaattctcagtgatttaatgcaataggagtttcttcttcatactccagtccagtgtgcatcctggaaccataatccttagggagttttccagggtcacataactagtgtaagtggcagaaccataatctgagcccaaacagtttatttccagagagtcagtgttctcctaacaactacagctcctggttccagtatctctgtagtcacagttggctaccttctatgacaaacagtccaaattcccaatggtttaacacaataaagtcctatttcttattcacttcatagtccaatgggactggttcttgttggcagtcttcttccacatgctccatcagggacccaggcttttgttccactctcctctgtccgcagagaactatctattcaactggcatgtgaggagaaagagaaagctaagatcagtcaggggaattttctggaccattccaagaagtcaagtaaatgcactacattcatatttcactggcctcactcagtcacatgaccatatcccagccctttgggagactgagacaggaagattgcttgatcccaggaagtcaaggctgcagtgagctatgatcacactactgcacacctgcctgggtgacagagcaagagcctgtctcaaaacaaaacaaaacaaaaaaaggtaccaaacaggaatggaaaaggaaagaaataccctggcctatgtctcctcctgctctcacatcatctaccagtgcctagatacaagggagactgggaaacataatccactatatgcccaggataaacagggaaaggatttggtcacaactttatatctcagacaaatccatgtgtaagaagacaacaaggaggaagaaaaagaagaggaagaaggaaaaagaagcagaagatagatcccccaggctttataatatctcataaggagttgtatttaccttcatctggtcctatattaatggaaattacattgcagctgcaaataggcagcatcactttctgaaatttctgagaaatacagattatcttccacatgaatgagctagtgtgtagaaacttcattcccaaaaggacagctaaccgagtctcactctatgtagaaatataattattcaccgactgtggggcatgtatttttccattccttcggagaacagagccagaggcaggaacctaaattgcagcatgagatatttttgggtaaatagaaatgtgagggagtgtgtgtgaatatttgaacagcattcctaagaaggtttgtggaatttgctctggagatttctagtgaacaatatagattcacttcggtccctaaagggaagagtatcagcccaactattagtatccaagtgttctgagagtgcagattacttaagcgctaagttttaagtcctagcaccctaatgtctggaatggagttagagtaaatcctacagcttctttcacagaatatataaagctaccttctagagcttgaacatcattagacatttttacttcacctgtggatcatcccatatacaagatgttgtcattccaatggatgaggagctgtacaaaatatggcaaccccctcttgcctcctttgaccatcaaagtgaatatataggcatagacttcccttcttacacctatttaagaacataaagaacttgtgtaagaacataaagaacctcaagaacataaagtggcacagattgtggggttcacctcaatatgttttccctttccccaggaccttgggtccccgagtgtttgtagtcctaatattcaagttgtaattccccagaccaataaaaccattgcaagctcacttcagtgctgcaaccttttggccactgctttgtgcttcacagcttgggttttgaatctgtaattcttctaagtgaggaaacgccttgagaggaaatgaagctcagaatgtcagattcacctcacatctttcttttctcaccaagatcctcacctattgagttctggctgccctagtaggtttttttgtttgtttgtttgttttctagacagactcttgctctgttgcccaggctggagtgcagtggcatgatcttggctcactgcaagctctgcctccctagttcataccattctcctgcctcagcctcccgagtagctaggactacaggcgcccgccaccacgcctggctaattttttgtatttttagtagagacagagtttcaccgtgttagccaggatggtctcgatctcctgacctcgtgatccgcctgcctcagcttcccaaagtgctgggattacaggcatgagccaccgcgcccagcccgccctagtagctttctagtacctgtcaacatagcttcaaaataaattgtcaaacttttctcgttgtaggtggaagcgtttgtacaacacccttttcagacaatagaattacatctttaggttctggaaggaaacctaagactctctatatagtgaagaagcctccaaaaattaagtaaaacggaggtgtttatagactaacaaaaacagggtatttttcaccaggagacctgaagtaaaaggagaaatttaagcgagagaaaatcaagagggagggatacacagacatgcagaaaggaataaagtatgagagaaataataaatgtaaatgaatattaactatattaaataataatgttttaataaatggctttcagtttgtaaagaattacaaaagatagtatgaagcaaatgcaattccaaggctcttacattgaacaggaagtagtaagcttactaacataaaaaaggctataaagagataaagatttatgttgccatctccagggatattattcaaagaaaagcaaaataatatgcaattatctagctaatagaagggggaaaaggaatgataacaaatacttgattgacccaaaaagccagaaagcagagaaaaataaagataggacaagttgggggaaacatcttttaagcaagatagtttattttaacccgtctatattacattaaatatgaagggactaaacccttcaactgaaatacagaatgtgtggctggatgaaataaaaaagaaataagaagcatatgttgcttacaaaagacatcccttaaatataatgatacagagaggttggagaaataatactaataaaagagaccatgcaaaaaaataaaattaaaaagccgatacagctaagtttacataaaaactcaagtgaatgttaaggcaagaagcatttttagagataaaaatgtattatactttattgaagaatcattccaccagaaagatataacgattcttaatatctgtgtaccaaataatacagcctcaacatagacgcaaaaattgatagaattaaaaggagacattggcaaattcacaactcttggtaactgttaaacaaaccaaaaggaataaaagaagatttgacaaaatagtttgcaaacttaaactaattgccatacataaaacgccgcacctaacaattgcagaatacacattcttctgaagtggacataaaatatttgctaacgctgatcctatcctagggtaggaatgtccctttttgaagacacctctcagacagcctgtccttgggaaaaccttctttggcagcacccctgctccggtctggagcagacccagttaggactccttagcatttaacattcctctacatgtctatatcttcttcctgcaaacactggggattctctccggaggactttttctggtcacagaaacatgctcagggccatgcacacttcagggtagaaatgccaaggagttaatgtgtcaggtacagttcccaactcataaaaaatgggaattgctgaataaatgtcccacttccttgcccttcagatgggaccaccctggggtacaacctataccacctctcagagaccctcatcaggaccgagctccagttgctgacagtagtaacctgttggaaatatgctctgcactggctgcttttttggtcctatctcacttcctcatgttcttaccattcctcctgagatatctcaggtaaaggacttgcactcaaactcttgtttaaggatcagcttctgagaacccaaactaagacagggccccatctgtgcttgctgatataactttaacttactaaattacagtgatccatctccttttgtgagcattttgatggtagagactatgcttttatctctgtggcatagcacctggcacatgacacctaagaagtgctcaactagtgctcagtagccattgtttaatcagtgaatgaagcctgatttgaaactcatcatccattagctctcagcacaaggctgcctgaacggatcacataggtaggttttgaaaaagagagagtagaaacaaatctgaactcttaggaggacaggcctagattacatgccacagggaatatccctaaacatactagtatgtccaaagaacccagaggacctgctatgcagatgcacatggcaccttcatggaagaaaagcaaggctgatgaaaagggatgtcagcattcctgagatgcgcttttgtccatcacaattttgcctggagccagccctgtttattacatgaagacatgctttgttctgttgggattttatttgaaatcaattcttaatttgattttaagggaagcagtgatgttgggaaatgagagcaccgtattattctcagagattgtccttgaaactaaatgggtctgccacatcactgcacgactctactgttttgccctatttataatgctttgtttatctgttgttcccaggaaaataacctcaccttgacctcttaagcatcttttgaatgtcagcaatatgagaattactatgccaggagttgggagatgagaaaaaaaaggtgtttaccatccagaaacataagatataatggaagagacagatgcacagacataaaatgttctcaagattgaagataaggcatgtaccaagaatcttaaaagcacaaaggatagacagtggacccttgaacaatgtgaggcttaggggtactgacctttacacagtcaaaaatccatgcataacttttgactcccccaaaacttaaactacctatggcctactgttgactggaagtcttactgatatcagagtcaatgacacatattttgtatggtgtatgtattatatactgtattattacaataaagtaaactagaggaaagaatatgttactgagaaaaattacaggcaagagaaaatatatttactattcattaagtggaagtggaccattgtatatatcttcatcctcgctttatgctgaggagactgaggaggatggggaacaggagaggttggtcttgctgtctcaggggtagcagaggtggaagaaaatctgcatctaggtgaacacatgcagttcaaacctgttttgttcaagggtcaactgtgtattcagactctgcctgggaatatcagctaaggcttctgatatggtttggctgtgtccccacccaaatctcattttgaattgcacttcccataatccccatgtgtcgtgggagggacccagtgggaggtaatttaatcatgggggcggtgacctccatgctgttgtcatgatagtgagtaagttctcatgagatttaatggttttataaggggctttccccccttttgcttggcacttctctgtccatgtgaagaaggacatgtttgcttccccttctgccataattgtgagtttcctgaggcctccccagccatgtaaaactgtgagtcaattaaacctcttttctttataaactacccagtctcggtatgtctttttgtttgtttcttttgttgagatggagtctcgctctgtcacccaggctggagtgcagtggtgcaatctcagctcactgcaagctccacctcctgggttcacgccattcttctgcctcagcctcctgcgtagctgggactacaggcgcctgccacaatgcccggctaattttttgtatttttagtagagacggggtttcaccgtgttagccaggatggtcttgctctcctgacctcatgatccacctgcctcggcctcccgaagtgctgggattacaggcatgagccaccatgcctggcctcgggtatgtctttattagcagtatgagaatggacaattacagcttcttcaagcttgttctctctaatacattctcgaagggcacaatttcccacagaaataaagatcccaaggagctatgagatcaagacacaaggttgtgaacaaccaaagactattttggaaaaaatgagtcttgttagagcagaaacatagagaaggtagagaggattggtagaaactgaggctagagacaggatcttgacatttgcttgtgccatgggatggcaatctgatgagcccatggtcccttctcagaataatttcaactataggtatttgaagttttcagaaagttgaaaaaattgtaatatgatagcatatatgcttctttattaacacattaaataacaagacctaacagtaggcctaatacctactacaattttaagtggtgatgagcataaacggtatttcaagaaagccactatactgtcctatgatatttaaatacctgtgatttccatgagtgaaagagtcacaggtactgctactacgattatggtctgttgcttacatgcataatttaggaaaatatcagtttccattcacagttaaaaaagataaagttaacagttttctggtcaaactgaaatccaaatggtcatagccattctggatgaggttaagaacacacaggtgagaaattggctgatgttccaagagttcagacttttatctacaagtaaatggaaactagtgagtgtttacaatggaggggttgctggaatcaacatttttttctaaacttaagtgtggtcacacccagggtttcatgtctaaggccacctgttgcagcctatggtgcaggccctgctcctagaaaatcaaagtagaaaaagaaagaagagaccaatatatctgagagtctacaagcctgagctgctcagccttagctcttctactccctctagtaaggacagagcgttatgccgaggctggagtgcagaagccggggtctgaatgtgatgcatcctattagaatgctgagtcacattagatttgttggaagagagatcccaagtgtggaggaaaacctgagcttcatattaaagctgaatacattatgaagtgaaagagaatatttttattgctttaaatttttaaaaaagacattcctttccttctgtcttccatgcaaaataattataacaacaaacacacacacacacacacacacacacacacacacacacacacatgcacataccctgattttgatttccctgtggttttcaggaaaacatatttattttcctctgtctttagatattcctcaatgataaaatgttagggtctctgaaatgctagccaacacccatttggactcagtattctctaattttattgtcagaatatcagatatgcattaatgcacaaaacataagcatattaaatatcagcaaatatacaaaactgtgaacacaattgagagtggattaacaaagtttctttgtccttctgggtattgaattatcaccaatcaagatgatttcatatcaaagccaggacagtgtttcatgtatatgtacatatttatatgtatgtgtgtatactgatctaccttttcaatattctcctaaagactttcctttattttcatattgagaaaacacaattcagaattcagaacaagtccctttctccctctatctcacttcccaaccatgttttccagcaaagctgaaattgaaactatcctgttttctttcctgtttgttaaaaaatgacatcaataatattcacacagttcccaggaataggatcctactaaactctctcttatttttctttctgtagaaattgtaattgtagtttattaggtccttcaaaaataataaatcataattttataaaaagatcaagtgtaatattttagtattcacattaaatatatagattgacccaatttaaataattcatatattgcctaaagcagtttgatatggtttggctgtgtcctcacccaaatctcatctttaattcccacatgttgtgggagggactcggtgggaggtaattgaatcatgggggcaggtccttcccgtgctgttcttgtgatagtaagtctcacgagatttgatgattattgtaagggggagttttcctgcacatgcttttttcacttgctgccatccatgtaagatgtgacttgctcctccttgtcttccaacatgattgtgaggcttccccagccatgtgcaactctaagtccaattaaacctctttcttttgtaaattgcccagtcttgggtatgttcttatcagcagcatgaaaataaactaatacacagtttttgttgccttactttaccaatttacaattatagctatgttgtaagtgttacagaattaaaaattaaacattgctcataacaagttgtcaccaaatagccttgtgactaacagaataagcagcaagacacatggtatcagttgactaatttgtttgttatccataaatattttggctgactatatagtttgcctttcttctttagaaacatgccttcctctcataaatgttaattttaagaatgtgaactgaataatgtaacagaatggggaagaaatgaatatttggaacaattaattattaattggagctgggtatggtgccatgcacctgtagtcccagcttctcaggaggctgaagtgtgaagattgcttgagcctgggagctcaagaccaacctgggcaacagagagagactccatctctaaaataaataaataaataataaataaataaaattaattattaacctgttaagttttatccattcatttattcaaatgttgaacaaatatttaaaagcacctactgtatgccatgctgtgtgctaagtactgaggtcctacttgtccttttggttttaagttagagctttgtcacccccaagctgtatgatccttattttattgtccttgcattagaaaagaatctcctctgtgcttctatcagtatgcagctggactgctttaactcttctcctcttttattttttgtcataacttgtcattttttaactcttctcctcttttattttttgtcatatatatttagttctgtctactcagaaagcttctaggatctccatagccttgcaattctcagtctgaggaaacccctttactcaattaaaatacaaacaatttgtaaatgtactgttttcctcaggagccagagttcctatttaggaggaattgtgcagcagatgttgtaagtggctcacccagatccccagttccccctccttgggccaccccagctgctgtgagtgctgactactaatgacctgcagctagctccttctctgaggcattactctcagccaatggaagcatctccacccaagatgctgaactcacaggtgatgactgacatgggttacaaacatgactgacatggggtacaagctccctttgcctgggaggcagcttatgctccagagttcccttgatattcccttgatattagactgagactagactccagctcaggtcacagcctgttcagctctcacttccacctgccctgctttcctgaaagcactccctcaataaatctcgagcatttaaatcactgcttcaggctctgcttctgcatactcaacttaaggcagcaagaatcctgccaaattttgccttcctttgtctgccaaaccaggacatctcagcctgttccatatgactttcctcatttcaaataacaaaatcagttaagattccaatgtcaaagaaatgtcaaagattactccacagcaagatctatagagggaagtacctcagagggaaacatataagaaagcttgtatcgtattcttcctctagtattgatccccaagtacactgacttcaggacacatagggtctcaagttctttttgaggggtaggcagggagagggttcaattccacatatgcctgttgaatctatgtatcttagtaatcagagactctcttggttagctcaggaaaaaaaaagggtgaggagtcattgttagaactgtggatctcaaggatagctaaaaataggccacagtagaagcagacttggaaactcagtcttactcccttcagcaaggccgtgccattctcattctgtttctgctgctagacttcctggcttttggggcttttctttcctcataattcccatttacatggggccattatgcagcccagagtcttctgtagcacaacatatcaaaatgtattacatcagtcccatttgcatgctaactagcagaatctcctagttttctgattctaaatccatgtttccaattagcccagtttaacttttctcaccagattttaagctccttattgggcagcttatgagcagactgcactggggtcagtggccctcccaggagtgctgggggttggtagggcaagtatgttgtctacaacaattaggcttccatgcggtaggattgtgggcaagggaaaatttcccagagggggtctgtgggcagaaatgtcccataaaaactataatacagtatactgatggacaagtaggagctcaaagaacagtttcaacttgattattgttagaacagataagaggaaaagttaggaggcagataaactgccattaaatcaaccagataaataatagtagaatcatttataaatactgttgacatctcctcagataacatataacctccaaggctttagagatgtactatccagtgtggaagttactagccacatgtggttatttaaattgaacaaaattaatggaaattaaatataattttaaaaattaactctgcagtctcagcagccacatttccagtgctcaaaacctccatgtagctcacggctactctattgacagatactgacacatgtccagcatttgaaaaaagttctcttggaatgcacctatttaaaagtattattgcttttatttaaggaaagagatttgagagattcatttcttttttaattctttttttttcttttccagtaaatatgataataaacacaatttgaatccctttcctggaaacacattaaggattaggtgccaggtttcttccatctgttataacaggaaggccaactggtattttgtagttattttaagtgtaataatatgtcaaacacactaaaaacattctatgtctgtcacttaatgttaagatttctctcttgcccatgttatttttttcctttcatatgggcaattgttttgcataaagatgaataggtagagagaaggggaggtgagcattattctttgagtctctgtaccttagagtcagaagggacctcaaggatctaatagacatttcccatctaatgcaggaattgcatttgctgcctcctttataggtgaagctattgctgctcaagaattaccaaatgcagactactgttcaagagccatctatttgtatctatacttcctatctgtatatgtgggcaaagtaaaaggaaaggaaatatattattgtaattctgagattgaacagatacttattttcaactttttgctttttaggatttactgagtgtattagtctgttctcaagctgctaataaagacatacgcgagactgggtaatttgtaagggcaagaggtttaattgactcacagttccacatggccgaggaggcttcacaatcacggcagaaggtgaaggaggggcaaagttacatcttacatggcagcaggcaggagagcgtgtacaggggaactcccctttataaaaccatgagatcttgtgagacttattcactatcatgagaacagcacaggaaagacccacccccatggttcaattacctcccacaggcttcctcccatgataggtgggaattatgggagctacaattcaagaggagatttgggtggagacacagccaaactatatcactgagtatctagtatgtgccaggatttcacatgtatattaaataaaataaccatacatactgaaatatatcaggcattcattaaatgttatgtatataaatgggttgcatttccagttattgtcatgtaagcataatttggagtaaaaaattttcatggaaattattttaaaaattgatgcgttagttcctaggctgccttcaaaagctgaattaatctataatatatggctatatcaagattacctctgtaggaaaatctagttttttttgtccttcctcgctgattatctctcttctttctttcatttgttcattccttcaacaaattccacattcagagactaggaacaaagctctttcttatctagatttctgtagtgtgtggctgatttttattaaggatcaggagttctttgacaggaacagggcctattttaaagtcaggggtaccttttactgaatggacaaaaactggaagcattccctttgaaaactggcacaagacagggatgccctctctcaccactcctattcaacatagtgttggaagttctggccagggcaatcaggcaggagaaggaaataaagggcattcaattaggaaaagaggaagtcaaattgtccctgtttgcagatgacatgattgtatatctagaaaaccccattgtctcagcccaaaatctccttaagctgataagcaacttctgcaaagtctcaggatacaaaatcaatgtacaaaaatcacaagcattcttatacaccaataacagacaaacagagagccaaatcctgagtgaactcccattcacaattgattcaaagagaataaaatacctaggaatccaacttacaagggacgtgaaggacctcttcaaggagaactacaaaccactgctcaatgaaataaaagaggatacaaacaaatggaagaacattctatgctcatgggtaggaagaatcaatatcgtgaaaatggccatactgcccaaggtaatttatagattcaatgccatcctcatcaagctaccaatgactttcttcacagaattggaaaaaactactttaaagttcatatggaatgaaaaaagagcccacattgctaagtcaatcctaagccaaaagaacaaagctggaggcgtcatgctacctgacttcaaactatactacaaggctacagtaacccaaacagcatgctactggtaccaaaacagacatatagaccaatggaacagaacagagccctcagaaataatgccgcatatctacaactatctgatcttcgacaaacctgacaaaaacaagcaatggggaaaggattccctatttaataaatggtgctgggaaaactggctagccatatgtagaaagctgaaactggatcccttccttacaccttatacaaaaattaattcaagatagattaaagacttacatgttagacctaataccataaaaaccctagaagaaaacctaggcaataccattcaggacataggcatgggcaaggacttcatgtctaaaacaccaaaagcaatggcaacgaaagccaaaattgacaaatgggatctaattaaactaaagagcttctgcacagtgaaagaaactaccatcagagtgaacaggcaacctacagaatgggagaaaatttttgcaacctactcatctgacaaagggctaatatccagaatctacaatgaacgcaaacaaatttacaagagaaaatcaaacaaccccatttaaaaagtgggtggaggatatgaacagacacttctcaaaagaagacatttatgcagccaacagacacatgaaaaaatgctcatcatcactggccatcagagaaatgctaatcaaaaccacagtgaaataccatctcacaccagttagaatggcaatcattaaaaagtcaggaaacaacaggtgctggagaggatgtggagaaataggaacacttttacactgttggtgggactgtaaactagtgcaaccattgtggaagtcagtgtggcgattcctcagggatctagaactagaaataccatttgacccagccatcccattactgggtatatacccagaggattataaatcatgctgctataaagacacatgcacatgtatgtttattgaggcactattcacaatagcaaagacttggaacctacccaaatgtccaacaatgatagactgaattaagaaaatgtggcacatatacaccatggaatactatgcagccataaaaaaggatgagttcatgtcctttgtagggacatggatgaagctggaaaccatcattctcaggaaactatctcaaggacaaaaaaccacacaccgcatgttctcactcataggtgggaattgatgaaaacacatggacatacaggaaggggaacatcacactctggggactgttgtggggtgggggggaggcagaagggatagcattaggagatatacctaatgctaaatgttgggttaatgggtaaagcacaccaacatggcacatgtatacgtatgtaacaaacctgcacgttgtgcacatgtaccctaaaacttaaagtataataataataaaattttaaaaatcaaaataaataaaaataaaaataaagtgaggtgtacctttataataacttaggcttggctgggtttcttctcgtagggtaaattagaaagcaaaattggaattagcttgtggccacttctcagtacccaaaccaaggcagagatgctctcagcttggatgttgcctagatgatgatagataaagctcttctctctttttcttgagaaaaacaatattattttttgatatactttcctacaaaaacctcaaattttaatccaaaaagaaattctagtccaagcaaagtagaggaggacttttgatatgattgcacctaaatatagaagaaaaggcatatgtgaaccacttaaaaataacataatcacttaaaataaaagttaaaaagggaaattcagtttctttaagatatttctagaaggaggcttgttatatatgtaacactgtcatgtgtatatagataagtatataagaaataaaaactagcaagtaaatatattggtaatctgagttcaagagaaaccatcctaaatacactttttactagatctaaaataaaaaccaaatgacattttacctccaaaaatggatattgcccactgtttgaatatttgctgtgtgtgtcttaaaagatgtgttacatatattgcattacagcatgtgtaattatgaatataacaatattttttgtaaatcacttttattatagacatgtccactgattctacaatcagcacaaggttggattttgtgttccattatacattaaaaatgtctcttatgatcctgtggatccaaggaaaagatctctcgaatatgccatgatattgggcaccaggaggccactgagtcagtgctgatctgcatgcaggctcctcgtgccttccccagcaatgcccagtatagatgtggatattcccttttatttctgcacttaggtaactgccttaatagatccagatcaaattaacaaaactgaaagcatcttaataaaatttatcagtagaacagaggtgatgatgtcagtcaagtaaatagcaccctggtatccaggaacagctgatacaacttctaacatgcttttgtttattggtttgagcatgccactccaatatgcaaaagtgtcgttctcgttctcagtgttatgctctcgtaaaattcacaggttccctttccaagtcgccagtccccttgttactggtccatctgttgactcccttacctaatcctggctattttccttcattataagaaagggcaaatgagaaagagtggatgactcagccttcatgatctggcatcctgctgaaggcaggtagatatcatctttgtgtataaacataacattatatttttcatctaatcatgaaggatttaaattcaggcaagaatagactcccttatacattcttcaggcaacatatcacaaaggaaaaaaaatgaaaatactccattaggctcaaaatttcctctgaaaaaacaaagacatagatatgtacatgctttatgtttaggtttctattaccactttggttggatttatgtgctttcagtatgaatttaggagtacatgtgctattgtcttaggttgtatgagagagctatgaatttaaattcttagatggataccctaacatgcggcttctctttttaaaggacaaattatgtgaaaaagcaattcttaaggaaaatagttctttcctcttgttctcagtggaagctgtcatgactcaaatggttcaaggagattctactcaaatttccatttctcagcttctgatctatttggtataattattaaccatatattcaaaaatgaatatccttaatactggcaagctactattacttaagtgattttagatactattaaacacattaggcaggaaatgttttgaaaaggtactaacagagcatgcaacaatttctcatttcatatgtggaaccatttggaataaacacacatacataaatctgcatatatacattatgggctttaagttacgaaattctatgttctagtgatctactgctgtgtgacaaatgaccccaaaacttagtgttacaacaaagacaactttatgatatctcatgattcagtgggtcaggagtttgatcaacatttggttgggtgatttttatattccacagggtattattagagcttccccagtggtatgcaactagaagatgaactttctggaaaatccaagatagcttcacatatgtagaccttggtgggaacagatgaagggttgggttcagctgggactgttgactggagcacttgtacatggctagggtagttgaattcaatactcagggtagttgaactcaatactccaagagcaagtgctccaggagatagtgaatggaagctgctagtctcttagggcctgaacacagactggcaaagcataactctgccacagtcactataggcattccaggctgaagaggaggagaattatactctacaatttttttttgacatggagttctgctcttgtcacccaggctggagtgcaatggtgcgatctcagctaactgcaacctctgcctcccaggttcaagagattctcctgcctcagcctcccaagtagctgggattacaggtgcacaccaccacgctcagctaatttttgtatttttaatagagatagggtttcaccatgttggccaggctggtctcaaactcctgacctcaagtgatccaccagccttggcttcccaagtgctgggattacaggcatgagccactatgcccgatctactctacatcttaatgaaagaagggacacaaaatttgcagccatgtttaatctatcacactctgccctggctgagagggagaaatgtggcatcatggttaagaatgtggaagactagatctgtcactgattcactgtaaggttgtcatcttttcatctaaaactaagaatagtaacaatactcatccatatctcatagagttatgctaaaaatcaatgtgacaagcaacatacctatacatacacacagatacaaacacacatacaaagcactcaacaactttttgctatttgtttttttgttttgttttgttttgcattttgttttgttttttcttgttcatctgagcttatggtggtggaggtggggagacattgtagccagaagtaagaaggccacttggagaacaaggaaatgggtatcattgatcaggcaatctaatgtcataactcaaaatgttgtggaacagaacttgtctccacggtagtatttcctacttggttttgctttttgtttttgtggtttcattctttctttctttctcttttttttttttcattccgccttttgctgttgttgtgcttttgttgaagttttttttttttttccaagttctcactgcattgaagaatgatgcctgacctttcattctttctgttattgtcactggactccagcttgctattagtgtcttcctgtgggcagtcaggaggctatatcatgcttgtttcaatacagtaaatggctaatttcaattatatgactcaagttatgccctcaaatggagggcatgtctgtaaatatctttgtaaagttatatcggattctttctcatttctgttattctgagaaatagtgctgaactctacttttcccttcctccttcccttcttcttcagctcatttacaactaaacagaaagcactttcctggcaggctagttaaggaaaaggctgagatattgtagcaaagagtcttacaacatcctatacattaaagacggaggcttatttctctttcttaatagcctgggccatggagtccacaacataacccaggggtccagtaaaactgaaaggctctgccatccttaagatatggttttcaaggttcttctaattatcaccatttctaagcagtgggaaggaaaaataggggccaagtgcagggcaagcatttctatatctttagaaaaatgaccagccatgcacagtggctcacacctgtaatcccagcactttaggaggccaaggtaggaggattgcttgaggccagaagttcaaggtcagcttttggacaacataacgagaccccatctcaaaaaaaaaaaaagaaaagaaaagaaatattgagacctggtggagtgcaactgtagtcccagctaatcaggaggctgaggtgggaggatgacttgagcccaggagactgagactacagtgatctgtgatcatgccactgcactccagtctgggtcacagagcaagaccctgactcaaaaaataaagaaagaaaagctagaagtcacacataatttccgcttatcttaatggcaagaattttgccacatgttcacagctagcagcaagggagtttgaaaattaaagtctccagccagcattcatgtggccaggaggaatggatttgtgggcccaggggagtgggtggatgagatattaaattgaatgccacatttcctgctttatcaaataactcagcattttaataactgatacacaatggtgactatccagttgtcctttcaagccttacaagttggccaggcacagtggctcatgcctataatttcaacactttgggaggccaaggtgggaggatctcttgagcccaggagtttgagaccagcctaggcaacacagggaaacttcatcactacaaaaaaaaaaaaaaacagaaaaatttagctgggcatggtggtgtgcacctatagttccagctacttgggaggctgaggtgggaggatcacttgagcccaggaattcagggccatgatcatgactctagcctgggcaacagaacaagactctgcctgttaaaaaaacaaaaagactcacaagttgtagtaccaaaatatgcctgccatacccattttttgagggattcttttttttagtaaaataattagacatttatgggtcaaggcagcatctcaatggatgtttttctgattgtaaatgtcttttagccttgtatttatgaggctacaatatgtaacatgaagctgaaggctatgaaccattttttaaaacaataactatcatccaagcatgaatattgcctttggtactacttaggacacaagacgataactacagaagaatgtgggcgaaataattaaaaaccacagcagcagcaaatctcttggttattttataaaaacgaaatgatgttccctaagggttaaaggctatctgacaaagttccattgtgtaatcaaaatgtttgtgtctgggtagataaatatagtatagatgctgtaatattgcagtttgtcagccatttttgatatatgccttgttgatttccatagtcctgattcccaaaacagctaagaatcatagaattttagagctggatgggactgactgaggtctgatttactggagggaaccattggaggttgaagcatttcttgtgatgacttttctcatgttctttataaagagggtgtcaacccactgaacagtttagggagaaaaggaaatgctgagttcctttcagtctacattaaagctctacactcttggttaggatctatggagccttgctcaggtgagataattgagccccagagaagacacgtcactctatgttgaccattctgatatgtcatctggggctctttctatggtacagtgtaatcctgatggaagggactttctgcctgtgacctatttataaaatcccccatcatgaggtaaaatatgcacttattcgtagctgcctttttctttcaatcaactttatgtttgtggaattcattgaacttttccaaatggttctttctccctgctatgtagcattgcaatgtaggaatatacctcaatttatttatgcattccactactgatgagcctatggataatttctagtttgtgattcttaaatgggaggcaacaaaagccagttacacatagaaaagtagaaatataaagcaatacctttaagcactcgtgttattcaaaaagttcgcatcttctcctactgagggaatatattttaaaactgaccctctcaatggtaatgtaagattccttacctttactcgagagctcagaaaaagcctgctggatatggtctccaaggtgtagtcctgccttacatttccaaagggaagggtaatttttgctgagtttacatatttcataaatctgacattactgaaagtaggatcaataagcccctttccattttaaaagtgcctgagatcccactgttctacaaacttacccaaattaaatagtagtattaagttgaagaggaaaatgcattttggcatttttcaaagatttgaatttcaaaggtttattattatgcataacagagagagggaaatgacaatgctatttaaataaacttcactagaaacttcacattaaatacctcttagacacagcttcaagcacagaaattaggagttattattctagcaatatttccttcctcgatgttattttgccagaccaacaaggggccagtcacatagtgggattgcttcgtaggaatgtaaaacttctagactaaaatcaaggttgggtaaaaacaaaatattgccccaaaccacatcagattttaccattgcaatggtttttgcttcattgatttatacatttaaaggagcaaaacttaatctgctataatgtattacaatagaatccatcttctaaactatgtgataattttattaacaactctattgactgacagtcttccttaaaaatgttactgctgaaaattcacatagtggctaataaccaccattgttgccattttatatttgcattccattccttggctcattaaatataatgtaataaacatatattaaaagccagaggagtttgacatgaatcctgcatctgccactagctagctgagtgaccttaggcaagtgatttcattctgatacttagcttttttctctttgatgtgaggctaatgctatttgctgacagcatagtgttgttatcagaaaaaaaaatgataagtatattagagtaatgcccattgttataacaactagaccccagaacttcattagcttagtccagtagaatttaatttcttgcttgcatgaagtccagtgcaggtgctcctggtcagcaagtggatttcctccatacagcagttcagggatccaggctccttctaccttgaagttttaccaccacccctgcactttgttcttctctgcatccagcctacataaaatccctaatctggaagtgaattatttcacttctgtgcacatgctattggccaggataagtcacatggcagtacctggatgcaaagggagctgggcacatgtttgcccagttgtaagctgggcagctaatttctagggtgagttccagattacagaaaggggagcatgtgaaatccataaacattgtgaagcttttgttgctgttgttgctggtgacattttaggtagtcttcttgttagcgccatcatcctaaaccagcattcccataatcatattgattgaacatggtgaacatcatcatgttatcttagaaaagatgaccaagaagtgatcacagttggaaatgcaagtgttattaactcccaatgcatggtgtatagcactgacaatattttgctgattttcccatcacataggcaccaaattatgctaaattataaattagatatattaaaattccagtgccaaaattattcaagaatcatagtaaaagacctctacagataaaccccaagcttctattgtagtatatagttatctgaagggccttctgtcttccaggaccagctattgtccagagaagagcccagaggggctgaagaaagcccccaggagaccatgctaaggagacatatcctaaatgatcatttcactgtgctaccttttgtggttgtaggagccataggcttttggcccctaaagattcactgaaaaatcactgacgtgaggaagattgattaataggagaaaagaaatacaaatttatttaatgcctttagtaggggcattaaataaatgcctttagaatgaaggcattctaaaatgcctttagaatgaagacccctcttccaataggttacagaaacttatattttatcttgaggttaaagaaagaatgggagcttgtatcctggtaagaccagttatgagaggtgggagaagaataattctattgaggggcaataaatgatagaaaggggaataaatattaacttgtaaatagccttctctagaatttacatagtcattggagacaattcattatgcccataaaaaaggtcttctcaggtgtggtcacatcttggtcttcttttctgcaacagataatgagataacagggaaaggaagaaaaaaacaattgttcaccttggtgggtctggatcttaggcagataaaggaacttcagcttctttgggagagactttgggtggtggagggagggaggccagaggatccttgaggcttcttcagttcagcatgtcaaaatgccatattttggggtattagtttctgagccccagcgtggtctaggaaaatgaatatacctggaaaccatacaatcacttaattacatcaaaactgaaatcattgttttagattaagtaattaaacaatcaaatcaattttcattccactcagagaaaaaaaaccaaagccctgcagtggcagctgatgccctcaatcacagctgctcagacttgagtgcacatcagaatcatctggagagctttgtcaaaatggatggctgagtctccctgcaccacccctcagagctactgagtcagtaggtttgtggtggaactccaaaatttgcatttttaaacaagctccccagtgatgctgatgctgctggtctgcgaccacacttgaagaactactgccctgtgcggcctgacttcctgttccatctctgtctcatgtccctctgtttggccctttgcttacccttcttcaggttcacaagcctccttgcttttccttgaacccaccaggaatatccacatctccaggccttgcacttgctgttgactcttactagaactctttggccagatactctaatgaacggtatcctttctaacttcaaatatttgctcaaatatcaccttcttaatttaaggacatcttctcatccctatccacacaatataaaaattcttttcccctgccacagcattcgccattcccttttctgcttttatttttttctcacgggccttatcatcttctaatatactacgtaattttcctatttacttgtttattgttttctctcctatcatgagagccccataagggtggtaaggatctttgtctgcctccctattgcgtcttcagtaccatggacagtgcccggcttcagcaggtgcacaaaaaatacttgtcaaagaaaagatggagagagccctaagggaagagagaaaggagagaaaattaaaataatcttaatctaccacagatttttgcttggaatgcaaatcacttgtcacttgcccacagccagtttactgcgtaatccacgtgcagtgcctctgttttgccgtataattctgaaagcctttagtgccctggttgcctttatccccctcatgccaggattatcctcaggttcttctggggtttgagagcctcaggtggcaggctgtaagtgagaatctgaaaagattttacaatagaagtgggcatcttcagatttggaagggagtgacagtcagaggctagttattattgtttttgcttctcttattgtgactctcagttcatctgacttaggctgcacaaaccaagcccattcttgccctttccctttggcatacttgctgttatagagaagcaaatatgaagagaagagtcagaaggtgagacttatacaccatagcaggggtttctatacatggaaagttcagtttgaagctgacatgtcaacaggactaccatttctatttgctcagaatgttagattgtttggggacaaggcaatatccaatgacagattctagtggtttaaaagaaagaaacaaacaaaaccctaaagaactatgattttttatctaaaattgtactacaactttgtagtatttaactactcagaagattccattctttactcgtaacagcaaattcccttcttatttggcccagtcttcttcaacaggaaagtttcttatacccaatactctatcgtttaaaattcatataatcaatgacattaaaagtggacagcttgacaacttatttctaccataactttaagaaaacgtacagctctgaggttttcagacaagtggtagcagtaacttatttttggaaataagtcgaacaccattgcagttgcccatatgaagcccatagtctcaccaggaggtggtgctagcatatgaccctcagatggaaaagagcagaattatgcaagtgtttgtgtgacccatctgaccaatctaaataaagcaaaattaaggttacataaggttctctggcttgatgtgaaattcctatgcatcagtttttatattcttgaagattgaaggaaagaaagcaggctctgatgttgttaaacttgacagtgtttccagttatgatttatggaagatctcattttgattgccagctgtgtgtacacatgcacacacacccacaatacaattttcaatcacctgagaagtttgataattagatgagaacaacacctaccacgtatctcatagttactgttgcaaagacttctaaagtctttatacccaattatatggggggctttttacctgttggtgggacatgtagctccttgcaattgcctttttaagagagggcagaagatctcagtgtgatatcttgagtggtgaattccagaaattacagcattcctctgccaactaccagaattagggtttattcttagtgtatctggagctggctcacatgagcttgtgagagccaattgtgctcatctttaaaaatattttttaattaaaaaaatgttctgcaatgtcacattggtagcttgaaattagctatgtgacagtatttgcaccataaaagttgacagacattacaatcagggtttgtttaccctttttccatagccagtttttaaccacttagaagcatgccactgttacttattgtggtcgtttgcaaacataatcctaattctctatgtctcacagtagccaatcattggcaatgtgacattgaagctcctccctttgaaaagcagactaatttccccacccattgaatctgcgttggtcttatagctttcattgtttatagaagctagtggaaccaatcttatgtgccttttcctagcctaggtcttaggaggtatttcttctgtgctctcagaaccctgataccaccacaagaacaagcccatgctaccctgctgaaagatgagagtccatgtggccatcctagaccagtcagtttccagacaacccatagctgaccacagaatatatgaataaacacaggcaagaccataagaaccactcaactggacccagcacaaattatcagcccacagtatttggggccaaataaatggttgtcattttaagtcactaaatgttgggatggtgtgatatgcagccaaagctaactgatcaactaatctactcataattcccagtaagttttaaacaaacaaataacagtgatatttccaggattaaaatatgggccagtatctgtttggggaaaccctacttcgctaaaagacttactgtgaaaatactttatctagttttcctagtcattaccttcagaaagtagatgggtgtatttggatattagtgtcattgatgtaaaatataaaaaaatctggagaatctgggactatctagacgatgtttacagatggtggaccacttcattctcctcttaaataagtattgcttcagccacaggagctgcttttgtttcttgaacaaggcaaatcattttctctcttgaggcctttatccttggttccttttgcctggaatgctctgtttcaagttcgcaagacctgcccctttttatccataaaggctgaactcaaagcattcccaacccaatatctgtatcacgagtaggcctgcagctgctctcctacaagttacactttcataatgctgtccagtagaaatataatgtaaggtacatctttaattttaaatactccagtagtcatattttgaaacacaaaagaggtaaaactaattttaattaatctgttttgttttatctaaaatgtttaccactttaatatataaccaatataaaaatattagtaatacaagcgatcaaaaaaataacctgtacagtattattcataccagctttatgtgtaatagccaaaaactagaaacaaccaaactatccctcaatgagtgagtggttaaacaaagtatggtacatgcataccatgaaatactactcaccaataaaaaagaatgaactattgatacatggaacaacgtggatggagtgctagggcctcgtgctgagagggaaacaggaccatcccagatggtcatataatctatgatttcatttatataacagtctcaatattatatatttgtggagatagaaaacagattacaggttgccagggcttagcaattgtgggggtagtgagggaacatgtaactttaaaagtatagcacatctttgtggggatagaacagttctatacactgattgtgctggtggttacaggaatcactacatgtgataaaatggcatagacatatatacatacattgtaccaaagccaaagtcctggtcttgatatggtaccctaactatgtaagatgtacaaatggggggtacaattcaagtaaagtgtacatttatgtgtgtacttttaaaaacattattacaaattcacagaaagttgtgaagatagtatggagagatttgtggactcttcatctctcttaacattttatttctttcctttacaataataataatagctaacaattatggaatgctcattccatgccaggcagtattttatgtgcttaacaagtattaactcattaaatgttcacaacatttgacatagatattaattagctgcactttattgaaaagctgaggcataaagagattaagtaatttgccccattaagtagaagaacaaaggtttgaacccagaagactgactctagacttcactgtgcttggtttttcttctcccttctactctataaagtctcaaacaccactaccttgcctatagtacttggtatacagaagtagttacttaatatttttgtaggagtgataaaaacatacaaggaaaaaatcaaagaggactgggtcctctctcttccactctgcccctgaaaagctgtgtgtttatctgggttccttagaagcatttgggatgcattcctgtcacatgctgctgatctcaccctgtgtgctgtctgcttgttagaagccactctgcttgaaccatgaattgtctctatcaagggaaatattttgtctcagtgactattcattcaacaaatattactgctcatctactatgtgccaggcacagataagatcaaaatgaaattaaggggcttacggcttgctaaggaagaccaactttgatttccaacaatgttgataggaaaggaaagtatctaggctttctgaaagccaacttgctagcatttaaaaaagttttaaacatgcttgctcttatgggatgaattctgtccacctcccaagttcatatattgaaatcctaacctccaatgcctccaaatgtgactgaatttgggaggtagggactttaaagagatgattaaattaaaatgaggtcattagtttggcccctgatctaatatggcccttgtccttataagaaaaggaaacttggacacagtcatatgtagggggaagaagtcatctacaagccagggagagaagtctcggaagaaaccaaccctcccaacaccttgatcttggacttccagcctccagaagtgagaataaaatttatcttgcttaagccacccaatctgtgttactttgttttggcagccctagcaaactaatacatatgccttccctccattatttccacatctgacaatctatgccacagatgtgctcgtatttatgcagaatgatagatacatctagcagagttctcacaggaatgatgtttttaagagccccagaacgtaaatgttaatcaataaaagaatcaatagatgaattatagtacattccattttataaaatgctatggagactttagaagcaagtgatatgtgtgctgatatggaaagatagccaaggtttgcttttttatttttttttaacataacaccccaaataggctaaatgactcaaaaaaaaaagcaagtcccacacaatgtacttagagtggtcttatgtttatgtataaaaaatcaggatgtgtgcagggataacacaaacaaatagaaaatggtctggatttgaaactttgtgataggaacaaagtagttcccttttagcttttgttttgtatgcttctttttgttagaaccatccacaactgtgagctgggataaggggtttggatttgtgtgtaatgacaatgagaagtcatagaaaaagtttaggagagagagcataatacagacatacattttagaaacattggtctgcctacagggtggtgaactgcttggatgagtaagtctagtggaagggaaactgagctagataactttgtaatagcccaagagagaaatgatggtggcgtggacagggtagacagaatgtgttcatgcatgtctgtaatttgaaataatttctgtagcccaatagacaaggaactgtgggaacaagtaagaaaaaaagtattctggggtctaaagtggcctccctaaggaagttccatttgatctgagaccagaaaagagactttgggtgagggtcaagatgagagggtggagacaggcaggggagggagaagttagaggggatgaggggctttaaggtaatgggagtgactaaggatggtaaggcttaatgaaagggagaaggaatttatacagttagagaggtaccaaggggtggtccccaggagccttgtaagctctgataaggagtttggttttgtttttaaggtcggtgagaagtcatagaaaaggtctaagagagaaagagaatgatacagacttatatttttcaaaagatttgtctgcctacagggtggaaaactgcttgggaggggcaagtttagttgaagggaaactgagctagataattttatagtagcctgagagagaaatgatgctgacatggagagagcagtgacaatgggtgtggtagacatctccagcaagacgtcatcatcctcatccctgaaatctgtaaggggaattaaggttgctaataagataactttaaaatagggagattatccaggctggtccaatataatacaagcatccttaaaagtagaagagaaagacaaaataagagagtcagaggaaaatgggaaagatgggccgggcacggtggctcacgcctataatcccagcactttgggaggctgaggcaggcagatcacctgaggtcaggagtttgagaacagcctggccaacatggaaaaccccatctctactaaaaatataaaaattagctgggcatcgtggtgcatgcctgtaatcccagccactcaggtggctgaggccagagaatcgcttgaacccaggaggcagaggttgcagtgagccaagatcatgaagatcaccaagatcacactacagcactccagcctggggggcagcacgtgaccctgtcaaaaaaaaaaaaaagagaagaaaagaaaaataaaaagaaagtgggaaagatggaataacagtcagagagagatgcagtatcagccaaggaatacaagtgacttctagaagctggaagagacaaggaaatgtattctgctctagagactccagaaagggacatacccctgccaatgccttgatttttaccaggtgagactcatgttatacttccaacctccagaaccgtaagaaaatacattgcattatctaagctgctaagtttgtggaaatttgttacagcaactttaggaaactaatacagtgggaattcagagagaggtatagatgtagtagacatctggggagcagaatggataggaattgatgatgattgaatgtgcaggatgatgcagaagagaaaggcaaagtgaactctcagaactggcttccacaatttaatgcaatgggatgctatctatataaggattgagatatgtaggaaagaaacagagcaagtaggagatcatgagttaagttttggagatgctgagtgtcaaaaatgagaataatccaagaggagctttggaagcatagacacagagattaagagagaggtttgggctctgtctccatatttgggctcattggcataaaggagaagtaaacttatagtaaagaatgaaaccatggtgaagtagtggcttttgattgttatatttaacacactttccatgcagtttttctgagtacttactacattgatgttactctaaaacccatgattttctctttgctattatctcaatattatgttgtcattctgccaatgtggaaatgaataaatttaaatatgttttccttaaagtgtgccttttgtcaatactggatattggatattgcagaaagttgagagggatgagttgaagccagggtgaggagttgaagcgaggagttagagcagaaaggtctgatgatgttgagggtctaaaaagtgtgcagttcagatggatgtatatgctaaatccaactgtgggcacagatttcagaatcagggcagaatcagaaagcttagataagaagaaaccagcagacaggtaagatccaaacatcatgatcagagggaagggtctaagacattcacagaagtagagaaggacaaataagaaacaaagattcaggtatgaagtcggggtttacatcccagtggtagaaattagaagccagaatcagatccacaaaagaagaaaaggggttacagctgtttctacacctgttgagaaggcaagtgtgtttgaattatgatccaggtcaaaaaaacaggcccaggtcacaaagcaactggataatgagcatctgtgcaatagaaatggggaccaggtacaaggataatcattgtattcacccaacaggctttgatacagtttctcctggagtgggaagcacccagtggaacatagtgactgtccaatgggtcctattaggagacactgtagtagatgaatccaaggaaaggaaactatgtcatttataacaactttgacacataatgatttttttttagaaaacaactttatttttagattatatgatgaacccatcccaagatttttagattttcctatggatcccaaatatgtaatctatgtttcattttccctaaaatcagcatagctaaaagactggagaatcttttgttcttgattctgaggctataataaatgtatatctgagcaatgacatctcagaaaggggatggtgtggttggtgccatcccctctgtaatgtgatttctgaaacagtcacctttctcccgtaagtgaaggaccagtcattaaacctaaattatatgaacacaggtaggtatttctaaagtacttaaatttccattattacatttaccaagttctgaaaaatagatctgaggctaagaaaagaaaaatctctcttcgtgagggagtcaataagaaaagactcactcttaagaggtacgttaaagtcaccattttaaaattctgtggtcttattctttctaccatgtctctagctggctgaatgactggaaaccttaatagttcagtaattatattaactcttcactttattagaccaacacagtttgacatatttctttcttaatcccaaatgttatattacaatttgagattttgaggggggatatggacaaaaggattttctaataagtgacctcctgctctgcctgccccaccccatcctgtgcgtgccccaccccgtcttcagtatggcttctgcctgccaactacttcctcccaggcaatcaaagcccctggggcatatccatgtcattatcttttattactattattattatgaaatatacacaacaaaaaatttaccattttaaccaatttttattgtactgttcagtagcattaaatacattcacactttcgtgtgaccatcaccaccatccatgtcattaaccttaaaatgttgccatgggtgggacacaggactgagccatgagtcattgttttgaaaaggttgaagaatgctggaaccttctcaatttgtaataggggtcaagtagctgctcctctacctctgcgtgtccacctgatgtgatgagagatggcacttcagccactgattggacctggcttatagtagagttcctactccattccctaaaatcctaatttggattgcacttgctaggagccagacacagatctaagccacttttcatgtcttagttcactgaatgttcacaacaacccactgagggggagaatattattattcccatttatcataggatactgaggcaaaaggaggcaaagccagttgcccaagaatgcagaactgataaatgtagcaggactcagattcagatccaggaagtccgggtccctgctttcaaccagaaccctaaattaatgagaaaatgggctgcctgaggaggcaataagattgtttgataaaattcttttgccgttaccctttagataattttgtaaagaaagtaatttcacagaatgcccatcttgcttttttgaacacctcaagctttatgcccaagagaaaaataaggcaacaaaaatatttttaaaatagttataaaggaatacatgcaagaaacgggggtctccatatggctaatgaagactttggccccttttaatgggtggtggcttcttgtggatattccctagtccctaggcattgctggtgagtcctgggaagtggggaccataagccccacaagtatgctacatgccagttagagtcctggcgggaagcagaaggtgcactccagaagggtttacctagcacatataagcaaggttctgagaaccaataaaaaatgtaggaatacccacaaactaaaaactgcaggaagatgtcacatctagccccacagggactgaaaactggagggaaaactggagggaaaagggaatggcaacagaagcttgtaaatattggggaattttggcaagaaaaccagagaaataagtttagcaagaaaacaagggaattttgcaaagaaaaccagggtaataaatacccaacctctttctccacctacaaattgcctgtaattggtcaaatccccctagaaaccaaagacaaggaagttctagtgacacagtcatataaatcaacttaaaggcacagagaagagaagagactgaatctgggtctggggagagagttgtagtgtatcaattcagaagaaccagcaaagtctcccttgcaagcagatattatgagcagtattaaccactcccttgtctatcatggaagcaacattcagatcatccagtcccttgtagggctttcttctaataccttctcattgagttatccaaacaattacaggagctatgcatatctggagttaactccatgtagaatggaggactctccatctgagagaggctgccagggctacacagcatgctgcaccatgggggctggactagtattcaagtcagcagagcatggaccagtttgtactcaaaatgtggactgccgggttttgaattccatttcagtttcttcctacttgaatgaacttgggcacatttttttttaactcctgtaggcctcagtttccttatctgtgtagcagaaataataatagtccttacaatatgaattactgtgaacagaatatgtgaattggtacatatagagcagtgtctgacacacagttcatcctagatgaatattatttaagtagcatcttctggtccctaatgaggtgctgcgccatcaccccaaccagcatgactcgaatagtaccaggtgctgctgcatttgcctgttatttcttctttgactccctactccctatgcctgttcgtagagacctgaattgggagatgcctgcatgttggagcttttagactaggcatatgcttaaggcagggcaacccactaagttagggacaaagctgggctgtccctgagaacaggttgagggctgagataaccatgaagatgtcagagcagaaatggggtattagagtctggggatctggatttccttcagtctcttctatgacttctaagacttatatttcatagaagaacatagggtgactgaaaccatccatcaaagcagtgtttgctgtgagttttgcagtaaactttcagaaacaaggattctgggatctgtttactatccttctctaggaactaaactaatattagcataataaagaccttgaaatgtcccacaaagggaccttatgtaattcattttttcccaaacttatttagctaaggagccaacttggttaatgctgaatcaaactgccaggtgaatgttcacatactgaaacaaataataaagtgtttaataatgaaacaaagaaatgaagaaatatttcaaattaattcttgaattgcctacttctaaacctatgtaactttctgtactgaaacatacctacacagctgtcatgaattaggtctgctggacactgaggagtggaaggaggtatgctgtgcccttggtttacatgggtgctgagcagtagggatcttttcttctgtaggagaggctgctcagcttcacctgctcagtgacacagtttgtaatttctcagagagggcctttatggagagacatgacaggacccaagaaacctcaccatccacaccaactgggttccctaagaaaggtaggatggtgtaactggcagagaaggtaaatacagggcagccatacattgctctcacaagtgattaaaagcttgtgttttgagattctaaatctccagttttacaagtttctaacctggactctgagcatctgtttcctcttctatgaaagggcgatgataataacacctcctctgcagaaaagagttagtctagtaggcttcaggatgcatatcctagaaaaaaagcctatttgcaaggttggcccttggctggcatctgggaacttggattttagcaagttcctcaccattccctgataaagaatggcttgctgtgcctaaactgtttatgcaaaaaaaaaaaaagaggtttatgttcaacccctgttttcctcctggaatttaaatatggactaagcaaggggtgcctatgtgaccagtacaaaataaaatcctcaggcccttcatctctgatgggcttccccagtagatggctttttacatgtgttgttacaatcagtcctgggggaattaaccacaacccgtgtgagttcactaggagaagacctctgaaagcctgcacctgtattctctggacttggccccatgcaccttttcccactgctaattttgttttgtatcctctcatggtaataaatcacagccacgagtatgactgtatgcagagtcctatgagttctcctcatgaatcatcaagcctgggggtggtctttgggacccctgacagagggctaccatgaatgtgggatgagacgcattgaacttttagtgccctagtaggtagagactgagcatgagcacatcagagagaagaaatattctgccaagaagatcttcacttccctactaggcgtccatctgtcccatggtgctaaaaggcactaggaccctgtctctgtctttcagctgagtgtgggcctcctccttgctaacagagtagtagccactgctgatagaatgtcacaataaatcttggagattttgtctctgcacctgtgaacttcccaggaatattgcttcaggatttagagctgttttgtgttttccttttaagttgttcctctgtaactctttaactcaaacttactggagaggtagaactgcatgtttgaccattaaaattatagttgtgctgtatcacacaatgcagtgtaggtagattaattcagctgtggatttcctctccgcaactctcttatttaaaaacagaaacagtattagttcattgatatcttcttttcaatgctctcattgagtggcatagcaaccactgggaaattcacaatgagagagggaataataagacatttctattctgcctctaaaaatatttggcctagcattttcagtgcttgttcaggtatgaatacaggccaaataactcttctcaaatgatgtttctgaaataatttccatcttgcatttttaaaaaatggtcatagctaaaatttcttgaactgtcaagcggactcagccaacaaatggccaaattatattttcaaatgtattttatgctcactaaataatttagagagatggtgtatagagctaattattggtgtaatttcctgccactgaaaacattccaggccttcaaccagatagtaatatcacaatcaaaactcctttgagttagcactcaattgtgtaacagctaatatagttacccagattggatgttgttacttgaaactttgaagcagctaaaatagaaacctaaaaatggcaagcatctgaagatgcaaggtagaggatgataatcagggtttttgttatgttagctctttctgtgtagcatggtttaaagcaaaaaaacatttatttgtgtcctgattctgtaagtgataattttaagttcagttcaactgtgtagttctgctggttttgactaggctcactcatgtggatagctgctggtcagctgggcagctctgcttctcagggtcgactagttgctggctggggagacaggtaactgggatacacagcccctcatcttccagcaggataggctggctttgttttcatgctgactgctgtttctaaggacagcaagagggcagacaagccacagtgtgcaggcacagtgcgtattggtgtcatattttctcttgtctcactggccatagcaaattatatatccaagtccagagtcagtgtgggagagtcctccttgaaggcatggataccaggaagaatgaaacattgtgggccatctctgcagcagtctatgacagcagggttcctttacttcttgacactttggtttgggtgtgcccaagcgtttaaggtcagctgacttgtatttattgacctacttgtagtaaagtacttacagaattgttttaagttataactatattttccaggaaaggtaatatatcaaggtagttgaagtccttatactctttcatccgttttcattacagcccttccaaattagtctgaagtaacaaccagaatgaatgcgatgggttttgacggagtccagggagtgaaggctgtgaatggtctgagcattgctgagagaatatttatgtaggtagaggatggaaaaggaacttgagaaaactggaaggcatgggaagaccctgcttaccacacagggacaagtgtttaaaagaagtaacagttttaacagaggatgaagggaagtaaagaagggagagtgagagaacatggagctccaaccgcacatgctatttactgaataaatgtgctgttcagggtgattttagtccatcaacaaatattttgtgagccctactataggccaggcaaacagtaatgaaaaagcaggaagcatcctttccctcatggatcttacattctagtagttaaaacagacactgagcacaccattatatgtaattaattaatcgcaagtggataaatgctgtgaagaatatatacatagaagtgtaggaagataggctgggcgcagtggctcatgcctgtaatcccagcactttgggaggccgaggcgggcggatcacgaggtcaggagatggagaccatcctggctaacacggtgaaaccccatctctactaaaaatacagaaaaattagcttgggcatggtggagggcacctgtagtcccagctactcgggaggctgaggcaggagaatggtgtgaacccgggaggcagagcttgcagtgagctgagattgcaccactgcgctccagcctgggcaacagagcgatactctgtctcaaaaaaaaaaaaaagaagtgtaggaatatatgacctacctattccagtccaagggctgagataagtcttcctcaacaaatcaacatgtaggccaaaacatcatctttcacaatgcccatgcagtgttgtaaggattcccaaaatgatgattgttctattcacagatgagtagatgcagagaggaaaaaagcaattatctgggatcacagtgaataggtgacctagctgggatgcaaacccaggttaccttatgtgttttcaacatcataaagcatactttgcctattttacatattttctagaggtattctggtcactggctatgtttcacctaggtaaactatgttgtttcaaacaggtaaagtacactgtaaaaaattgtaaatataatatacagtgaaaagtgagtcttcctccctgccttcttcccctggtacccttctctgcaggtaatatcagtcaactttcttgtatatttgtccagaggtgtataattaatacttgaagtaattactttgtgacagatgtttttccaagtgctttgtaaatatttacttgtttgatcctttatgtggtgactatcattatcaacccatttttcatgtatgcatatattttattccccacaaatgttttggattttaactacataccaaactttgaaatgtttctccctgtgtactggtcagctttaataatgctatataacaaacaaggccaaaatctcagtggcttcattacatacatttatttctcatttgcgagtcagccgtggctcaataatatctcagttgagcttgcctggtcttggcttcagactggaggttgggcttgtgtctgccctcatgcatctctccttccaagacctaggctgaggaagtgtcatctccctgagagagagatctttgtgacaagtgacaggagcacaaggaaaaagtggaaacatacaatgccttcaaaaacttctgccctgaacttgccaactgtctcttccatccatagtcaaagcaggtcacatggccaaatcaaagtcaatggggcagggaactatcatccacatggcaagtttaaggagggaacaaaaaaaaattgaataaaattatctaatttacacaccctgaaaactcaacttttatcttttgcagctgggatctagaggaataactgaaaagatgttgccctgagtgaattaacaatagctttctcctggtcttctgctggcaagatataaatggctgcctcttttccatttatatttcccaggttttggcatcatgtttgggaagaaaaagaaaaagattgaaatatctggcccgtccaactttgaacacagggttcatactgggtttgatccacaagagcagaagtttaccggccttccccagcagtggcacagcctgttagcagatacggccaacaggccaaagcctatggtggacccttcatgcatcacacccatccagctggctcctatgaaggtatggtgagggctccatctttggctgggcttaagaatcatgctcttatttaatgcactgcatcagctttataagcaagctataaaatgcctaaaccacatattttttaacattcacagattattcacaattattctttcatgattataattttcaccatttcttataaattgcagcccatgaaagggaactttggctttagatgtgatttttgaataatcagacacataaaacacgatcacaaaataggacatttttatttcgactttaaatggtgtttttgttagactttatactgaagcatattatccatccactctggttaatattagcaatagcctctgaccaacattagaccaccttttgttgttttccatttgctctcaaaatatttaaacttttctttcatataaaatttactaccaatagattttaataatggtattgataaatatatatacatatgcatatttaggcatatacatgtatatgtttatataatcaaataaatatgtgtgtatatatacacacaaacatatacgcacatatttatttgtctttatagctttcaaatttgtctttatagcctaaaagagagctatttcaaataaaagcaagtaatttgagtcctagaatcagatataatttatcttaaatatgtaactaggaaatgtttggcaataaatgctcataaaagtaattacatgtaagcagaaagcagacccaaaaagatttcagttatgccaagttactacaaagaaaatccagggattgcagttttcttctgctcttcagaatgataagaattctatagactagcaggcaattgcccaaaagttctctatgggagttaaacatcatggaaacttataatcatattatccagtggatcattccagagtttcataatctacacaaagcttgaagttcagattctttccttgttcattatattagtagacatctgtgctatttgaatatgttattctgtgttaacatgaatactgcataaactgtatttaaataatatgcagaacaaaactgaatacatttttaaagggatgcaaagaaagagaacagctgatacattatgggaaatgcttatgaaatatcacctgacctgactaaattagaactgtgattccattgttcatgcacagtgctggtcattctattctggtattgaacatatgagtctgttctgcccaaagatcaatacagagtcttataattctgcattttgtgagtttgattttctggtagaatttcaaaacttttcaaaccaactaaaatgttaagtacatacacacacaaaaaaatggatatttgagaagataaagctttgggaacaaaaggaggctgtcattttaaacagatatcaattgatatttattagaaactgactaaccaactctactagtttgcatgtggaaatatcctactattgaacataattttctccctaagagaaggctagcacttcactaatgatcatataataaggtggaccatatctttaaaaagttgggattagaaaaaaatctgcttgtttagtttcattatttttcttatatcccaagtttttacaaaacccagaaaatgcaatgagcagtgtgccctgaaaagtcttctactatttatcaccatggggaaagaaaaattctcacatgaaactaagacttattttcttaccctggagtatcattatcattatcattggataagaaacattctagatggaagcagagtcttaaagaaacattctcacaccagttagaatggtgatcattaaaaagtcaggaaacaacaggtgctggagaggatgtggagaaataggaacactttcacattgttggtgggactgtaaacgagttcaaccattgtggaagtcagtgtggcgattcctcagggatctagaactagaaataccatttgacccagccatcccattactgggtatatacccaaagtgagaggtgacagcatgctggcagtcctcagagccctcgcttgctctcggcacctcccctgcctgggctcccactttggtggcatttgaggagcccttcagcccccccgctgcactgtgggagcccctttctgggctggccaaggctggagcccactccctcagcttgcagggaggtgtggagggagaggcacaagtgggaaccggggctgcatgcggtgcttgcgggccagctggagttccgggtgggcgtgggcttggtgggccccgcactcggagcacccagccagccctgctggccccgggcaatggggaacttagcacctgggccagtggctgcggagggtgtactgagtcccccagcagtgccggcccaccggcgctgtgctcgatttctcgccgggccttagctgccttcccacggggcagggcttgggacctgcagcccgccatgcctgagcctcccacccactccatgggctcctgtgcggccgagcctccctgacgagcaccaccccctgctccacagcgcccagtcccatcgaccacccaagggctgaggaatgtgagcgcaccgcacaggactggcaggcagctccacctgcagccccggtgcgggatccactaggtgaagccagctgggctcctgagtctggtggggacgtggagagtctttctgtctagctcagggattgtaaatacaccaatcggcactctgtatctagctcaaggtttgtaaacacaccaatcagcaccctgtgtttagctcaaggtttgtgagtgcaccagtcgacactctgtatctagctgctctggtggggccttggagaacctttatgtctagctcagggattgtaaatacaccaatcggcactctgtatctagctcaaggtttgtaaacacaccaatcagcaccctgtgtttagctcaaggtttgtgggtgcaccaatcgacactctgtatctagctgctctggtggggccttggagaacctgtgtgtcgaaactctgtatctaactaatctgatggggaggtggagaacctttgtatctagctcagggattgtaaacgcaccaatcagcaccctgacaaaacaggccactgggctctaccaatcagcaggatgtgggtggggccagataaaagaataaaagcaggctgcccgagccagcattggcagcccactggggtccacttccacactgtggaagctttgttctttcgctctttgcaataaatcttgctactgctcactcttcggggccacactgcttatatgagctgtaacactcaccgtgaagatctgcagcttcactcctgagcccagcgagaccacgagcccaccaggaggaacgaacaactccagacgcgctgtcttaagagctgtaacactcaccgtgaaggtctgcagcttcactcctgagccagcgagaccacgaacacaccagaaggaagaaattctgaacacatctgaacatcagaagagacagactccagacgtgccactttaagagctgtaacactcaccgcgagggtccgcggcttcattcttgaagtcagtgagaccaagaacccaccaattctggacacaaaaggattataaaacatgctgctataaacacacaagcacacgtatgtttattgcggcactattcacaatagcaaagacttgaaaccaacccaaatgtccaacaatgatagactggatcaagaaaatgttgcacatatataccatggaatactatgcagccataaaaaatgatgagttcatgtcctttgtagggacatggatgaagctggaaaccatcattctcagcaaactatcacaaggacaaaaaaccaaacaccgaatgttctcactcataggtgggaagtgaacaataagaacacatggacacaggaaggggaacatcacacaccagggactgttgtggggtggggtggggggaggggggagggatagcattaggagatacacctaatgctaaatgatgagttaatgggtgcagcacaccaacatggcacatgtatatatatgtaacaaacctgcatgttgtgcacatgtaccctaaaacttaaagtataataaaaaaaaataataaacattctagctgaagacccaggacatgtgccgtcttgttagagtgtatgcattatttggttaggacaaagtatcatttacttcaccacggtttttcacttgtccacaacctagcaggtcctgtttgagagcaaagaaagaggctttcatcttagtggtaacacatcatgttctgtggatgtgggacttagcagaaccttctacctaatttcttccatttcctaggtgggctggcttgttatcctttcctctctgacaatcagtcccactgagagttggagcaagatttcctcacttctaagctgctaaaccttgtgcttctcttactcactagtatgggctttccagcctttgtgccaaatgcatgagcattcagtccatatgaaatgaaaagactaaaatattagttatagacacttaaatgttaactgggtgatgactggaagatacatctcaagtatagagcaggttctggatttctgcaaatgagacttgaggcacaattgtgccgctgagggaaaaaaataaaataaaaacagacaaggagtgacaaaaacaagatccagctaggttggatctcacccataaagagtaaagttgatcaaatccatttaactgaaggacacccaaaaaatctagtacctcatactaacactaaactcccataaagcaaatcctatctttctccaaatatgagtgtaaatatgtctccacatatgagttcagttaacatcactggcagacagtagacaatattggtggtcaaacagaatcctgtgactaccagactcttacaataagtaaaaattaaaaggtttaaggggacaaactatgttatatagtttgtatgatcccattttttgaaaatggcttctctggaaaaatcttacaagtcacctaagaatgacatatgataaagtatagagaatatatctcagaagtcaaaaaagtccttgaaataagaagtaatgaataaatgggcagaaaagactggatatagtcatgcatgtttaaagccattttggcaaaattatttaagttaaatagaaccaaccattctagttggtttggtcaaagtgtcctttaaattgagaaaggaaaatgagaaaagagatcatgacataaaaacagtcaacggaggaagtgtgtcgcccctgatgttgtggaaaatgccagttcttcagtacttgtcagcaagctttgccaagctagttccgagtggaaatctgccgttgccaccaacactcttccttgattagttaggatttcagctgttcctgtttagaacctcaggcttacccattgtcacgcttaccaggacacacctctcctctgtgtagacttatttaggccagggcttctccagccatacagcagatcagaatcacctgaagagtttgataaaacacagattgctgggctccattctcagaatttctgattcagtagaactgggatggggcccaagaatttgcatttctaataagtttccaggtggtactgatgctgctggtctgaggaccacactttgactttgatttaagtagtataaaagaatgtatccacataaggtataatcaatgtgagtttacaccagccgggatttccaaactgtcagaaatttaaccagaagccacaagttagcttgtacttgtgtcaacagtttgtgtgcccaaacttttgggctagccatgtaggccatgtagggctagccaagataacctgaataccccaagggggaggttccctttaaaacatttggcaaatatcattgagaaactccttttatatctaatgtcatttcgtccatgagaacatcataagcaatattaccaaaatctgtgctgaattagatgcattatgtaaagggcaggttgaatggtcgtatttgtttgtgattggccagtaattctcaaacttagactttcataagaatttccctgggggcttgttatagctcagattcccaagccccaccctaggtattctgattcagtaggtctggagtgagcctgataacttgcatttctaacaagctctcagatgctgctgatggatagatgtactttaagaactgctgttctaggtggactaaagccacaacataatgatcaccctttccctttgttacagcctagaaatcatttgaaaatgctaaaaaacaaaatacagaggataatatttaaaagcttttgtagcttctacctaaaatgaaaaaaagcatatcttaatatttgcttcatatttctttttaattttcaggtagaattatacaaataatattagatagttttattcggcatatgtgagttttatgaaatgatgtcataatgtatataatctctaacttgcttttttacttaatagtgttttgagattaattcacactgatgtgtagaaaatgaatttatttaaaaattactctagcttttttttcaatttatccatttctctactaagggatattgtcaagtatttgctattataaatattaataatattgtcatggacatctgtgttcatgtcttcttgggcctatggtgaaaagttttcttgcagtggttctcaaaagtgtggtctaaacctatggggggttcctcagactcttttagggagtctacaaaattaaagtttgctttttttaactctcgtttcctgtgtatacaatggagaagctacattacatgtgataatgtcattcattggtggatgcttaatggatcatgtgctttatagtcttatgttttaaattttcttgggtgggcacatgcctgtaatccagcactctgggaggccaaagcaagagtgcttgagttcaggagtttgagaccaacctgggcaacctagcaaaacctcgtctccactaaaagtaataataaaaaaaatattagccaggcatgctggtgtacacctgtcatcccggctactctagaggctgaggtgggaggatcgtttgagcctaggtggtcaaagctacagtgagccacgattgaaccactgcactccagcctgggtaacagagcaagaacctgtctcaagtaaattaataagtttatcagttttaatatctaatgcagtgaatattaatagatataatataaactaaggtctcttttaggtcctcaataattttaagaggttcctgagacaaaaagtttgagaaccactatcttggaacttatattaaatgtgattatctgtggcttctgcaacttgcctttcacacactactgacttttttgttgttttaaatgaaaatagagacttttgtcttttctagttggccagcaatcttctaatctctacaaccttttcaaagattattgaatacaatattccatcccttttcgtgtatactatataatttctctaaaccagctgttttgaagttttttaaattgttagatccttatttaccatcccttttctaatttagccttcaaagtcttcctaacaatcttctttttatcttttaaagattgaagagtcacagagaagaccaatacaaagctggctctgacttagttttgtttgtctttgtagtcttcggtattttataatctttcaaagctgaggttctgtctcttccttttttatcgactgctgctgagtactgctttaaaaacttctctattcacaaaaacaggaaaaatcaagagaggtaaatctttaggtctggccatcccattcttgaggaccactgttttattttctctcccatcgtagtttatgttgaaagctcatccaaaattgaatctaatttaattactctgaaattctctacctttctctatttttgtgatgctcaattttgcattgtatttagagctatatcatttttcttagttttatttccatgggattctctctttcctttaatatttttaaaaatctgctttcccaaaatacagggcaactatctgacattttctagcattatttccctttgacctaaggtaacagttttccccaaggttccttctcaattacgcataactgatgtggtcttccttgttggtcagaattaagccagaaaggagttatttcttttagtgcctcctttactttctgagagttgttgccaagcagtttaaactgtttccagatgtttgccatgctcctagctgagtgaagctcccagcaattctgagagctggggctgccccctcatgacttttctggctcccttaacaggtctcttcttaaagctcacagattaggttccccattactttcctatcctcttaaatcagtgttttttgtttgtttgtttttgcctctcccttcttttagacctgcttctgctaaaatatagtgttgagctagaagcatcttttttgcaacatgaagtccagtgccttcatgttcactgagctgggtcaccccttttcatttctaagttctcatctggatgaacatcagggcacttctaagaggtaacaatggagccaggagaagaccttggaaagggatatattcaaaggaaggaggaagaactagtcccaagaccctgtggcaggaacaagcttgaggagtttaaaggaaaaacagatcatctgttgggtagagcagagcatgatggggagaggggtgggggatatggccagatggacaagtagagattggatggcacggggcacttttgaaagccacagtcaggagtttgctattttgtgaagcctctgtgcatgaaggacacaggtctcagatcaagaaattgtcctaatacaaaatgaattgatttatttaagggtcagacccaatgtatagtgtaccaaatatattgatctaatatgtaatcctgcaggtgttagaggtgtgttagtcaaatggttgtgaacagttgggatgcaggaagttcttccaacttaatgattatcagaagacatttaataaaaattcaaaccttcttaaagaagaaagagtgtatatgttataaagacagttaatttcattgcaaagtaaaagcaattataaaatgaaacccattagacagattacctatgttttaaaacctcttgtcaaaaatgccacttcagaagaactgcattgagttcttcagaaccaaggccctcagcgggaggagagattgctgatcctttgttaaagttgtttatattatttttatacttgcaaaaccagtgaggacaatggacttggacactcacatttctcttggtgcctgttacttcctggaatccctccttccagtgtcagtatgccttttcagccctcccaccaccacaaagactgtggaatagaaacatcacactggcccccattttctcatttgcctaaattcaggagatgtgggcaaggtaatcttgtttccaaataaaagaagaaatccaaaaccaaagctagatttattatatttcttcatttttatgatcccatccaatctggaaacttcagccaagcagtttttgattatattaatgtggttattttttgatgtgcaccatgtcaacacaccatctttccccataaatgtctctttctcacaccctttaaaagtaacaaactacatcaaccactaataagaaccaactttgtttacattagtgttttgcagggttttacataagccactttaagattttcttaaactttgaacaaacttaagctcacatggcctagttattactctaaaaacaaaagctgatgttttcagagaagacagtatcaccccagggaatttatccctgtgggtacccaccacttcttctatttggaggagggccaatgttaaccacacacctccaggcctcttggcagggccatcctggtacccagagtgacagctctgtaatcacaaatgggtgatgtcacatctatcacacctgcagctccctggaaccagcagagaataactggagattcatggtccatgatcctggcatttggaagactgctcctagggtcccaactaaaattgggtcactgagacctcatgaatgaatgctctcagttactccccatcaaggagtaactgtaatctcctgccaaggattattagaaaataatccttctaaatcaaaagaaaaaaggcatgtggccctctgtgactcccactcatgcttggaacttgaaagatttaatctcctgccacctcaagaccaaggtaaacacaaattgaatgtaccattttaaagcaattgtcagttataggttatattgaaaagttatttaccaccaatggttagatctatccctgaaggcctcttctcatggttcaaaatgctgttcccagcattcctgcagtggctgtgttcccctactgtaactcctgggaggaagtgatagagaagaacccacacagggaccgcatatactgagggttcctgtgagtacctccacttgagtgttttaccccaactctactcccagagcagaagctgatgtgaaggcaatgcaggtgtcaaatctgtagtaatctctagtttttgttgaaatctcttaggcaacaggcctgaccaaaatcaagagtatcattaccaaatatcggatggtgttttacagagtaaaaaatgtttaagatttattcttttgtggcacttataataacaaatagttttaacccagttgtacagataagtcttgactaagtatgattgattagtgcaaattcacccaatttctaagttgaacccagttatttatttatttatttagagacagggtaatggagtgacaccctgtctcaaaaactaaataaataaactcaatttttcatttttctcaccacagtctcatacaggactgagtacttccccatcccttgaatttgcatgggactcacatggccagtggggtgatgtgagcagacttctaatggacagctaagatttccctctgtgcttatgccaacatcataagaatatgccttagatgacagactgcctgcatctctgaaactggaagcagagccacccagccccaacctcattgtatgaacctgagtgtgggtggtttgttgtgcaaccttataggacaactgactgatactatcttacttagccccataccagccttttcaattagagagtagtaactccgtcttttgggtaaggaaagcaagtcttagaaagtttaaaggatttgtttaagatcccacttctgtaactggaaaggcccaggactcaaaccttatacctatctgactccaagacctgggcttgtaatcaccacatcactgtgcctcctggtaataagtatgtttgggcatgaaaaactgttatgtgcataaacgatgctaagtcagaaaacaaaattgcttccttcttaccttttcaccttgtagattgtgtaactaagactcaggtaggcacagtgttttttcctaagaccacagagcacattaatagcagatattatttctgtgggtaaggaatgcttttgattcgatcatgtagaaatcccagatttagcccatgagctctcccttgagaatctgacaaggacatctttataaatacctacctgactatgattccctatcattactgccatatgactttattttttagttgtagagtactcagagccctttgcagagtctttcattatgcatgtaatccacgcagtcttctcaagtgagttaggcagaatatttaaataggaaatgtcaatgaaatcatgcaacgagaagcaaatgagtgagctttcaaacgtgtgccactcattccttctccactatggcctggatgctccctgagcccatcatattttaggtcaaacagcataccccccagatcacaagcctgtctccagaactgtgttggtttcccatagagtggccctctctgaactcctgaaagctgaactcatcaaataaggacagctgtgctttgatagctgggaaattctatataatgtgaaatgatattctaggggacatcttcttttccccagctcttctctcccagcagtggtgacctaactgaaaataatccttctaaatcaaaagaagaaaggcatgtggctctctgtgactcccactcatgcttggaacttgaaagatttaattttatctctcatttagcatcttatagaaattgggacactaagatggcagctagaggctgctttagtggaactatcaaatcctatcctttttagtgttttattgtgtaattttaagtggaagcattatgctgtcaactttatactgatgataaatctatcactttaaaatcatttccaagcagcacatcactatctgattggatgctactactgttacctgtaaggttaatggctgcctaagatggtacctgtctaagtgtttgctagaatcactgaatccatcacctgttgagaaccttcatatcagctttctattactgcatgacaattaccactaagttaatggcttcaaagaacacatatttattatcacagtttctctgagttcagagtctaggcatggcttaacaggatcttcttgctcaaggtttcacaaggctgcaattagggggccagctgggcttcattcccatctggaggctcaaatggggaagaatccacttccaagcttatttagtcagaattctgtgggcagaattcttttctttgcagctgcagaatgcgtgacagcttgcttcttcaaggctagcaggaaagcaagagctagtctgcttggactatatatataataatgtactcacagaagtgatatcccatcagtgttgccatataatgtaagttaaccatgagagtgacattccatcacctttgacatgtaacgacacagaagggacagcctcttacctttaccatattctattggttagaagcaagccacagatcctgcctacactcatggagaggggataatcaaaggcatgatcaccaggaggtgggaatcattgaggggtcaccctagggtctgtctacctcgttcccaatgtcactcccacatgcaaaatacattcatcccagcccaagatcccaaagtctcatctcattgcagcatcatctcaaagtccaaactctcatcatctaaattgtgattaggtattgatggagtgcttgggagtaatctattaaatacagctgttggggcacaattcctttccatccatggacctgtgaagccaaagagacaaattatatgcccccagcctgtccaacatgtaatggtgggacagcacaggataaccggtgtaaaaattctgattcaaaatgggggcagggtgagggcaagtgagtggaaggaaaaagggaattactggtccgtgactcttctgaaatccagccaggcaaatgttaggagttccctgattacatttcaaggcatgggaatggccctctgtggcttaggctcctccctcagggttcttggttctgcccttccttttcatgaaaggcagcacatgtttgcatcctgagtagttttatcagcatgttttccactgctagtagaattggaagggtctgaaagcctcctttatttcatactctctttgtccctttcaggccaagtggccagtattcctgatgaaacaattccttaagaactttgtagagtttgtgtggattcactgcagttcactccattaggtgatgccacatccacagatattttaaagataagccttcctgttccttggcctccttctgagatgactgagggacgatgcccatatgcttccttgaggttttctggcttgattgagaagatctgtagggcagacctttaatttcttaaaagaccccttcgtgtaactgcatagttttgtcttttgatctttctaagtttctaacaaagagctctacagtctcactcagacttttccctagtgcatgctttcctgagagtgaacctcttcattttagcatctgtgccatccagagatcatcggaattttttacatcatcagattctgattctgttttttgagaaaaattgtcctcccaatcttcctctttcctcacacattttactacaagcagaaagaagaaaccagagggttccttcaacacttttcttggaaatctttatacagccaagtatcactttcaattctgctttccacacgacctcgggagactatttcaccgaacttcacattacaaaagattctcccttctccttccttcagtttccgattagtgttcctcacttctgagccttcaccatcagcatcctcaatgtccagttttctactctgagtctgttcatgtcatttaggctttttctaccatgttcctcaaaattcccccagccttcacccacctggttacaaaaccccttccacattgttatggattgaactgtgtccctcccaaactttatacattgagatgctaatccccaatgtgacattgtttaaagatatggactttaagaagtaattaaggctatgtgaggtcacaagggtaggaccctaatctaatagggctggtgcctttataaaagaaaggaagagctcggtcatggtggctcacacctgtaatcccagcgctttgagaggccaaggcaggaggatcacttgagtccaggagttcaagaccagttcaagaacaacatagcaagaccctgtgtgtattttttaaatatttcaaaaaaaaaaaggaggaggaagagacaccagaggtgcccacccagagaaaaggctatgtgaggatacaggtaagaaggcagccaccccagtctgtgttattttgttatagcagcccaagcaaactaatactcatgtttttaggtatttgctatagcagttccccacttccaggtatcaaaagctatactagttttctattgctatgtaacaaattaccataaatataacagctgaaaacaacacacatttgttagtatccttgcagtttctatgggtcaggagtctggcacagcttcactgactcctcaggtctcagaggctgaaattcaggtgtcagctgggctacattccccactgccttcccatccagaggcttgacagggcaagaatccacttcgcaaaattcatttccttacagttctgagggaatggggtactgaggccagaaaggagaagaagcctggtttgttgatgaacaggcaaccccataggcaagccgactcagttgtgccagggatgctctgagatcacatggagcacacctcacaagtagcccagcaaggagcaaggaagttgggacatctacccaccaactccaagctttcttggttgtgcgttgctcccaggagttaatttcctggcaactccacacttggccatgcattccccaacagccagaggacaccttgtggtagagagactcagggaaacttcagcatggataggaaatttctgcaggtggcctctggggtggactgaggggataaaggcacagcaccggtggcaactgctatcagtataatatcagctttcatttccttaacagatgtttattgtgcacctactatgttctgagagaaaaaacaacagtatttggtcttcaccttcacagagtgcacagtctagtgagggagaaagaattgtaaagacaaatgaaaacataatgttataacagaagtataaatattttgttatgaaagctttgagaacaaaatgactatacttcctacaaaatttggagatgtaactttggattttatggaagagtatagtcttatattcatacctaatgaatgaatatccctatgtaaacaaatagtaactttacaaagaggcagtgacaatgtattgcttaagagccctctggagccaaaccatccaagataaataaaaaaatcttgattcttcatattccagctatatgacctagtgcaagttacttaacctctgtgagcttctgtttcctcatctgttagaagtggatataacagtactacctcatagggctggagtaaggattgaatagcttaatacattaaaacattagcagtcaagaaataaaaaatgaatcaaaccaaatggtcacatgtacaaagtataatgaaaattcccttttttaagaagagaacaatattttgagattttctctctcaattaataaaacaaaaattatacatgtatgtcttcatcattttaggaataccatttggaataatttatatgtaactctctaaaccgtacaagtgtgagttctcagagttcttataaatttgaatggaaaggccaggcgcaatggctcatgcctataatcccagcactttgggaggcagaggcaggaggatcacttgaggtcaggagttcgagaccagcctggccaacatggtaaaaccctgtctctactaaatttacaaaaattagcctggtgtggtggcgggcacctgtaatcccagctactcgggaggctgaggcaggagaatcacttgaacccaggaggcggaggttgcagtaagcagagatcgtgccactgcactccagcctgagtgacagagtgagactccatttaaaaaaaaaagaaaagaaaaggaaaagaaaaaacattgaatggaaaaataccgcaggtctctttcacttgtgggtgcattcaagtgtgaggtttctaagggcaggcttccagtggtatctttcacctgaaacctgcaggctgctgcagtgctgcaggccattgctcctaccatgaagcaggaggcagagcaatgactacttgcagcagcatctgacattagcagccctcacctcccgcctcatgggtcagcagcatctttgcaggacatgtgagctgtgaggctggaaacctcattggctggaaaccccaagccaagttcactttctgaagtctgtaggggtggggaaaaggagcactttttaaacaacactaaggagaggtgcatgaggaaatagctttcgtcaaatctccatgtcctaaagccagatcccacttagcttagagcagaaagctggagaaacagctgaatgtgataaaccttctctagaaatgaaaccctgtggtatgtttagcagatatttttatctatagaagaaaatgtgcttcctttgagagttatttctaaaaacctttcatttttatgtttttaccacttgagagcactgagtattcatcataggcagcttagcaaaattagaaaactggagaaaaaaataaagacctgtcttttttttgaattttgaattttggtgtattttttacaccattttaacacagacacatgtgcgtacacatacacgaaagggtgagattgttgactgtatttacacagttttgatttcattttttcaccaaatttcaggggttaccaaagtttcttggttctcagtaacttgtcacagcacctcctagaaaaaaaaataaataaaaataaaagccattccatttattaggtaattcagtccaaactgtaagtatggcataacaacaaatacatgtaaattgaaatttgcaaaatacttatatttcattctgaaacactggtgaaatgtgcaggcctgctggacaccgcctcacttctccaaccttggaaccaggttggataccacaccctcaccctcatttcctgttccacattgattttcacacaggactttttgtttgtttgtttttggtcatggcaactgccagatacactgctttacatttgatgtcatcaaaatacatgtagtgcaatctaatgttgaaagatgcactagtaatttagaaggtgtctcacaagttaaatatcgttgttgccttcaaaattttaaaatagcctgccacagcattgtaagttcgctgcagttagttccctggggtaccttggcatatggtttggaaactatgaacttagtttccttttgacagcaagattatttttattatttttcttttttttctttttctgtttttgtttgttttggatttgcaatattattttaagtgtccacattagaattctacagatgtgccataattttaaaaccctcttattgttggaaatttatgttgattgttagtttatttttattgataatttcattgtcttcaaacgggtaagaaactccattgttttatgtaaaggtcgagtctttgaatccacaattactacccatagttatttggcctatttatattcttaccttgcagtttcagagcccagtgagcccaattagctggtggcacctgcagtcctgaatgctatgatttttcaccttccaaatcctatgatcttcatggttttgtcactgatttgattttggaaacagtgagcctaaaacaggaaactattttttaacccatcagctgcgcttcagattttcaagttaatgaataaaaataaagccaaggaattgacaagcctataaattgtcagcaaccagcaaagctaggagaacacagggaaggaatgaagacagagccaggcacagtggcatgcacctgtagttccagctactggggagtctgaggcaggaggatcatttgagctcaggagttcaaggctgcagtgagctaggatggtgcctgtgaatagccactgtactctagcctgagaaacatagcaagaccttctccgtaagaaaaagaaaagaaaaaaatatgaagacaaataaaaatcattgtgaaattcaaaaaagtcttacttttgataacttcacaccaaaaacccgtgcacagcactatgccctacaccatccatgggcgtggtgtagcaagggaagatgtgaggggactacccagcatggcgcatagcttcttggcacagcgttgtagaaagtggctcatatttgaagttagctcttcaattaagggtagatcctcaatggattattcttccattagctttaaactagcatgaatcaagtcaaatgagagggtgtccgggcagagagtgagaacatcaggatgaaggagttatcaaatataaggagatggtgttgaagctagagcttatgaaggtggtggaggtcattgatggtggagaaggaagtggtttggaggtgatggaggtggttaaaggtggtggaagtggtggtggtggaagctgtggagacagtgatgccagtggagatggctggtggatgtggtggtggcagtggtggtagtggtagtaataatacttttttaaccttcatgaatggctacttgtagatgtaaatggcccgcatctattttatgcccagttataaagttctgttgcccacaaaccaatgtgaagaatccttacttaattgcttataacagattcataatttgtttgtttgtttctgtgagatgcgaactcactttgggatgtaggactggggctcatagttagtaacatatttgaacacacattaccccatctaagcccctgtgaggtaggtattatccttcttttacagactgggtgatctgaggctcagagaacccaaaggactcaccaacagtcacacaactctcaaatagcagagctgaggcgtgctccacccagctcaggagactccagagccagggcctttccacaaagcacagagctgtgtttgagatcgctgtgacttgtcaagggtataatgggaacactagcacattataagacagagaggtaaggagaaaacatgaaccttaattttaagaaaacagataagttggaagaaattctgttacctttataatatttctaccccatcctggacatggcataaaaatctttctagaagtaccttccaagtgtacaaattctactcttttctaaggtattcaccatatccataccttttttaggtattcaacattcaatttgttcagcattattgaatacccgtcatgggctaggcatgatgtcagtgcttgctgagatgtgaaactgagtatgtcagtgactgagatattgataaccaagacaggctaaaatttctataggactggacaaaagttgcattttctgatacagttctcctatgccttacttatttttatagcaaaaactctttctgccaaattttgagataagtatgtttaagtctttctctagaattgtgattttcctatgtttttcttacaatattttattgtctaatattggacctcatattcagcattgggttaaaattgtcttgactacaagacataccatgaccctaaagtgggattgttggatcataaagcgtatgtaccattgaatttagtaagtactgacagatggctctccagaatgacggtaccagtgtactccccaccagtaaagcacaaggttgcacaccttacattctcaccatgccctggtattataaactgaatatttgcaggtctaaggaggatttcattgtggtttttatttcctctcttttcactattgtgtttgagcatctcttcatatcttgttaactgttggaccattcatttggtgaattgcatattcaaatcctttgtcaactttcctgttgttttgcgagaattcttgcattactaagagtttctttaatgcatttaactttaaatgcccccttgtcttatactttcaaacctgatttctctttgtttgtacctatcttagtctaatactgtacttttatttgggtatctgtaagcattgtcttataaacagcatatagttgtttagttttatgttgttttgtctttattcagtctgacatttattggaatttggtacaataatttggtacatttatatttagtatcataactcatctacttgatttcatttcttccctattacttaatgctttatttaatgtacatttttattttctcctcttccttatttcttgctgatttgatcatgttgcttttcattctgttcctctcctttattaacttggagtttccactgttccattccactaatggattctccactttctgtgctgttgtgatcagataccttccctttttactatatttttcaacaaactcccaactcactcccccaataatattaactgtatctctccccacaaaatgcaatatgtcctccctatttctgattccaataagatgagacaattagaaaaatgtcttccattcctcttcttccctttctccactccttgtgacacagagactttgaaatacttttatttccctaactcctagatgttgatggactcattttatacatacagcactaaattattattagaattttctgcatattgtgttttaagagcccttatttagcaattttataacacatcattcctagacagtcttacttatccattcagaattaattcaataaagagagagagaaacagagagagagatacagactgagcacacaccaggtgtgttgcttaacacagcagtccccaacctttttggcaccagggactggttttgtggaagacagtttttccacagacaggggtgcggatggtttcaggatggaagtgttccatctcaaatgatcaggcattagattctcataaggagcacacaacctagatccctcacatgcttggttcaccatagggtttgcaaccctatgagaatataatgtcgctgctgatctgacaagaggcagagctcagctggtaatgctcaccggcccaccactcacctcctgctgtgctccctgcttcccaacagtcaacagacactccgggtatgggtccatgacccgcgggttggacacccctgaattaactgaattaacaagtttccccctcctcctcatgtttacttcatcttgaggtcaatgtctagattagcactattccgtagaactttctgtgatcatgtaaatgtccactagggtacctaccagatacatggagatatggagctctggaaatatgttgaggatgactcagaattgaatttttgatttgattttattttaattaattttaatttaaatagtcacatatgtctagtgggtaccatattgaaagggaagatctagatcatctgttatttggataaagtcttgcctagcattacctcaaaatggttagagataactgtaatatatgatcatgttttgctttcttaaatctaacatataatcacgtatatatttggttttggttttgctaatgagtgaattatatcgtggttgagtataggtttcccagtcctttcctttcaggctgggcttgctagaagcagaacctcagatggggattcttctgcaagtgacttactgagggagatgtctcaggaggcacccgtgacagaataaggtaggcaggagagtgctgaagaatattatggacagagctgtggtttcagttccagtctagcctgagcctgagaccatggagtatcaatggcaccatggggttgtctcacctggagatagcaattctgacttttattaccatttttcaataagtcattagctgagaccatccccaaagaaaggcataacaccgcaggccccgctgcatgaggctgtgagcactctctggataactgtgggctgttctcaggaggagtggcctgccattgcccgtcagccaccacactcagacctaagtcaactgcaggactgtacttatttaaacttcaggtttttagataataagcccattggtattctcattctttcttggaagtatgtaagagtttttttcattcttagaattaaggaattttctaggatatgcctacataagtatttttttttttttttttcaaatcagcccattaatattctctctttcctggaaatatgtaagagactttttttattctaagagtgaaaaatttttcaggatatgcctaggtgtgtcttttttttttttttttttaatcagtcctgccctgaacttaatgacatttttttaagctgtctattcaggtccttagctcaaggaaattatcatctattatttgttaaattctgcccgccatctgttcctttttattcttctgacactcccttattttgcatgttaagtctctttttcccctatgactttcctctctttatatttttgcataggttttgagacatctcttctacttgtcagtctaaacctcatgtctcagcagtgaccatcttctccttcaatcaatgctttgaattgcctatttccagacactctcttttgtgtcgtgcttgaatcaacagagatgctcttgttgctttttacattcaagttgtcatctgtctcttccggtagctctgttttgttgggtaagagtttaatctgttctgagccattgttctctctctctctctctctctctctctttctctctctctttctctggctgctggatacctttggttacaatatattaatatatatatattttttctggctattttctttctctgtgtggtagtagtcccatgattagaggaagacaaaatggaaactacacctggggactgatggcgtcatgcaggcagactgtcaattccccattgagatgcacggaggaagccctctcttcctccaccatctctcagactctcccaacctccagggcagagactctgtatagcttttaaacaccacatctccagagctgggaaatccatggtgccatagttcacggtcttgggtccccatatcactgtgagccatgttcttcccaggacctgagcctgctgtccacccatgagcagctagagcgcaacctcttccccatccaggtggaagcaagcccacaagctctctctgttgagatgtcctcagcacccaagccttgctggtccaactgcccatcagccccagacgccatgtggtcaaaagaagtggacaccctggagttgaacatggaggagagggagaattgtttaggtcatgatggtcccagaatcctcctcctgctttccgcaatccttctgaagagacctgaattcaaattttcattgttcccttttaggatctttgtttcaacaaatgccagccaaataccaagcccagtgctgaggacacaacattgaataaatcagggatgagtccttccttctaggaatgtttactccaaaggggaaataattgtccatgtgatctagctctaattacagagtgttgtgaatgggcaaataaaggctgtggtggagggcagtgttgctgattcatatttctgccctcactttctgtacccaccttgttacaacagaacttccgtccatgttatctgtacactaaaggtgtccatttcattccatctcatccttggacaacgtttaacaagttgcatgtgaaaaaaggtggctgagatgatggaaaatctagaattcaggtgatctgatgattggctgagagtactgatgattggctgagagcactaatgattggctgagagcactgatgattgtctgagagcactgatgattggttgagagcactgatgattggttgagagccctggtgatcttctgtcaaagaatcaagggagggaagtaaacaattactttcttcaaacattgaatgactgtcccacagcaggggaattaataacctctaggaatcccctggaaggaaaaataggaagaggtaagtgaaggcagcacggagagactccagtactttacctcaatagggcaagacttggaaatgtgttatccagcaatgcaaggggctgccttggaaaggcgagctcctcatctttaggaatgttcaaatatttatccaacggcacccttaaaagggacctgaacacagcagacagggcaggagaaaaagccaggaagaactgggagttgagaagtttgttagggatttggtgtctgagaaatgagagtattgggcagtgaaaaggggtaagagaagtgtggccagggttgccaagtgtgtctccttcaaagagaagcgtggaaggaatagaattgtttttcagttttatcttgccatgtcctagataatagctaagtttcctgagatcaaaagcagtcatggcttttttcttccaaactttctttgtttctattttctctggtctacctaaagtaaaacaacagtaaacaataggatctggccaggcacagtggctcacacttgtaatcccagcactttgggaggctgaggcaggcagatcatttgaacccaggagttccagaccaggctgggcaacaaggcgaaaccctgtttctactaaaaatacaaaaattatctagttgtggtggtacacacctgtagtcccagctactcaggaggctgaggtgggaagatcacttgagccaaagaggtcaaggcttccgtgagccatgatcccatcactgcactccagcctgggcaacagtgagattaaaaaaaaaaaaaaaaaaaaaaaaaaaaaaaaaacgaaagaaagagagagaaaagaatctgagagaagccactttctaccttcctttcttcctcttaaaacctccagtcctgaaacatatgatatgttatgaaaatatcgattcatctcagcttttccttaagttgcttgtacttatagaactgtacctgtcaccctgctcaataaagatttgatgattgaattaaaaataagttccagccgggcgcagtggctcacgcctgtaatcccagcactttgggaggccaaggtgggtggatcacgaggtcaggagttcaagaccagcctggacaacatggtgaaaccccatctctactaaaaatacaaaaaaaaaaaaattagctgggcctggtggcacatgcctgcaatctcagctactcgggaggctgtggcaggaaaattacttgaactcgggaagcagaggttgcagtgagccaagattgtgccattacactccagcctgggtgacagagcgagactctgtctcaaaaaaaaaaaaaaaaaaaaagttccaggccgtaatagtctgtcctgtgtatcaacatatgtctcagcaataacactgggcaggggatcttaacagaaaactctgtagccacatggagtggagctatgttttatcaatttgattataatttagaggggttaaaacttgggggggtacctgactctttggagttatgtagtatttactgtatgttctggcacttcctagagctgttccttctcccttgatagtgtcgcccagcgctgtatgttccttggagtatgctgtcatctagttgtgtgagagccacattacaagcctatttgtcacatgcttacataatgtttatttttccttatactaatattacctccactgaatgttaattcgttgtagcgtttcttttgataggttcgaccttttcttctgagatgtatcttcaatttcaggacacaatttagaggcttaaaggtatctttgatgctttgacaacaaacctcatgtaaacttccactttttttatttttcgtgctttggtagaagagctgttcagataagaattgggattcttgccgggcgtcgtggctcacgcctgtaatcctagcactttgggaggccgaggcgggtggatcacgaggtcaggagatcgagaccatcctggctaacacggtgaaaccccgtctctactaaaaatacaaaaaatcagccggccgtggtggtgggcgcctgtagtcccagctactcgggaggctgaggcggaagaatggcgcgaacctaagaggtggagcttgcagtgagccgagatcgcaccactgcactccagcctgggtgacagagcgagactccatttcaaaaaaaaaaaaaaaaaaaaaaaaggattgggattcttgggtctcatccctgcaaattgagtaagtctctcagcctcagttttatcatattacaattaagcaattaagcgcttaactgtgctagctgtttccaatgtcattacattaaccaactggtataaaaataaaatcgtctgggaagaattacttaccgtacagatctggggcccaatccccagagttttgactcaatgtctttgagaaccactgggctaactcgtttctttccactgattgaaattttaaatggttaaagcagcctggctttctcaagaaaagtacacgggcgccacctagtgtccctgaaaagttcgtacagctcacacatcagatgtattgaagctttaacttggcagggttaccagttataaatgcaatcgaatttgcattttaattagtgaagccgattatgtattgagataaacaagtgttgtgccatcgactcacttggaataattagtttgatctcacttttgatttatgacttggcatttttctggactcagttatgcctctcaggcttattctgtctgaatcacacacaattgcaatagtccagctttttgcaattatccgtatttttgtacaactccagaaaaaccaaagtgaaagaataaacaaaaagattttcccagaagaaaatggcaatttctaacttcttcctatgcctactggcattgtgggacagactgccaaggaattcaaccacattccacccactacccacccctggacttctatccttagactctcactgcccccagaatagggaaggttatcaggcccctaaggaaggacgcatgctttgcttggctctctgggccttgtctgggcactgcaccccagtgtattatcaccctgctgtgccatagagccccacatcccacagtgcaagccacaggctagcagcagcagcatcccctgggagcttgttagatgtggaatctcagtccccagcccagccctgaaggatgagaatctattttcgcaagatgcccgggggatgtatatatgtgttaaagtttgagaaaccctaccatagagaacacaaaggctttagccgaagagaacgctgattcccttcccaacatgctacttgtctttgccgtgtttccagcagcataaaatagtctggaaggatgaacattgggcacatctggcgttgacattagaatctgctactcgctactggcgtaatttgaggagcacctgaaactctctgtgctttcagttcctcatctgtaatataggaactgccctttttaattcaggaagttgttgtgggctaacatgtgtactgtatttgacatcatgcatggttcatagtgataaatattaatttcatttctacccatcccctgcttccaggtagaagtacaatggagtgtagtttcaacattgctttattttaaaaattagccacagtgtctttgacatgcttcccatcagaggtggtgtctaccttccctccccttgacgtgggactggctcctgattctcttgtaaccagaagaatggggaagaagttactctgaggcttctcagcactagaaaaggaaaggccatgtagctttcaccggttttcttgggttgcttgctgtgggggcagcatgcagcattgagaaaactccatgctggagaggcctcatgtggtgctccagcagactgcctagctgtgcccccagacaagagtcaccatccgctgccaggccacgggagtgagcatcatggatgtctggcccttgtgccctcaggtggctgcagccccagccaatatctgactacagtgatcgacaactgcccagcagatgcccttccaaaattcctgacccacacgattatgcaaaatcaaagagttgtttgggggtgatttattatgcagcagcagtaactggaacacactttggttggtggtttttgttatagtttttacttcttcaacgaaggacttattctaagtcaagattcgacaaactgtagccctctggccaaatctagcctacatctgttttaggttattgtgaattttctttaatagactttgctttttagagcagttttaggttcactgcaaaattaagtggaaagtacagagatttccatatgccccctgcccctacacatgcacagcctcccccttgatcgacattctctaccaaagtggcatgtttgttgcagtcaaagaacctccatggacacgtcattatcactcaaagtccatagttcacatcagggttcacttttcatttgcacattctatgggttttgacaagtgtctgagctgtatctaccattgtcatatcataaagaatcgctgccctaaaaaatcctctgtgctctacctgttcatccctcccacaccctaaccccggacaaccactaatccttttattgccaccttagttttacctgttccagtgagtcctgtggttggaattatatattacatagcttttctcagattggcttcttttacttagtaatatgcatttgtttccttcatatcttttcatggctcggtagcacatttgtatatatgttaatacattgtggctacaataattttgaacaaattgatctgttagattaagagtaacaagttaaaaatttcattttaccttcacttatttcttctctgatgttctttcttttgttatgtagatatgaccttctgacctataacaatttttattgccttgaataacttctttgaacatttaacaaggcagatctgctagcatcaaattccctgaatttttgtttgtctgagaaagcctttaagtctcctttacttttggaggataatgtcacagagtatagaatgctagactggtggagtttttttctcaacacattcaatatttcactccactgtcttcttgcttgcatggtttccatgaagtcagatgaaattcttacctttgttcctctaaggaagtaaggtgtttttcacctttggcatcttttgggatttttttccttatctgatttttctgtagtttgaaaacaatgtggctaggcttaattttgacattcatcgtggttggtgttctctgagcttcctggatctgtggtttggtgtctgattttcagtcattattgtttccaactttccatctgttcctttctctctgtcttctccttttggtattcctattaaacatataccttttctaattatcccacagtccttggatattctatgcctttttcattcagcattttttccctttgcttttcagttctggatgtttctattgagatatcctcagcctcagagagtctttctcagctgtgcccagcctactaatgagcccgtgagaagcatccttcatttgttagtgtttttgatctctcgcatttctcttttattctttcttagaactttgatctctctatttacattgcccatctgttcttgcttactgtccactttatccattagagcccttagcacatgaatcatagttattttaaatccccagtctcatagttccaacatccgtgccatagctgagtctggttctgacatttgctctgtcttttcagactgtggtctttacctttttttatgccttgtgattgtttgttgatagccagatgtgatgcactgggtcagaaacggtggcaaataggcctttggtgaggtgtggggaagggaagggttccatagccctgtgattaggactcagtgtgtagacctgtgattaggacccagtctgtcttgagcctgtgccctggactgtgagcttcattcagcttctcagtttttccctgagctaggacagggtggctagggggaaatggagttgggtttttattttcctccaggtccattaggctctgcaaaaaaattaaaaaccaacagtttaagctctgataaagtgatttctattgagggaaggccttgttaagaactaaatgctatggcatatttttaagtggtttctttttccctcctcctgacagaagcatgaacagatttttctctgatagtcactgtgagaacctggttaagctcctggagataaaactcacaaaagggtgggtgccccctcagattgggccctcctggagattttaactcccagacctgtgcacactgagctcccagcaactcctcaatcccagttcagatttcctacccagtactggttcccatggacagttctgcctgtgggtttctgctctggtaagctgtgatcttctgtatcctcctgtctgcctctccaaatttggaggagcagtttgccctgtggccttacttctctgacagaactaagaagattcttgaaattttctgtttgtttagtttttaaattattgttaggatggcatggcaacctccaagcttcctacgtgtcactggaaaatggacatcccatctgtttttgtcaataaagttttattggaatacaaccacactcattcatttctacattttctattgccacttctgtgctgtggcagagtagttaagtagttgccacagagacttgatggcccacaaaacctaaaatattagctctttggacttttgtagacaatttttcatcatgtctgttctaggtaatagggattgaataataggtaaaataaagtctatttttaatagattgtatagtctaatggggggggagatacagtaaaaactaataaacatcacgtggtgataaatgtcatgaggaaactagagaaaaggggaaaaagtgacagggtaatggggtggtccaagaaggtaaagtaacatttcagcaaagatgagaatgggttgagggagcgcctctgggggtagctgggtgatatggtttggatctttgtaacctgctcaaatctcatgttgcattgtaatcactcatgttggagcctgggcctggtgggaggtgattggatcgggggggtggagttctcacgaacggtttagcaccaaccgtcttggtactgtcctcatgataatgagtgagttcttgcaagatctggttgtttacaaatgtgcggcccctccccactctctctcttgctcctgctctggccatgtgacatggctgtttctcctttgccttctgctgtgattgtaagtttcctgaggtctccccagaagctgagcagatgccagacttatgcttcctgtacagcctgcagaaccttgaaccaattaagcctcttttctttataaattacccagtctcaggtgtttctttatagcagtgtgagaacggccaaatacactgagaaatatgttctccagacggagacgacattaagaggaaatgcgctaagtgtgccaggaggaacaacagacagggcccagtgagaactggagccgcgtgtaggagctgagggcagggcggccccagaggacgggcctcattgagagcattagatttgggggtctgctgtctgctctgtttcagtgtctaatcagtacatatccctgcagcattttccatggggggccagacaccctgtgcctcaggggcatgggggtggcagaatgacattgttttgcctgcaaagaggagaacagagtttttcaggacggctccctagaagagacatcacagtggtagttcctgcacaaagaatttatggaggaagtgttcccaagaaaaactagtaattgcatgaaggaaataggcaggaaagggacaaaagtgagtaatggacagtctcaagtaaattcccacctctgtctgattcccagggagctctggagcacaagtaactccttagactttcttctctcttgaggcagagttgggctctcacactcccatatagggagggaaaaactttttccactgtcttctgggttcttcagctgaggccctgcaaattagagttatgaaagataggtgaagaacaacaataaaaacaaacagacaggctgaagtttattagcacgtgcagcttgcatatacacgggagaactccgtggtgagtaactcaaaggggtgactagaccttggggcttatatggcatcttaacaaagatggataattttgtagagaagtgacaagaaaagagcttttgatgattggggtggcaaattgtgggaaggtaaacacatggggaaacatataaaagatagggttagtgagtaaggtgtgtttgtgtagactctctcagtaccatctcttctctgataataaggccgttatccccttcctggaatggcagtttgggtgggggcagggacaccttcacaacaggactttatgttctgtttttgggcagataggaggagagtagagagttcattctgaatccactttttctcaattgcctccacttaaaataattcctattccagagtggcatattactccccggcattaccacttctctgcctatgcaggcaaagggattccaataggccaagaacagtccattggagatgaatatcttggtcctttcctgctgctgtaacaaaatacagggaatggaaaatttaccaacaataaaaatttatttctcgcagttctggaggctggaagtctaggatcaaggtgctagcaggttttgcgtctggtgagggcacagtctgcttccgagatgtccatttgaatgagcactgaaggacaaaaaggccaaaaggggcaaaggccatgtcctctcgtggcagaagagatggaggggaaatgggcccacctagctcccttaaccttttataagggcactaatctcattcatgacatagccacctcctgacagccccacctcacactgggatttcactcccaacctgtgaaatgtggagggacacatacattcaaatgacagcaataaattacgggcacagaccattagaaacaaagcacacagatgccgagggagggacacgccagaacagttaaagaaatccaagcgcatctatgtagcctgtcaacagagtctgctacaaagaccaaaaaggaaatgccttaggatcttttgcacatgccgacatcttatatttgcctatttgttaattcaagcatcaggtacatcccttgtttgctatttggctaaatatattaatatatatgatcttaagtaataatgctgcaagcacactgcaccatgcagaatcggtctacagtcatgggatggaaccacatcaaaaccccatgcccagcagttttgttttagaattccagagaaacacaacagagatagctgccagtacaggcttactgtgcaaggccacattcaagctctacctaggggcagcatgcaagggaagtgtttagtaatgattatctggaggatgcaagctaattcttccatttctttgatgccttccatagtggctataccaattctgaagacattcaatataaaaataactgtgaagtttattagccaattatatacattcggctttgcgtatggcggtgtgatttcagctctcaccaagatactttcaggaagatgactttaagaccttgcggttgcttttatctatcttcacaccagggggcaggcttgagtcgtttttaattcctttgtgtgttgttcttagaaaacaaaggctgtgtcttatttaaaacaaaacaaaacaaaagctactacacttggttacctcaaaggaaaagtaccatttggcacttacttctaccgtcacaggctgattacagaccttccatagaatggatttagagtgttacatatccatctgtcctgtctggatttctttccactccatttggaaccatttttgaggatccatttgaattttaaacaggaagacataatgtctatgctaaagttccaccgactaatatacaggccaaagtttccaaattcctaaaattgaaaatttgtcaaaatttatagtcatttttaatgttttgtgttgctggtgtaaaatcttccaggatagtatggccagtgcccaaatgcaaaaacaccctcatgttcctgcctctgatggctttgaccactggtggctgagggttggcagcagaactccaggactgtgcctttggtggactcatttgctccatgtgacccccaccataaacgcaggccttgcagaaggaaaggaagagtttgcatctcggcctgctctgtccccttgagtgtctttgcctggaaattgtcagtgttttcacacttatgttttgccctcgacctggatatgtgggcacctatgcgaggttaaactgtgatactttgattttcatcaatctggaaagtggtggcactagctcagttgcttagggcaggacaaacggggttgcaactgcaggtgatgtcttcatttgactcattctcttggctgtgactctacctgaaagccaactcatcagcttgcaaattacagccactgaactgggcctggggaggcaaaggcaagtctgtcttcattactagagaggctgtgtaaagtgctttccccttcccacccagcatcatgtggttgccaatgccttgttacacaaagcccaggtgtctgactgcaaagagaagcggttctctcatcctgagcccttccaccagggggttgaattcatatgttcccgacaataagtacaacttggtgaagaggcatcactaattcacgaaatctgcagatacgttgctttattcttcatcatgttgaagatccttcatttctaaggttttccttttctcttcttttattcttgttgccatcaccagctgcatcagatagatgaaggagcagcagctggacagtttaatttattagggctgtcagggccaatctactgctgataatagaacacagtagaagtggatctgtctagtaaagcacatttaatctaaatgattcaaatagtgtttcagcagcttcctataaattaattcattttagaatccccctattaacttgcttttaatttcagaatatgtgcacattgcacacacatttttctcttaaaaataccctgtctgctcatgcagagaagatatttgatatttaaaatatttttcctctgattttgaaaacaagattgaaagtcaaattaaaagacatcacttgagatcatggatgtctgaacaccatgtcttacctagagacaatggaggattacttgccagttcaaatccacaccaggaaggaatgcttccacctcaagttttggactgactggctgtcctccaagcgctggagtcctcttggctaaggcattcatagaaacttcctaacttttcattatttcaaataatctcaaatttacagaaaagttacaagtaccatataaagaactttatgtcctgaatctattaagttgccaacctgacattctgtcattccaaataccatagtgtgttacctacctgctatagaatgaatacttgtatccctccaaaatttgtatgttgaaatccttaccctcagtgtggtgatatttggaggtggggcctttggcaggtgattgattaggtcaataagggcagagcccttatgaatggtattagagtccctgtcaaagagaccccatacagctcccttgtgtgaggttatagtgagaagatggctgtctgtgaaccaggaagtgtgccctcaccagacactgttcctgctagcaccttgatcttggaattcccagcctccagaactgtgagaaatagatttctattgtttaaaagccactcagtctgtgacattctgttaaagcagtctaaatcgactaatagtaccaaaaagaagcatcattttctttttctccttttcttttttcttttctttttttttagacagagtcttgttgcattgcccaggctggagggcagtggcatgaagatcatatctcactgtagcctcaacctcccacgctcaagtaatcctcccaccttagcctccggagtagctgggactgcaggcacatgctacctaccatgcctggctaattttttaaaatattttttgtagatacagggtctccctagtctggtctttaattcctgggcccaagtgatcctcccaccttggcctcccaaagtgctgggattacaggtgtgagccgccatgcctgcgttttctacacagccacatgccaccatcaacattaagacattaacactgagaccttactgccacttatttatcaggtcccaagaaggttctccagttgccctaataatgaagatccaattgaaagtcacatgttgcatattttgtcatatctctttttagtcttattgattccagaacagttcctcagcctttctttatattggccttggcacttctgaagagcacaggccagttgttttacagaatgtccctccattttcctttggctgacactttctcatagtcaggttcaggtgatgcacctttggcaggactatcagagaagccatagtgggctgcaccattgttccagattcagtgtgtttcatggctggcggcgctcactctgatcactcagtcgaacatggtgtctgccaggcttttcaactccaacgttactcttttcccctctgtaattagaaggtatgggaggaggaagttagtagagaatatgaaatatagactttcatttttaaaacataaatcagcaaaagatcttttatatcaaattttagttaattttctaaaattgattggctttcttaagtctctttgttctagatgcacacagaaggaaattatctcttggtgtctctgtttctcatttcatggggaggcaggtcctacaatgcttgaaaaccaaggcctatactctctcacccctatgtttttaagttttaattgacatttaatgtattctttaagtgcacaagttatgaagtgtacagcccaacacattttcacaaaggaaacacatcagatcaagaatcacgatgttatagacccaccccagcagattctcaccttgtgtctgcttccattctcaatcacctccatgggtcaccgttgccaactatacattttgctggtttttgaacttaattttcatggaattctttagtacctgtatgattgtgcctgatttctttcactcaacattatgattatgagattcagccttattcatgccactgtatataattgtagttattgcccctcctgggctacggtccacatgtggggtcttttactccttccttcacttgacaagcacctgtgtattagtccattctcacactgctacaaaaaattaactaagactgggtaatttataaagaaaagagtaattggctcacctcctctgcaggctgcacaggaagcatgatgctggcatctacttggtgtctggggaggcctcagaaaacttacaatcatcggggaaggcagaggtggagcagatacatcacatggctagagcaggaggaagagagggcggcaggaggtgccacacacttttgaagactgagatcttgtgagaactcactcattatcatgagaatagtaccaaggggatggcactaaatcattcatgagaagttcaccccccatgatccagtcacctcccaccgggccctaccgccaacattggagattactatttgacatgaaatttgggcagggacacagatccaaatcatatcaacctactaagcatccagaaatttaagtaaagtgagtaacactggtgcttacacttttgaccatctagtctgtctaaattctcttcacttttttttgagatggagtctcgctctgtcacccaggctggagtgcagtagcgccatctcggctcactgcaacctccacctccaaggttcaagccattctcccacctcagcctccccagtagctggtattacagctgcatgccaccacacccggctgattttttgtacttttagtagagacggggtcttaccatgttgaccaggctggtctctaactcctggtctcgagtgatccgcctgccttggcctcacaaagtgttgggattaaaggcatgagtcaccttgcctggcaattctcttcacttttaacccagtggggaagcaatagtagttgtccactgtatacctgacactgtgccaggtgttaacacataccatgtctcacttaatccttcctccaatctagcaatatatcattatgtctgttttttagggaaggaatctgatatttacagagaggtttccatagttcgcataagaccttatagttaggtgacagagcagatattcaaacctacatctatccatgtgcaaagctggctacagcagtcatgaagatgccctgcttcatatcccctgaacacacctgagtttacctgcagctttggtggactcatctcatgcacactaacagtttctctctgctctgtgtccaagggctttctctgacatgacaatcctgcccagaaatgccaggcaatgaatgcccccaaaggtaatccttaaccaatgtggaaaggaagttcttggataaatacccccgcttctctgacctgcatttgacatggctctgcagggcatctgctgcagcacagaggcccagttgcccacagcattaaccagctcaatggggcacccaatgtggatttctctcattttcattgcactttcttcagtctgtcatgtggctttctggaatctcctcacaaataaagaacctgcactaaagtccttcttccttgagttgctttcaagggaacccaaattagaacacttattctctctacagttccttgtactggggtacctagtgcttctgaataaagctgcatcccagataacgccccagtgaggagttgtacaagcctgggcttttcctccctgccatagaatgcctagcctacagcattccatggccccttggaggcggaaaactgacaagacaaccaggtatcttttctctaaatgtctaaagagggaggagaaagaggtgtcattttggtgtgtgggtggcaaagggacaaagcagatcaagctggagggactcacaaagcaggaggagtcattagaggtcacaagtttggatgcaggggtgtgaataggatttctagctaagagaacaagctttggctctagagagacctggcttcaaaacctgattcctccacttaaactatagctttgtgaccttcactgagtggcttgtcctccatacttcagtgtccttgtgccatatgctgttgtgtcaaactcctgttaacctcaatagcaaaggggaccccattcaagaggccagagaagagacccagagccagaaaatgagacatggggttttattagggggctcacatacaggggagagggtccagtggcaggaaaaccgcaattgcttgcaaatagcatgcagtttatgtagcattttcacatagcaccttccccctaatgacatccagctggcaaccttcatttaacccaaaactcaggacctcagtgccctaggcagcttgtgttccacagaagagcccaggggctctgatatttgtcattgacaaggaactaatcaggttggccactcccggatgccctagcttggaacacacattcaggtgcatctgccatacagggttattctaaggatgtgtttaagttattgctatcaggtgtgtttaccctgatatcaataatttgtggtgtggtgatcataattctgccaatcttataggattttttagcggattaaatttgaaaatatatgtaaagctcttaacacaactgtcaggaagtaaagctttttttatgacgttagcaatgtgaatgtagcagtgaagatgtgggttctgtagctagacggcctgggttcagaggcacagcttttctacctcctggctcaagactcagtttccacacctgtaaaatggggataatagtgcaaatctcatacggttgttatggagattatatacagacagagagggagagcaggcacttcaaagggtgtgtatagcagcacagcataagcactctaatgttatctgtaaatctagaaaaataacttgaatccataagctatctggagtcaggaaagccctatcatgggtgaggatgtacacagttttgcttaactaacgtaccagttggggtattagctcatcaagaagaacaatatggaaagacagtcttaaatgtttttctcaggcagaagctgtatgtgagctggggatcagccagggagaggcagaaacctttgggtgccaggaggagctgattctctgatataaatagggcagaacttggagtcttcagggaatgaggtggaatgaatggaagggattcacagaagcgggaagaagaaaacagagaaggtggagttgacacagataaagaaaatggatggctacctcctctcttccaggtaaagcttgctgctgccaatgtgttctccctccccaactagaaacacttcccaggaaaggccaaaagtgcttgcattacttcctgagagctccatctacccagaaagtcactgtgtcattggcaggtagcttgtaggccatttggccattttctacaccggaggcgtctataatctttcccctccagggcaccattccttccagcttgctgcactttgtgatgcttccgtgttgaaagtagtccctcccaccttcctgggttctttgctaagataaaatgaggcaaaataaatcaacttggctttcacatcaaagccacctctgtctaaaccatctgtgacttagaggaattagaactgaaattcgtgagtgatgacatctttgttttgtttgcttgctttttggtttttgttttgttattttctagttagagtagattgaataaatggatagttttgcctgctaaagctctgttacatgtacagttcaggcaagatggggaatatccttggattcagttctaattattccctcagaggaaaccgaactttgattgatgaagaggatcctttctgtgttcagctcagatgctgtggtcacgtttgactgatttggagcagctctgaatctcagctctgtcaaatattgcatgtaaaccaagcttcttactagctactaatctagcatggaaaagtcctgcagtgcaaaatgatgagaatagaatgatctgcaaaaagaacttttagattctcagaaactgactgacgaatcagcctgaagcactagccataaagataattaattcatgacaggtgagcatctgtagtaagcacatcccagacaccaaggggctgtgctccacctgaatacaatgcagctagccatgggcactattattccctaggttgtatggtagcaacattaggagcaaggggtctgcattcagacattactggagtttattttatttatttatttatttatttatttatttatttatttatttatttatttttgagacagagtcttgctttgttgccaggctggagtgcagtggtgtgatctcggctcactgcaacctccgcctcctgggttcaagtgattctcctgcctcagcctcccaagtagctgggactacaggagtgcaccaccacacccagctaatttttgtatttttagtaaagacggggtttcactatgttgaccaggatggtctcgatctcttgacctcgtgatcctcccgcctcggcctcccaaagtgcattcctggagtttaatcccgagttcaccagtgtattagtcaactatggttgccaaaatgctacatagcaaacaaccctacaaatgctaatgacttataacagcaaacatttctttttcttgctcatgggtcttaggctggtagggttactctgctttgaggttcaggtcacttagagttggctgcgggttttaggttgggttcaggtctgttctggacgcctcttggcattttttttggaccagcaactgcccaggtcaggtttttttcagggcagatggcaaagactcaaggaaccagtcgatatatataatagcctctgcttttgtcagttcaccaacattccagtggccaaaacaagacacgtggccaagttcaaagtcagtgggtcccactctactgggaggctcttaaaaagtcacatgcagacggtgagatatatcatcatcacagggaaggagggaagaatcgagagaatatttcacctgctacaaccactaaaaaagctatggaacctcagtcaatgcttgtaacttcttctttctgatttgtaagatgaggtgtaataataatataagtctcctagggctattgggacgatcaaatgcaagagacagatgatagcacataaagattaataaaggggaactttcttgttatagaaccaaccctttctgttttaaccctttttgtctcctgacctctttcccccattctggtagcaactctcttcctcaatttctaaattcttggaaattctgcttgctttcctctcatttccagggctaattcaaattattatgcttacacatgaaattaaggcaatgtgaatatctttgcagactctcccttgtaataggctgtgggaaagatagatgggaatccagatatcagctttattattataaagccccatttactaaggaggccttgtacaatatacacctctatctcaccctccagcttagatataagagagagctctgcaaggctgatactggtcaacctgctacagaggagtcactttacacacacttacctgcctgtttggggtcagagtgggactcttaacctccagaagagaagttctgggtccttaccccatggaagtagaaaacacaggctatgcagacaggaaaactctccagaaaaaatgaagagaaggtcctttgagccatgcatgggccctaaccgttctcctttctcccctgccagataagccacactccctgccacatgcagatgagtaggggaggtgggagagaacagaagtattattccagtggaagactctgcagggaaatgtgaggacagcagagaagcagttcccctgacaacctgttttgcagataagaaaggttaatcccttgctgggagctttctatccagcagagaaatcagcagccttagagtcagtggcctggactaagatcaataatggttatctattatgacaaataagtctgtgtaacaaagcactctgaagctaaagagcttaaaatacactcaaactgcaaaacattactgagagaaagaagacataaataaatgaaaagacatcctctgttcatacattggaagacataatattgttaaggtgaaaatgctacgaaagtcatccataggttcaatccaatccctatctaaatgtcaacagtgttttttacagaaataggaaaaccatcctaaaattcatatggaatcccaaagggcccaaatagccaaaactctcttgaaaaagaacaatgttagaggattcacacctcctgatctcaaaacttactacaaaactacaattaaaaccaaaataatattataatggtataagaacggacatctaggccaatggaatagaatatagcactcagaaacaaaccctcacatatgaggtcagatgcttttccacaaggcaaccaagactatttaatggagaaaggataatcttttcaacaaataacgccgggagaactggatacccaacagcctaaaaaatgaaggtggccccttatcttataccatatacataaattaactcaaaatgggtcaaaatgtaaccacagaagttaaaactataaaacttttagaaggaaacatggcggaagcttcatgacattggatttagtaataattccttgaaaattacaccaaaagcgcaagcaccgaaagaaaaaaaatgataaattagacttatcaaaatgcaaaactttttgcactgaattacactgttaagagagtgaaaatccgtagaatatgagaaaatagttgcaaataatatatctgataagggattaatacccagaatatagaaaaatctgtaacaactcagcaacaataaaaagaaacagcccaactcaaatacaagcaaaagacatagctagatactgctccagagaagatatatagatggctaataagtgcaggaaaagaaaatcaacatagtctgagaaaatgcaaatcaaaaatcacagtgagatactaagtcacacccattaggatggctatgattaaaataatatttttaatggaaaataacaagtgtttcaaggatgcggagacattggcatcctcatgcagtgctgatgggaataccaaatggtgcagctgctgtggaaaacagtttgttgattcttcagaaggttaaacgtggacttaactctagcaactccactactcagtacacccaaaatagttcaaaacaaggacttgaacagattattgtatatcaatgttcacaggagcattattcacgacagccaaaaggtgcaacaatccaaatgtctgttgacagatgaatgaatggataaacaaaatgtggtagatattattcaacttttaaaaggaatgaaattcttatacatgttacaacatggatgcaccttgaaaacactatgcgaagtgaaataaaccagggacaaaagaacaagtcttgtgtgatttcacttatatggggaagctagataggcaaagtcatagaaacagaaagtagattcgaggttaccaggggctggagggaaggggaattattgtttaatgggtacaaagtttctacgtgagatgatttaaaaaaagttccagaaaggaatagtggtgatggttgtacaacactgtggattgactgaataccaccaaattttatacttagaaatagttaaaatggtaaattttatgtatattttaccacagtataaaaagagtttaaaacaataagcatttattcaaatttaaaagaaaaaaacatacagccccatcaaaagtgggcaaagaacatgaacagacacttctcaaaggaagacatttatgcggccaacaagcatatgaaataaagctcaacatcactatcattagagaaatgcaaatcaaaaccacaatgagataccatctcacaccagtcagaatggtgattattaaaaagtcaagaaacaacagatcctggcaaggctgtggagaaataggaatgattttacactgttggtagaaatgtagattagttcaaccattgtggaagacagtgtggtgattcctcaaggatctagaaccagaaataccatttgacccagcaatcccattactaggtatatacccaaaggaacattaatcattctattataaagatacatgtatgtatatgtttcttacagcactatctgcaatagcaaagacacgggatcaagccaaatacccatcaatgatagactggataaagaaagtgcgatatgtatacaccatggaataccacacagccataaaaaggaatgagatcatgtcctttgcagggacatggatgaagctggaagccatcatcctcagcaaactaacacaggaacacaaaacgaaacaccgcatgttctcactcataagtgggagctgaacaatgagaaaacatggacacagggagataggccaggaacaacacacactgggcctgtcatggggggagggaaagcatcaggataagtagctaatgcatgcagggcttactacctaggtgagagattgataggtgcagcaagccaccatggcacatgtttacctatgtaataaaactgcacgtcctccacatgtatcctggaacttaaaataaatttttttaaaaactgtttattgttcctcacaattcagcaagttggcagtttggcctgtgcttgacccatggttctagcctcatcagggctcactcgcatcagcttctgagttggctagaggctggctggcctagcatggcctcagctggaaagactggtctctgttcctcgtgtctctcatcattcaccaggctagtacagacttgtacacatggtaaccaggcaggattcaaagctgcacagcctctagaaacgtagacttgattgggcacaacatcatctctgctgtattccattggccagagctaaacccaaagtcaagggaaagggaaacagactccatctcctgatgggaggaggtgcggagtcacactccaaaggacatgcatacagacagaagaggagaattggaaccacttttgcatcaacctactcgagtttggttctgacattgttaagaatgtgtcaggacaccaaatggcatgtgacaggatgtgctcatgtaagtaacctaggatttgttcacataaagggtcagtgcttgttttaggaatttaaaagtgaaggaaagcatacacaaacctcagttcatgcatacagttttctctcccagtaggtaagcacctatcagattgtttttcaatcaagccagctgctttgcactttgtaggagctgtaactatctggattagctgagacatggagagagccttcaaagcaacagaattttgaagctagaagggtgccagggaaaccctctcagtacagataaggaccatagccccagcaagggaaagtgatttggtgaaggcttcatagttgccacagatggaacttgcactagaatccagattgccagatttataatccagcactctctctaggacatggtgctgatttcattttccctggaatatgtaaaccccagttcagccaccaagggaacacaatctattctttgagaccttgcaactaagagcatgctctgcagccagcagtattagcctcacctaagagcttgttagaaattcagaatattgagccttatgccagacctgctcccttggagtctgctgtttaaggagatacctggggcatttatgtgccaagtagggtttaatacgcactgcattaggacacgggttttcaacctcaaatactaggaattgcctgggagaggtttaaaactaccactgcctggtctttaccccagagattcttatttattaagtatttgctcttgggtgcagtctgggctaaaggatttttaaaagctcctcaagccacgacttctctgtagtctttttttttttttttttttttttttgagacggagtctcgctctgtcgcccaggctggagtgcagtagtgctatctcagctcactgcaagctccgcctcctgggttcacaccattctcctgtctcagcctcccgagtagctgggactataggcgcccaccaccacgcccggctaattttttgtatttttagtagagacggggtttcactgtgttagccaggatggtcttgatctcctgaccttgtgatccgcccgcctcggcctcccaaagtgctgggattacaggcgtgagcctccgtgcctggcctctgtagtctatttctaagatgctgaagacattggcagctttttatatagttttctgtctgtgcagtggggtagtattttcctaaagagtaaacagccttcagaagaccctagttgattgacctgctccagcatcctgggtcagatgtaagatcaagggattttaaaattgctgaggactacttaaatacattttcataagggaggaaattaaagaccagaagtggtcattgagaggaattttttaaaaatcaactttgtttcccaagtggaatagcattaatgtacaatgaaattccatgtaataaaggtgtggcaaattacatagttcaacagtgaaggccaagaaacacgtaaattatggaaggaagaagagacagaagggaatgcaagtagactacaggccttacacatgaacaaaatcaatagttaagctataaatttgatttttttcaagcctctggggagtccatttgaaacagttacaagatttctatcaacagaaaaggggagtgggaaaagcagaatttcctctaggaaagccaagctttctaactctaatttgtaaataagtgtgtcaggaggcctttctagggggacacttaatgaagtagcgaacgatgtcttcaagaacattcctgggccaaaacacccctggattttatgtttcttgttaaagaacccctggattttatgtttttgttaaatctgtggtctttatgtgaaaacaactgatagaatacaattttggaaacaggaaagataatgttgtttgagtctcttctgatgtgtgtgttttgctttatctaagatataactaagctttatctgagatataacttagagaagaggagcttcaatttggcaaggcagcttagagaggaaagaagtggatcatctctctttgttggtacaagggtttgtgctgttctgttagtatcattgagggaatcaatgggattattcagtaaactcttttcagagtcctgcattctggacttaaacttgccctcttggataaacacatgggataaatacgctccatcattcagtcacagaatgcattctaggagcataatgtccagaggtgcccttggtgttgtgaatcaatcatttttccttttctgttttgaattgctgtttctttttctattaaatgaaagaatgaccctattaatctgaagtttaggggctagattttttttatttgatactatctctctctctctctatatatatgtatatgtgtatatatatgtatatgtgtatatatatatgtatatgtgtgtatatgtacatatatgcacacacacatctaaatacacctaaatatacatatatctaaatgtatgtgtatatgtgtttatatatatcagtaaattctctccgtaataaatatatgattttatatatacacagagggaatggactgattttaaaatccttattgagttgaaatggtttggatcatgaaaaggggaccttaggagtaaagactaaatcaaaggcaaatggttgtggaccaaaattagggctgtcgaagcagaagttgattaaaaagatttattggaagtcaaatgtgagaaatgacctgggaaaacacaccagcaaagttgggaatgatccaaagtctgttacaagttggaatgcttttataggaaagtatgggagaaaggagagggactcccatatcagagttgtcctttttcattggagggtacaatacagggattattgtcattgtctacagattgcaacatacaggctaacacatctatgtgcaagacaatcagtgaaatttcctgattcagatacaaaccagcaaaacttcataattcagaaacaaatcagcatccttttcaatgtcagtaggttactcattaatcagtgtgtcaataagataaggaactcagataattctttactcaggaacaggatgttgccatgaatcacaagacctccccaaggcagactaatttagaagcctgcaaatgtgacctgcaggttgtcttggttgaccagtgctgtccagacttcaatgtatattttaattacctggagattttgttaaaatgcagattgtgattcagtagatctggatggggctgcaattctgcattcctaacaaacttccaggtgatggtgatgctgctggcctagggaccaagctttgagtggcaggaggagaagttctagcaatatggttcccaaacttggctgcccattggaatcccctgaagagcttcagaaattactgaaccctagaacgatgatcagagatgtaattggtctgggaagagaccctggcctccaccctgatgattccaatgtgcatggctgggagcccctgcaattgaagctgccagatgaagcggaatctctcactgccttagcctttcctacaggttattagaaatcctcaaggtgcattcttaaaccttccattgggtttcaaaggaacaaaatattttaaatgtgaacacatctcttacccctagataaggagggggagagagagagagagagagaaagagagagagagagagagatccttgggataaatgtctcattttgcaaaaagaaaagacatgatttgattctgtatatagcttatctaaatgaaagaaatgttgcagagttagcgtttctgcggtgcaaaaggaaagcaccttaaggaatcgccacactgtcttccacaatggttgaacagagttgaacaatgagaacaaatggagacagggaggggaacatcgcacaccggggcctgtccggggagtgggggtcaagaggagggagagcactgggacaaatacctaatgcatgtggggcttaaaacctagatgtaacaaacctgcacgttctgcaaatgtataccagaacttaaagtaaaatttaaataaataaataaataaaaaggaaagcaccatagacttgcaggtgtctctgggtaagccaagttagtacatagagatgcatcaggctccactggtgggattggtaaagagattgaggatgaagggagcctaacctaccaactctttttttttcttttgatacagggtctcactctgttgctcaggctggagtaaagtggtatgattatacctgactgcaaactccaactcctgggctcaagggatcctccgacctcagcctcttgaatagctgggaccacaggcacttgccaccatgcccagctattttttttttctttgtagaaaaaaaaagggatctagctatgttgcccaggctggtctggaagtcctggcctcaagcagtcctcctacctcagccacacaaagtgcttggataacaggtttgagccaccgtgcccagccctgccaactcctaatagtatgaatagcatcatttctttggcatttgctaagagttactggaatatttcagctacgggcttgtatctctgattgggtttaagagatgggattttcttcccctctgaaccagccacccacccacagccacatcccacaacacctgttctctctcccttagactttcagtctttgaccttgctgcattctccttacttatcagcactctcctttaataatctcctttcagcttaaactgcatggttcatccacattctagtgcccattgctacctacctacttgcatgacagctgcctggcaaacacccattctgagtatatccaatgtcccacctttgcagaagctgcccatagagtgctgcatatttgctggacaagtcaagcaaacttttggtttggtgcaattccaatgtattttgacaaaggaataatttatgtgtaataagatatacaaatcataaacgtaaagtatgccaaatttttacaaatgtatgtattcttgtgaccacaaccaagatcaagatatacaacattttcatcacccagaaagttcccttgggccccatcccagtgagcacctttcccctaggggtaactagtactctgactttgatcaccaaagagaaattagtaatgaatatcaaggaatgtttggtaaagctgactaaggtacatgtgcttgtttgttacatggatattacacgcatagtggtgggggttctgcttctagtgtacctatcactgaaatattgaacattgtacccaacaggtaatttttcaaccctcattccttatccaccatctcccatccctgcttttagagttcttagagtctattctctccatctttatgtccgtgatgaccttaatatttaatggggttagaagtttcaaacttttgagacatatggaagacttggattgcaacttttctcttctattttttgcataaggcatactcttttcttggcatctaagaacagtcaatttctgaaatatgaattacagtgcatagttacaataccaactttatagtttaaaatgttatttgactgcctactggttttatttccagattaacaagtttcttaacatttattatgcctccaagagataatttccacatttgtaaagattgtttgaaatgtcaggtaaaacaacaacaacaacagaaatttaaatttaggacatgcaaaggtaaatttttcttggctaaatttaaaataaatatgccttgtactataagagaatgcaatacattgtggcccttagcattgcatattagcaatatttagagcataatttaatcagttggttgatttggggaggtagttgcaattgatatgtagactacatgttttattagttattacatttttaaatgtcagaatcttgagtaaatgcaatgtaacatttcctttgttatagagaaagaattttcaagttgctttaaaggctaattatttcaaagaccagtcaacttatttttccccaatttccataaattctgagttttgaacatcctttctttaaaaagctttgtttctggaaatattttatattctgccccccacagaatatgcttacatttctttcattgtatctcaaagggtttggagcttgtctttctgggttttgactatcctttctactcctgagagttgaagttgagggtgctgacagggtgttgtggttttcatattctatagagaggcagcatttgtaacaggtaggtgtgagccttgctttcaaacctcccacctgctagctgagtgacactgggaaaattccttggcctctctgagcctcactttcttccttcttataaaggggtaataatgccataccttggagtcatattatgattattatatattggaaaaatacttctaatcatcttggcacacagcaagtgtttaatccatggtagtgactagaaactcttattaaaccaatttacggactgatggcagtcattttattcgtcagttcacaaattctttaaagttcagatcatatggaggacagggcatgagggaggaaatgatggattcttgtttatgcccacaacacctactgttgcctaaggtgaccttaccacccagcaagaacttggccagtctgcttctcgaattgctgttccagaaaagctcatttgcgaacagtgttgggttgtttatttgggttgcttattctagtcaattgacttggccaacaaactgcatttcttcaggagtggggagaactgtgtttcatgtcctcctctgtgacagaactgcagacagtaaacgaagtgatgcctgtggtgccatatgactccctccctctaagaaaccacattcagcttttggggaggcttatttcttttttttttttttttggcacagcccatgaatcaaatactagcagagttttccctccaaagatgagacctgtgtatctagatcagggttcagcaaactttttctgtgaagggcctgagagtaagtattttacaccttgcaggccatatagtctctcttgcaactcaaccctaccattgtggtgcacaaatccagccatactctcaccatatgtaaatatttacaaaaacaggcagagggccagatttggcccatgagtcacatggcttcctgacttctgctctagatcagttctgctcaatacaagtaaaatgcaagccacgtatttaattttacatttcttagtagcacattaaaaacaattcttggccagtcctggtggctcatacctgtaatcccagcacttcaggaggctgttgcatgagatcacttgaggccaggagtttgagaccaccctgggcaacataatgagacctcctctctacattttttttttaattagccaggcattttggcacatgcctgtattcctagctacccagaataacttgagcccaggagtttgaagctatagtgagctatgattgcaccactatactccagcctggaagacagagcaagaccttgtcttcaaaaaaaaaaatttgtaatatattaaagcatcttgatttttaaagatgaaatgtttttaagtcttagaatagaaagagacagatgccttacctttatgagtaacctaagtttgctgagaccagcctagccaacatggtgaaaccccatctccactaaaaatacaaaactaactggtcatggtggtgcatatctgtaatcccagctacttgggaggctgaggcatgagaatcgcttgaacccgggaggcagaggttgcagtgaacagagatcacaccattgcactccagcctgggcaacaaaggtgaaactccatctcaaaacaaacaaacaaaaaaacagaaattggctttttggattccatgtgattaagattattacataatttgttaaagagggattttctggttagaatcccttaccactacctccactctgaaatatgttgcctgaattatcttgttttccagggatatgcaatgctcaaatgttcacccaattagccccaaccactgttgttaccaaataaaaggtatatgtagagaactgaaagaaaatagataaaatatctattggtaatagcgagtgaaaatatttattgaaaatatctaatgatattacaaaatccaaacaagagttaccgtttcttcctataaggatctaagtagatcaaatctgtttgttgtgactatgttacctgatgtttccagctacctgccttaataaaaacaaggaagacagttctttaccaacagggtaagaatttagatagacagtttaaaagtacataagttctgagagaatctttgtttatacttcagttgtggaaatatttagcttattaacctcctgagctatatttacctgaaagacaaaatgaaacacgtttgaggcacacagaagatactgcttttgaaaaagagatgtgaggtcctattagggcaaagctgttaattacacacttcattattgtcaaggttaatacagtctgtgagggcagaggcactttttcatctcttttcggggaacaaggggagacagctgttttcacagacagatccattatagcttatcaccagccaacagcccatggaaggagcactatgaaatatggccactgtcactgggagcctgaatagatgctgagctggtccagggtacaatggaaagacaatgagcaccatggcttcaaactcaaatagattgggttgcattgatttttaatgcctggaggttccatggcataaaatccaccaatcctcttgcagttattgtattattgatctctataaaagtgcacaattttgctttaatgaacaccagagacggacaataataattagtggttcttctgtttggaagcctggttaaaccgtgttggaaaactctgcatgtttggattttcattattactgctttgccacttatgattctataaagtgaaacctgtatcagcaaaacaaatactgccttggttctgatttaacaggtcttcttttcttatacttttttgggtggggcactaatttgtttttaacaaacatttacattttgcaaccccctgcttattaacactgagctaatagttttcattgaaacggaagagtgtgtgctttatttaaagtgtgatacatttataccatcaacaaacattttcaaatgctaaccatggaaatgaaatccaagaaaagactgtcttaggacatgccttagtttctttgcccttttgtttggttggttggttggcttttagatttttctttcatttttttactattgggtgattaaaatttcttctttctaaatgtttgtaaccacatcatatcttttgtagtgttcggattttgttcatcaataactctaaggcttctagccttatagtaagagcatggtgctaaagaagcaggtgtaccaaaatggtgaaataaatggaagtcatcgttttggttaagctggagaaatgtttcatcttcaaagggaatgagaaaatgaacatgtaattcccaatgtgtatatatagtgtaaacgtacttagcgctatgtatgtatatatagaatacaatacatatagtacatttaacactatattatagatagttaataaaatgcatatttaatacaacttatatatatgtatgtatatacatataatgctgactcttcttatacagaaaagcatacctcagtctaacttttttgttataaaggtaaccctgattgtagaagatctagaaaatgcattaaccccattgtatacacagtgtaaccctccgaccatcatcctcatcacctgagagctttttggaactctagcatcccaggccccacccagacctactgaatcacagtctccctttaacaagatacttgggtgatttgtgtgcactttaaagtttgagaagcactggactgaaatataaatcacccctagtcttcccaccctgagatgacagccactatcaaaattttactgtatttctatcaacttttcttttctatacctatctttccatttaaatcttgaaagtctaacttggttaatttttctgactcatacattacgtttttaagaagaataactatagaatgctgattgttaatttgaaagtattccgctttttagatctcagaaggaatatttatgtaacaattgtaattaatttgagatacatctgtttaaattcaagaaatatgtattttgtatctaaagttactaatagtactttaaagtctcccacatcaattatcccttttttcccaccatatgaagctagatttatttcttctaggtgttgtttgtagcttcctttcttcttcatttattatcaacctaatttaaaagtaaaatttaccactgcagcccgccaccccgccctggccaatagttaagtaactagcacttataagtactttgtatacgtcaggaatcatgccaagttttttacacatattactgtattaaataatcacattaactctgtgaggtaaggatctagtctcaatgttttatggattaagagacagtgacttaaaaaggtttgataacacttaatattacctatctcataagtgctataagcctaagatacaaaataatgataataaatactacatttttaaacaatgtttcttatcaattggcagtctttgtcatttcttctaaatagcatttagcttatagcagtattaatatttttctcgggatacctggtgacaagtgcacagaaagagcaggtccccagttctgaggccacacattaattcacatgatgaaatagtgaccctctaatctcctatagaaatgttaatgaacacacttgggagacaaaaaatccctatgaaattgtattgattccttttgtttattaatttcctaaatataagatatttatagcacgattaatgactgaaaaagctctatttaataggccttcattatgttgttcttagtaatctaaaaatattgataggtagtataggcctagacacatagatactatagttctgttttttatccatctatagatattttaggataaatagatgcctctttttgacatgtaaattttattatttcagtatttctcaaaaatgaagtgttagtttaaagtctacccaatcaactcattaatcaaattggaagggttttagtgtaaaaatagggagatttaagggacctgtaacttccaaaggaggtgttcaataatgctcttatctcaaaggaatcacacagggaatgattttttttccttctaaattacctgtgtcctagtggtgaatttctaacactaacagtctaccccccatcacagacatttgttaatttgtgaagatgaactaataggttttttggacataaattccaaacaaataattgggataccaatttgatatgagcagagagcctttcttctgtattcacaatttccatcctgaccccaaagatggtagcatctgaaggcatacagatcacctctgagcctcagttgtccagtaaatgatgagctggtgattatctcttttttctctctctctagttacatctattgcttcctcctagatgtgcttcatagttcttttcttccagaaactttccctagacttttagcttgcttttctctgatcattttttaaataatcttcatgcttttcttagccttcattgatgaatgtgtagaaatgttcttaattcatatcatatttacaaatttccgtccactgaaggcagtgttttcgttgagggcattaggatgttttaaaactgttctctagcacctccttctatctgcagctacaattctagggggaaggggatcactaaataccatcaagtgataatatccctattacaaataagagaacaggtaatgaaataaagaatgaaattaaatcacatagcaagtgtgcactgaaattgaaattggaatctagaattatccatattccgtacattcagcaactaatttctgagagtggagcgcacgatagaaatatgcaccacaatgaccaatggccaatttgctgggttgtgatgtcacacaccaacgttgcattgcttactggtaatattttgtttagcctaaaagagtctaaagcataccatagaaagagacctagactgggagaaaggagacttgggttcaaatcccctgtttgttaaatattgttggcaaagtcaactccaggtttcaggttttactcagtaaaaagaagaggtgacctagactctttccaaaattaagatgctggaagtgattccatggtagcagactgaatattttgtgtattcagagatgggaaggcatggggggcaggcaagaaagaataattggaatattgtttaagatggatagaatttaaagaaataatatttttctaggatatagagctaaaggtaacattgaatatgaaaaaaatctaatatagttaagcattgtttatcaagttcagagaaagtctggataagataagatttttgctaaaggattatccgagtagatccagtgattcagaagggccctttaagaccttggtgtgccttacactcattacccatgtggcacatctgtcaacacaaatgcctgggattaaacacaccaggaagctcttttggaggaaccatgaattcctgatgacattttgtttgagtaacctgcctattgttcataaagagagggagtagaaaatgtgcaagagtataatttctgtccaaaaggaaaaacttttagttgatacactgtgatacaaacaaactgtttctgttttcttttaaaaaacagtgcaagacattattgaataggaaaggaggccagaagtttaaatccatagaaggaagcagaacagatgtctaagagggcttgaaggccaccctgaggaaatagagaagtggtaaacaaggccaggcgctgtggctcacgcctgtaatcccagcactttgggaggccaaggcgggtggatcacgaggtcaggagtttgagaccagcctgaccaacatggtgaaaccctgtctctactaaaaatacaaaaactagccaggcgtggtggtgagtgcctgtaatcccagctactggggaggctgaggcaggagaattacttgaacccgggaggcggaggttgtagtgagccaagatcatgccactacactccagcttgggtgacagagcaagactctttctcaaaaaataaaataaaataaaataaaataagtggtaaacagggaagatacaactgaaatgaaataggaagctgctgaaagagacttgatatttttaaggctgagttatagctgtttcatctttcctctgccaaggctgctcatcttttactcttcacagttattgtttctcccaagttaatctgtggggtgaggatggatgttgttcctctgaggaaaggtcccaggttatatacatgattctttctttctctaagggaggctccaacagagcatcccctactttggcaatcaatccatcaatttatgcaccaccctcaacaatgactacattccatataatgaattccacactatggaataaaaaaggaataggcaaaaaaaagactaggatattagtttatgaacactgactatattatgctcagtgaaagaagtcaagctgaaaggctccagactgtatgattccatccatatgatattctggaatagacaaaacaataaggacagaaaacagatcagtgatatcaagggtttggggagagggtaatgtttgactagaggaaactcaggagatatttttgcagctggtggcactaatttgtatggcactctaacagtgaatacatgatgctttgcacttgtcaatacccatagagctttgcagcacaaagagttaattaatgtacgtagtttcaaaaaacaacaaaaaataaaccagaagatcagaggatcctgggatagaatgcagaatgtgaccaaagaatccaacagctatacatttataacataacctcattgaagggggtgcaggaaaaaagacactaacttaaggaactttggaaacagtgctttgaatgcaaactataagtgtaaaggaattttaaagaactggacttaaacatggtacttgagttgacaaagttgtttctcatggtagagtgagttaatctgaaactgctgaacagttatactagcactgaataggcaaatgactggcagatggtggggcccatgttttttactgttgcagaggaagttacaggtaaggaaatggcaagaatgaattatgtggtacttgattagattcagagacattaatgtggatttatgtttaggttaatatagatacagatggatggatataattataggtgtgtgagtgtgttcatgagttagtctacaaacatattatttcggagctctgtttgctgaggcctggcaatgacacccagtagcaatgagtattcctggaaccaaatcttaatttctaatactattagcgttccttggagaaatgcctgatttggggggaagggcagggaaaataaaagataaacctggagcttcttgtagggacagaaagtaaggaagtgctcaaaaaataaaaggaagagggatcatcaaagggacacagcagccagcctgagagaactcccagtgacaaaagctggaaccatttgagcaacaaggcaaataacagagtgttggcttataacccaaagtataaaataaataaccataagtccatccatccttatatataaataaatgattgaatgagtaggtaaatgggagcaaataagcaagtctcctgtgcagaagaattccaactactttatatagataccctacccccaccacacacacacacccaagaagataaagcttaaatccctgacccttgagtagacgctacacttactgacttgttcccaaagaatacaatatacagaaagctagggaaggggagtgtgactgtgtgtataagaaacctggaaaatatgacctcacccaggtagtcagggctaacatcatcattcctaagtcgtgtcgacgatatgtaccctaaacattatgcgatgaaatgtgacttgactctctgatcttcctcctcaaaccaaataacctctgtttaaacatgggaaaaacaccagaaaaccccaatggaggaacattctccaaaatacctcaccagtactcctcaaaactgtcaaggtcatcaaaaaataaggaaaatctgagaaactgtcacagatcagaggaaactcggaagatataacaaataaatgtaatgaactatcctggatgggactctagaaccctaggagaaaattaatgaaatctataggaacatggagtttccttaatagcaatgcaagatattaactcaaggggaaactggatgagaagtctatgagaactctgtattctctttgcaacttctctgttaatctaaaactattctagaataaaaattttatttgaaagaaaagggtatagtacaaaactattagtccaggactcttggaaagaaaaaaaataaaccctttgtttcaccatggatagatctataaaacatcagctccaggacaactttgacttcttcctccagtttgaaatcttctattgtaccatccaagatagttttgaactccttgctacttgctcaaaagtatttttctcctaaaatcctggaagtctgaagtcacaacactttgttctttgttcttttatccacccccatcatatttgcctgcaaatcctgtcattgtcaaggcacggctgacgggctttgtaagtggtttgcgatgtctttcagttcctggtattcttgtgttatttaggttcattaaagggaacaatttgtcactaagatccaggtgcaaccatcattactaagccatccaaaattcagcggtgatgttacacagaagtgtggccataacaaacaagcagtatagtataagtatatatacttaataagaatatcacatttgatcataaatcacacaaataatcttcagtctgtttcctaattacagtttcaggaagtgcctgcctcatccataaaatttcataacctcagttattatgtcttccatataagggcaaaaaatgtacacccatttctagttatgttaatagttgggtgataatgccttctggttttgaaaatcatctgaataatgaatgtaaaacagtaataaatattattttttattaccaaacagttatagagtgttaaattataattatcagctcatcattaggtggttagggcttattatttaggtagaagaagtagggggaagggatttgcctgtaaactggtaaataaaacagttatattctatgtagtctcctacatggctttttgatggttatttgatgtctctgttgagataggtaagaagaaaaatgcaatttgagattgtttactatttgatttatttcaccactgaagtcaatcaacacattttctttatcattttgtgttaagtgttcccggggtcttttttcttctgtagtttaatttggatggagtgggggtgggatgccaccatcaatttaaatccatgaaatattttctttaaacaatattccctctattattcccagacaatcgttagaggaaacaaaccctgcaaggaaacctccatcaacggcctgctagaggattttgacaacatctcggtgactcgctccaactccctaaggaaagaaagcccacccaccccagatcagggagcctccagccacggtccaggccacgcggaagaaaatggcttcatcaccttctcccagtattccagcgaatccgatactactgctgactacacgaccgaaaagtacagggagaagagtctctatggagatgatctggatccgtattatagaggcagccacgcagccaagcaaaatgggcacgtaatgaaaatgaagcacggggaggcctactattctgaggtgaagcctttgaaatccgattttgccagattttctgccgattatcactcacatttggactcactgagcaaaccaagtgaatacagtgacctcaagtgggagtatcagagagcctcgagtagctcccctctggattattcattccaattcacaccttctagaactgcagggaccagcgggtgctccaaggagagcctggcgtacagtgaaagtgaatggggacccagcctggatgactatgacaggaggccaaagtcttcgtacctgaatcagacaagccctcagcccaccatgcggcagaggtccaggtcaggctcgggactccaggaaccgatgatgccatttggagcaagtgcatttaaaacccatccccaaggacactcctacaactcctacacctaccctcgcttgtccgagcccacaatgtgcattccaaaggtaacgtttgctacctcctttccctcacgtgtgcaaaaacctccacaggggtggtgctggcacgacctttttatacgattggattgtaaagtacttgtttgccttttaagggaaaatcaatatcaaaacccaccctatctccacttgtgaggccccagggcagaggtttaggataaggcatatatattcattaaaataaaaatctatcttttgggccctcctacgtggtttctcattaaaaggcgaataaacttattgatttgaaagatagaattctgatatatgagtgataaatatgatgtgactgtatttgactaagttccctgttaaggagtggatcagtcatatacaatcatagagcattagatcgagccatccagtggttcctacatcctggcaaagtttgcatagacctgctaaaaaggaaagaaactcagtataacatggtaatttttaaataaagccaattttgttcaactcaggggatggcccttcatattaagagtatgtactttctactatatttgatttgaaatattggtttttatatgaaatttgggggtgacagatggttgctggaccgctggaccatttttgtccccttttcactttgttttgtagtaattcctcttttaaattttgtttaaatgttagcagctccaagtttgctttgaaatttgactggtctatgatatcctaaagtttgggaatcactgccggagaagttactgaaactaaaagcacaggcaaaggtctatgggtccgaaggtctaaagtcttggaaaatacctttgagaatgtgcaagtgatttatttttccctgtcatttagaaatgtttttgctaccttacctgtccagcaaaagaacatggaccgtagggtgtggaatgtggattacatagaggtcttgaagcaggtgagagggacatgacttctcacagggcagtcatgtactttatttataccaaactcttgacagtttggtgagcagcacagaaaactctggaacccaagcctgcctgcattcacctgatttattgattatagatagtcgatatttttcctttgcttcttatttctcattaacctcatccaagatgttgaagagaagccatttaagtgtattgttggagttacttcagccattctttagaagaaacaaaaaagctgtcaccttagttcccaggatataaaattaaaaacaaatcttgataaatagaggcctgagatatgttcctccagttaaaattcacatagtgacagtgttttttttttatctcgtggtattaatgtacttgaacacccacactcaggatatcatgactgctttctcttcctgtgtgcttgccagggtgactgttggaaagaggtctaattaaccacaagcaaaatctacttgaactcatacgcatgtctcatgatacatgagatagtttattccatttcttgtgcatatcacttgtgcatttctgtgagtgatatgaaataaataagttaacatgttctatagctgaattgagttacatcctgaggctttgaatatcagtggttatttttttatctaagattttgtgtgcgtgtggcttatttgcatatttgtgttagggagttgtttggggtcatagttacagagaaaacattgcccagatgatgctgcattttggtcctggagccttatttggctcttcctggatgttggaaatgaatattgggtgggctacacatgtctatagtccaaatagttttagaggtcaagagttttagaactcttaattctggagtttcagtcttctctcttttttatttttatttttttaaaaaggatgaactggtatccctgagtccacattttcacatagcatgggcttttgggatttaatgtcaccaccagcaatccctgcacactcatttcctagaaggggaaactgaggcccagagagccacatgacacacttacagtttcataattagttggttgcaaaacaaggacaagaactcaagtctggtgtttctccctgctcaatattttcctattatttgggcgtatgttatctcggggactcaactgcatttgccaagaaaatccatgagaagtcccagtggccaccagaactttcgtctaagtgacagaagaacgagggtgggaggggtgattcactttaattccctacaggctccctctattctgcagacttccaaatataagacaaagcagacctgccctgaaatctggctctgccagcccagaaacaatgcacaaactattgtggctcaccaccaccaacaacaacaacaaaaacagcataatcactcatgggaacttctaccaagggtgttcagcccttacgatcagaaatacaaagaagcaagctgctgctccatgtttcacaggaaactcgtgagagattcatgagcaggtttttgttttgtgggaggataagaggtgggaggcaggttagtaattaaaacgttcacttgattaaaaatacaacatttctctgaaaagacggaggtgcctgtgtctttagaacggtactcttagagtgtaccaatcacatttggatttccagctgagggaagaaatagcggtcacgactcagatgagtcggccttgcttgcagagactcctaaaggaggctgggtcatgttttcatctccagtaataaaaggaagcacaaaacttgtgtaggtctccagatgaagcagaaatgaggagggtcatgtgtgtgtacacatgtgtgtgtgtacacacgtgtgtgtgtttgacacagttttacattttgtcctaaagaaaaaaaaagattaatgttaacccacagaaaaaaaaaatccttcaatgttatctacctacttaatttccctgctagcaggacaggaatcaagaatgttatgagtttgttgatgttggcctcctctccctacatttaagtgtattttgctggaactaagcagatgaaaccctggggttgggagggtgggaagcaaagccaaacaaaaaccatatagcaccatacccaccacaaaattatgaaaactgccagatgtggagagagaatgctttccaccctcgcctgcagacaaatctgccttccacggaaggaagctagctggagaaattgccgccatgtatttgactttgctgattcctgtgctctccagagaccaggcagccacctacttgtgtctttgtattggcccagcacatccgttccctactgagaagcccccagcataacaatcccaccagttcattaagcatttgctgtgtaccagatacatgccatgttttgttgagcctcacagatctgtaaggcaagaattcttgacccctttgtacagatgaggaaatgggttttaagaggcttagtatgctcacagcagagctgggatccaactctttgggcttaagccttttaccagacattgcatgcatttccagtagcacaggggcagcttctgtgggtgcaaagcctgcaacctgcctgattataagctccttttaatacctgaagcaatgaagttgcattagcagttaattagcaattaatttcacaactaggttgcatcatcccctttcactctgtagcagggtccaccaccttgacactatgaacattttggacctagtaattcttcattgtgggaggctgtcctgtgcatcatagggtgtttagcagcatccatggccgttcccctctagacgtccatagcatccctccctcactcctaacaaccaaaatatacccagacactgccaaatgtcctgcaggcagaagagggcaaacttggcccagttaaggacaactgttcaatagctatcatttatcaagcacgttttggcagttaaagatatagattttggagataaactgcctgggtttgagtcttgacattgccacttattaactgggtgaacaagttgcttaactgtgcctccacttctttatgtacaaaataaggataaagacagcccgtgtctcattatatagtatgagggttaagtaagttaataaattacagcagcttaattactaatatgagtaatctacacatcagtctacatgtggagcatgtagatgaatggtggcagtgaggtgagggttactgtgttccaagcactgtgctaagcattttgcaaacatgactcaacttaatctttgcaattccagaagaattcaggatcacctctgctctacagttgaggaagctgagactccaaatctttatggacttccccaagtttataaaggcaaataactgagtcaggattccaaactaggtcccactgactccaaagtctatgtgttccagaatattctcatttccgtagaagaaaatccaggggcagaaaggtggacatgaagtctccaggcccagacacaaatcctagaggtagcctcaccttgctggtgaagaatgggtggtgactgacaggtggatttgctgtgcccatggaatgctgtttattcttcctgttcttccatcatgtaggatggaatccttgagaagttccttaatgtcacccagagtgtaaaccgtgggtaaaagctggaactatgggctgtaaaggaagtgttaggtgcagaaatccctaaggtgctcatatgcatttatttgaccttaagtggtagtcttgtttgattttttaaaagataatccagattttctcacccattacagaaaaatgcaagttctataatcagattccctggattcaagcgcttagtagctgtatgatcttctctgtgcatcagtttcatcatctctaaagtgtttcagtacctactttgaactgggcatggtggcacacaactgtagccctagctactggggaggccaagacaatcctttgagcccaggagttcaaggttgcagtgagttataattacactactacactccagcctaggcaacagagtgagaccctgtctaaaaaaacaaaacaaaacaaaaaggatgggaagaaaaggatggtgtgatttcccggcacttccttctggggtcaacagttcaatgtgttttcactgcttagctatcactttgctttccctggaaggaaaaagcacttgaaccagtgggcccagtcttcccgcatcaataatttactgattcactgttctttgtggtacttgtcatgcccagattcaaccatggctccaacaacagaacagcaacttcttttctactgaggatccctgcagaactcggttcagagatccattcagggtttaatgggttatggaatgacagtgctagtgaaaactgatgggctgcactaaaaatctgagggggaaagagagaagcacttgatcacttaatataacaatggatatatttagatttgttttctttacctacagggtcttcattaaggctcataaaacatccaaggactgactgagtgcactatctctaacgtctgtcccttctgatggagactttaggtgtcctttgcatatgaatggacacttcttcccatgaatgctcttgtgattcaaaataaattgtattttggtcattatgaatgggttttcccattaccaaattaaaaacaaaatgaatttaacctggcattgtccgttcctagctctctcagaggccctgacatatttgaacaatattctgaggctgcaaaccagtccccaaagacgaagagtccctccacatcactgcaggcgatgacggatcgcaggtcagaccaatctgctctgtctcactgctcttgaatagaagacacattctttgagtttgaagcagcaactgtggggggttaattggatatgccaattcagaggtggaggaagagcctggaatacccgtatgcctgaccctgcacatctcagatttattcattgcttcatatagctaggctttgctagtttgtagggtcatgagccacttgtccaactctcagtgtgcatttgcagggcacaaccagagagacagacgagttatagggttcaatgagtcccattcccctaattgccagtctctatcctgtccctacagagaatgcaagttaatgattctctccagaattcttacaagcagtcgttttcccttcccaaagccaagcctggtcctgttaaattttcctttgagtcaagtgaggacaatttaagatgtcctttccatgctcagataatagaggtactgtgattcacttttatcttgcatttgatttatggagagtaagcagcacccagcctggcgttttcattttcgagatgagcagtctttgttttattgtgctctgaaaaggtgtattggagcatcacattcaggcactggaaccagctgccgcccactccctgtaactcgccatccttcacggaacattccgagtcctcattctgtgggtacagacccacactgtggctgtgtccacacaccccagtcgagcacgctccagccacggcccccctgccctcagaacagaaggccacagtgccaaatagatgctcctgcagtactgttttttccttctcttctttctctcaccctcccctaaccaagctttcttccccccaagacttggcataaaatggtcaaattatgttctactgcagaattcaatttcacagttcagttaggtccttgattacactgccaaattcagattaatgtgcatttaactaacatggatcaggggaattctggctgacaaccagctgtaatcggagcaattgatagctgtgtggagttttatgccccgtccaccccagcgtagctctggtgaaattgattgattaagtgaattcatagctataattatttataattttgatacatcacaagcctgtgtctgaccctatctttagagatgttgagctcattgtagaaaggtagtccggctgagttgttaactggggattatttattcccttatataatatattcttttttgctctttttcttttttgatcatgtttgaatagagacatgacaaagcacattttttaaagaattaatttatatggtatacaaaaaagtgtcaggaagctaaaaaacaagtgtatgttattagccatttcaaaaaaaaaaaagaggaaatcacaatcatgactgcttgttcattttgtcaatatttctaaagattcctgtagagagaattccttcttaaaatgtatccaattttcaatgacaattaatgacctggtaaccctgcagctaagcagtttagcagcctgggtgataatttgcaagacaattgacctggtcttctctcatgacatcatgccatatggaaatccccctgaaaactttgcagaccatggtctcctttgtaaggagttactcgttcacaaaatcacagcactgttctctttggagggatgggaagaatgcctttgttgttttttctcttacattgcagtttcccacccttgctctgcactccgtgcccttgctgtaaggtctgcaagggtaaactatgtacataaatcttttggaaatggtttggatcatgggtgatgagagatatgctggatttgcttattaaatatcattcctgtcactagtcccattccactgcattgtgttttcactaaataatcagatcccaaatccctttcacaataccaaagataggtctgcttttatgaacttcaagcctactcattttttttctagaatgtatttgtaaatcaggtcatagaaaagattgcacagtttaagctggactgctttgcacatctttctcacatgcatgaacatgtgtgtgtaaaacagtaggaatggacaaattcatcatttatgttttctgccttccaaatataggctatcatgagaaagacaacttagaatggaacacttacatgactatgaaagtccaagagctatgtgaggaggtctcatcgcagaagaaagtttgcactttcttccttgaaagaacttacagaagaaagcctgagtgatgttgtaacaatgcagagttctgctttgaccaaaacaaaaaggaaaatgatgaatgatgtacatagataatcaactcgttttgctgtgatgactactcttttttttagctcatttctctaagccaaaactgctcaaaacaaacaagccaaaaaaaaaaaaaaaacccttaatttgtctttcaccccctctcctagacattatcatccatcatgttctggtccaattcttgcattttgccccaggctgcccaaacatctgggcccccagtgaaacaccctgaccaggctttatctctgtggttatcatcattgcccactctgcccgcccctgttaagaccacatcccccccccccatcattcatgcctatctctgtgaatttttcaaggacccaagtactgaagcgactctgtctggatttgaatcccatttctcccttcttttagctttgggatctttgcatgacctacttaaacccactgtgcctcagtcttcttatctataaaatggaggaattacagagcttatttaattgtccagcttgaggtttacgtgcaagaatatacacagtgtgcttagaacagtccctggcatgtagaaagcactcaataagtatcaaacatcattattcttataaatcaatgaatgttggaaatgccaaggcaggcaagggagccggcctgtgggccagtgtgctgggcgccctcccactgggagtgaaggactgaccccggcagaagaactaggttatgtcgtccccttggaagtatggagctatttgccctgcattgcttgggggcagtttcgtctccttgtgcctctattttatcaaccacaaaatgggattcagttgccccctgcttgattcccagaataaaaagaacaagacctgttgaaagtgtgtgggctggtgatacgcaccatcatggaaccccgcatgtgggttaagatccaacagaaggcaatggttcagcaccttggtggccccaaatggggtcacccatgcatttctccttcacagagtcagctatttgtcctctgagggaagactacatgtcccctcacttgaagtgaacagttcattctctgatgagtatgccttgagtttcattcaaaacattgtctcttgtgatactagttgttcctcaaccattgcttaagagtctgtacctcttaccagcagccgtgaaagacaacagcacctttttgtctcatggcctattttgaagatggttagctttcagcaaagagctctgcattccatctttgcctggaaattgtccccttggtgcaggcaggcttcttctttatgtcatatgccagctgttaagggtggattctgataatgctgctgacaggtgataaggaagaaaagtggcctaaattagtgcaaagcggtgtttgtgattcctgcaggatgcagctttcccatttttaatatatcttcagagatcagttatacatgcttgtggggaatgaattcaatttcgttgaaagtacaggagccagtcctccaaggaagacacagggtgataatggcaagtgatggctcccaggacacactttcagaaataaaagccaaaacactatttcccttcacaccctggccttgctgactagcctagacttgactaatctggagcttattgaaggaagaatccagttgtttaacttacactgacacttctctctccccattccatcgtagtttaacttacactgacacttctctctccccattccatcatacacacacactcacacacacacgttttccttgtgtagcacaaatacagaggctttgaaaagaatcagcttggttttgaatattttaactatacattgttcaatatattaatgttaactacatcttgtcttattgctttgagtattaatatataggaatatatataccacaggggcattgtatgtcttcaataaatgctagctagtttgataatattgcagttactatttattttattttgcattctcattactgtgctaagcattttatataataataacaccaagtagaacttactggccagtcattgtttgaatactttgatatgtatttgatcctttagagttcagtgaccaaaggaagtaagtattattattgttccattttacggataaggaaacagactcacagaggctgatttgactaaaattatcagagtcagaaggaaactaagactcagagaggttgacttgactaaaattatcagtcagaattcattcctatgcccgtcaaaagcatgtgttctctctgcaaccagggcaggagggcagcttagcagctaccctggttcctgctggttgatgccaaggctgtctgagtggtgaattcacctagtaaagtgtagtgtttttgaagctggaagggctgcattgctgaatgttcagcaggccaggcactgtcattgtagaaccaatgggcagtgttgggggactgagaatggtgctatggtagcaattttaaagagcccactgtgtgcagttattgctccatgatgtgatagaatgactaccttcttatgcatcgtggtggggcaggagcttgaaacacctgcttgcctctaccatgtcaccagatccaatgagcaacatgccccatcccacttctcagtggcatttgacaggattcattagcccttccttcttgaaatactttcctcccttggctccaggacaccttgcttccttagtttcctttctacagcacttgtcactccttctctgctcccttttgcaagatccctctgctctttctggtatctagacgttggggtcctcagaccatctcttacacattcagtatcatggcttttaatatgaactaagcaccgatgactctcagattcatgtctttagctttaactcttccctggacttggggcatgcctaagtctcctaagtgacacctccacatggatgtctaatcactctcccaaactttacatggctaaaagaactcttaattttccaccatgtattagctatcaatattgctgcataacaaattatcccaaaacttcatggcttaaaacaaactgcctcatgaagcaaaaaatagtgctgttcaactaatttcccctaaactttaaataacaaacatttactatttcacagtttctgtaggtcacaaattgagagggcttagctgattgattggttctgtcttggaatctctcatgaggttgcagtcaaggtttcaaacaggaacacagtcatctgaaggatcaactggacctggaggatccacctccagaatggctcacatggctgtttgccagaaggcctcaatttctagctagctcttagcaggagtcctcagtttcttgctgcatggacctctctattttattattattattatttttattatttctgagacaaggtttcactctgtcatccaggctggagtgcagtggtgcaatcatagctcactgcagccttgagctcctgggctcaagtgatccttcagctttagcctcccaaagctctggcattacaggcatgagccactgcacccaacccttccaaaggattcttgagtgtcctcatgacatggcagctggtgaaagccaaaataaatgatgtaagaaagcaaaggaacttgccatcatctctgcctttttctttgatcacacagattgatcccaatacagtgtagaaggggactacataggaatatgaatgtcaggaggcacaggtcattgagagcaatttggaagctgattatcacaccccacaagccctgttcctgcccctggattcctcatctcattaaatggcctgttgcttaggccaaaatcctgatagccatctttgattcctgtctttcaagatctcacccaatccatcagcaaatcctgtcagctccatcttcaaatcctgccctagatttcaccatttctcaccccatctacaataaccaccctaatctcagcccacattaactgtgataagctgctggcatccctgtttccactcttgccaccaaagtctctcctccagcagccagagagtcagactgtgtcattcctgtctgcttagcaacccccagtgcttcccacctcactcggagcaaaagccaacgtctttgccaaggtcttcatgatctgtcccccggcagcttctcggcccatcttccttagtggacacactccactatagctgagcgtctgctcttcctcacaaactaagcctggatctgcctcagaatcctgcatgtgccactcccactgcccaaaccacccctcctaggaaagggatcttgctttttccctcactcagccaggcttctgctccaacggaaccccctcagagagtccttctgtgactaccctatccaagatggcatctcattactttcaaccccctcactctgctttatttttcttaatattattcattactactgaaacacacactgtatatttactggttttctttattattatctatttccttctctggagcataaaccccaggagggcagagatatcattcattttgactgttggtggaccctccccctcacacctaaaacagtgcatagcacatcatagttgctcaaatattaatttttgaaataaatgaatgaatgtgctgcatgggaacaatcaattcatctagaatagtgccatccaatagccctttctgcaatgaggaaaatgttttatatgtgtactgtgtgatgtggtaactactgagttcttaaaatgtgactagtgtgattgagaaactgaatttttaatttttaatctatttttgtttattttaaataggcacgtgtagcttgtagctactatattagaatagatctaggtccttggggtggatggtaggggagtgggataagttaatgccattccatttacacccagtagagtgatagagactttctggggcactgcattaagaaagagcctcaagtccaatcaacagatcttttctgagtaccttctatgtatccagcactgtgttgtcatcgagatcttccaaaacagagtgcattgatggattagcactttctgccctgggctaggagcataccacatgacaagatgtggtatctttaccatcctggcctcagcaattccaggagtggccagagctccttcctgattctttccaggaagtgactgctggacctacatattgtatcatataccagtaatcccctcactggcttccccattctcctccaataaatcccctcatcccactcttccaagtcagagtggatgttctttgctgggatctctttcgggtctagcatcattttcatttcacacatacccctgcaaagtctgatcactctcaattatatttgtacttatttggtgagaacattgcacatgttcaccctgtggttcacaccacttagcacagggagacgtattttcccctgtacctacatctgctgtgttatacctgtgttatacatatatgtccataccaccctctagtatgtgaattcagaagcatgggaaaaggcctcctacccctgaaatgtctcccaaaaatgctagctgaaaactaatgtcctttaatgagggatggggtttttcattactgaacaagaaaatagggcttaacaagcaagtacaccttagatatgacacacgtgaagtaacctcctgctgtggtgcacgtgcaccacagaaggcccttccctaggtgtggccggtcctgttggtgccccagccagatcctctcatctcccactggtgatcctcattctgccagctgtcagtcagctcactcaccactcggctgtggcattcagatccagcatgtgaatattctctatccatgtctgtcttcccaggtggattacgatcgagcacagatggtcctcagccctccactgtcagggtctgacacctaccccaggggccctgccaaactacctcaaagtcaaagcaaatcgggctattcctcaagcagtcaccagtacccgtctgggtaccacaaagccaccttgtaccatcacccctccctgcagagcagttcgcagtacatctccacggcttcctacctgagctccctcagcctctcatccagcacctacccgccgcccagctggggctcctcctccgaccagcagccctccagggtgtcccatgaacagtttcgggcggccctgcagctggtggtcagcccaggagaccccagggaatacttggccaactttatcaaaatcggggaaggctcaaccggcatcgtatgcatcgccaccgagaaacacacagggaaacaagttgcagtgaagaaaatggacctccggaagcaacagagacgagaactgcttttcaatgaggtaaagagtgtgtttgggtcatcctttctctcctttgtaacatttatttcccatttcaaaagttatacacatgtacattttaggacattagaaaagcactgaaaatataaagacaaaaatagcgtcccctgtaatatttccttctagtcttttttctatgcacatatatggctgttacatctataaagtacttcatgtgtatcagccactgtactaagcattaatctagcttgatcctcacaacaactctgtgagatagaaagatgttatcaccatttttacaaggaaacctggactcaggttaagagatccggctcagggtcaagcaggttgactgtagagctaggatctgaatctctgtctagcagattctaactcttatgctacactgtatctacatgagtatgtatatattttacactactatgatatcataatataccctccataaacaatttttaaaacagtagaccagggtcagtttaccaatggactatcaccctttaggaactttaaaatatttttcctaactataaaaataatgaattcttcctgtagaatacatggaaagtaaagaaaatgtcccacagatatcccagtcaaggaatagcctacattaatatttcagtgtttcctttctatatttttctgtttacaaatttcttttgtttgagataattatatatgtagtttggtaactctctaaatttcaattaatattaacatgaatactaataaaattaatatcataaaatatattgacctgtattacttatatttattaatgtaaagtagatttaatgaccattttaatggccttataatcttctatcatatacatgtatcaaatttattaaaccagtcactgatttaataaatttgaggtaactttagatgtatgtggttctgtgactttctaaatttcacctaatgttaacattaatactaataaaattaatattaaataaaatatatcagcatacattacttatttttattaatgtaaaataaaatatattaacgtattctattagctacatgtcaaaacacaattttaatggcttaataatcttttatcgtatacatgtatcatatttaaaaccagtttctgatttatactgtttccaatttgtcacttttattattttgcagtgagtgttgttacacataagtctttggcttcatatctgataatttattagattcctagaagtaggatgaacagatcaaatgatatgtactttttttagataaatgatatacagtgttaatttctagaaagtttgtaccaatctagaccctcaccaatagtattgtttcaccttcactagcgtcattgagactgtcatgttatctctgttcatctgctagatatgaataaaccttactgttttcattagcacttttattactagtgtagctaaattgaattttatgtggttataggccatttttatttcttcactaaatcatatgttctgtacacagttttcaattagggtgctagcatttgttttattgatgtgtaggagctattgatatgttaaggaattaacccttctctgtatatttgttgtactttaatgtttaccttccactttctcatttaccttttaaacttatcacttaagagctctaaaataggtggggaaacatgagtaattatgttagaaaacacataaagtattttggagtaatattcttttagtaattttatgagagcattgaggggaaagataagtttttgtggatgagagatctaagtcctaaaatatcaagtcagggtatcagtgttagatgttagtctccaggaaagcatgtacaatccacatcttctaactcccagaacaatctacggctgcttgacctctttgtctttctattgtaaggactcccattctctgagaagtaagaaggtgtttggtccataaacaggcactgtacaaatgcactcagctgcaattttgaactctgtttatcatggaggctcatatgaagcccccagtggcctccacacatctccagctcccacgggtgcttatttcttacacacgctccaaatctcacagagagttccaggtgactgggcttcagcatctttccagtccaccattcctggtttctggaatttattttccaaaccacaatgccagaacacacagagcaatatgatgtagataaataattctgtagtttattatttaacactagctctgaaaactcgagtggaaacagccacagggcagcaatttctggaagctctctccagcaagcaggctccttagcctttttaatttctatgccatacttatttttatcttcaatatatattctttcaattcagcccaaatatctctgaagttcaacccatacctacttccaaggagaagttaagttggtcttgtgtggctccagttctaataatagatagtttacttcattcacttgtgggactcattccctcaattactacgagttggtgaccctttgctaggataaccagcagaggactgtgaacagtgcattctaaaggagacccttagagatatctgatcatatccccaaagatggtcaaccaaactccctcttgtgaatgcaaatcttgctttggttctacttgttttcattttctctctgtgtctctccaatgtcagagtgacctgccattttctctcaaatttgctaccttgattccttttatttctactagctacccttgatgctttttgtctgtaatagattttctgaataaacttccccacaatcagtacctctcaatcagtttacttacaattgtagttgtggccacaagagaacaaaaagcaacttcatcttcacaagtcaaaagtatatttcccgtttccccaggtcgtgatcatgcgggattaccaccatgacaatgtggttgacatgtacagcagctaccttgtcggcgatgagctctgggtggtcatggagtttctagaaggtggtgccttgacagacattgtgactcacaccaggtaggcttctttattatccagagaattcgaggtgcttcttattcttctccaggataaagccgcagtgactatgaactcttcataaattgcaggcactttggagtcagggaatatgactaccctttcccctacattccccagagggcccattatgctctcaggcatgtatagttcattcatttggtgaatgttctccctgccctcagaaggacattaatacccattgatttaaaataagcaacctgctaagaatgaagcttcagtgacaaataatagaatgtcctgtcgcctatatagatttctaaagctgaatttcattctcctgagttactctaccattgctgcatgctaagacaccaaggataccaggggaaggaagggacagggagaggatcacttgaagttttgtatcaaaataatgaatctgggtggcacagagaaattctgcaattctgttttaaaatatgcttgcaagtaacgatggttaattaccagcctattgcttcagctatttgcctccctgatgtgccagttccagccatttcattaagtatttattgagcaccttttatgtgccaggctgttttgtaagtggtagagatgcagaagatacaaatgaatgtggctcagatatatcccctcagggacatcactgatgagaaatacattataatacctgtgcagaattaagaagatatatatatgtagcatagaagaatacctaaatgtctctctcagaaagagtcaataaatatttctatttaatataaggtgacatctgagatggattttgagagcgaataggagttcactaagaaggcaagtaggaggaaaagggcattgaaggcagaggaaagaatatgtgctaggacagtgcctcttcaatttcagtgtccacataaatcactgtggatcttatcaaaatgcatatcctgactcaatagagagggtgtaagattctgcatttacggcaaactttcaggcaatggcaatgctgctggttcatggaccatacttggagaagcaaggttctgtggaatagacatacaaaatagcatgttacatttgggaaactttaactttggttttgatggtaaataaaatgccaaaaagttaaggaaggattgtcatgaggaagaggtattgagtgttagctgatgttattgaaatggtacctgaatcaaaagtggatccaggtacataattggtggaaaacaaggtaacaagaaagtactgggaaaccctgtgaagtataaaacatgctgttttttctccactgaaatgagatgttacgtggctctacagttctcggtctggagtaggctatctcaaaataaagctttgtgagtgaagacatttatcaccaacaattcagctggcagactcttgatgttgtagcaataagacatgcccaacaaaaaaacacataagatcatcttaattggtagaaacaaatatactggaaagatctaagcataaggcaaatgtgatatacattttttgcttttcacatataaagcaaaaaataaaaatatattcaggggagaaacctttttgttgccttccagagcttcatacagccactcaggtctccagctctacctttgtcaaggttcctttccattcttattttgtatccatggaaaccactcaagacagtctgtccctaaaatccattttcttttctaattttctcatccaggattgtcaactgatttcccttttaaagacccagtaactccagaggcaaaacaaaattgtgtgtcaaggaacaccatgaatacttggtattcgaaatcttaaactcagaattcagagagcttcctttaatagatattgaataactgctttactgaatgatttattgatcggttgataaaacatctaaccccttgcaactccaagtgctgtctgaggacctgggagcttgttagaaatgcagaatctcaggtactaccccagaccaactaaatgagaatccatgctgtacccaaatactcaggtatttcttatacatactgaagtgttagcattaacttaacatcttgttacgtcagatccttctaatttccaggaaacagagggttatagaagggaagggaaagtgccaagcaccatcaagctcttcatatgttaatcatgtaaccatcacatatttctatgaggcagatgctgatgtacctgtctgccaggtgatgaagctacaattcaaacaggaaaaatattattggccaggcatggtggcttatgcctgtaatctcaatactttgggagaccaaggcaggaggatcacgtgagcacaggagttcgagaccggcctgggcaacttagcgagaccctgtctctattaaaaaaatttaaaaattagctgagtgtggtggcatgtccctgtagtcctagctactcgagaggctaagacaggaggatcacttgagcccagaagttcaaggctgcagtgagctctgattgcgccgttacactccagcctgggcaacagagcaagactctatctcaaaattttaaaaggtatccttcttaagaccacacatttaataactgatacagccaggatttgaatttgaggttactctagaactgaagggttttttattccatgccaagcttttttcctataggaaagaagtagaagaagaaatgcaccaacgattctccagctggccatgctaatttcaaagacactggattaggaagtatttgtcacgtgttccagtcaaataggatctttctgttagtgactagcggttagattcaagtaatttttgatatagtcgacctgtaaagaatctttatacctctttcagataactcaaatttcacggaattcaagagaaaataattttattaaagatggtggttcttggttctgaattaatttgaaggtatatgtgagcttgtccctgctgcatatactgtaaacctctgaggaatgtgtttttccacatgtgaaaatggtcgtgtgaaaaccattcaggataatgaccaacataccacaaaacccagaacacagaagttgtaggcttcctctgtttcaggcaaaaacatttcttgtcacctgcctttgtatgagactggtgctgtggaatcctcttttgctttctttttttttttgaaacagagcctcactctgtcgcctagctggaatgcagaggcgctatctccgctcactgcaacctctgcctcccaggttcaagcaattctcgtgtttcaacctcctgagtagctggaattacgagcgccggccaccatgcccagctaattttttgtatatttagtagagacagggtttcaccatgttggccaggctggtctcgaactcctgacctcaggcaatcctcccaccttggcctcccaaaggccaaatcctccttgaggggaagggtgtgtttcttcatactcatcaactttatacctttggtattctcctaattggctaccactttataccttacttttgaatcccatcaggattttctagcatcaactaaactgaaatgaaaattcaaagccattcatttccttattctgctgaccttagatttacctgggattactggagtttcacaagtttgtccttaaatttgggctctggtcatttacaccctctgtctctcctgacagtctcctctgtttggatgatagttcgctttaggacctcttttacatttattatttaacatgggatagggaagaagttttgcacatagatgagttaggcagctgcctaaatgagtcattttgagggcactgaacaacctgtgccatccaaataattctcacctagattcctctgtggctgaactatttcactcattaggaaaatccattcattcaagtggtgagggcagtgtgggaaaatgggaaagtgggaaagtcggaaagcgtgcgttctggactaaaaaagaggtggggtttgaatcatgacctggccatttcttagctgcgcaaccttgggcagttagctgacttccccctccccctctaaaactgtggagaaggaaaaactttctccacaggggtgcagtcatgagatatgggaggccataaccaatgctttgcttcgtttctcattcccatggtccatttattaaaatactatccattcacactctgttattagtaacttcctaggtccaatttccttttgagagaagggacctgaagcgacaaaaacctctagctccttggaggaagaagagagagggtgaatgggaagtagggtgggtcccagagggaggattagagatgcaaagaccactttgcctgagctcagccttgaaaactgtggctgtccccatttctaagatgaagaaaataagacccaaggaagttaaaggattttcttatgatcatttaacagccacgcctgaaaccttcatctacctattcagtgtgctgactctgtgggaccctggaggcttgttgatgcctatggaattatctgttaagagttcatggagccccgcacagtggctcctgtctgtaatcccagcactttgggaggctgaggcgggtggatcacctgaggtcaggagtagttcaagaccaggctggccaatatggtgaaaccctatctctattaaaaatacaaaaattagccgggcataatgatgggcgcctgtagtcccagctacgtgggaggctgaggcagagagaatcacttggacccaggaggtggtggttgcagtgagccgagattgtgccactgcactctagcctaagtgacagagcaagacaccatctcaaaaaaaaaggaagactgcactccagcctgggtgacagagcgggactccatctcaaagaatgaatgaatgaatgaatgaatgaataaataaataaataaataaataaataaataaataaatgagttcctggaagtgcatttgcatgaatgaccaaaaagctgacaaatagtgatctctttcctttgacttatatttggtccctgtcattcttagtgccaagccttttttctgggagtcagatcccaaagacttccttcccttgtctgcaggtttttcagatatgggctggagcacgtggactgtttcactaaagcacaagggagctgcctctcttgagcatttcaaagatgttcttaaacctgtcttgttccttaaatgttatttcctttccagaatgaatgaagaacagatagctactgtctgcctgtcagttctgagagctctctcctaccttcataaccaaggagtgattcacagggacataaaaagtgactccatcctcctgacaagcgatggccgggtaagatgttcatgtttggcccgttcgtagtttttcttgtcactctgtctgtcatgggcataactcttttagaaacacttttttataactctaatttttcttagtcacaagtcacctacttctcttcccagaacacttacaaaaaagaaattaccatcccttcctctgtgtcaccttgttagtcagaattgcctgactttgatggataatttgcctccttaacaatgagtataggaagtaatagaaactatggctatttcttttccttttctgccacatctgttttttgttggaggagaagagctgactcttctaggggtgcatccaacctattcacatgaatacatgtgtccacattaccttccagagcccgtgcaggtaacagatgatcaactccacttgggtaatttgaggatggtgtaataaaggacagtttacaaaagcgtgggcagatgtagaaaaatcataaggaatagtgtatgaggattagtgcagtactttataatggaacattactcctactaccccatgcctgaagtggtgaggcaaggtcctggctactagaatctggaaatggagattgctatagatttcggagctgcctggagggtgctctgatctttggtagaagccaaaccaaggtgaacctgcaggatgagagctgagagaataaatcctccaacctcactcttcttccttctagtgtcctactggtgctccctattggctgaaatcagctaggagccaaaatgcagtcaatataggttggtttcctaaggcttggaggagggaggaaaatggatccagtaggacaaacagaggatatttatcacactggcaaacctctaggcccagatttccgtatttcacatcactgcaacctgtgcaagggattctgctgaaactcatcagccacaccagggcacagagcatggaatataacagccaaggaaataaccagttgacattccaaggaaggcttagttaaatggaagagtttcaaagcatagctctataccattaggctctccggagccccctgaaactcccaccagaggcaatggccttggcatgaatggagccaaccattcccttgatgttcctgtcttttacacctgcaacccctgccacatggaaaatgttcatgtcatatcagaaattgcccaggaatgatcagagggcagccagaagaaactgtccaagcactttggcttttctttcttgttgttttaaaaagattgtaaatattattgtagaaaatgtagtaaactcagaaaagtatagaggataagacaaagatcactcctaatcccattaccgatttttcccgtatttccatgtacatgtatacacaatttgcaaacttgggaatgtgtttaatgaggagttttaagtcctgctttttgcattgctttaaccattgcattgcttctcttaagaaagaacggtacagtagaagcatgtatcagtcaggttttgctacatccactcaacagttagtggcttaaaacaaccaccatttatttgctgacttatctctgagccagtatttgtgctgactcctctgggacaactcatctctgtaccagctcatctctgcatcatgtgacatcagctgagctccctcttgcagttgtgatcagcctatggtgatagccttactcatttgcttggcagtcagctggctctcagcagggacagtgcaggtaacagggccaagtatcactcatctccgagtcacctggacttactcacatgatacctgtagcaaagttcccaagagcaccaaaggaggcaagccctgatgtgtaggccctttcaaatcctgtgcttatattatatttactaaatattcaagaccagactcaaggagtagggatataaactctgcctcttgataggagaagatgccaagtcacattgcaaagggatgtggatacaggtagaagaagactgtgtatgcattttttcttaatcttccatatgagtggacagctatgagaaaaggtcaagaatcctccctggcctgactccggaaagcacctacgcaaggctccacggtgtcagaatgcaatttgcaaagcattaaactggtctagcagagcaatttcaagaaagactttctttccctgtttctagaaatttatggtacaggcttccttgctgtaagatcatatccagtatcaaaatcagaaatgagactaagaagtcattgtttaatcaaattacttaaagatctcccctcccctcaatgtgcaagatttttaaatcattgagttgtcattctcaagagcagaacctttgtctcaaacacctcctgcatccctgtgcaccctgtaaagagctcacataaatagacacattgtagatgcttttagtccttattggtttgttctctcattcatttactgaacatttgctgaatgtctgctacgtgtaacagacagttctaaatcctagggatgaacaaagcaaaatcacttcctctagtaagcttatttggggactgggaatgtgataataaacaaatagataaataaacaaacaaaatccccaccaaacaaggggtgataaatactaagtttcctaggattaatttaggcatggtaaggagaatggggagtgatggaagtgatgggtactgtcttatggaggctggtcagaagatccctctgatgtggtggcgttacagcagagacatgggggactggaccctgtggctgccagaaacaggagcattccaggcagaggaagcaagagcatattgagtggatcttgtgcctcttggccattttgtccagagtttactcattcatgttcagaacagtgcatcatattgactccagtggtttctccagtatctgtaggaaaaggaaacttttcttttaaaagagagcagaaatggagtgtggctactccaagaaaaggcttagagtgtgttattatatgagtctattagtgtacaacattatatagtggagaagactttttcaattatctattgctgttataacgaagcatctcaacatttagagtcttaagtcaacagtcattttagtattgtctcttatagctcagcaaaacagtgctgctctgacttgtggttagataatgcctggggccagagttatctggaagtcatcctcactcacatgtcaggtagtcagtccaggcaggctagctggagctgccaccagatcacctacaaaataggtggcctccccatgtggcttgagcttcctcatggcatggcaactgggtcccaagagtgcgtcccaagaaaacgaggtggaagtgcatggcatttttataacctagcctcagaatcaaataacattactttcaccatggtcacaagcccttccagattcagggggcaggggtttagagtcctcctcagtgtgagaaaaattaagtcatattgtaagaagagctcatgggataggcaaaattattgttggcatttttggaaaatactgtctgccacacaagagatattgggttctcttcctgtttgaagatgttcaacatcattggcctttgagaaatgcacattaaaactacaatgagctatcactgcacacatttaagaatgactaaaatgaaaaatagtgataacaacacttattcatacactgctcataggaacttaaaatggcacagccactcaagaaaatactttggcagttctttttcaaaattaaaaatggacttaccatacgaccatcacttgcactctgaggcatttatccagagaaatggaaacttattttcccacagaaacatgtacacaaatgtttatagcagttttctttataatagttccaaagtggaaactacctaaatgtccttcaaagggcgaatgattaaacaaatagtattaatatatccatgtcatggaataccacttaacaataaaaacaaacaattgatacacacgagaacttgggtgaacctcagggaagttatactgagtgagaaaacaccaatctcaaaaggatacacactatatgattccatatatttatatatatacacacacacacacactcatgaaataacataattatagaaatgcacagcagatgagtggttggcagggttttggggctgtgggaatgtgtctgtaaagaacagcaccaggaagtcctgtgctgatggtacagttgggtatcttgattttggtggtggttataataggttggtgcaaaagtaattgccattacttttagtggcaaaaaccacaatgacttttgcactgttctacacagtctcccagacttcttcagcaggatcaagctctagttgcccaccgtagtaactcaccgggtaactagccttcccttggttagctgactagttggcaacacatgatacaatttcagagtatacttatatacactacacacacaatgagttcatttgtaactgatgaaatttgaatacgttctataaattagacctaatcaatttacaagtgttgatattgtactgtagccgtgtaagatgcgaatactagggagagacgggtgcacgttgcaacttcctgtggatttatcattatttaaaaattaaaaagttggcagggcgtggtggctcacatctgtaatctcagcactttgggaggctgagacaagaggattgcttgagcccagtagttcaagaccaacctgggaaacatattgaagtcccatctcatctctaccagaaaactaaaaaattagctggatatggtggcatttgtctgtagtcctagcaccctggaggctgaggcaggaggactgcttgggccctagagtttaaggctgcagtgagccaagatcataccactacactccagcctgagtgataaagtgagaccctgtcgccaaaaaaaaaaaaaactaaaaaattttaaaaattttttaaagttaggaaaaatgaccgagaattgctcaggtttgaaagaatgaatgctttctgtcctctcaaaagactagataaattatcttttatcaactaaaagtaaatcattcaattattatctatggcataagtaatatctaacgagtcactaatttgtgttaagaagggagaatgtggtgtttgggatttgaaagaggaaaataaatagtatctcacagagtggcttaggaatgaataatgtaatgtgtgtaaaaccttcagaagtataccagggacaaggtagatgctaagtcagaaggcatttcctctgcccttgtaattaagtgttgtgtagaatcatcatgacctttccttggcctagggccaacatttgataaatgaattacttaggggccagtgagcagtgagtgactgactttgacaaattcattggccggaattcaaacccatgacccgctaagtccaataagtttatggaaaagagacacacacaagtggtctttgtgatctctttttctttttactataaattgattgccacaaatcaaaacttggaagatgtcatataaaatgtcagattcctgtcttccttgaaaaatcaaagggtctggcaacactgtgcctggattcacacatcatgataagggaagtggtataaggagggtgagttgtaccacagcaaatgccgttggtgccctgcccagatcttcctgcaacttcctatttcagtgatcattagtttcacctccaaatgccaccatctgcatctttatgcctgaagacttctttttctggccactgttatctgctttttccataacgttctctaactttactgagcatcagaacccactggaaagcttgacaagaaacagattcctgggccccaaccccagagattctggttcactcaggctacgtggtacccaggaagctgcatttctaacaggctctcaggtgaggctgatgctacatacattgtacactgagtgccattgccttacaaagaagcacccagaaactaggcacacaagtagcccccttttgcattagccaagaccctagagcagacacagaattctttagagtgcatgcagtttacagagcaagggtttttacattctatctttaaacctgtagctgccctgtggatagactctcttttgcccactctcactagcccccaaccctccaccattcttttgtgggaaccctgattcctggccaggacacgtgagagatggataggatctcatgtttaaattgcagctttgtctgagttctaaactaaagttccttgtgtggtccttgtgtaacaagctacattttctccttttagggggcggacaacatactagtggtagttctcttagcctaactgcattagtgagacgaaggtggtttcctaagcagcccagttgcacaattctacccaaagtaaggctcagcttagcgtctatgtgatttgcttttaagatctgcctacagtgctttattgtactcattacttgagttctcattatctacttgcagatttatttggacaaatgttgtgtgcaattatgttcttttagaaaattgtcccttttatgtacttggatgattaaagtatcatgcaaaagcacttatactcctacagcagcagaacaggcctcacttccaagtgtcgagtttcaattgctcatctcacattttgtcagggagatggttattaggatgccagattttgaggggtttgggcatttttcctgccagcaaagagaaacccacactagatatactgaaatttaaaactgccaatagcaaaatgctgatacattcacctccttaaccattcaggtttcttaaatttataggcaaacaaaactggaacgtttgttcctcctctactctctgtgtattggggttgcaaatttacttacaccctagttttggaaataaacacacacacacacacacacacacacacaccacaattatggtttctatgcttcctaaaactaacccagacttcttcacagagtcctactggctagattctttttaaaaatatctattttaattcaatgcattaaatttagttggcttgaatgatttgaacattgtctatcaaagtcaactacattattatgaggtataaatgatacctttataaacccagtctttcagatttaaccaccacctactccctcctctcctctccaccttcacttattcccaagatggagtgtgggaggaaagacaagcatggctcgtgggacctcagggtgaaggagataagaagcatcttcttgttcctttctcctccctgccttgctttcattggactctcctgctacctggactggtgaccaggtagcaagattgactggtcctactggtggctgggctgtccaggggctcacctctgctgtacatcacagttgcagcctcttcactcacgggtgaaaaggtgccctacaggctgcctccttgtcccaatcacaggcttgtgccagacccttccccattccctcactgagatttggaggaacttccgaacccctgcacatcccacacaggccatggaagacagtagcattgccacaagctcacttccacccacactatagcgccgtgtctccatggagtacattttacatgctggtgtaaaacgaaacagtatctgagacaggtgtcaatcaatttagaagtttattttaccaaggttaaggacatgtccagaagaagaagaaatgaaaaacatggattcatagaaactgtctgtggtctgtgcctttctccaaaggttagtttgagggaaaggggaaaagtgagctggaggggaaagagggagggtatggtcatccacttgttgcaagagaaaaggagcaggtaggagaactgtcaattatgtattgatctcatgctcagcaaatcggcactttacagaatatgatgtgaacatagagtcactacctatggaggtatttacctatgacctgtagctgtctaattaggaacaaaacagaaggtagcttcttgcaggactcagcgtttagttcaatttttcctcttggcagaatgaattggcgtcctgagtttttattttcctttcacacggggtatggccaaggctggtgttcaatcagttttaccagcatgtctacacctgtttttaaaatattgcaagattttcatgctagttggttgagagccactgctttaagtccttaggctatcccccatcactccagccccttcccaagaccctcctcacctcattaaaaatggtaatccttcccagggtaactctaggactttgcttgactttctatctaggttccaattggcctacgttaatctggttattgaatattttgaatattacctctgaacacatgcttgtggggcttctcctctcccttatttgtatggggatgtatgcaacacagatatttccaaagcccaccaccatctgcttggagtgaaaaaaagtgatatatcataaggaggccgggacctgacatagtcaacatcactcctctttctctccttctgcaaattatcccagactacaaggggtggactgtaggatttttacttctgtgcattatcgactcctcttataatagggcaccatggattatggaccacgtatcacaactacaaggccctttttttcatactacatgtgaaattctatgatctgagtcctctggggttgattcttgataagaaattgtgaatatcacctctctgctaaaccagcttctctagcatttctttgggaataatctaatcccttccaattcaaatcgtaccaccactggtccaaatcactggccttaagccaattagtgagaatatcatgaacaaggaaatgaaaaaaaatcacattaataatttccaaaatgttattttcatttcatctgtgttccaattttgattctagcaacagctatcttcagtgtgcttcagatgttttcagaaagagcttcaatgtgtcttctgtcacctggtatactattatgtatgtgtgtcagtataaaataaatctagtagcatttgttttctgagtttctgctgtttgtttgcccaactcttaaggaacacagtaaggctctgcaatcagaaagcagagttgttgtccccaaggaatgtcatctctagctagagatggaaaatcagcaccagaaaacagttcaggattgagtgcatgccaaattctctgaatttggaaaagagggtgcagcgcaccagcatggcacatgtatacatatgtaactaacctgcacattgtgcacatgtaccctaaaacttaaagtataataataataaattaaaaaaaaaaagagagtgatcaacgtgggctcctgagaattctggaggatgttagatttgataaagcccttgatgtatacctaagagttgaatatgcaagaaaaatcatcaaaaaaactcagagttgggaatggaccataagataaccacctctttctgatttgcccagaactttctgggttttagccctggatattctacattctaggaacagccctggtccctaggaatggttttagtgtgtgcagaaagccatgaggaataaaattggcgacacgaaaaggcaccagattacagagggctttaaaaaccattcaggtggactcgtgtgtttgcgccagcagtaggaagatggggtgtggtgaaaatgttaaggcttttgtcagtatgctaaagagattagactctgtgcaaacaaatgcccccttatccccacctgttcatgtgacctcctccctcttctgtttcttgactgaactgtgatctatgattcaagctataatgtgtataccaacagcctgtggctcttgttaaagtggaaactgactttagtaggtctggggtggggcttgaggttctgaaaatctaacaagctcccaggtgacactgctgctgctgctattgatgaagatgatgctttaagtaacaagaccttggatcccatcccatccctcctgaagggatgctttaaaaaccctgtcatcagttatcctcactctctcctctttcttcaacatctaccactctgggaactcccttctttcagcctgtaactttgctcttcagagcagcagagtggtggttaaattcccgaatcctggctcttccagttactagctctgtatccttggcaaagtggcctaaccacttctgtgttttggcttacttctctgtaaaatggagataataatatttcctacctcgttagtttcttgggagctgtcaaatgagatacatgtaaagtgtttaaagtgttacctggcaggtagtaagagctcagtgagtgtattcatttcctaggactgctataacaaattgctaccaattgagtggcattttcttctctctgcatgaaatgtattctcatgattcaagagtccagaggtccaaaatcaagattttggtagtgttgattccttctggaagctctgagggagaagttttatatacgtttctcctggtttctggtggttgtggcaatcctcagtgttgcttggcttgtaaactcatccctccagtctctgctttgccttcacatggtcttcttatctatgtgtctgtgtgtcctctcctatttttatatcagtcatgcaatttagggcctgtccccaatccaatatgacctcaccttcactaattagacctgcaaagactcttccaaataagatcacattctgcactttggatggatgtgaattctgggagatattcaacccactgtagagaatagtaataattatcattgtcatcattatcatattcaccacgtttgtaaatgctggatttcttctatctatattaaaatacatctcatttcttgaaaagcactctgactgaaatctacctgactgctctctgtattccaccactgactcaacctgaaactagaggctactagggatgccctcattgctgagcacaacagcaactcccggttcttacctggtttcatctccctgttgtgtcctagctgctgctatccctaccttggctttcccaatttattctccgttgtttctctgatttcttgatgtctttctcttcggtctcctgtaatgcctcaatttcgatcaccctaggtgcccagtatttcatatttgaccatcttcttttctcatgctatggcccttcaagggtttcactaccactcatatcccaaggaccttctcatcctaccctagcgatggcaatacccagatcttgatagtactgggaggtcaaaactcatcacatgataaacctaaagtgtgtacatttcacactgtgtaaatttttaaactgttaaaaaaacacattgaactccagttaatgctaaattaggagtgaggtgtactgctgtcagtaattcagtttgaaatgcatcaaaaattagatgaatagatggatggatagagagataggtgtgtgataaagcccacacagcaaaacattaataattatagaatctactcatgcagatagggatgttcactctataattctttcagctttgctatatgtttggaaattaccataataacatgtgtggcagggggaatcttgtctctgggtcagacctccctgctgaatcctgatctttataatatattaatgcacttgttcttcattctccaaggactctttcacttcttatgattgaggttcatcaatttatgcaacaattttgattttgaaaacctatcatgggccaagcactattgtgggtgttgaagccacagagatagtttttttttttaactctaaccaggaagacttggggtgagatagagacaagagtcaaggatgattttcttggattgatgttggtgtcattcactagaacaggactgataggaaaggggtcagcttttggagcagagagagggatgctgagtttggaagtacttgtggtcatccatttagaaatatacactagttggaaatacaggctttgaatggaggagcaaggtctgaactaagcatagaaagaaaaacagtatccagtaagagaatctaagaagagaagatgtgagagaggaaaccagaaccctagggaaccccaatatttaagaggcaggcggcagaggtagagccagaaaaaaaaggttggaagaggaagttcagagaggtgtgaagagaattaagagagaattcataccctggagactgtactatttacctttaaaaattttttcacttcaagtggtcattctgaaccattgttttcttttgtgttctattatcaccaacaactctaagtggctctttgatgtcactttcataaggctgtttgaagttcagaccaggtcatcatgactagaaattatggcttccagaaataaactgaatcccggtcatctagaattcataaaccagcagggtgcctaacacccagggtgcagtatgttcttaagagtacatgagctctaggaaagaaatttccaagtttttttcctccttgatgatatctaaggtaaataacatgtatatacactgagcccttaggatgaccaccttaaacccctgacaaccaagtgccaacacccaggttctatgcctgtgtttccacttgcattgatgttcatgttggtagattcaactgtcatggacaaaggagcaaaccaaaaagggaagggtatccagtactaaaagtgacatttccacatcctggataagcaaaggccttgcaatatttcaaatatagaaaagagatggttctttgattcacctcatggtacatagaaatctaaatgtgctcaactcaaaagaatctaaattacatcagtttgttcaaaattcatattaagaatagcaacttatgtatatgaaagcatcacctgttacattatattaaacttgtaggcttgggatgtaacaaatttgttgtttgtggtgtatttaatttgtaaagattttttgttgttgttccatcattttatagaagtaatataagcagcaggagttgagacatttggaagttgatcctggtgggcccgggagaagtttctccattaaaatggaccatgcctttccaataaaagggtctgacagtccctcctctgatatgtccaaggcctgttgatgagactgttttgttttaaatttgaaactctgtatgtttgtattttcgtgtctagcagacattgttttaaaattgctagtttgttgcattcctttccttttcagataaagttgtctgattttggtttctgtgctcaagtttccaaagaggtgccgaagaggaaatcattggttggcactccctactggatggcccctgaggtgatttctaggctaccttatgggacagaggtaagggggtcacagtcataggcgtggcaggactggggacaggctcagggacccattggttctgagatagttggtgaccacaggagatgggggcaatgacttttagctccacatttgatccccactcactagatgaggcacagagtagataggctgtggcaggtgggagcctggccacccctggggcagatcactgcagtatttatccaagggctgtttattgcagtcctacagtggacactgtgggaaagtcactccaactctgatcccactctcagtagcaggcacacacacaaagcaaggcaatgcaacttgcaaagtggctggagacatgcagatgagcaggcatgagaattcagagaaaggaaagaggatcaaagaaagtgccacaaaatagaggtgatatttaaatgacccactgttttcaacacagtttacatctcacagtgaaggacctacactgttttctcataggaaaaaaataatatgtgaatcatagtcaaaaacatggcacaatttctgtttccactggaatgaaaacaatcactcttaatcttttaagaggtagggcctggaaatgtgaaaatgcagggccagaggagtagaggaagggccttcttgggaggtaatagcatgggtggaagcttagaggcacataaggctacatacagatggaagccaaggggtatgtttcggctaggcatagtgtttccacactttatggtgtggatggtgaatctgggagaagagaagcatgagaagacatggccgtgagctaaggacatcagcataagttaaggcagaatctttctctttctcttgtttgcttgaccttggatgagtcctcaacttgtttttctcactgtggtcctgagagagaaattctcggtgaaggtcactgcaaggggtaataatagcaatacgttcatcagataagaggtataatgaggccctaacaggaaactttcctaacaggaaagtgagtgggactgggagtttatagcacagtgatggctcctcattgtctcgacttcccattctttactacttgcttgtcatctcttccttttcttttctccaatccctgaaaatgcttttgtaaagtgaggttttgtgcatgctgttttgaaatgataataaaaggtagtggaaggaaatcccaaagtaggggagaaaagagctatttttaaaagcaaattaataaaagaatcagaatatattctgcatcctgcaaggtagccctttgcagtttgttacaaatatgtcaaaactttcttatttgcttatggtttaaatacaattttcttctccattttcatagcattaaaccggatgattttctcatctgccctgatagacagttaactactaaagtttacagagtaaaaatgaaagcactaaactttagaatgttaaaatattcatttatgtctgatgggtggaaccttcactgaagttgtccataaaaggcaaagactgttagcaaaaactcggtaacccagcagtttagcctataaatctatattgtaaatgttgagaggttacaagtgccatagtcacttgtgaatgagtatgaggttctgtggacacacttgacatttcagaatgttctccttgcctgagataaccagtagggtgtgcctaacaggtggacatctggtccctcgggatcatggtgatagaaatgattgatggcgagcccccctacttcaatgagcctcccctccaggcgatgcggaggatccgggacagtttacctccaagagtgaaggacctacacaaggtgagaaacagcagctgttgctaaagttcagaatagtttttggattttcctgtatctctctggtttttaagactggcttaccacatccagcaggaagatcctggtttatgagcatggactttggaaatagtcacttaggtttgaatctctgttcttctattcgtttactgggcaagtgactcagcctttcctatgccaaagttcttcatctgtaaaatgggttatgattggcctgtatcccaaagggttggttagaagagtaaatcaggtgctctatgccatactcattcattcagcatctattacatgctggggacatctaatagcttgggatgtcaccgtgcatgaccccagcttttctgactctggggtctagactctgcattctagttggggaaacagatagtaaaccataaacaaacaaataaataaaataatttcagagtgtggtacatactatgaaagaaaaacatactgtgatgtgacagagtgtgcaggagaagggattgaaagctggttgtatgagtctgtttttacactactataaagaaatacccaagactgagtaatttacaaataaagaaggtttaattaactcacagttccacatgcctggagaggcctcaggatacttacaatcatggcagaaggcgaaggggaagcaaggcatgtcttacatggtggcaggagagagagagcgcacaggggaaatgccagacacttataaaactatcagctctcatgataattccctcactatcttgagaataacatggggtaaactgcccccatgacccaattaccttccaccaagtccctcccttgacatgtgaggattacaatatgaggtgagatttgagtgggcacagagagccaaaccctatcactgatttaagctgaagagtgaaggaagccctttctagggagggatcaaacgagctgggacctgaataggaagaaggtaccagccatgcaaagatcttggagaaaaaggtcccaggcacggggagcatcaagtggaaaggccttaagccaagaaagaaggcttacattgctggagtaaagtcagaaaggggaaaaaggtaggtcttgtcagagagatgggcaggagctagattgtacagggcaatgaaggtcatggttaatataatgcatgtaagagaccgagtggggtcttttctgttttccaaggattgccaattaggacgagctcagaaaataatgatgcttcatccaagaatcaccactgtaggagctgccagatttccaaaggacttgctcagaggatcatttctgcaggactactactataaacactggccccatacacacctctcttcctatcttatgacaataaaagagagtgtgtggctgccttttggaggagaattatttgtaatggtaatatctcctccttcctcacccctaaatcttagggaacagccccagtagcatcactcatataggttgtgtggacctacaaaaacataaacaaaaaaagagagattgaaatttttactggattggactaagtctaaataatgaaaagtggctgggtgtggtggctcacgcctgtaatcccagcactctggaaggccgaggtgggcggatcacgaggtcaggagatggagaccatcctggctaacacggtgaaaccccatctccactaaaaatacaaaaaattagccaagcatggtggcgggtgcctgtagtcccagctactcaggaggctgaggcaggagaatggcgtgaacctgggaggcagagcttgcagtgagcccagatcgcaccactgcactccagcctaggtgacacagcgagactacgtctccaaaaaaaaaaaaaaaagtgactaaaaatgtatcatatgaaagtagtctcacacccagtgagtgccaatgttcttcaaaaccatgtacaagaacaactttaatcctaatggtttcaaaaaaactggaaaccagtccgaggttgtttcccaaatggataatgtgtggtataattggacagtgaatactacacagtaatttaaaagatataacttttgccacacgtcggagatgaatctcacagatacaacactgagtgaaagaagccagatagagaagaaatcaaatatatgactccattttggtaaagtacaaaagtaagcaaaactaagttatgctataaattaaaatagtggtgaccttttgcagggagataggggttggcaggaaggaaacctaaaagtagcttcctagagtggtagaaaagttctatatcttgataggagtggaaagtaaataggggtaaatgtatgtaaaaatccattgagccacactcttagtatttgtgcagcggaacctgggcatgctggcacatgcttgtactcccagctactcaggaggctgaggtgggaggatcatttgagcacaggagtttgagcctgggcaacatagtgagacctcatctctaaatatatacataaagtaagatatacctttaaaaggaaaattacaagaccttcaaagaaaaaaatgagatgtgttcgatatccttacagaagcttgtttctcttaaagaaaggggttccacagagcctgtgtggttgcagttgttagaaaaataaaacggctccctgtttatactatgcttcatcaagcacgtgtgaccagcacctgcacatactaaatattttgtaatagctgctcttatcactgtgattgccactgttattttatgtagtaaataagttttcattcttagttgagctgtcatttacacctattatttcacacttgaaaaaggtaaggaaaacacgaaatgttaacaaagacaagacatgcttttgctttcacaacatctttctccttaaggtaactttctggaatttgaagttgttgataggatgaatggggaaaacattttggggactgggttaggtgtctgtgttagtcctcagattggtatctgtgtgttttcacaggtttcttcagtgctccggggattcctagacttgatgttggtgagggagccctctcagagagcaacagcccaggaactcctcggacatccattcttaaaactagcaggtccaccgtcttgcatcgtccccctcatgagacaatacaggcatcactgagcagaggattcgtgtaggtggcaaagctagatgaggacatgagaataattcaggagaacaaaaggaaacacagaacatgcaaaaggcctgtgcattctagaccagccaattggtgggacagcgtgatgaccggcagggttcaacagaccagggcatcttcttgtgtcttaaacaggcatctctccactgacagccggtgtggtcacttggagcacggctttaataagtcattattatatttttcagcccttcatccagcaaatcagaaggactcagtacaaactccgttatgatatatcctagccacatgcagggtaacatgtaggattttctatattgaaagaatacttttctggcaaaaaaaaaaaaaaaagaaagaaaggaaaacaaaaagcacttttttcttaatggtagcagtataatgtattttgcaacgaatttgtaatttttctgtacgatagttttgataatttatagtactttgatgtcatgtagccattgtatcagttgaagtaatacttgtttactagaggagtttgaacaaagcctttcctacttttttatccctttaagagaaccaatgattctttaggaactttgaatactgaatgactctcaatcaccgtcagctttagtaaaatctctttcttatcctaacaagtgtcttatttggtggaagaagaattaagagtgatggtgatggtgtgcacgtttcattaatccaaccaaaaataatgaaataaaatttgagccacagtataccactccttgggataaagttaaatatttttaaagatcacattttccatgaacgcctctagtagcaaaccattcttttgcacaccacaatgtttccctcagtgccctttctcaaatgggtacaatgttcccttgtggccaaatttccctcccagggagcaatttcagtgctaggatcattggattcagttcccaaaatagaatgtttcagtgagaccatgagaattccaggctcacaaagggagaggagagaacagggcaagacgtttggtttcatttgtcaccatttttaaaactctgtatgctagcacaccaaactcttgtctatatttacctttgtaccacagtattaatcgctattgttcatgtatcgtgctggaagtctgaactgactctagaggatgaattagcaagagggtattttaccaggtatgatctgacttcagttgtgcccatgttataatgtgtttccgacataggagagtcgtgctgctgtctagatcttcttgaatgttgataaaaatgaatgactactacaatacattttgtgttgcttgttggatgaatttgcatgttaactgtaggccaatatagatttgcctttaaaactctggaagagctacatagtcatcattagtttctattaattatgcatcagacaaaagccatttgttaccaaactgggaaaacagaggcttttcttaactatttcacatactgtaacaaatatgaatttaaatttgtgatagcgctctggttgctctaagcataattaagaatttttgtaattaataggttgctaattatttatcactgctaaaaaggaaaaaaggcataaaatgaccttctactgattagattttcagttttctttcaaactggaaatgcctccataaatatgatctatgattttgcttcataaaacagcaaatcaatgttttatgtaaaatattaaagcattaatataaatatgtgagaataaaaacaatctaaatccagaaaatggcagtcctaaatgttcatgagacagattgtattaatttaaccaggactatgtagaagtagaaagaaaagaaaaagaaaatcttttttaaaccagaataaacattaaaaactattgcagaaaatagtggattttggattccaaacattttcgacagtgtaatggaaatttttctgtaattttcttaccatcgggtattttttaaagtattcattgagtttaccaaaagttactgtagcttaaaaggttttgtgagcactaactattggcagaaactgcatttgcaaataaaaataaatgtttgcctttt

SEQ ID NO: 3 - protein, 745 aa, Homo sapiens
MLPFAPQDEPWDREMEVFSGGGASSGEVNGLKMVDEPMEEGEADSCHDEGVVKEIPITHHVKEGYEKADPAQFELLKVLGQGSFGKVFLVRKKTGPDAGQLYAMKVLKKASLKVRDRVRTKMERDILVEVNHPFIVKLHYAFQTEGKLYLILDFLRGGDVFTRLSKEVLFTEEDVKFYLAELALALDHLHQLGIVYRDLKPENILLDEIGHIKLTDFGLSKESVDQEKKAYSFCGTVEYMAPEVVNRRGHSQSADWWSYGVLMFEMLTGTLPFQGKDRNETMNMILKAKLGMPQFLSAEAQSLLRMLFKRNPANRLGSEGVEEIKRHLFFANIDWDKLYKREVQPPFKPASGKPDDTFCFDPEFTAKTPKDSPGLPASANAHQLFKGFSFVATSIAEEYKITPITSANVLPIVQINGNAAQFGEVYELKEDIGVGSYSVCKRCIHATTNMEFAVKIIDKSKRDPSEEIEILMRYGQHPNIITLKDVFDDGRYVYLVTDLMKGGELLDRILKQKCFSEREASDILYVISKTVDYLHCQGVVHRDLKPSNILYMDESASADSIRICDFGFAKQLRGENGLLLTPCYTANFVAPEVLMQQGYDAACDIWSLGVLFYTMLAGYTPFANGPNDTPEEILLRIGNGKFSLSGGNWDNISDGAKDLLSHMLHMDPHQRYTAEQILKHSWITHRDQLPNDQPKRNDVSHVVKGAMVATYSALTHKTFQPVLEPVAASSLAQRRSMKKRTSTGL

SEQ ID NO: 4 - protein, 719 aa, Homo sapiens
  2 mRNAs 1 Protein (1)..(719): Alternative splicing of Exon 2 results in two different mRNAs coding for the same protein.
MFGKKKKKIEISGPSNFEHRVHTGFDPQEQKFTGLPQQWHSLLADTANRPKPMVDPSCITPIQLAPMKTIVRGNKPCKETSINGLLEDFDNISVTRSNSLRKESPPTPDQGASSHGPGHAEENGFITFSQYSSESDTTADYTTEKYREKSLYGDDLDPYYRGSHAAKQNGHVMKMKHGEAYYSEVKPLKSDFARFSADYHSHLDSLSKPSEYSDLKWEYQRASSSSPLDYSFQFTPSRTAGTSGCSKESLAYSESEWGPSLDDYDRRPKSSYLNQTSPQPTMRQRSRSGSGLQEPMMPFGASAFKTHPQGHSYNSYTYPRLSEPTMCIPKVDYDRAQMVLSPPLSGSDTYPRGPAKLPQSQSKSGYSSSSHQYPSGYHKATLYHHPSLQSSSQYISTASYLSSLSLSSSTYPPPSWGSSSDQQPSRVSHEQFRAALQLVVSPGDPREYLANFIKIGEGSTGIVCIATEKHTGKQVAVKKMDLRKQQRRELLFNEVVIMRDYHHDNVVDMYSSYLVGDELWVVMEFLEGGALTDIVTHTRMNEEQIATVCLSVLRALSYLHNQGVIHRDIKSDSILLTSDGRIKLSDFGFCAQVSKEVPKRKSLVGTPYWMAPEVISRLPYGTEVDIWSLGIMVIEMIDGEPPYFNEPPLQAMRRIRDSLPPRVKDLHKVSSVLRGFLDLMLVREPSQRATAQELLGHPFLKLAGPPSCIVPLMRQYRHH